(19) United States
(12) Patent Application Publication
(10) Pub. No.: US 2022/0249463 A1
(43) Pub. Date: Aug. 11, 2022

(54) METHODS OF ALTERING CARDIAC REMODELING USING COMPOUNDS THAT PROMOTE GLUCOSE OXIDATION

(71) Applicant: IMBRIA PHARMACEUTICALS, INC., Boston, MA (US)

(72) Inventors: Andrew D. Levin, Newton, MA (US); David-Alexandre Gros, Rancho Santa Fe, CA (US); Jaikrishna Patel, Cary, NC (US)

(21) Appl. No.: 17/614,826

(22) PCT Filed: May 27, 2020
(86) PCT No.: PCT/US2020/034609

§ 371 Date: Nov. 29, 2021

Related U.S. Application Data
(60) Provisional application No. 62/855,372, filed on May 31, 2019.

(51) Int. Cl. A61K 31/455; A61K 31/336; A61K 31/4458; A61K 31/495

(52) U.S. Cl. CPC A61K 31/455; A61K 31/495; A61K 31/4458; A61K 31/336

(57) ABSTRACT

The invention provides methods for altering cardiac remodeling in a subject by providing a compound that shifts cellular metabolism from fatty acid oxidation to glucose oxidation.

### CROSS-REFERENCE TO RELATED APPLICATIONS

This application claims the benefit of, and priority to, U.S. Provisional Patent Application No. 62/855,372, filed May 31, 2019, the contents of which are incorporated by reference.

### FIELD OF THE INVENTION

The invention relates to methods and compositions for altering cardiac remodeling in a subject.

### BACKGROUND

Cardiac remodeling is associated with a variety of cardiovascular diseases, which collectively account for more than 17 million deaths worldwide each year. Cardiac remodeling, also called ventricular remodeling, includes changes in the shape, size, and/or function of the heart after injury. Cardiac remodeling is commonly triggered by myocardial infarction, in which necrosis of heart tissue leaves the damaged tissue vulnerable to the pressure and volume load of the heart. Myocardial scarring initially provides some benefit by compensating for the weakened tissue but eventually leads to cardiac dysfunction. For example, cardiac remodeling contributes to chronic conditions such as congestive heart failure and arrhythmia that arise as complications from heart attacks.

Existing treatments to combat cardiac remodeling have limitations. For example, angiotensin converting enzyme inhibitors (ACE inhibitors) attenuate remodeling by reducing blood pressure but impair kidney function. Beta adrenergic receptor antagonists (beta blockers) also limit remodeling but pose risks for treatment of diabetics because they can cause hypoglycemia or mask the symptoms of hypoglycemia. Consequently, there is no adequate therapeutic intervention to prevent cardiac remodeling in many patients with cardiovascular disease, and their risk of morbidity and mortality remains high.

### SUMMARY

The invention provides methods of altering, e.g., reducing or preventing, cardiac remodeling by providing a compound that shifts cellular metabolism from fatty acid oxidation to glucose oxidation. The invention recognizes that compounds that promote glucose oxidation increase energy production and reduce cardiac remodeling in certain clinical conditions, such as heart failure. Providing such a compound to patients who have cardiovascular conditions associated with cardiac remodeling blocks cardiac hypertrophy and improves cardiac function. Consequently, the methods of the invention minimize the risk of morbidity and mortality from a variety of cardiovascular diseases.

The compounds that shift metabolism from fatty acid oxidation to glucose oxidation may be derivatives of trimetazidine. Whereas trimetazidine itself can cause Parkinsonian symptoms for a portion of the population, the methods of the invention overcome this issue by delivering the molecule in a modified form. Without being limited by any particular theory or mechanism of action, it is also believed that delivery of trimetazidine as a component of a larger molecule may improve its efficacy and mitigate its side effects.

The methods may include providing compounds that include a molecule that shifts cellular metabolism from fatty acid oxidation to glucose oxidation linked to a molecule, such as nicotinic acid, that serves as a precursor for synthesis of nicotinamide adenine dinucleotide (NAD⁺). Such compounds can be metabolized in the body to allow the individual components to exert distinct biochemical effects to increase glucose oxidation relative to fatty acid oxidation and improve overall mitochondrial respiration.

In an aspect, the invention provides methods of altering cardiac by providing to a subject that has developed cardiac remodeling or is at risk of developing cardiac remodeling a compound that shifts cellular metabolism from fatty acid oxidation to glucose oxidation. The compound that shifts cellular metabolism from fatty acid oxidation to glucose oxidation may be trimetazidine, etomoxir, perhexiline, a PPAR agonist, a malonyl CoA decarboxylase inhibitor, dichloroacetate, or an analog, derivative, or prodrug of any of the aforementioned agents.

The compound that shifts cellular metabolism from fatty acid oxidation may be represented by formula (IV):

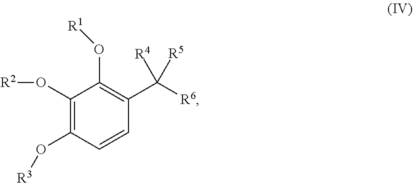

in which:

R¹, R², and R³ are independently selected from the group consisting of H and a (C₁-C₄)alkyl group;

R⁴ and R⁵ together are ═O, —O(CH₂)ₘO—, or —(CH₂)ₘ—, wherein m=2-4, or R⁴ is H and R⁵ is OR¹⁴, SR¹⁴, or (CH₂CH₂O)ₙH, wherein R¹⁴ is H or a (C₁-C₄)alkyl group and n=1-15; and

R⁶ is a single or multi-ring structure optionally substituted at one or more ring positions by a heteroatom, wherein each ring position optionally comprises one or more substituents.

One or more ring position of R⁶ may be or include a substituent that includes a compound that promotes mitochondrial respiration, such as succinate, fumarate, malate, oxaloacetate, citrate, isocitrate, α-ketoglutarate, pyruvate, acetone, acetoacetic acid, β-hydroxybutpic acid, β-ketopentanoate, or β-hydroxypentanoate. The substituent may be or include a linker, such as (CH₂CH₂O)ₓ, in which x=1-15. The substituent may be or include a NAD⁺ precursor molecule, such as nicotinic acid, nicotinamide, and nicotinamide riboside.

The substituent on a ring position of R⁶ may be

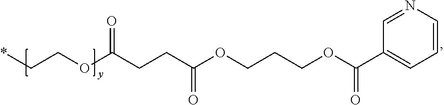

in which y=1-3.

The substituent on a ring position of R⁶ may be

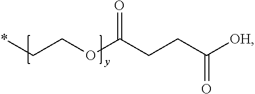

in which y=1-3.

R⁶ may be

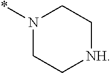 [SMILES: *N1CCNCC1]

The compound of formula (IV) may have a structure represented by one of formulas (IX) and (X):

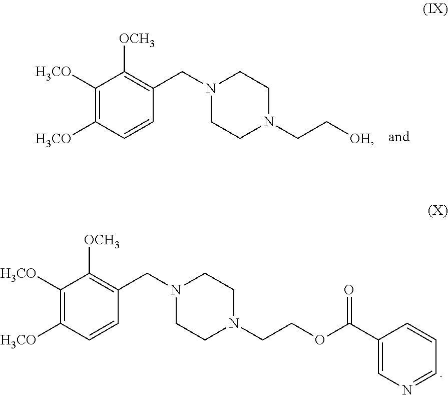 [SMILES: COc1ccc(CN2CCN(CCO)CC2)c(OC)c1OC ; COc1ccc(CN2CCN(CCOC(=O)c3cccnc3)CC2)c(OC)c1OC]

The compound that shifts cellular metabolism from fatty acid oxidation may be represented by formula (V):

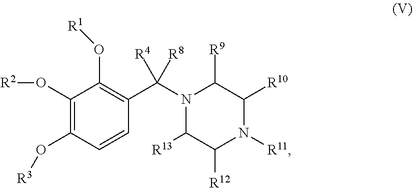

in which R¹, R², and R³ are independently H or a (C₁-C₄)alkyl group; R⁴ and R⁸ together are ═O, —O(CH₂)ₘO—, or —(CH₂)ₘ—, in which m=2-4, or R⁴ is H and R⁸ is H, OR¹⁴, SR¹⁴, or (CH₂CH₂O)ₙH, in which R¹⁴ is H or a (C₁-C₄)alkyl group and n=1-15; R⁹, R¹⁰, R¹², and R¹³ are independently H or (CH₂CH₂O)_{z}H, in which z=1-6; and R¹¹ comprises a compound that promotes mitochondrial respiration.

The compound that promotes mitochondrial respiration may be an intermediate of the citric acid cycle or a molecule that can be metabolized to enter the citric acid cycle. For example, the compound may be succinate, fumarate, malate, oxaloacetate, citrate, isocitrate, α-ketoglutarate, pyruvate, acetone, acetoacetic acid, β-hydroxybutpic acid, β-ketopentanoate, or β-hydroxypentanoate.

R¹¹ may include a linker, such as polyethylene glycol. For example, R¹¹ may include (CH₂CH₂O)ₓ, in which x=1-15.

R¹¹ may be

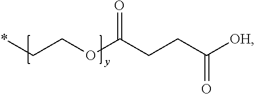

in which y=1-3.

R¹¹ may include a NAD⁺ precursor molecule. For example, R¹¹ may include nicotinic acid, nicotinamide, or nicotinamide riboside.

R¹¹ may be

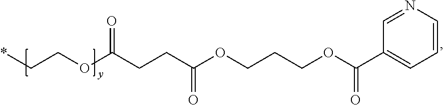

in which y=1-3.

The compound that shifts cellular metabolism from fatty acid oxidation may be represented by formula (VI):

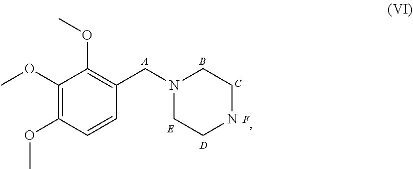

in which:

at least one of positions A, B, C, D, E, and F is substituted with —(CH₂CH₂O)ₙH and n=1-15.

The compound may have a substitution at position F. For example, the compound may be represented by formula (IX), as shown above.

The compound that shifts cellular metabolism from fatty acid oxidation may be represented by formula (VII):

A-C (VII),

in which A is a molecule that shifts cellular metabolism from fatty acid oxidation to glucose oxidation, and C is a NAD⁺ precursor molecule. A and C may be covalently linked.

The molecule that shifts cellular metabolism from fatty acid oxidation to glucose oxidation may be trimetazidine, etomoxir, perhexiline, a PPAR agonist, a malonyl CoA decarboxylase inhibitor, or dichloroacetate.

The molecule that shifts cellular metabolism from fatty acid oxidation to glucose oxidation may be PEGylated with an ethylene glycol moiety. The molecule that shifts cellular metabolism from fatty acid oxidation to glucose oxidation may have multiple ethylene glycol moieties, such as one, two three, four, five, or more ethylene glycol moieties. The ethylene glycol moiety may be represented by (CH₂CH₂O)ₓ, in which x=1-15. The ethylene glycol moiety may form a covalent linkage between the molecule that shifts cellular metabolism from fatty acid oxidation to glucose oxidation and the NAD⁺ precursor molecule. The ethylene glycol moiety may be separate from a covalent linkage between the molecule that shifts cellular metabolism from fatty acid oxidation to glucose oxidation and the NAD⁺ precursor molecule. The molecule that shifts cellular metabolism from fatty acid oxidation to glucose oxidation may be a PEGylated form of trimetazidine.

The NAD⁺ precursor molecule may be nicotinic acid, nicotinamide, or nicotinamide riboside.

The compound of formula (VII) may include nicotinic acid that is covalently linked to a PEGylated form of trimetazidine. The nicotinic acid may be covalently linked via the PEGylated moiety, i.e., via an ethylene glycol linkage. The nicotinic acid may be covalently linked via the trimetazidine moiety.

The compound of formula (VII) may have a structure represented by formula (X), as shown above.

The compound that shifts cellular metabolism from fatty acid oxidation may be represented by formula (VIII):

A-L-C (VIII),

in which A is a molecule that molecule that shifts metabolism from fatty acid oxidation to glucose oxidation, L is a linker, and C is a NAD⁺ precursor molecule. A may be covalently linked to L, and L may be covalently linked to C.

The molecule that molecule that shifts metabolism from fatty acid oxidation to glucose oxidation, the linker, and the NAD⁺ precursor molecule may be as described above in relation to compounds of other formulas.

The compound of formula (VIII) may have a structure represented by formula (X), as shown above.

The compound that shifts cellular metabolism from fatty acid oxidation may be represented by formula (I):

A-L-B (I),

in which A is a molecule that molecule that shifts metabolism from fatty acid oxidation to glucose oxidation, L is a linker, and B is a compound that promotes mitochondrial respiration.

The molecule that shifts metabolism from fatty acid oxidation to glucose oxidation may be trimetazidine, etomoxir, perhexiline, a PPAR agonist, a malonyl CoA decarboxylase inhibitor, or dichloroacetate.

The compound that promotes mitochondrial respiration may be an intermediate of the citric acid cycle or a molecule that can be metabolized to enter the citric acid cycle. For example, the compound may be succinate, fumarate, malate, oxaloacetate, citrate, isocitrate, α-ketoglutarate, pyruvate, acetone, acetoacetic acid, β-hydroxybutpic acid, β-ketopentanoate, or β-hydroxypentanoate.

The linker may be any suitable linker that can be cleaved in vivo. The linker may be an alkoxy group. The linker may be polyethylene glycol of any length. Preferably, the linker is represented by (CH₂CH₂O)ₓ, in which x=1-15.

The compound may include a NAD⁺ precursor molecule covalently linked to another component of the compound. The NAD⁺ precursor molecule may be nicotinic acid, nicotinamide, or nicotinamide riboside. The NAD⁺ precursor molecule may be attached to the molecule that molecule that shifts metabolism, the compound that promotes mitochondrial respiration, or the linker. The NAD⁺ precursor molecule may be attached to another component via an additional linker. Preferably, the NAD⁺ precursor molecule is attached to the compound that promotes mitochondrial respiration via a 1,3-propanediol linkage.

The compound of formula (I) may be represented by formula (II):

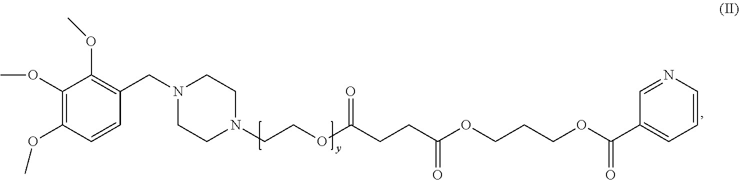

in which y=1-3.

The compound of formula (I) may be represented by formula (III):

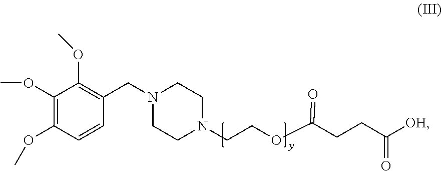

in which y=1-3.

Any of the compounds described above may include one or more atoms that are enriched for an isotope. For example, the compounds may have one or more hydrogen atoms replaced with deuterium or tritium. The isotopically enriched atom or atoms may be located at any position within the compound.

The cardiac remodeling may be associated with a disease, disorder, or condition. The cardiac remodeling may be associated with a cardiovascular disease. For example, the cardiac remodeling may be associated with aberrant subclavian artery, aortic regurgitation, aortic stenosis, arteriovenous malformation and fistula, atrial septal defect, atrioventricular septal defect, bicuspid aortic valve, cardiomegaly, cardiomyopathy, coarctation of the aorta, complete heart block, concentric hypertrophy, congenital heart defects, congenital heart disease, coronary artery disease, dextrocardia, dextro-transposition of the great arteries, diabetes, diet, double aortic arch, double inlet left ventricle, double outlet right ventricle, Ebstein's anomaly, giant hepatic hemangioma, heart failure, high cholesterol, high-output hemodialysis fistula, hypertension, hypertension, hypoplastic left heart syndrome, hypoplastic right heart syndrome, interrupted aortic arch, levo-transposition of the great arteries, mitral regurgitation, also causing left atrial volume overload, mitral stenosis, myocardial ischemia, obesity, outflow obstruction., partial anomalous pulmonary venous connection, patent ductus arteriosus, pentalogy of Cantrell, persistent truncus arteriosus, pressure overload, pulmonary atresia, pulmonary hypertension, pulmonary regurgitation, pulmonary stenosis, rhabdomyomas, right ventricular volume overload, scimitar syndrome, Shone's syndrome, tetralogy of Fallot, total anomalous pulmonary venous connection, transposition of the great vessels, tricuspid atresia, tricuspid regurgitation, use of tobacco, alcohol, or other drugs, valvular heart disease, ventricular dilation, ventricular hypertrophy, ventricular septal defect, volume overload, and Wolff-Parkinson-White syndrome.

### BRIEF DESCRIPTION OF THE DRAWINGS

FIG. 1 is a table summarizing the effects of various compounds on mitochondrial function.

FIG. 2 is a table summarizing the effects of nicotinamide on various mitochondrial functional parameters.

FIG. 3 is a series of graphs showing the effects of nicotinamide on oxygen consumption rate and reserve capacity.

FIG. 4 is a series of graphs showing the effects of nicotinamide on extracellular acidification rate.

FIG. 5 is a table summarizing the effects of a combination of trimetazidine and nicotinamide on various mitochondrial functional parameters. FIG. 6 is a series of graphs showing the effects of a combination of trimetazidine and nicotinamide on oxygen consumption rate and reserve capacity.

FIG. 7 is a series of graphs showing the effects of a combination of trimetazidine and nicotinamide on extracellular acidification rate.

FIG. 8 is a table summarizing the effects of succinate on various mitochondrial functional parameters.

FIG. 9 is a series of graphs showing the effects of succinate on oxygen consumption rate and reserve capacity.

FIG. 10 is a series of graphs showing the effects of succinate on extracellular acidification rate. FIG. 11 is a table summarizing the effects of compound CV-8816 on various mitochondrial functional parameters.

FIG. 12 is a series of graphs showing the effects of compound CV-8816 on oxygen consumption rate and reserve capacity.

FIG. 13 is a series of graphs showing the effects of compound CV-8816 on extracellular acidification rate.

FIG. 14 is a table summarizing the effects of compound CV-8814 on various mitochondrial functional parameters.

FIG. 15 is a series of graphs showing the effects of compound CV-8814 on oxygen consumption rate and reserve capacity.

FIG. 16 is a series of graphs showing the effects of compound CV-8814 on extracellular acidification rate.

FIG. 17 is a table summarizing the effects of trimetazidine on various mitochondrial functional parameters.

FIG. 18 is a series of graphs showing the effects of trimetazidine on oxygen consumption rate and reserve capacity.

FIG. 19 is a series of graphs showing the effects of trimetazidine on extracellular acidification rate.

FIG. 20 is a table summarizing the effects of compound CV-8815 on various mitochondrial functional parameters.

FIG. 21 is a series of graphs showing the effects of compound CV-8815 on oxygen consumption rate and reserve capacity.

FIG. 22 is a series of graphs showing the effects of compound CV-8815 on extracellular acidification rate.

FIG. 23 is a table summarizing the effects of a combination of succinate, nicotinamide, and trimetazidine on various mitochondrial functional parameters.

FIG. 24 is a series of graphs showing the effects of a combination of succinate, nicotinamide, and trimetazidine on oxygen consumption rate and reserve capacity.

FIG. 25 is a series of graphs showing the effects of a combination of succinate, nicotinamide, and trimetazidine on extracellular acidification rate.

FIG. 26 is a table summarizing the effects of a combination of trimetazidine analog 2 and nicotinamide on various mitochondrial functional parameters.

FIG. 27 is a series of graphs showing the effects of a combination of trimetazidine analog 2 and nicotinamide on oxygen consumption rate and reserve capacity.

FIG. 28 is a series of graphs showing the effects a combination of trimetazidine analog 2 and nicotinamide on extracellular acidification rate.

FIG. 29 is a table summarizing the effects of a combination of trimetazidine analog 1 and nicotinamide on various mitochondrial functional parameters.

FIG. 30 is a series of graphs showing the effects of a combination of trimetazidine analog 1 and nicotinamide on oxygen consumption rate and reserve capacity.

FIG. 31 is a series of graphs showing the effects of a combination of trimetazidine analog 1 and nicotinamide on extracellular acidification rate.

FIG. 32 is a table summarizing the effects of a combination of trimetazidine analog 3 and nicotinamide on various mitochondrial functional parameters.

FIG. 33 is a series of graphs showing the effects of a combination of trimetazidine analog 3 and nicotinamide on oxygen consumption rate and reserve capacity.

FIG. 34 is a series of graphs showing the effects of a combination of trimetazidine analog 3 and nicotinamide on extracellular acidification rate.

FIG. 35 is a table summarizing the effects of a combination of succinate and nicotinamide on various mitochondrial functional parameters.

FIG. 36 is a series of graphs showing the effects of a combination of succinate and nicotinamide on oxygen consumption rate and reserve capacity.

FIG. 37 is a series of graphs showing the effects of a combination of succinate and nicotinamide on extracellular acidification rate.

FIG. 38 is a schematic of the ischemia-reperfusion (IR) method used to analyze the effects of selected compositions on coronary flow.

FIG. 39 is a graph of coronary flow of after IR.

FIG. 40 is graph of left ventricular developed pressure (LVDP) after IR.

FIG. 41 shows images of TTC-stained heart slices after IR.

FIG. 42 is graph of infarct size after IR.

FIG. 43 is a schematic of the method used to analyze the effects of selected compositions on cardiac function.

FIG. 44 shows hearts from mice six weeks after transverse aortic constriction.

FIG. 45 is of graph of heart weight relative to body weight six weeks after transverse aortic constriction.

FIG. 46 is graph of heart weight six weeks after transverse aortic constriction.

FIG. 47 is a graph of fractional shortening (FS) at indicated time points after transverse aortic constriction.

FIG. 48 is a graph of ejection fraction (EF) at indicated time points after transverse aortic constriction.

FIG. 49 is a graph of left ventricular end-systolic diameter at indicated time points after transverse aortic constriction.

FIG. 50 is a graph of intraventricular septal dimension at indicated time points after transverse aortic constriction.

FIG. 51 is a graph of left ventricular mass at indicated time points after transverse aortic constriction.

FIG. 52 is a graph of isovolumic relaxation time at indicated time points after transverse aortic constriction.

FIG. 53 is a graph of the ratio peak velocity flow in early diastole vs. late diastole at indicated time points after transverse aortic constriction.

FIG. 54 is a graph of left ventricular developed pressure at six weeks after transverse aortic constriction.

FIG. 55 is a graph of the rate of left ventricle pressure rise at six weeks after transverse aortic constriction.

FIG. 56 shows microscopic images of cardiac tissue t six weeks after transverse aortic constriction.

FIG. 57 is a graph showing the level of cardiac fibrosis at six weeks after transverse aortic constriction.

FIG. 58 is a graph showing levels of CV-8814 and trimetazidine after intravenous administration of CV-8834.

FIG. 59 is a graph showing levels of CV-8814 and trimetazidine after oral administration of CV-8834.

FIG. 60 is a graph showing levels of CV-8814 and trimetazidine after oral administration of CV-8834.

FIG. 61 is a graph showing levels of CV-8814 and trimetazidine after oral administration of CV-8834.

FIG. 62 is a graph showing levels of CV-8814 and trimetazidine after oral administration of CV-8834.

FIG. 63 is a graph showing levels of trimetazidine after oral administration of CV-8972 or intravenous administration of trimetazidine.

FIG. 64 is a graph showing levels of CV-8814 after oral administration of CV-8972 or intravenous administration of CV-8814.

FIG. 65 is a graph showing levels of CV-8814 after intravenous administration of CV-8834 or oral administration of CV-8834.

FIG. 66 is a graph showing levels of CV-8814 after intravenous administration of CV-8814 or oral administration of CV-8814.

FIG. 67 is a graph showing the HPLC elution profile of a batch of CV-8972.

FIG. 68 is a graph showing analysis of molecular species present in a batch of CV-8972.

FIG. 69 is a pair of graphs showing HPLC elution profiles of molecular species present in a batch of CV-8972.

FIG. 70 is a pair of graphs showing HPLC elution profiles of molecular species present in a batch of CV-8972.

FIG. 71 is a graph showing X-ray powder diffraction analysis of a batch of CV-8972.

FIG. 72 is a graph showing X-ray powder diffraction analysis of batches of CV-8972.

FIG. 73 is a graph showing differential scanning calorimetry and thermal gravimetric analysis of a batch of CV-8972.

FIG. 74 is a graph showing dynamic vapor sorption (DVS) of a batch of CV-8972.

FIG. 75 is a graph showing differential scanning calorimetry and thermal gravimetric analysis of a batch of CV-8972.

FIG. 76 is a graph showing dynamic vapor sorption (DVS) of a batch of CV-8972.

FIG. 77 is a graph showing X-ray powder diffraction analysis of samples of CV-8972.

FIG. 78 is a graph showing differential scanning calorimetry and thermal gravimetric analysis of a batch of CV-8972.

FIG. 79 is a graph showing X-ray powder diffraction analysis of samples of CV-8972.

FIG. 80 is a graph showing X-ray powder diffraction analysis of samples of CV-8972.

FIG. 81 is a graph showing differential scanning calorimetry and thermal gravimetric analysis of samples containing form A of CV-8972.

FIG. 82 is a graph showing differential scanning calorimetry and thermal gravimetric analysis of a sample containing form A of CV-8972.

FIG. 83 is a schematic of the method used to analyze the effects of selected compositions on cardiac function.

FIG. 84 shows hearts from mice six weeks after transverse aortic constriction.

FIG. 85 is of graph of heart weight relative to body weight six weeks after transverse aortic constriction.

FIG. 86 is graph of heart weight six weeks after transverse aortic constriction.

FIG. 87 is a graph of left ventricular developed pressure at six weeks after transverse aortic constriction.

FIG. 88 is a graph of the rate of left ventricle pressure rise at six weeks after transverse aortic constriction.

FIG. 89 is a graph of fractional shortening (FS) at indicated time points after transverse aortic constriction.

FIG. 90 is a graph of ejection fraction (EF) at indicated time points after transverse aortic constriction.

FIG. 91 is a graph of intraventricular septal dimension at indicated time points after transverse aortic constriction.

FIG. 92 is a graph of left ventricular mass at indicated time points after transverse aortic constriction.

FIG. 93 is a graph of isovolumic relaxation time at indicated time points after transverse aortic constriction.

FIG. 94 shows microscopic images of cardiac tissue t six weeks after transverse aortic constriction.

FIG. 95 is a graph showing the level of cardiac fibrosis at six weeks after transverse aortic constriction.

### DETAILED DESCRIPTION

The invention provides methods for altering, e.g., reducing or preventing, cardiac remodeling in a subject by providing compositions that contain compounds that shift metabolism from fatty acid oxidation to glucose oxidation. Glucose oxidation and fatty acid oxidation are energy-producing metabolic pathways that compete with each other for substrates. In glucose oxidation, glucose is broken down to pyruvate via glycolysis in the cytosol of the cell. Pyruvate then enters the mitochondria, where it is converted to acetyl coenzyme A (acetyl-CoA). In beta-oxidation of fatty acids, which occurs in the mitochondria, two-carbon units from long-chain fatty acids are sequentially converted to acetyl-CoA.

The remaining steps in energy production from oxidation of glucose or fatty acids are common to the two pathways. Acetyl-CoA is oxidized to carbon dioxide (CO₂) via the citric acid cycle, which results in the conversion of nicotinamide adenine dinucleotide (NAD⁺) to its reduced form, NADH. NADH, in turn, drives the mitochondrial electron transport chain. The electron transport chain comprises a series of four mitochondrial membrane-bound complexes that transfer electrons via redox reactions and pump protons across the membrane to create a proton gradient. The redox reactions of the electron transport chain require molecular oxygen (O₂). Finally, the proton gradient enables another membrane-bound enzymatic complex to form high-energy ATP molecules, the source of energy for most cellular reactions.

In many types of heart disease, the overall efficiency of energy production by cardiac mitochondria is diminished. In part, this is due to an increased reliance on fatty acid oxidation over glucose oxidation. Glucose oxidation is a more efficient pathway for energy production, as measured by the number of ATP molecules produced per O₂ molecule consumed, than is fatty acid oxidation. However, other metabolic changes contribute to decreased cardiac efficiency in patients with heart disease. For example, overall mitochondrial oxidative metabolism can be impaired in heart failure, and energy production is decreased in ischemic heart disease due to a limited supply of oxygen. As indicated above, the final steps in ATP synthesis, which include several redox reactions and oxygen-driven proton transport, are common to both the glucose oxidation and fatty acid oxidation pathways. Thus, shifting the balance from fatty acid oxidation to glucose oxidation by itself is not enough in many circumstances to restore full cardiac efficiency because downstream processes are affected as well.

The invention recognizes that shifting cellular metabolism from fatty acid oxidation to glucose oxidation reduces cardiac remodeling in certain clinical conditions. Conditions such as heart attack, hypertension, congenital heart disease, and valvular heart disease may be accompanied by both decreased cardiac energy production and cardiac remodeling. The invention is based in part on the finding that compounds that increase energy production in the heart by promoting glucose oxidation also lead to reduced hypertrophy and improved contractility of the heart.

### Compounds

The invention includes providing pharmaceutical compositions that include a compound that shifts cellular metabolism from fatty acid oxidation to glucose oxidation. Compounds that shift cellular metabolism from fatty acid oxidation to glucose oxidation are described in, for example, International Patent Publication No. WO 2018/236745, the contents of which are incorporated herein by reference.

The compound that shifts cellular metabolism from fatty acid oxidation to glucose oxidation may be represented by formula (I):

A-L-B (I),

in which A is a molecule that shifts cellular metabolism from fatty acid oxidation to glucose oxidation, L is a linker, and B is a compound that promotes mitochondrial respiration.

Component A may be any suitable molecule that shifts cellular metabolism from fatty acid oxidation to glucose oxidation. Such compounds can be classified based on their mechanism of action. See Fillmore, N., et al., Mitochondrial fatty acid oxidation alterations in heart failure, ischemic heart disease and diabetic cardiomyopathy, Brit. J. Pharmacol. 171:2080-2090 (2014), incorporated herein by reference.

One class of glucose-shifting compounds includes compounds that inhibit fatty acid oxidation directly. Compounds in this class include inhibitors of malonyl CoA decarboxylase (MCD), carnitine palmitoyl transferase 1 (CPT-1), or mitochondrial fatty acid oxidation. Mitochondrial fatty acid oxidation inhibitors include trimetazidine and other compounds described in International Patent Publication No. WO 2002/064,576, the contents of which is incorporated herein by reference. Trimetazidine binds to distinct sites on the inner and outer mitochondrial membranes and affects both ion permeability and metabolic function of mitochondria. Morin, D., et al., Evidence for the existence of [³H]-trimetazidine binding sites involved in the regulation of the mitochondrial permeability transition pore, Brit. J. Pharmacol. 123:1385-1394 (1998), incorporated herein by reference. MCD inhibitors include CBM-301106, CBM-300864, CBM-301940, 5-(1,1,1,3,3,3-hexafluoro-2-hydroxypropan-2-yl)-4,5-dihydroisoxazole-3-carboxamides, methyl 5-(N-(4-(1,1,1,3,3,3-hexafluoro-2-hydroxypropan-2-yl)phenyl)morpholine-4-carboxamido)pentanoate, and other compounds described in Chung, J. F., et al., Discovery of Potent and Orally Available Malonyl-CoA Decarboxylase Inhibitors as Cardioprotective Agents, J. Med. Chem. 49:4055-4058 (2006); Cheng J. F. et al., Synthesis and structure-activity relationship of small-molecule malonyl coenzyme A decarboxylase inhibitors, J. Med. Chem. 49:1517-1525 (2006); U.S. Patent Publication No. 2004/0082564; and International Patent Publication No. WO 2002/058,698, the contents of which are incorporated herein by reference. CPT-1 inhibitors include oxfenicine, perhexiline, etomoxir, and other compounds described in International Patent Publication Nos. WO 2015/018,660; WO 2008/109,991; WO 2009/015,485; and WO 2009/156479; and U.S. Patent Publication No. 2011/0212072, the contents of which are incorporated herein by reference.

Another class of glucose-shifting compounds includes compounds that stimulate glucose oxidation directly. Examples of such compounds are described in U.S. Patent Publication No. 2003/0191182; International Patent Publication No. WO 2006/117,686; U.S. Pat. No. 8,202,901, the content of each of which are incorporated herein by reference.

Another class of glucose-shifting compounds includes compounds that decrease the level of circulating fatty acids that supply the heart. Examples of such compounds include agonists of PPARα and PPARγ, including fibrate drugs, such as clofibrate, gemfibrozil, ciprofibrate, bezafibrate, and fenofibrate, and thiazolidinediones, GW-9662, and other compounds described in U.S. Pat. No. 9,096,538, which is incorporated herein by reference.

Component L may be any suitable linker. Preferably, the linker can be cleaved in vivo to release components A and B. The linker may be an alkoxy group. The linker may be polyethylene glycol of any length. The linker may be represented by (CH₂CH₂O)ₓ, in which x=1-15 or (CH₂CH₂O)ₓ, in which x=1-3. Other suitable linkers include 1,3-propanediol, diazo linkers, phosphoramidite linkers, disulfide linkers, cleavable peptides, iminodiacetic acid linkers, thioether linkers, and other linkers described in Leriche, G., et al., Cleavable linkers in chemical biology, Bioorg. Med. Chem. 20:571-582 (2012); International Patent Publication No. WO 1995/000,165; and U.S. Pat. No. 8,461,117, the contents of which are incorporated herein by reference.

Component B may be any compound that promotes mitochondrial respiration. For example, component B may be an intermediate of the citric acid cycle or a molecule that can be metabolized to enter the citric acid cycle, such as succinate, fumarate, malate, oxaloacetate, citrate, isocitrate, α-ketoglutarate, pyruvate, acetone, acetoacetic acid, β-hydroxybutpic acid, β-ketopentanoate, or β-hydroxypentanoate. Intermediates of the citric acid cycle may become depleted if these molecules are used for biosynthetic purposes, resulting in inefficient generation of ATP from the citric acid cycle. However, due to the anaplerotic effect, providing one intermediate of the citric acid cycle leads to restoration of all intermediates as the cycle turns. Thus, intermediates of the citric acid cycle can promote mitochondrial respiration.

The compound may include a NAD⁺ precursor molecule. NAD⁺ is an important oxidizing agent that acts as a coenzyme in multiple reactions of the citric acid cycle. In these reactions, NAD⁺ is reduced to NADH. Conversely, NADH is oxidized back to NAD⁺ when it donates electrons to mitochondrial electron transport chain. In humans, NAD⁺ can be synthesized de novo from tryptophan, but not in quantities sufficient to meet metabolic demands. Consequently, NAD⁺ is also synthesized via a salvage pathway, which uses precursors that must be supplied from the diet. Among the precursors used by the salvage pathway for NAD⁺ synthesis are nicotinic acid, nicotinamide, and nicotinamide riboside. By providing a NAD⁺ precursor, such as nicotinic acid, nicotinamide, or nicotinamide riboside, the compound facilitates NAD⁺ synthesis.

The inclusion of a NAD⁺ precursor allows the compounds to stimulate energy production in cardiac mitochondria in multiple ways. First, component A shifts cellular metabolism from fatty acid oxidation to glucose oxidation, which is inherently more efficient. Next, component B ensures that the intermediates of the citric acid cycle are present at adequate levels and do not become depleted or limiting. As a result, glucose-derived acetyl CoA is efficiently oxidized. Finally, the NAD⁺ precursor provides an essential coenzyme that cycles between oxidized and reduced forms to promote respiration. In the oxidized form, NAD⁺ drives reactions of the citric acid cycle. In the reduced form, NADH promotes electron transport to create a proton gradient that enables ATP synthesis. Consequently, the chemical potential resulting from oxidation of acetyl CoA is efficiently converted to ATP that can be used for various cellular functions.

The NAD⁺ precursor molecule may be covalently attached to the compound in any suitable manner. For example, it may be linked to A, L, or B, and it may be attached directly or via another linker. Preferably, it is attached via a linker that can be cleaved in vivo. The NAD⁺ precursor molecule may be attached via a 1,3-propanediol linkage.

The compound may be covalently attached to one or more molecules of polyethylene glycol (PEG), i.e., the compound may be PEGylated. In many instances, PEGylation of molecules reduces their immunogenicity, which prevents the molecules from being cleared from the body and allows them to remain in circulation longer. The compound may contain a PEG polymer of any size. For example, the PEG polymer may have from 1-500 (CH₂CH₂O) units. The PEG polymer may have any suitable geometry, such as a straight chain, branched chain, star configuration, or comb configuration. The compound may be PEGylated at any site. For example, the compound may be PEGylated on component A, component B, component L, or, if present, the NAD⁺ precursor. The compound may be PEGylated at multiple sites. For a compound PEGylated at multiple sites, the various PEG polymers may be of the same or different size and of the same or different configuration.

The compound may be a PEGylated form of trimetazidine. For example, the compound may be represented by formula (VI):

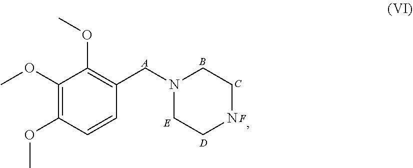

in which one or more of the carbon atoms at positions A, B, C, D, and E and/or the nitrogen atom at position F are substituted with —(CH₂CH₂O)ₙH and n=1-15. The carbon atoms at positions

A, B, C, D, and E may have two PEG substituents. In molecules that have multiple PEG chains, the different PEG chains may have the same or different length.

The compounds of formula (I) may be represented by formula (II):

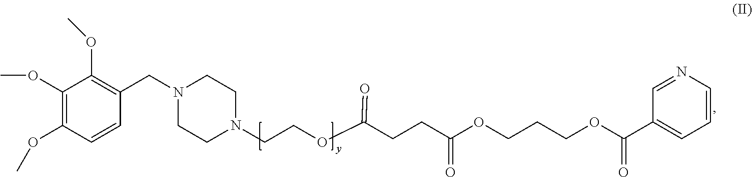

in which y=1-3.

The compounds of formula (I) may be represented by formula (III):

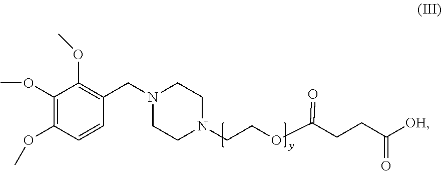

in which y=1-3.

The compound that shifts cellular metabolism from fatty acid oxidation to glucose oxidation may be represented by formula (IV):

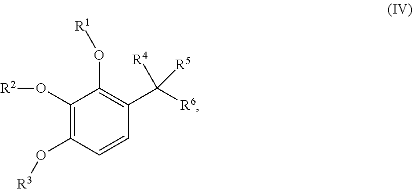

in which R¹, R², and R³ are independently H or a (C₁-C₄)alkyl group; R⁴ and R⁵ together are ═O, —O(CH₂)ₘO—, or —(CH₂)ₘ—, in which m=2-4, or R⁴ is H and R⁵ is OR¹⁴, SR¹⁴, or (CH₂CH₂O)ₙH, in which R¹⁴ is H or a (C₁-C₄)alkyl group and n=1-15; and R⁶ is a single or multi-ring structure optionally substituted at one or more ring positions by a heteroatom, in which each ring position optionally comprises one or more substituents.

R⁶ may be a single or multi-ring structure of any size. For example, the structure may contain 3-22 atoms, not including hydrogen atoms bonded to atoms in ring positions. The structure may include one or more alkyl, alkenyl, or aromatic rings. The structure may include one or more heteroatoms, i.e., atoms other than carbon. For example, the heteroatom may be oxygen, nitrogen, or sulfur, or phosphorus.

One or more ring position of R⁶ may include a substituent that includes a compound that promotes mitochondrial respiration, as described above in relation to component B of formula (I). The substituent may include a linker, as described above in relation to component L of formula (I). The substituent may include a NAD⁺ precursor molecule, as described above in relation to compounds of formula (I).

The substituent on a ring position of R⁶ may be

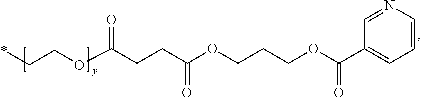

in which y=1-3.

The substituent on a ring position of R⁶ may be

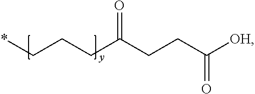

in which y=1-3.

R⁶ may be

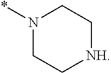 [SMILES: *N1CCNCC1]

For some compounds that include trimetazidine prodrugs, analogs, derivatives, it is advantageous to have the trimetazidine moiety substituted with a single ethylene glycol moiety. Thus, compositions of the invention may include compounds of formulas (I) and (VIII) that contain linkers in which x=1, compounds of formulas (II) and (III) in which y=1, compounds of formula (V) in which z=1, compounds of formula (VI) in which n=1, and compounds of formula (VII) in which A is linked to C via a single ethylene glycol moiety. Without wishing to be bound by theory, the attachment of a single ethylene glycol moiety to the trimetazidine moiety may improve the bioavailability of trimetazidine.

The compound of formula (IV) may have a structure represented by formula (IX) or formula (X):

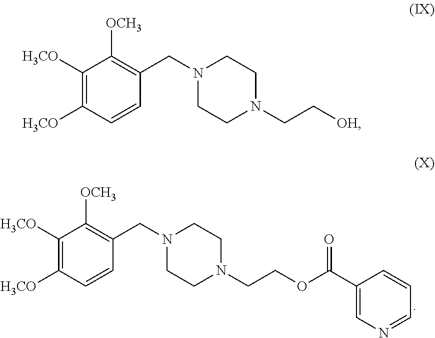 [SMILES: COc1ccc(CN2CCN(CCO)CC2)c(OC)c1OC ; COc1ccc(CN2CCN(CCOC(=O)c3cccnc3)CC2)c(OC)c1OC]

The compound that shifts cellular metabolism from fatty acid oxidation to glucose oxidation may be represented by formula (V):

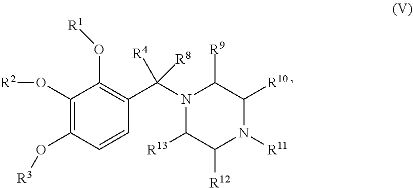

in which R¹, R², and R³ are independently H or a (C₁-C₄)alkyl group; R⁴ and R⁸ together are ═O, —O(CH₂)ₘO—, or —(CH₂)ₘ—, in which m=2-4, or R⁴ is H and R⁸ is H, OR¹⁴, SR¹⁴, or (CH₂CH₂O)ₙH, in which R¹⁴ is H or a (C₁-C₄)alkyl group and n=1-15; R⁹, R¹⁰, R¹², and R¹³ are independently H or (CH₂CH₂O)_{z}H, in which z=1-15; and R¹¹ comprises a compound that promotes mitochondrial respiration, as described above in relation to component B of formula (I). R¹¹ may include a linker, as described above in relation to component L of formula (I).
R¹¹ may be

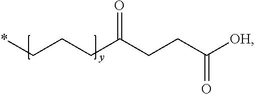

in which y=1-3.
R¹¹ may include a NAD⁺ precursor molecule, as described above in relation to compounds of formula (I).

R¹¹ may be

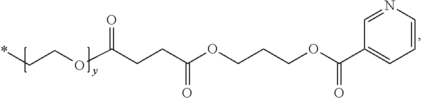

in which y=1-3.

In some embodiments described above, the compound includes multiple active agents joined by linkers in a single molecule. It may be advantageous to deliver multiple active agents as components of a single molecule. Without wishing to be bound by a particular theory, there are several reasons why co-delivery of active agents in a single molecule may be advantageous. One possibility is that a single large molecule may have reduced side effects compared to the component agents. Free trimetazidine causes symptoms similar to those in Parkinson's disease in a fraction of patients. However, when trimetazidine is derivatized to include other components, such as succinate, the molecule is bulkier and may not be able to access sites where free trimetazidine can causes unintended effects. Trimetazidine derivatized as described above is also more hydrophilic and thus may be less likely to cross the blood-brain barrier to cause neurological effects. Another possibility is that modification of trimetazidine may alter its pharmacokinetic properties. Because the derivatized molecule is metabolized to produce the active agent, the active agent is released gradually. Consequently, levels of the active agent in the body may not reach peaks as high as when a comparable amount is administered in a single bolus. Another possibility is that less of each active agent, such as trimetazidine, is required because the compositions of the invention may include compounds that have multiple active agents. For example, trimetazidine shifts metabolism from fatty acid oxidation to glucose oxidation, and succinate improves mitochondrial respiration generally. Thus, a compound that provides both agents stimulates a larger increase in glucose-driven ATP production for a given amount of trimetazidine than does a compound that delivers trimetazidine alone.

The compound that shifts cellular metabolism from fatty acid oxidation to glucose oxidation may be represented by formula (VII):

A-C (VII),

in which A is a molecule that shifts cellular metabolism from fatty acid oxidation to glucose oxidation, and C is a NAD⁺ precursor molecule. A and C may be covalently linked.

The molecule that shifts cellular metabolism from fatty acid oxidation to glucose oxidation may be PEGylated with an ethylene glycol moiety. The molecule that shifts cellular metabolism from fatty acid oxidation to glucose oxidation may have multiple ethylene glycol moieties, such as one, two three, four, five, or more ethylene glycol moieties. The ethylene glycol moiety may be represented by (CH₂CH₂O)ₓ, in which x=1-15. The ethylene glycol moiety may form a covalent linkage between the molecule that shifts cellular metabolism from fatty acid oxidation to glucose oxidation and the NAD⁺ precursor molecule. The ethylene glycol moiety may be separate from a covalent linkage between the molecule that shifts cellular metabolism from fatty acid oxidation to glucose oxidation and the NAD⁺ precursor molecule.

The compound of formula (VII) may include nicotinic acid that is covalently linked to a PEGylated form of trimetazidine. The nicotinic acid may be covalently linked via a PEGylated moiety, i.e., via an ethylene glycol linkage. The nicotinic acid may be covalently linked via the trimetazidine moiety.

The compound that shifts cellular metabolism from fatty acid oxidation to glucose oxidation may be represented by formula (VIII):

A-L-C (VIII),

in which A is a molecule that shifts cellular metabolism from fatty acid oxidation to glucose oxidation, L is a linker, and C is a NAD⁺ precursor molecule. A may be covalently linked to L, and L may be covalently linked to C.

The molecule that shifts cellular metabolism from fatty acid oxidation to glucose oxidation, the linker, and the NAD⁺ precursor molecule may be as described above in relation to compounds of other formulas.

The compounds may be provided as co-crystals with other compounds. Co-crystals are crystalline materials composed of two or more different molecules in the same crystal lattice. The different molecules may be neutral and interact non-ionically within the lattice. Co-crystals may include one or more of the compounds described above and one or more other molecules that stimulate mitochondrial respiration or serve as NAD⁺ precursors. For example, a co-crystal may include any of the following combinations: (1) a molecule that shifts cellular metabolism from fatty acid oxidation to glucose oxidation and (2) a NAD⁺ precursor molecule; (1) a compound that promotes mitochondrial respiration and (2) a NAD⁺ precursor molecule; (1) a molecule that shifts cellular metabolism from fatty acid oxidation to glucose oxidation and (2) a compound that promotes mitochondrial respiration; (1) a molecule comprising a molecule that shifts cellular metabolism from fatty acid oxidation to glucose oxidation covalently linked to a compound that promotes mitochondrial respiration and (2) a NAD⁺ precursor molecule. In specific embodiments, a co-crystal may include (1) a compound of formula (I), (III), (IV), or (V) and (2) nicotinic acid, nicotinamide, or nicotinamide riboside.

The compounds may include one or more atoms that are enriched for an isotope. For example, the compounds may have one or more hydrogen atoms replaced with deuterium or tritium. Isotopic substitution or enrichment may occur at carbon, sulfur, or phosphorus, or other atoms. The compounds may be isotopically substituted or enriched for a given atom at one or more positions within the compound, or the compounds may be isotopically substituted or enriched at all instances of a given atom within the compound.

### Formulations

The invention includes providing pharmaceutical compositions containing one or more of the compounds described above. A pharmaceutical composition containing the compounds may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated to form osmotic therapeutic tablets for control release by the techniques described in U.S. Pat. Nos. 4,256,108, 4,166,452 and 4,265,874, the contents of which are incorporated herein by reference. Preparation and administration of compounds is discussed in U.S. Pat. No. 6,214,841 and U.S. Pub. 2003/0232877, the contents of which are incorporated by reference herein in their entirety.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The compositions of the invention are useful for improving cardiac efficiency. A variety of definitions of cardiac efficiency exist in the medical literature. See, e.g. Schipke, J. D. Cardiac efficiency, Basic Res. Cardiol. 89:207-40 (1994); and Gibbs, C. L. and Barclay, C. J. Cardiac efficiency, Cardiovasc. Res. 30:627-634 (1995), incorporated herein by reference. One definition of cardiac mechanical efficiency is the ratio of external cardiac power to cardiac energy expenditure by the left ventricle. See Lopaschuk G. D., et al., Myocardial Fatty Acid Metabolism in Health and Disease, Phys. Rev. 90:207-258 (2010), incorporated herein by reference. Another definition is the ratio between stroke work and oxygen consumption, which ranges from 20-25% in the normal human heart. Visser, F., Measuring cardiac efficiency: is it useful? Hear Metab. 39:3-4 (2008), incorporated herein by reference. Another definition is the ratio of the stroke volume to mean arterial blood pressure. Any suitable definition of cardiac efficiency may be used to measure the effects of compositions of the invention.

The compositions of the invention may contain an agent that shifts cellular metabolism from fatty acid oxidation to glucose oxidation and an inhibitor of PDK in a single formulation. Alternatively, the compositions of the invention may contain an agent that shifts cellular metabolism from fatty acid oxidation to glucose oxidation and an inhibitor of PDK in separate formulations. The compositions may contain a NAD⁺ precursor molecule in a formulation that contains an agent that shifts cellular metabolism from fatty acid oxidation to glucose oxidation and/or an inhibitor of PDK, or the NAD⁺ precursor molecule may be provided in a separate formulation.

### Methods of Treating Diseases, Disorders, and Conditions

The methods of the invention are useful for treating any disease, disorder, or condition associated with cardiac remodeling, also called ventricular remodeling. An accepted definition of cardiac remodeling is a group of molecular, cellular and interstitial changes that clinically manifest as changes in size, shape and function of the heart resulting from cardiac injury. Cohn J N, Ferrari R, Sharpe N. Cardiac remodeling-concepts and clinical implications: a consensus paper from an international forum on cardiac remodeling. Behalf of an International Forum on Cardiac Remodeling. J Am Coll Cardiol. 2000; 35(3):569-582, incorporated herein by reference. Cardiac modeling includes physiological remodeling, which occurs as a result of exercise, and pathological remodeling, which is caused by injury to the heart muscle. Physiological remodeling generally provides beneficial effects and is reversible, whereas pathological remodeling is deleterious and largely irreversible.

Cardiac remodeling is a component of cardiomyopathy that includes a variety of categories of changes. Ventricular hypertrophy is thickening of the wall of a ventricle of the heart. Ventricular hypertrophy can occur in the left ventricle, right ventricle, or both. Cardiomegaly refers generally to enlargement of the heart. Dilated cardiomyopathy is a common form of cardiomegaly in which the ventricles become thin and stretched. Other examples of cardiac remodeling include concentric hypertrophy and eccentric hypertrophy.

Cardiac remodeling is response to heart damage, commonly caused by a heart attack. The death of cardiomyocytes in a region of the heart causes thinning and weakening of the tissue and dilation of the adjacent chamber. The cardiomyocytes do not regrow but are replaced by fibrous tissue in the process of myocardial scarring. The scar tissue provides structural strength to the damaged tissue but lacks the elasticity and contractile capacity of cardiomyocytes. Consequently, the remodeling results in a decrease in pumping efficiency of the heart.

Cardiac remodeling may be associated with myocardial infarction. Myocardial infarction may result from coronary artery disease, coronary artery spasms, high blood pressure (hypertension), smoking, diabetes, lack of exercise, obesity, high blood cholesterol, poor diet, or excessive alcohol intake, or drug (e.g., cocaine) use. Myocardial infarction may cause cardiac arrest.

Cardiac remodeling may be associated with hypertension. Hypertension may result from excess salt intake, excess body weight, smoking, alcohol use, or kidney disease.

Cardiac remodeling may be associated with congenital heart disease or defects. Congenital heart defects may be classified as hypoplasia, obstruction defects, septal defects, or cyanotic defects.

Cardiac remodeling may be associated with valvular heart disease. Valvular heart diseases include aortic valve stenosis, aortic insufficiency, mitral valve stenosis, mitral insufficiency, tricuspid valve stenosis, tricuspid insufficiency, pulmonary valve stenosis, and pulmonary insufficiency.

For example and without limitation, the cardiac remodeling or risk of developing cardiac remodeling may be associated with aberrant subclavian artery, aortic regurgitation, aortic stenosis, arteriovenous malformation and fistula, atrial septal defect, atrioventricular septal defect, bicuspid aortic valve, cardiomegaly, cardiomyopathy, coarctation of the aorta, complete heart block, concentric hypertrophy, congenital heart defects, congenital heart disease, coronary artery disease, dextrocardia, dextro-transposition of the great arteries, diabetes, diet, double aortic arch, double inlet left ventricle, double outlet right ventricle, Ebstein's anomaly, giant hepatic hemangioma, heart failure, high cholesterol, high-output hemodialysis fistula, hypertension, hypertension, hypoplastic left heart syndrome, hypoplastic right heart syndrome, interrupted aortic arch, levo-transposition of the great arteries, mitral regurgitation, also causing left atrial volume overload, mitral stenosis, myocardial ischemia, obesity, outflow obstruction., partial anomalous pulmonary venous connection, patent ductus arteriosus, pentalogy of Cantrell, persistent truncus arteriosus, pressure overload, pulmonary atresia, pulmonary hypertension, pulmonary regurgitation, pulmonary stenosis, rhabdomyomas, right ventricular volume overload, scimitar syndrome, Shone's syndrome, tetralogy of Fallot, total anomalous pulmonary venous connection, transposition of the great vessels, tricuspid atresia, tricuspid regurgitation, use of tobacco, alcohol, or other drugs, valvular heart disease, ventricular dilation, ventricular hypertrophy, ventricular septal defect, volume overload, and Wolff-Parkinson-White syndrome.

The pharmaceutical compositions may be provided by any suitable route of administration. For example and without limitation, the compositions may be administered buccally, by injection, dermally, enterally, intraarterially, intravenously, nasally, orally, parenterally, pulmonarily, rectally, subcutaneously, topically, transdermally, or with or on an implantable medical device (e.g., stent or drug-eluting stent or balloon equivalents).

### EXAMPLES

### Example 1

Protocol

The effects of selected compounds on mitochondrial function were analyzed. HepG2 cells were dosed with test compound and in real time the extracellular oxygen levels and pH were measured using the XFe96 flux analyzer (Seahorse Biosciences). XFe Technology uses solid-state sensors to simultaneously measure both oxygen consumption rate (OCR) and extracellular acidification rate (ECAR) to determine effects on oxidative phosphorylation (OXPHOS) and glycolysis simultaneously. The cells were then subjected to sequential exposure to various inhibitors of mitochondrial function to assess cellular metabolism.

Data interpretation.

A compound was identified as positive mitochondrial-active compound when it caused a change in oxygen consumption rate (OCR) or extracellular acidification rate (ECAR) in the absence of cytotoxicity. Cytotoxicity was determined when both OXPHOS (OCR) and glycolysis (ECAR) were inhibited.

Definition of Mitochondrial Parameters.

Oxygen consumption rate (OCR) is a measurement of oxygen content in extracellular media. Changes in OCR indicate effects on mitochondrial function and can be bi-directional. A decrease is due to an inhibition of mitochondrial respiration, while an increase may indicate an uncoupler, in which respiration is not linked to energy production.

$$OCR = \frac{compound OCR - non mitochondrial OCR}{basal OCR - non mitochondrial OCR}$$

Extracellular acidification rate (ECAR) is the measurement of extracellular proton concentration (pH). An increase in signal means an increase in rate in number of pH ions (thus decreasing pH value) and seen as an increase in glycolysis. ECAR is expressed as a fraction of basal control (rate prior to addition of compound).

$$ECAR = \frac{compound ECAR}{basal ECAR}$$

Reserve capacity is the measured ability of cells to respond to an increase in energy demand. A reduction indicates mitochondrial dysfunction. This measurement demonstrates how close to the bioenergetic limit the cell is.

$$reserve capacity = \frac{F C C P OCR - non mitochondrial OCR}{basal OCR - non mitochondrial OCR}$$

Mitochondrial Stress Test.

A series of compounds were added sequentially to the cells to assess a bioenergetics profile, effects of test compounds on parameters such as proton leak, and reserve capacity. This can be used to assist in understanding potential mechanisms of mitochondrial toxicity. The following compounds were added in order: (1) oligomycin, (2) FCCP, and (3) rotenone and antimycin A.

Oligomycin is a known inhibitor of ATP synthase and prevents the formation of ATP. Oligomycin treatment provides a measurement of the amount of oxygen consumption related to ATP production and ATP turnover. The addition of oligomycin results in a decrease in OCR under normal conditions, and residual OCR is related to the natural proton leak.

FCCP is a protonophore and is a known uncoupler of oxygen consumption from ATP production. FCCP treatment allows the maximum achievable transfer of electrons and oxygen consumption rate and provides a measurement of reserve capacity.

Rotenone and antimycin A are known inhibitors of complex I and III of the electron transport chain, respectively. Treatment with these compounds inhibits electron transport completely, and any residual oxygen consumption is due to non-mitochondrial activity via oxygen requiring enzymes.

Definition of Mechanisms.

An electron transport chain inhibitor is an inhibitor of mitochondrial respiration that causes an increase in glycolysis as an adaptive response (e.g. decrease OCR and increase in ECAR).

The inhibition of oxygen consumption may also be due to reduced substrate availability (e.g. glucose, fatty acids, glutamine, pyruvate), for example, via transporter inhibition. Compounds that reduce the availability of substrates are substrate inhibitors. A substrate inhibitor does not result in an increase in glycolysis (e.g. OCR decrease, no response in ECAR).

Compounds that inhibit the coupling of the oxidation process from ATP production are known as uncouplers. These result in an increase in mitochondrial respiration (OCR) but inhibition of ATP production.

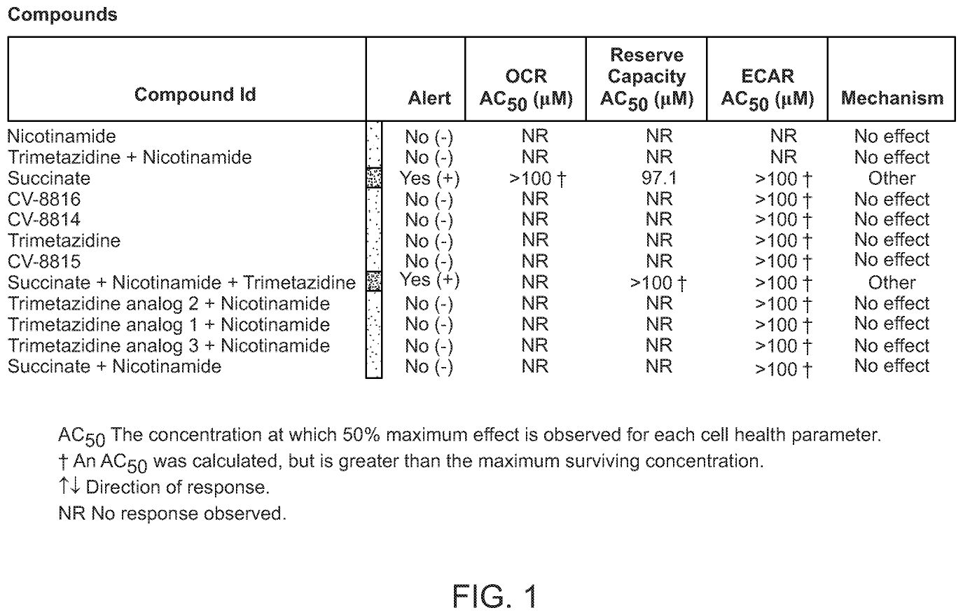

FIG. 1 is a table summarizing the effects of various compounds on mitochondrial function.

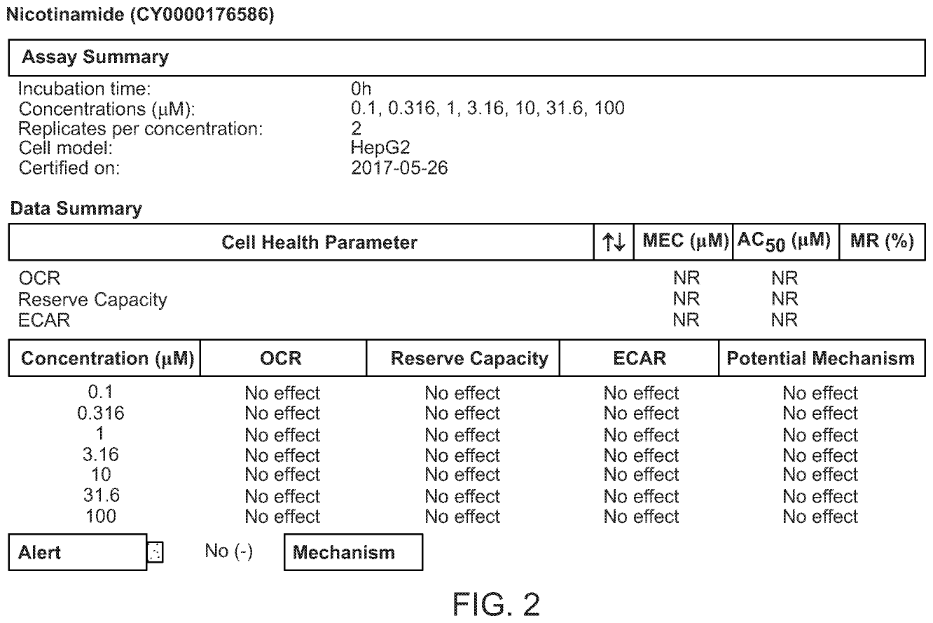

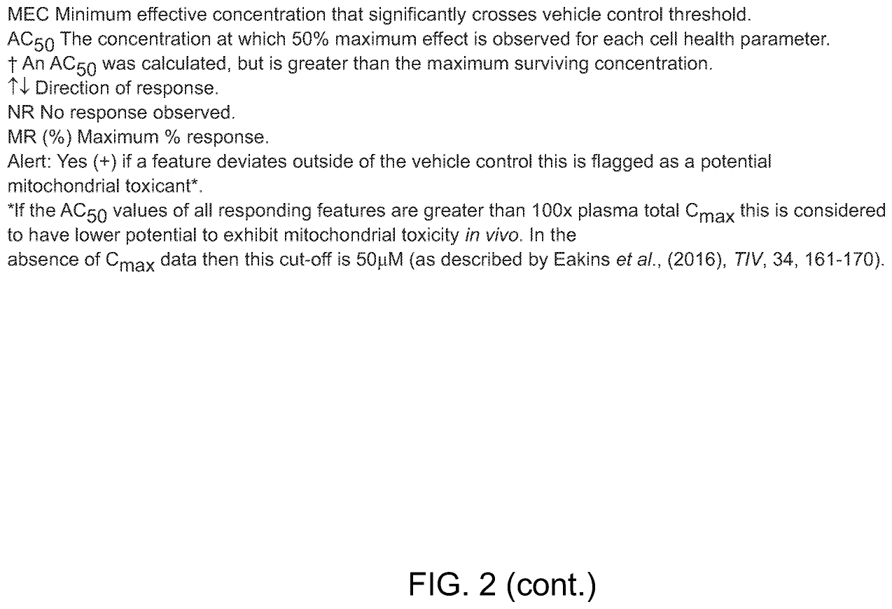

FIG. 2 is a table summarizing the effects of nicotinamide on various mitochondrial functional parameters.

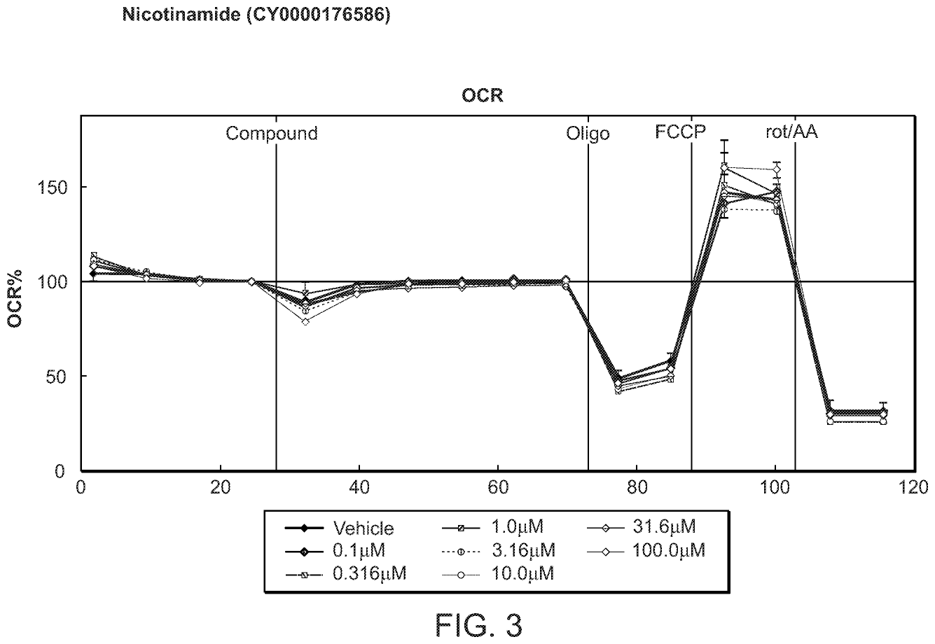

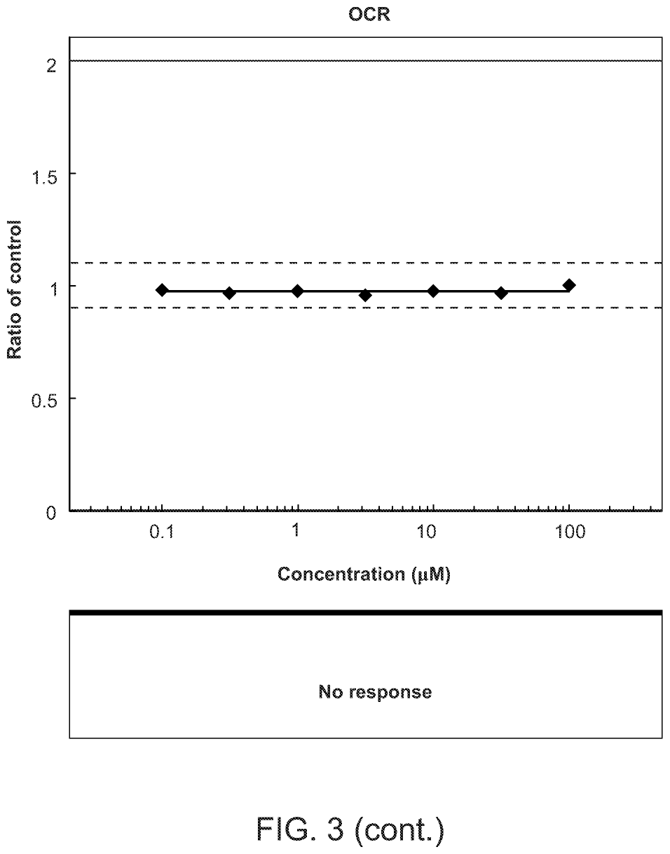

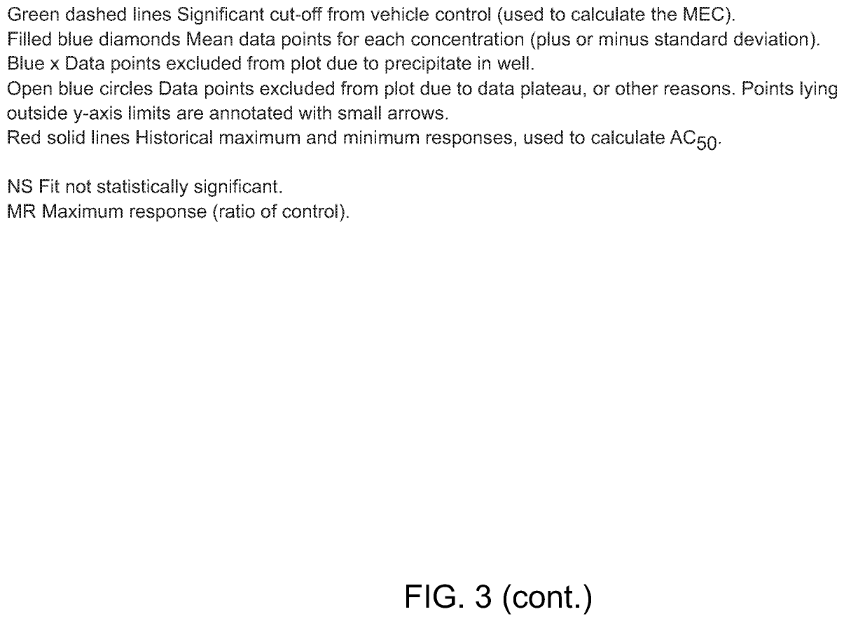

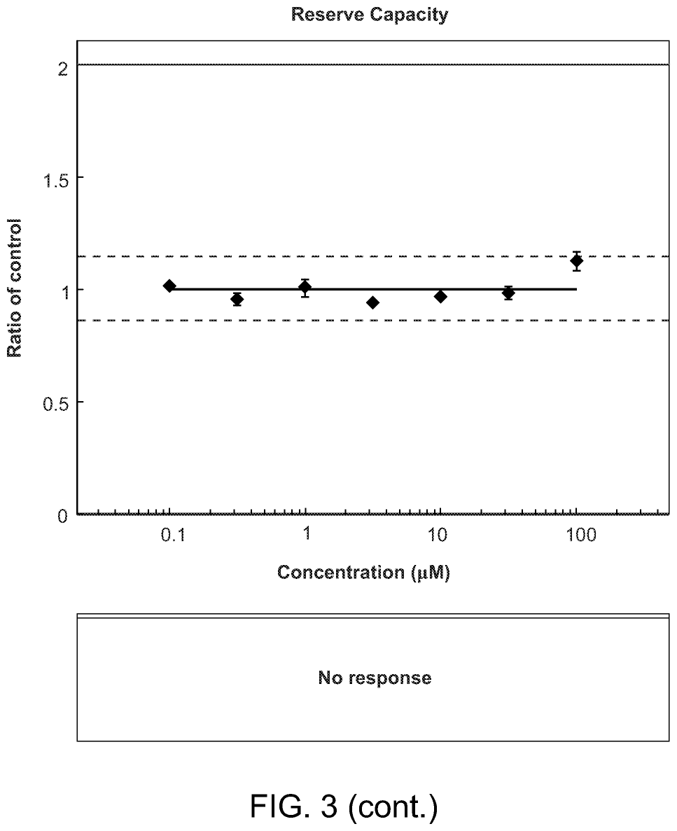

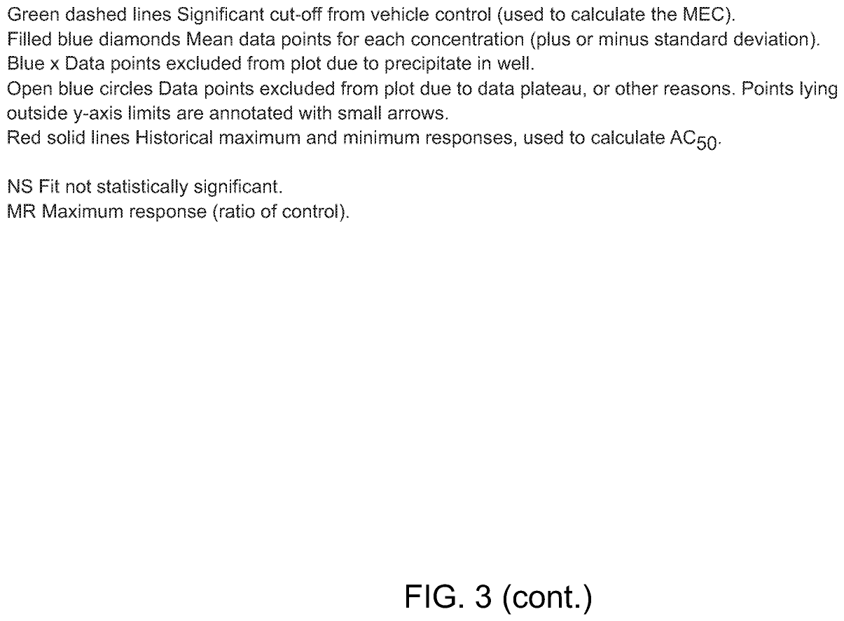

FIG. 3 is a series of graphs showing the effects of nicotinamide on oxygen consumption rate and reserve capacity.

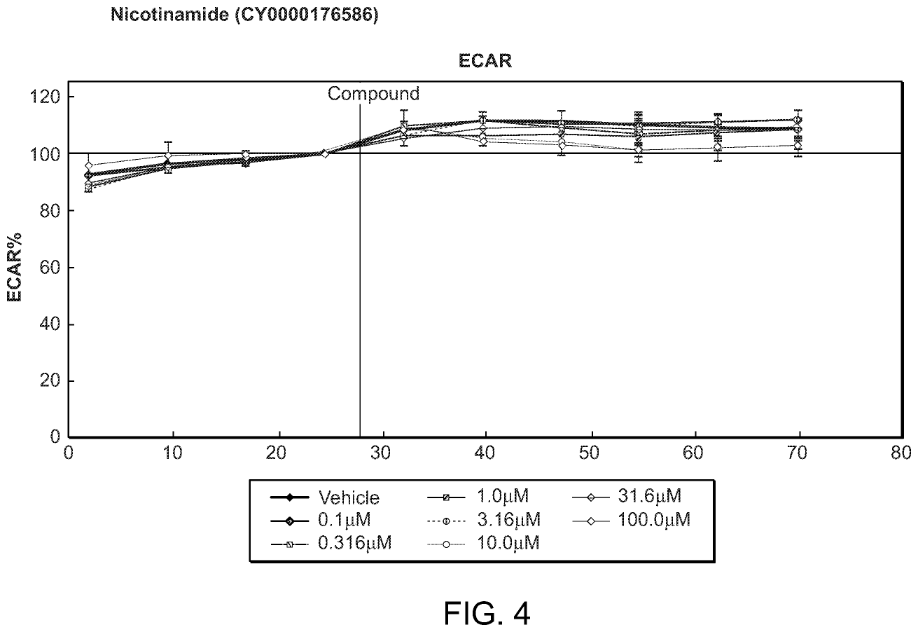

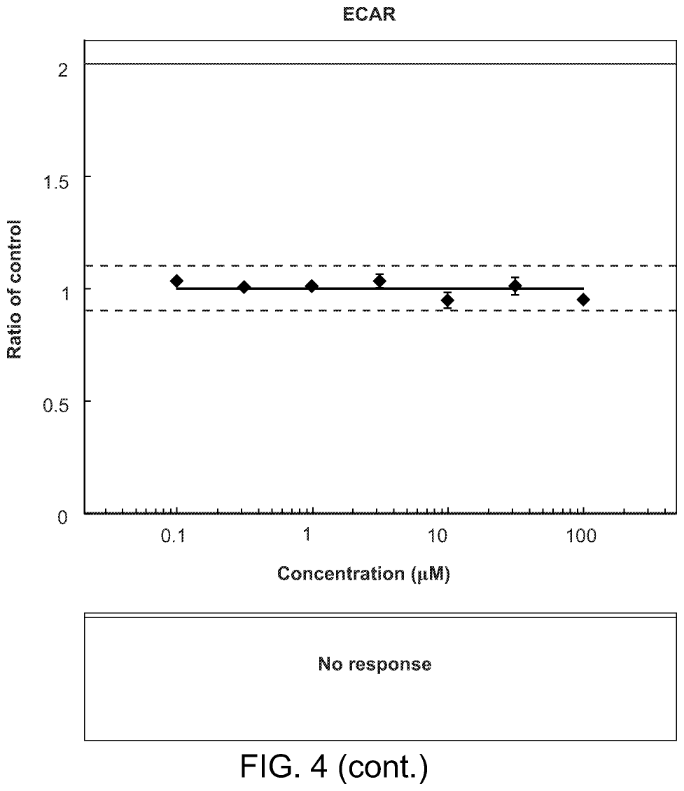

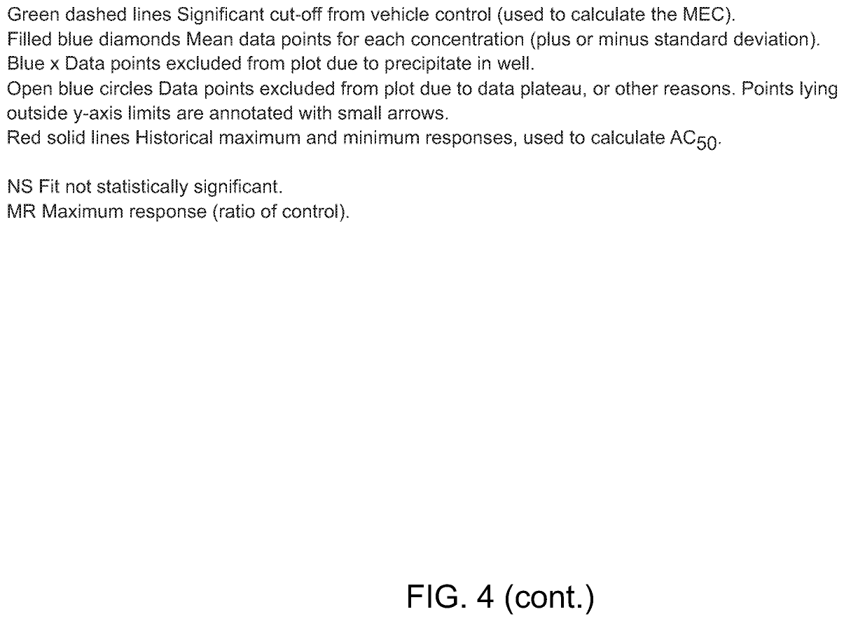

FIG. 4 is a series of graphs showing the effects of nicotinamide on extracellular acidification rate.

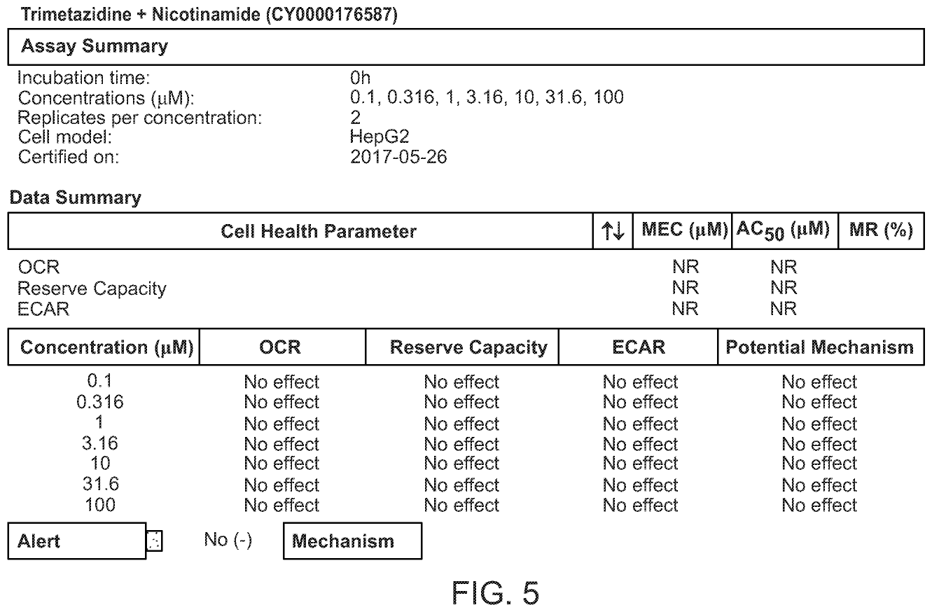

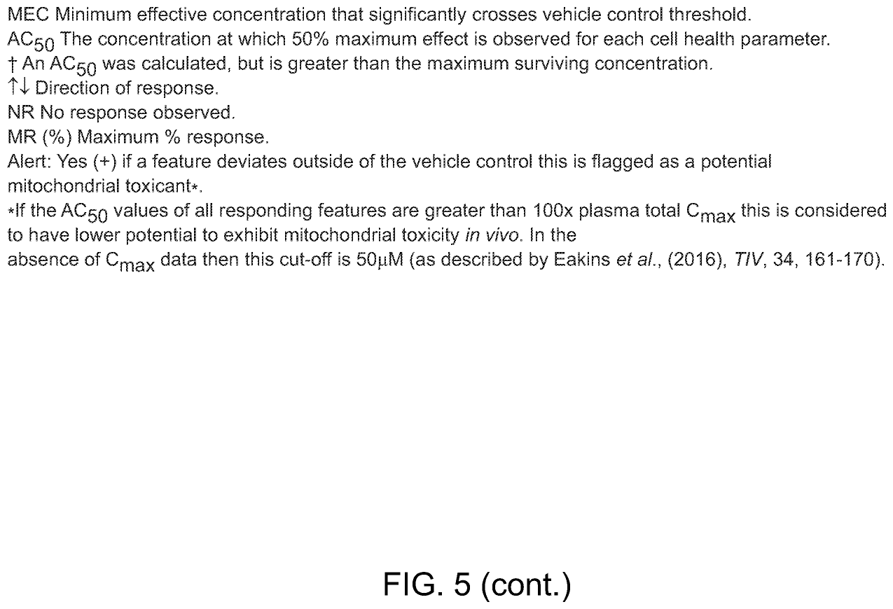

FIG. 5 is a table summarizing the effects of a combination of trimetazidine and nicotinamide on various mitochondrial functional parameters.

FIG. 6 is a series of graphs showing the effects of a combination of trimetazidine and nicotinamide on oxygen consumption rate and reserve capacity.

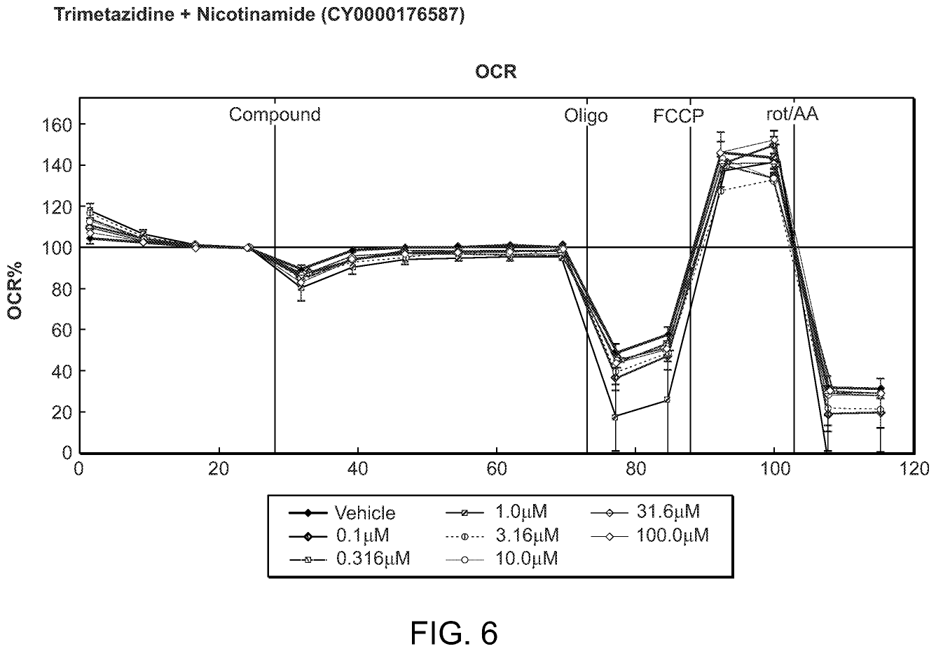

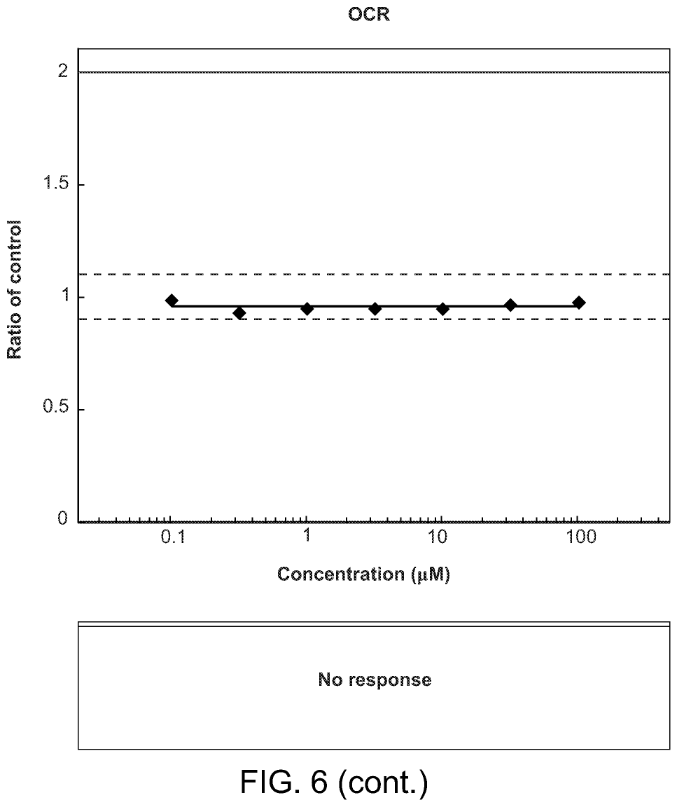

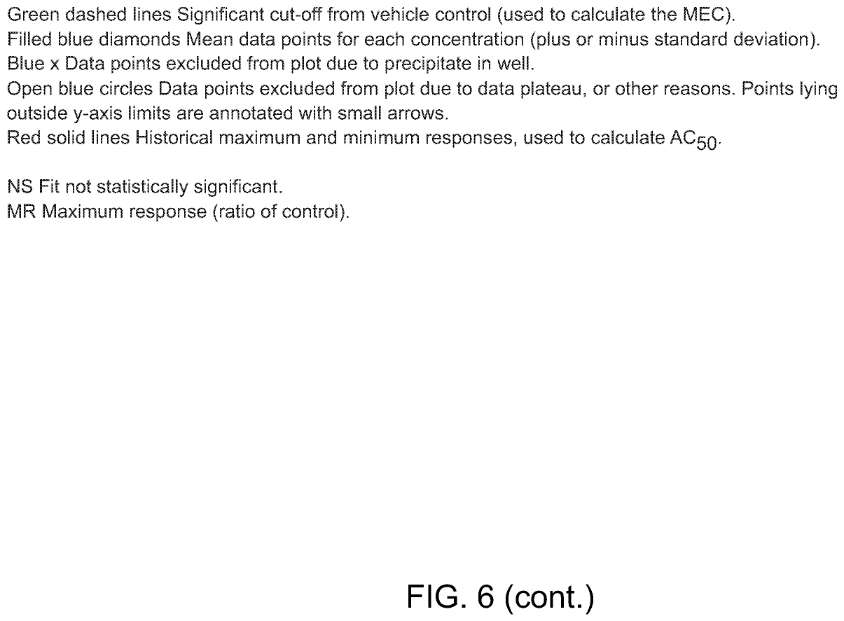

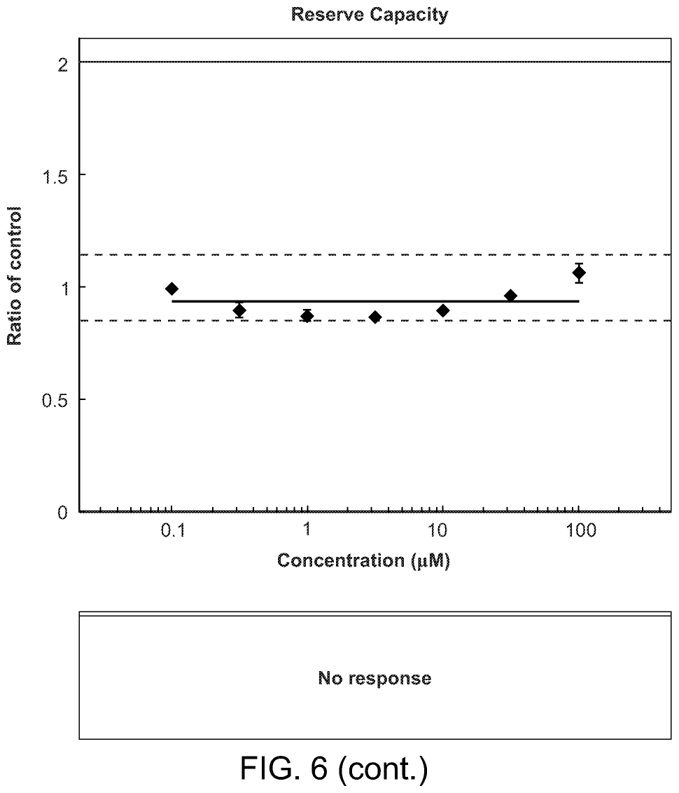

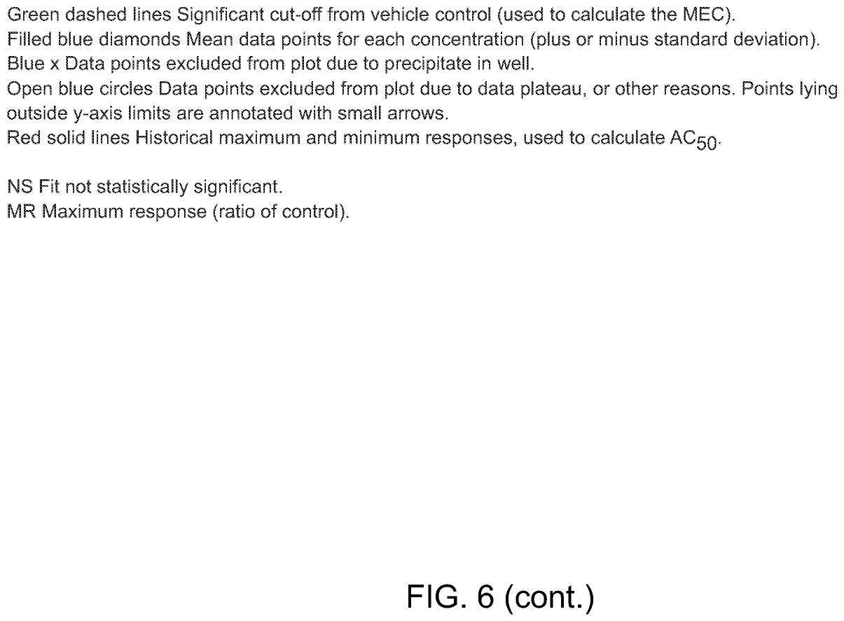

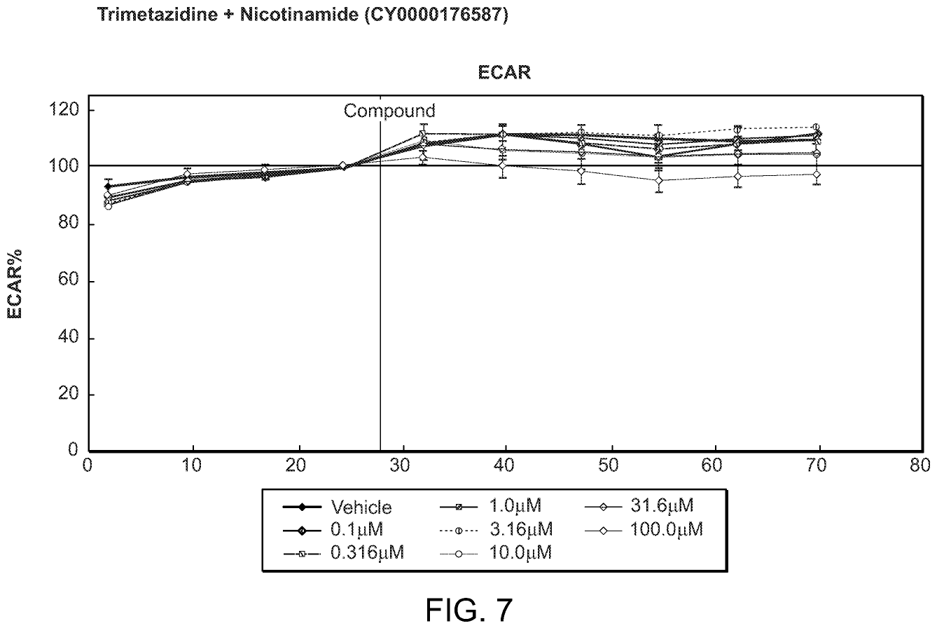

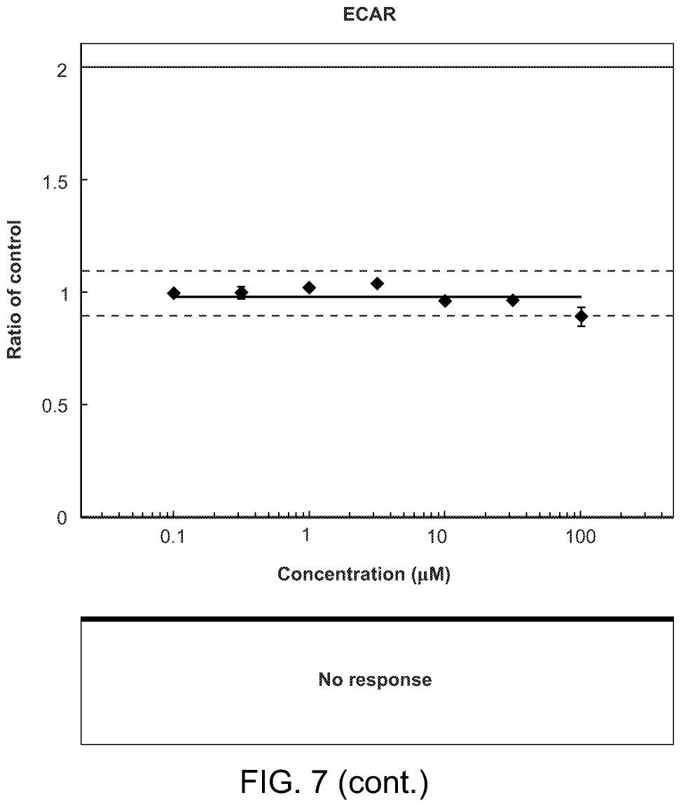

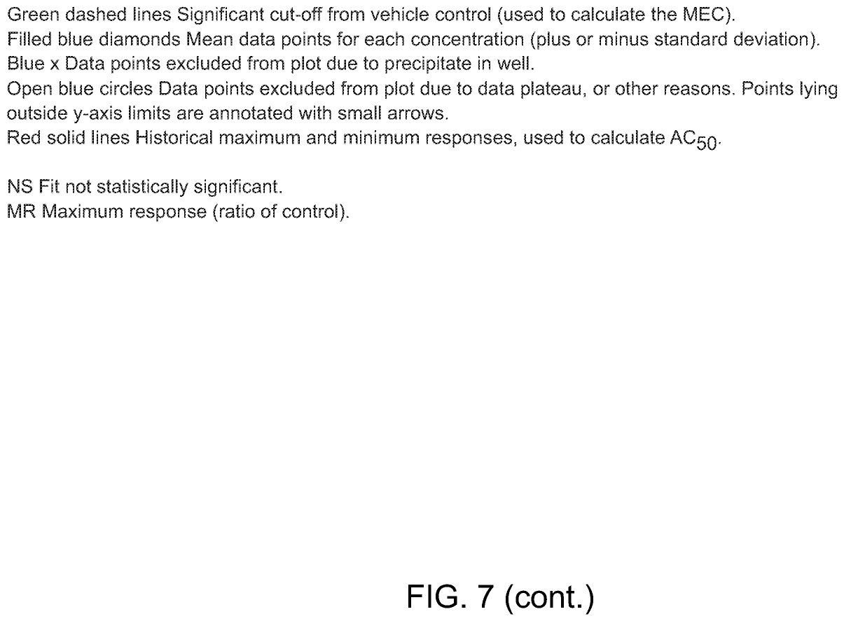

FIG. 7 is a series of graphs showing the effects of a combination of trimetazidine and nicotinamide on extracellular acidification rate.

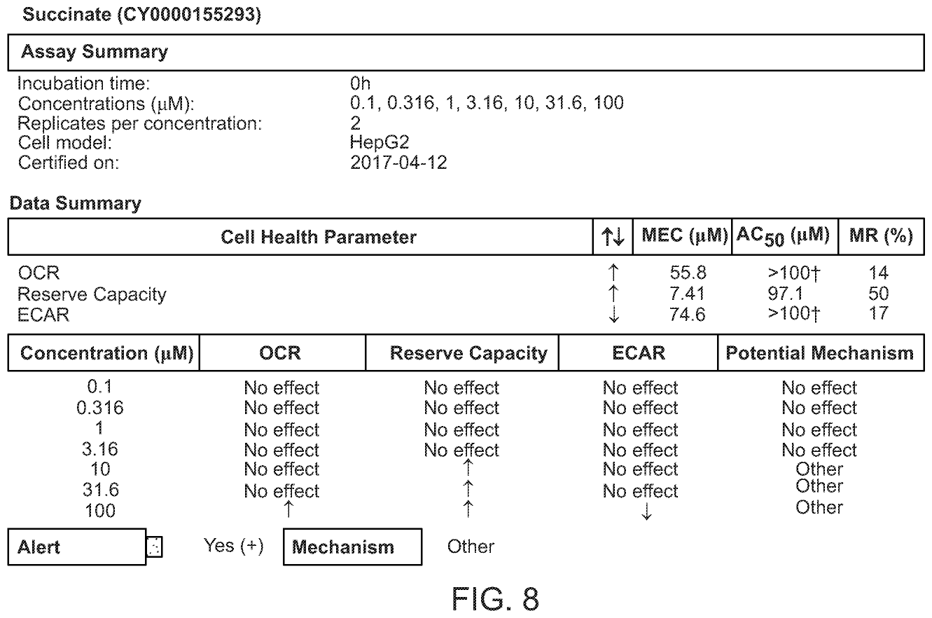

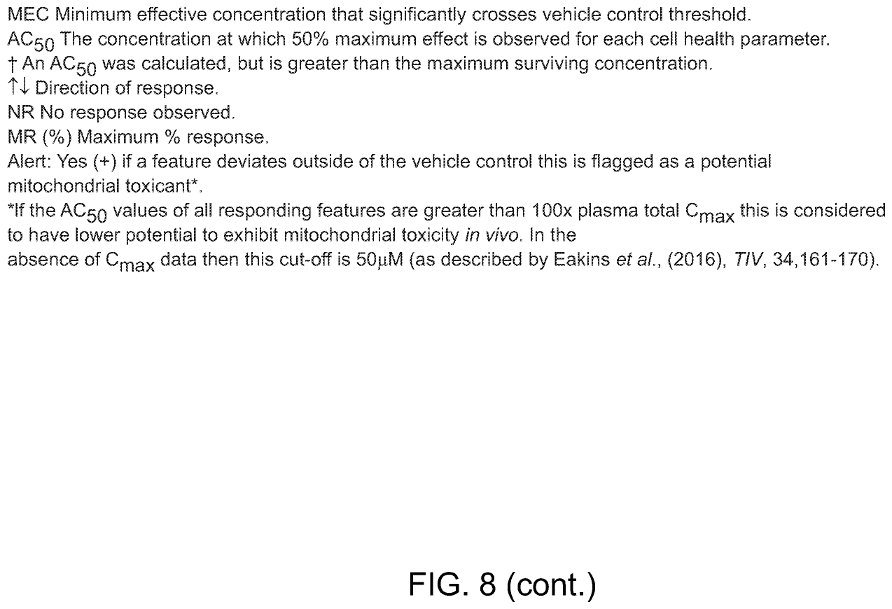

FIG. 8 is a table summarizing the effects of succinate on various mitochondrial functional parameters.

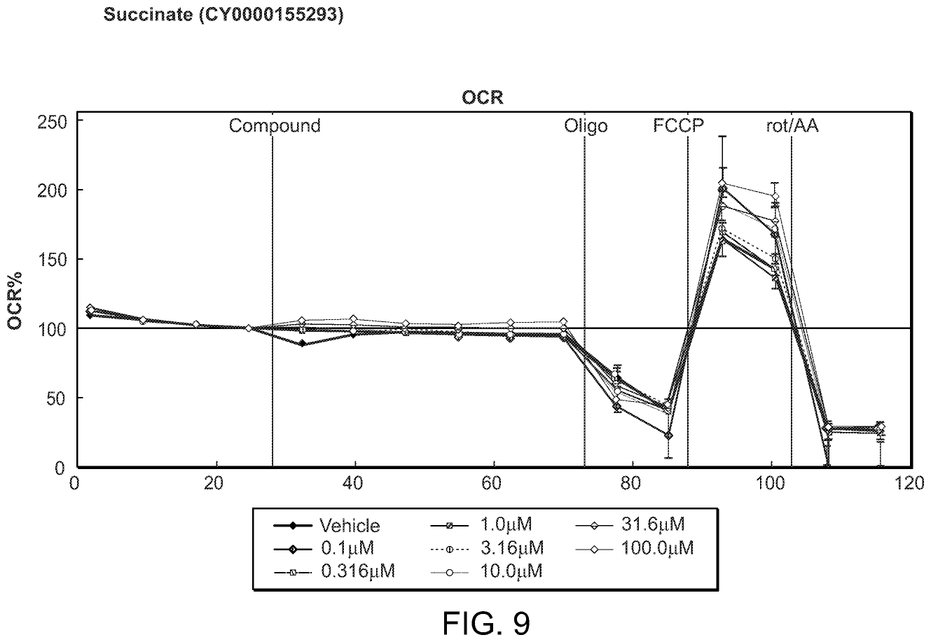

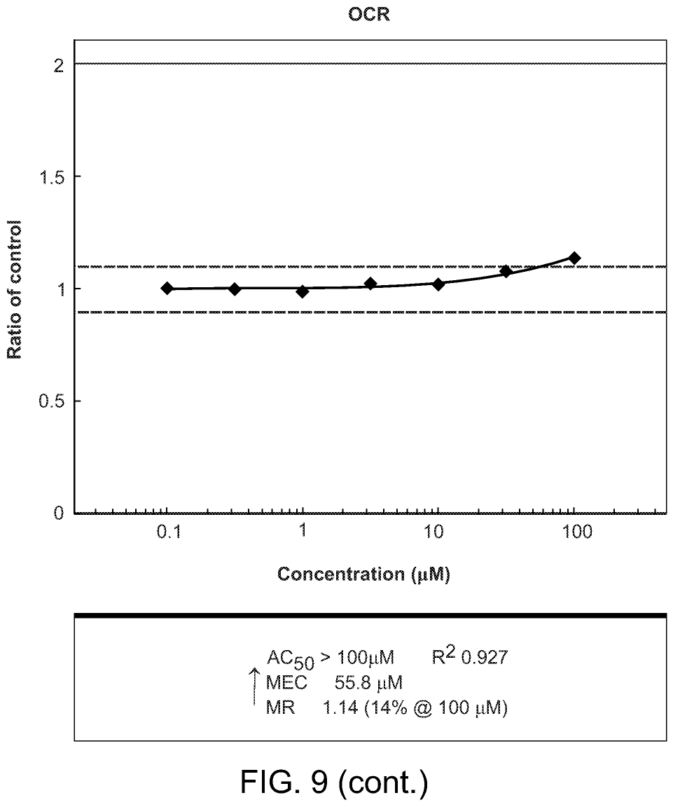

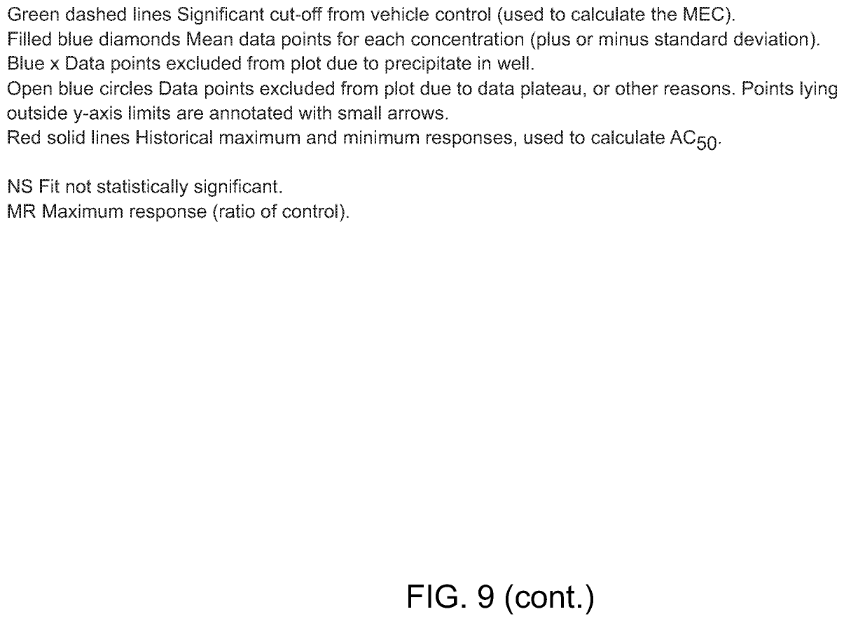

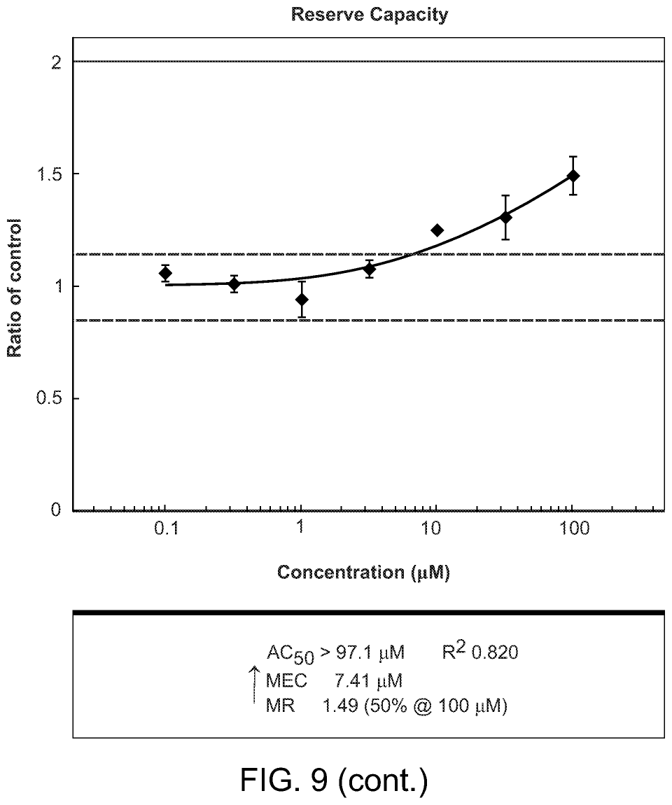

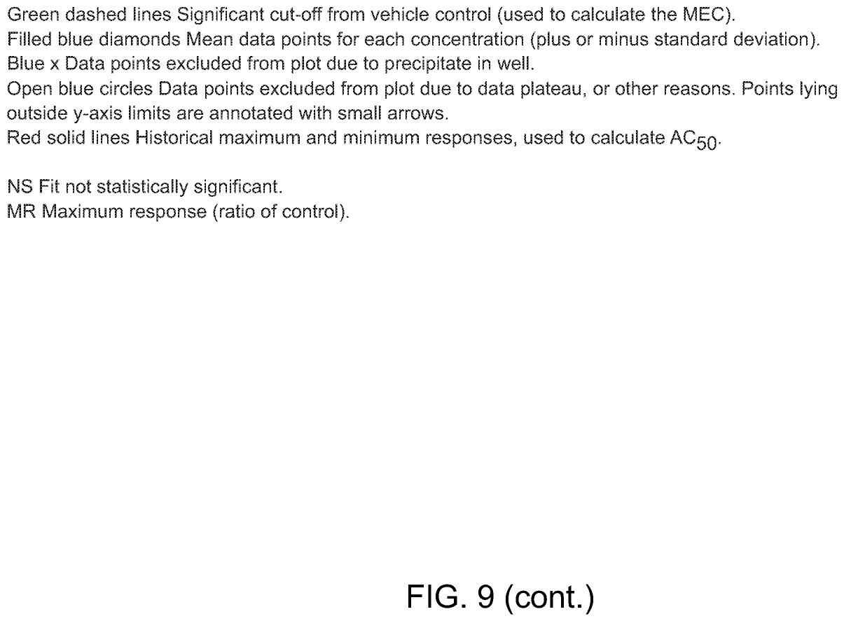

FIG. 9 is a series of graphs showing the effects of succinate on oxygen consumption rate and reserve capacity.

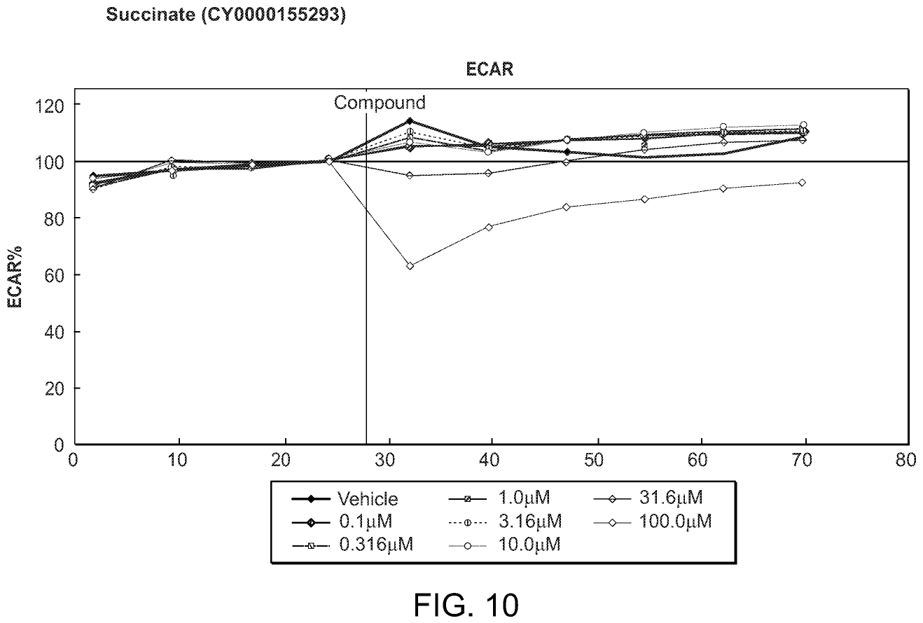

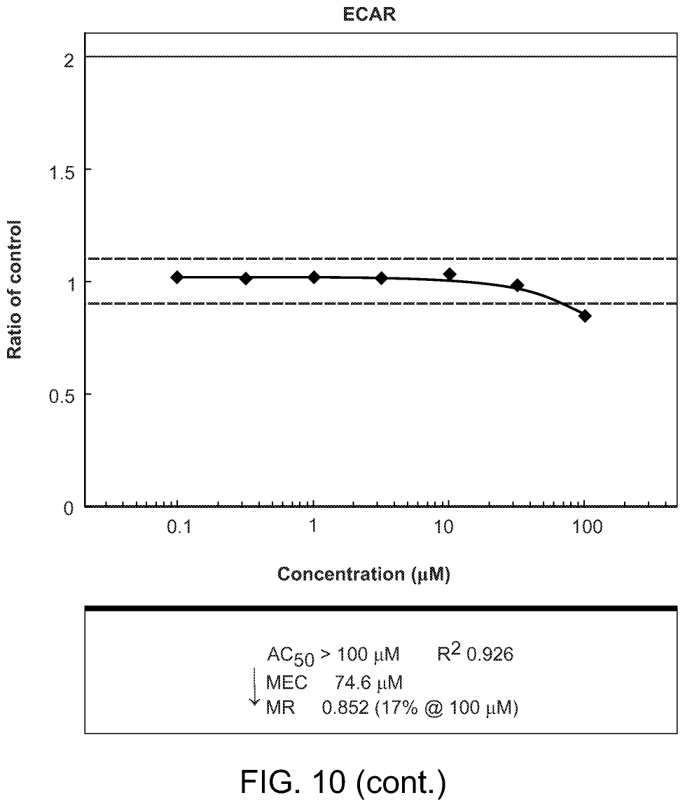

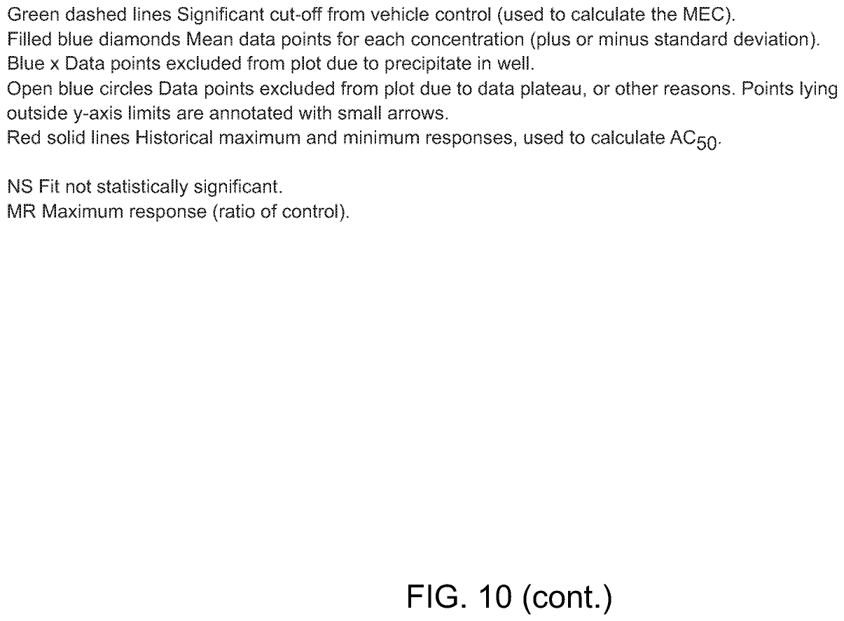

FIG. 10 is a series of graphs showing the effects of succinate on extracellular acidification rate.

FIG. 11 is a table summarizing the effects of compound CV-8816 on various mitochondrial functional parameters.

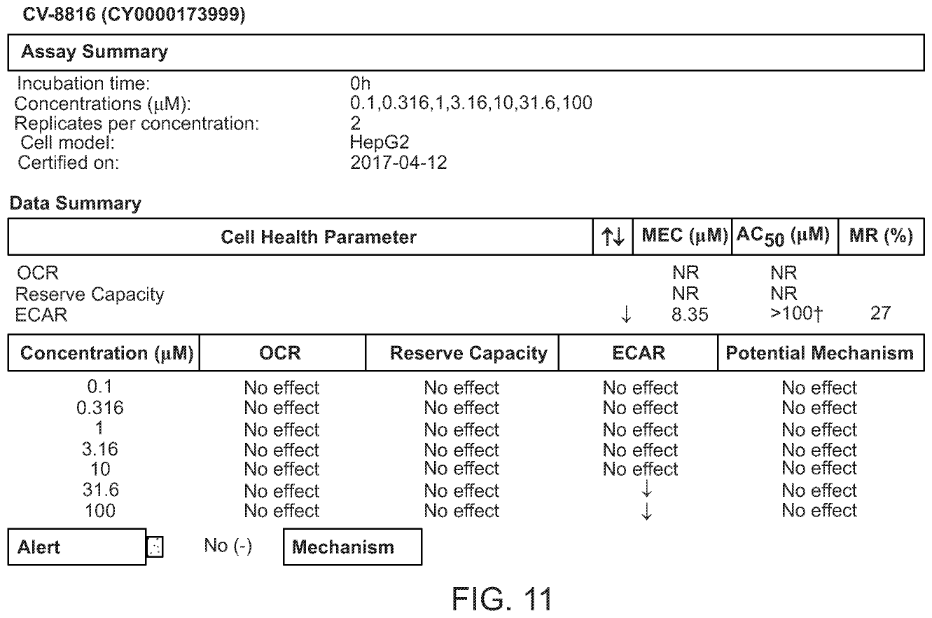

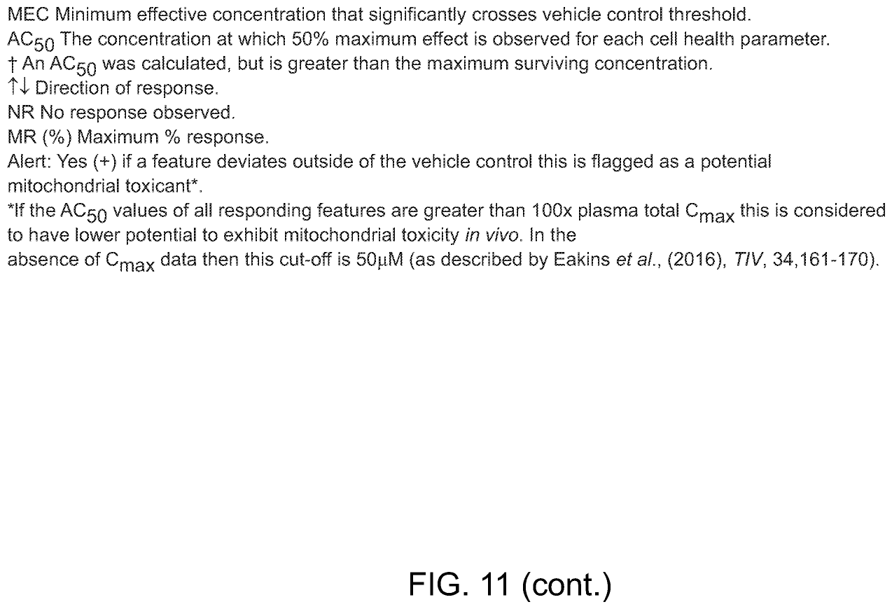

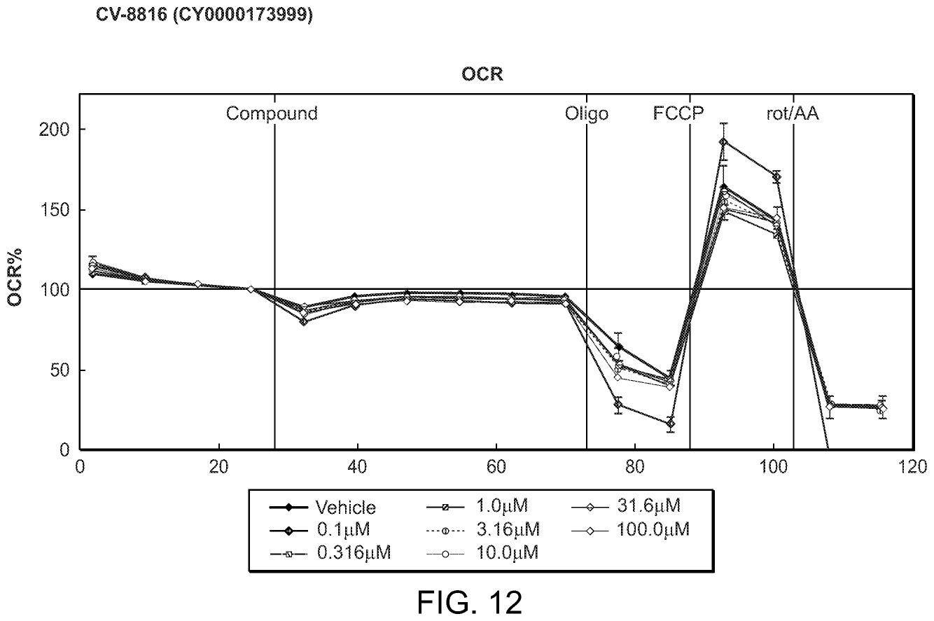

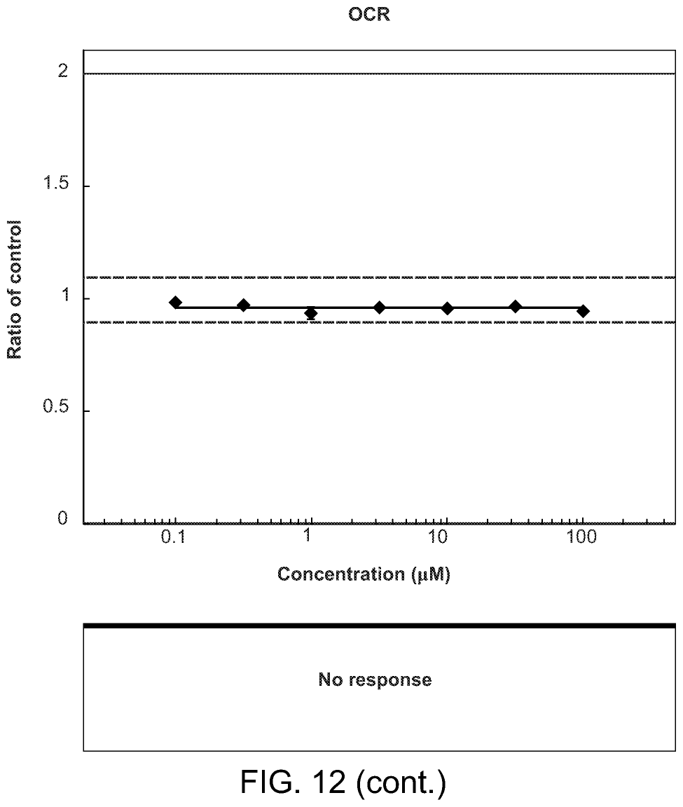

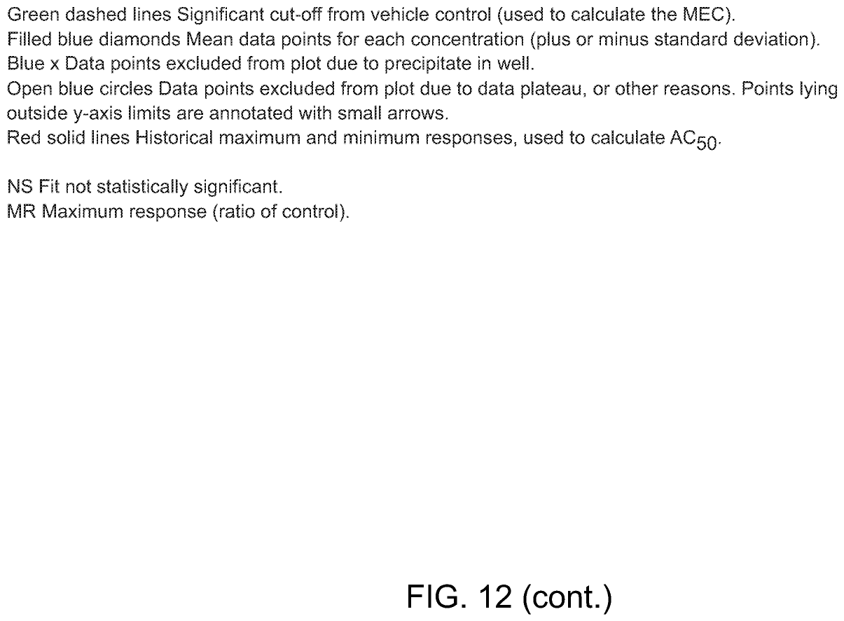

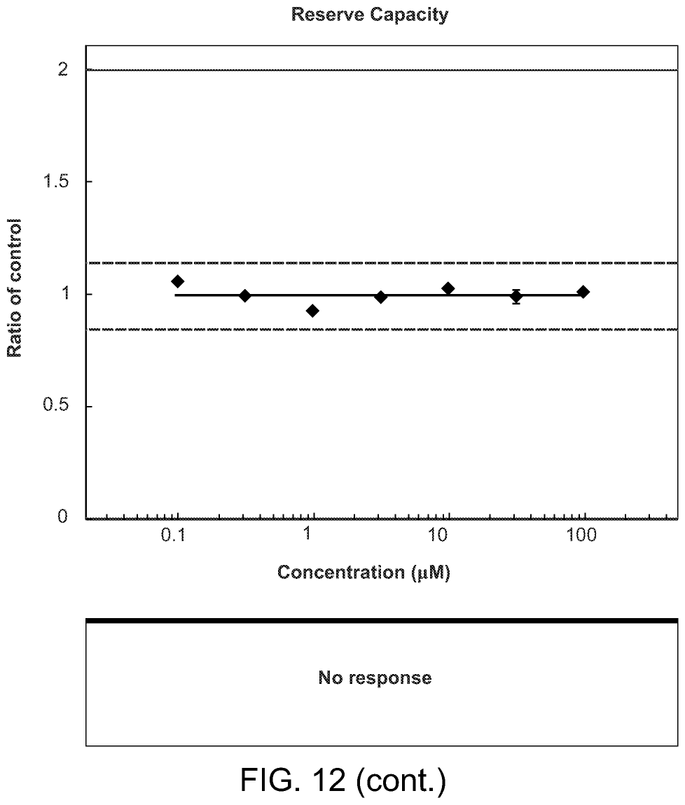

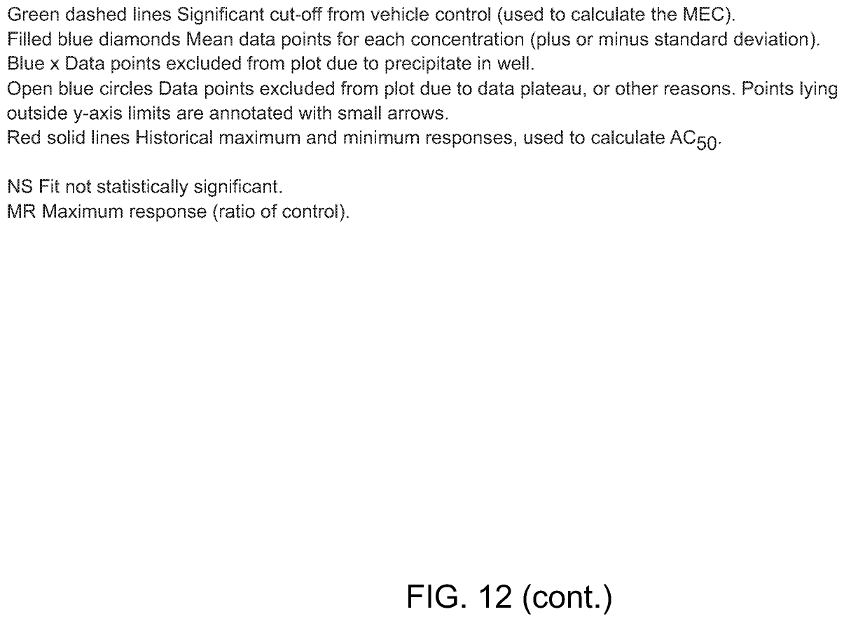

FIG. 12 is a series of graphs showing the effects of compound CV-8816 on oxygen consumption rate and reserve capacity.

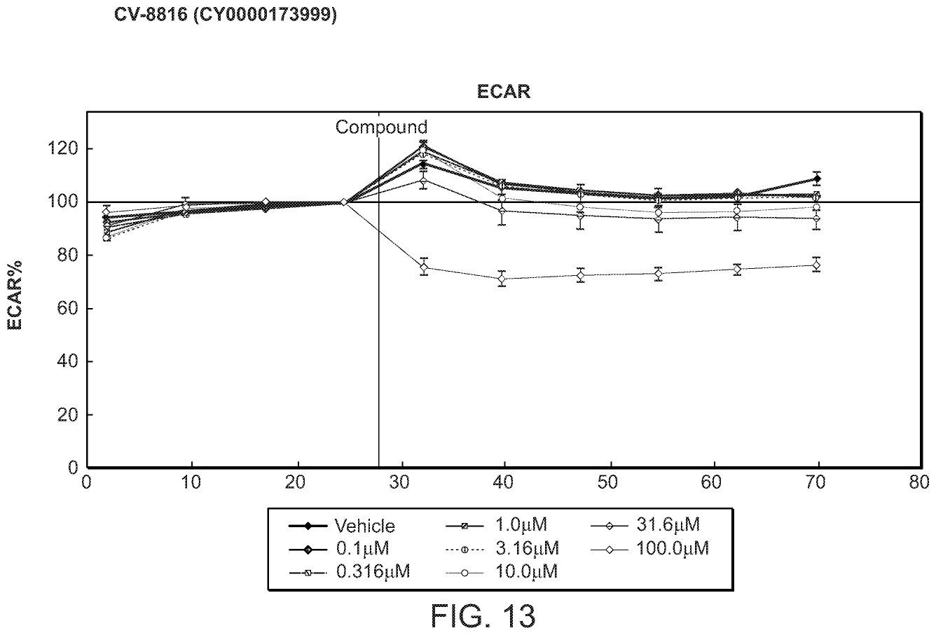

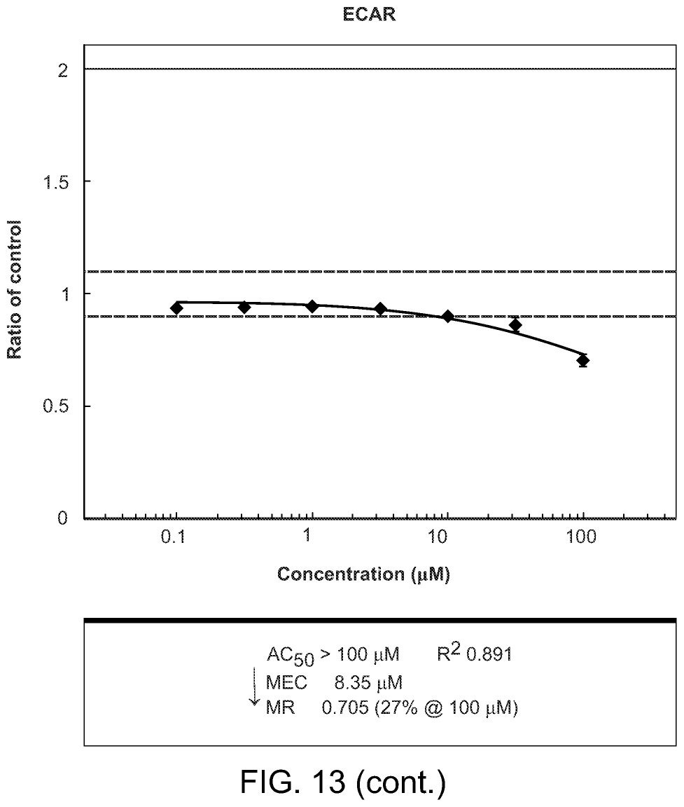

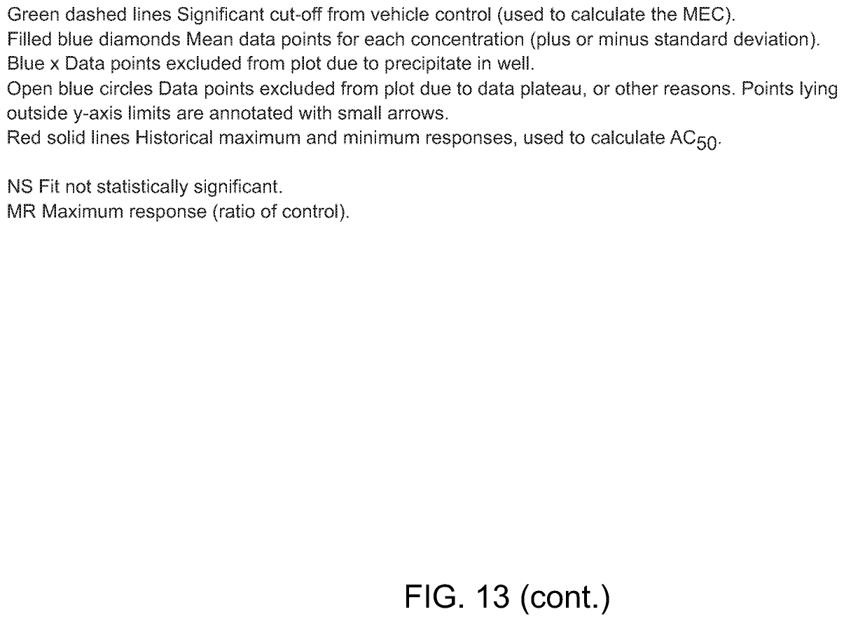

FIG. 13 is a series of graphs showing the effects of compound CV-8816 on extracellular acidification rate.

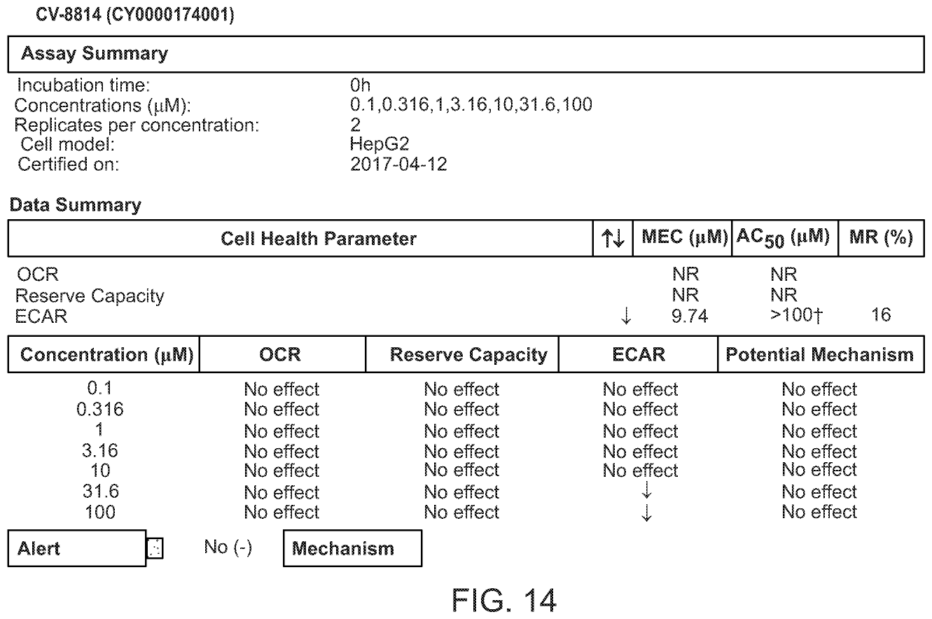

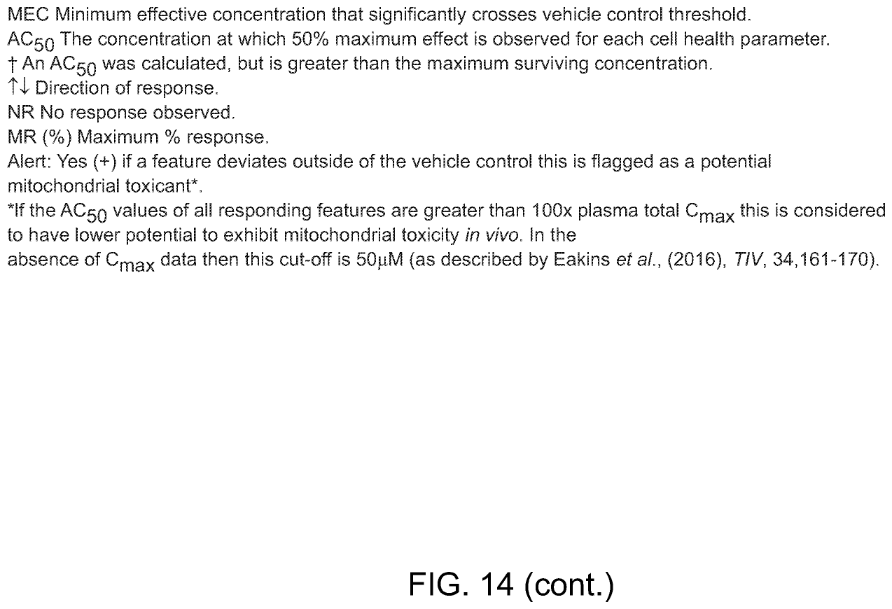

FIG. 14 is a table summarizing the effects of compound CV-8814 on various mitochondrial functional parameters.

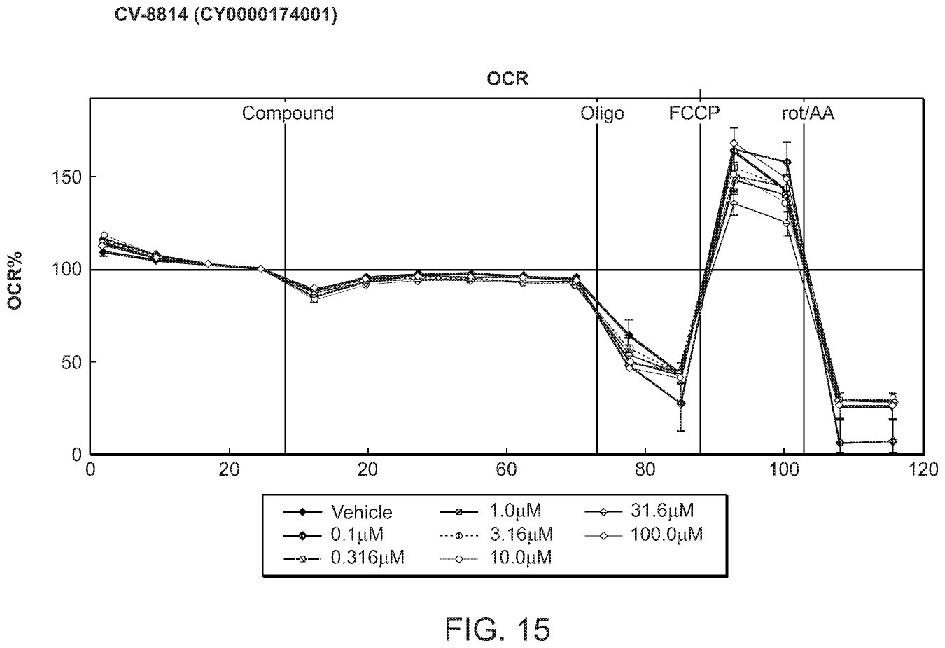

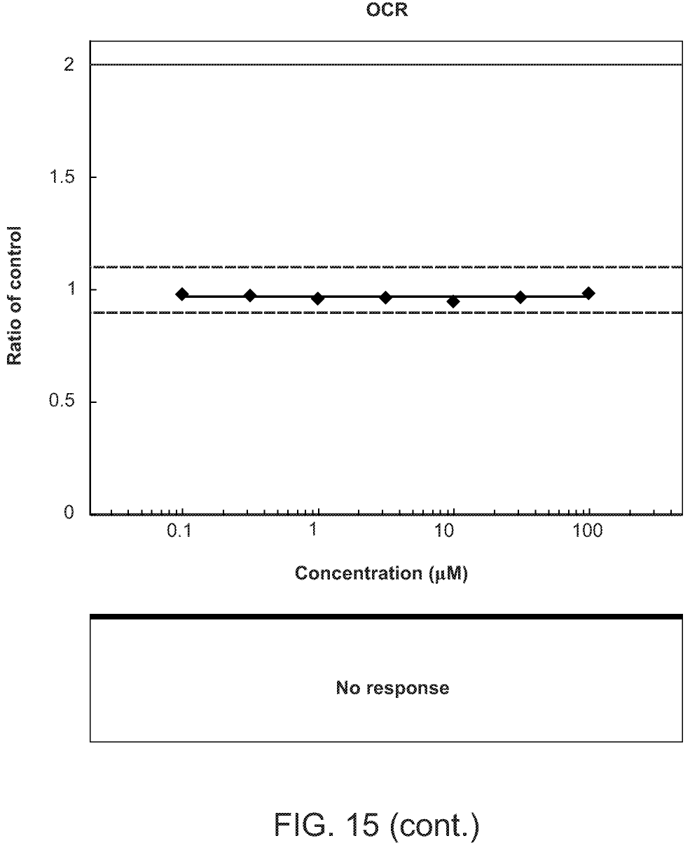

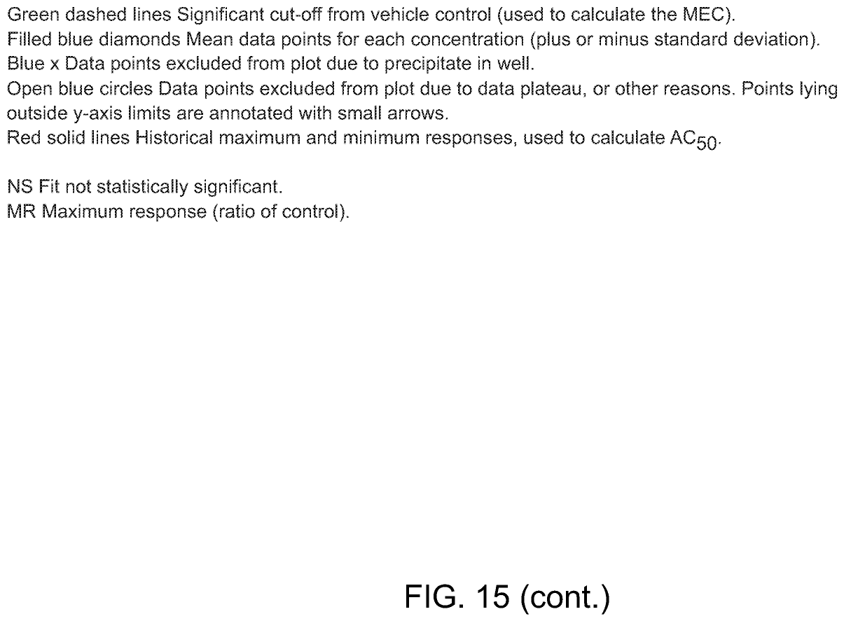

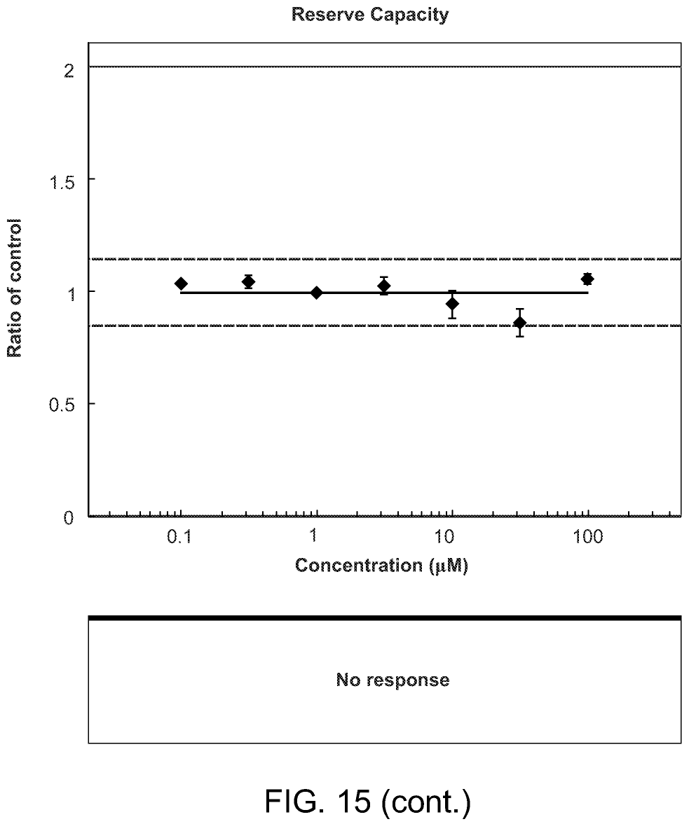

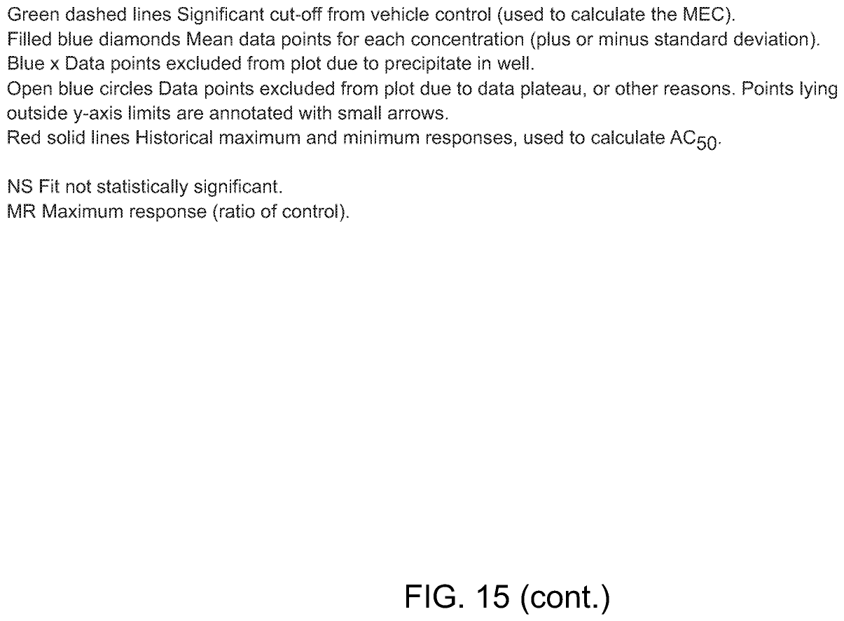

FIG. 15 is a series of graphs showing the effects of compound CV-8814 on oxygen consumption rate and reserve capacity.

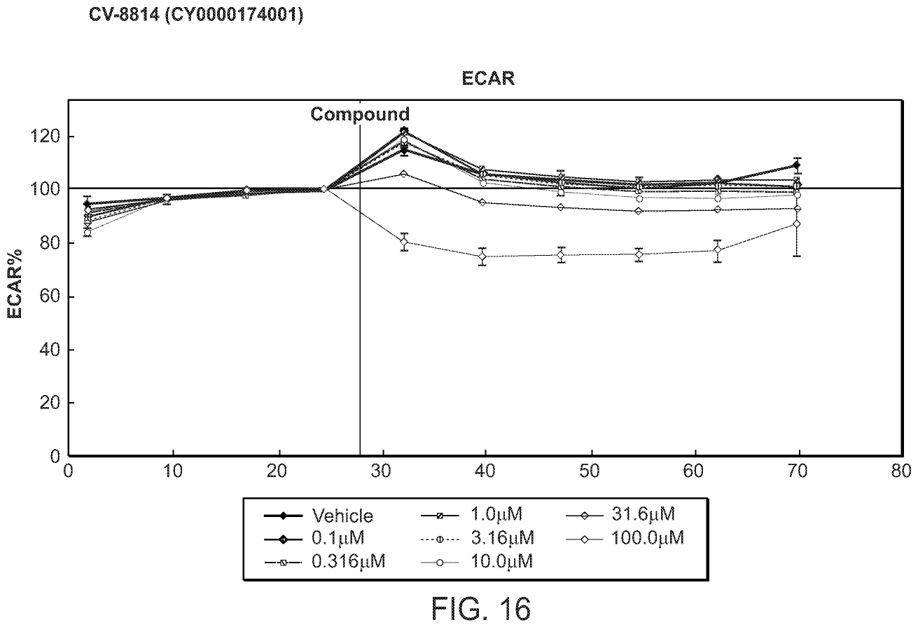

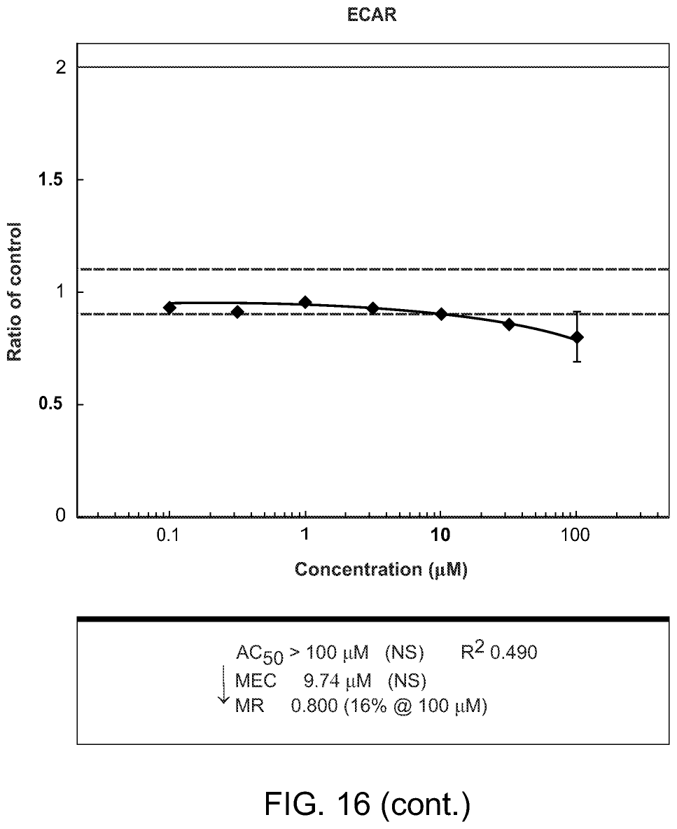

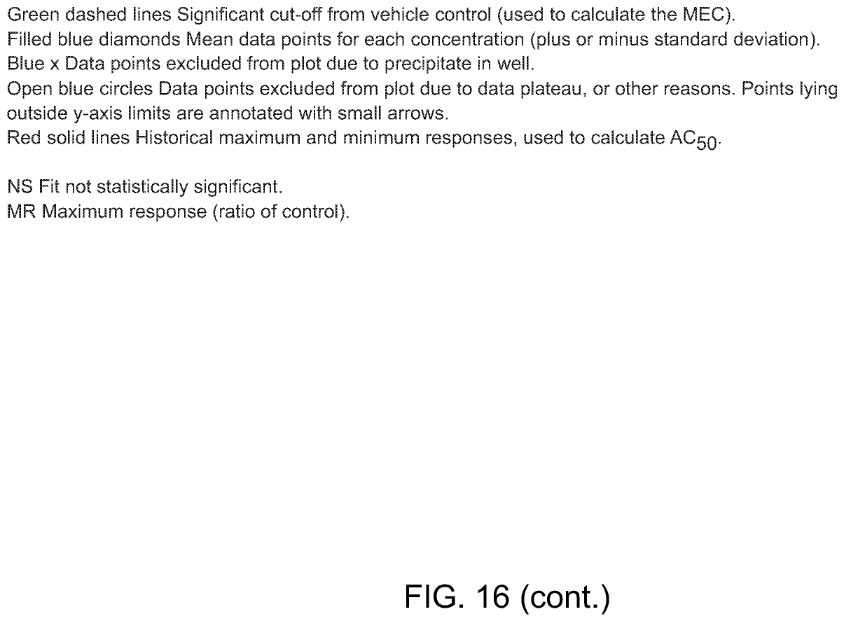

FIG. 16 is a series of graphs showing the effects of compound CV-8814 on extracellular acidification rate.

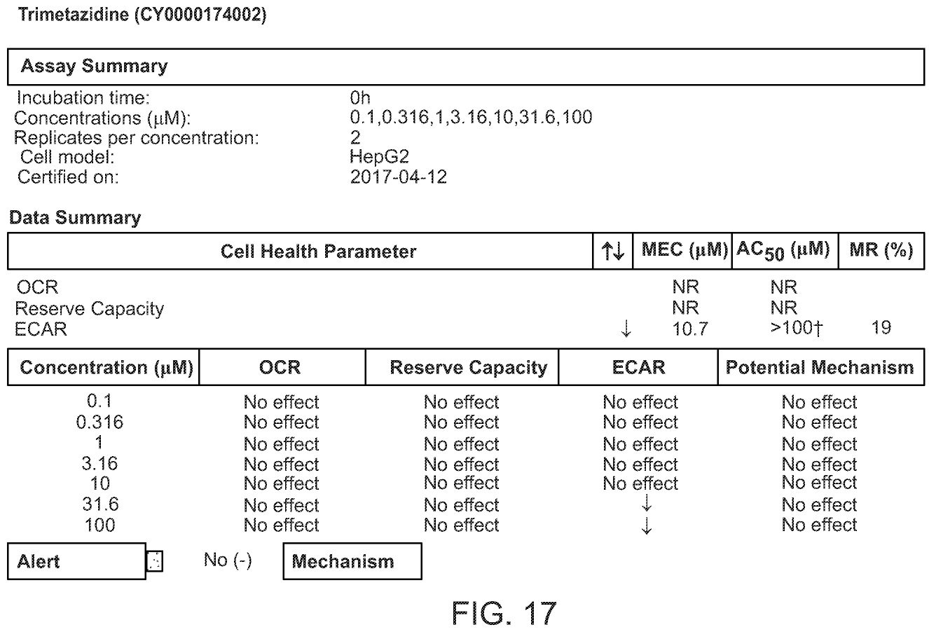

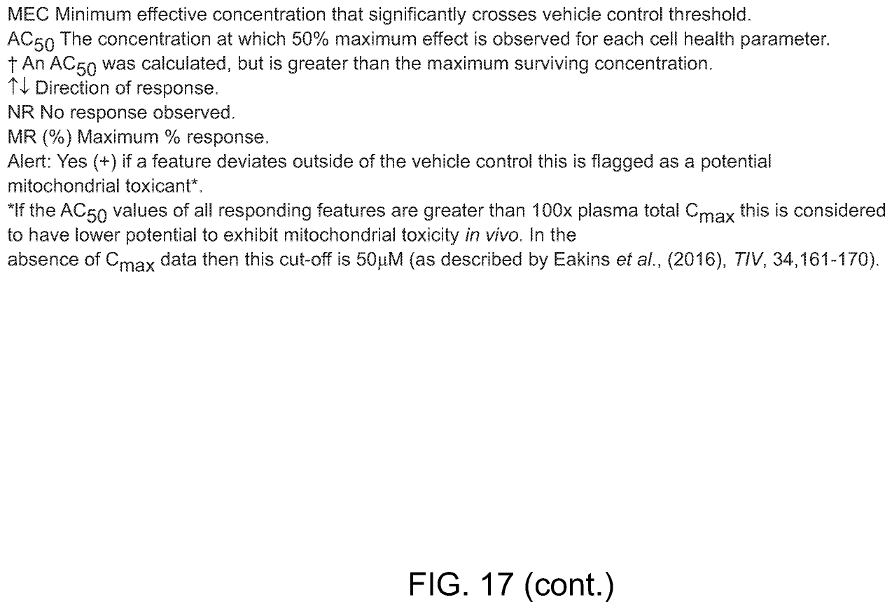

FIG. 17 is a table summarizing the effects of trimetazidine on various mitochondrial functional parameters.

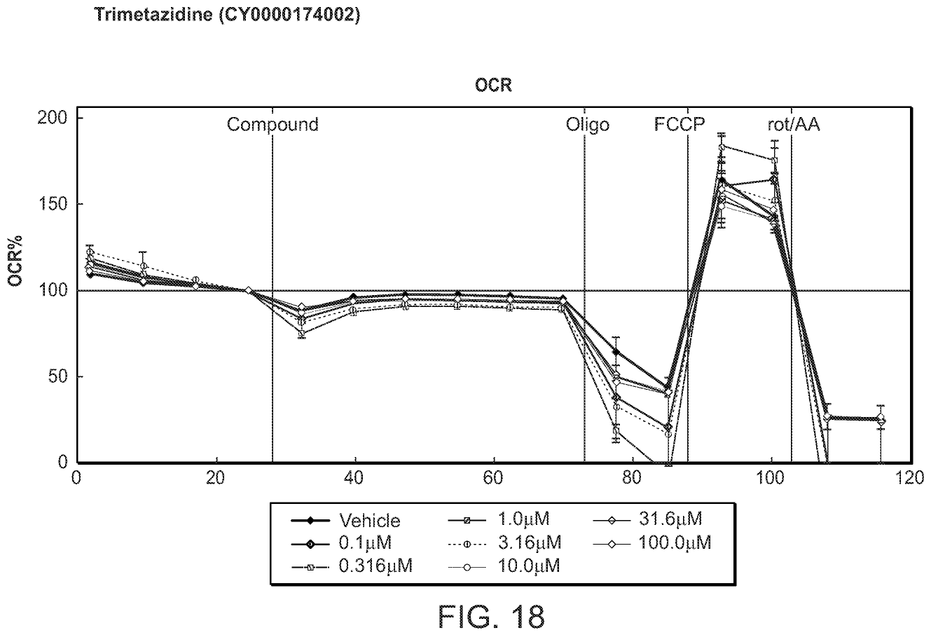

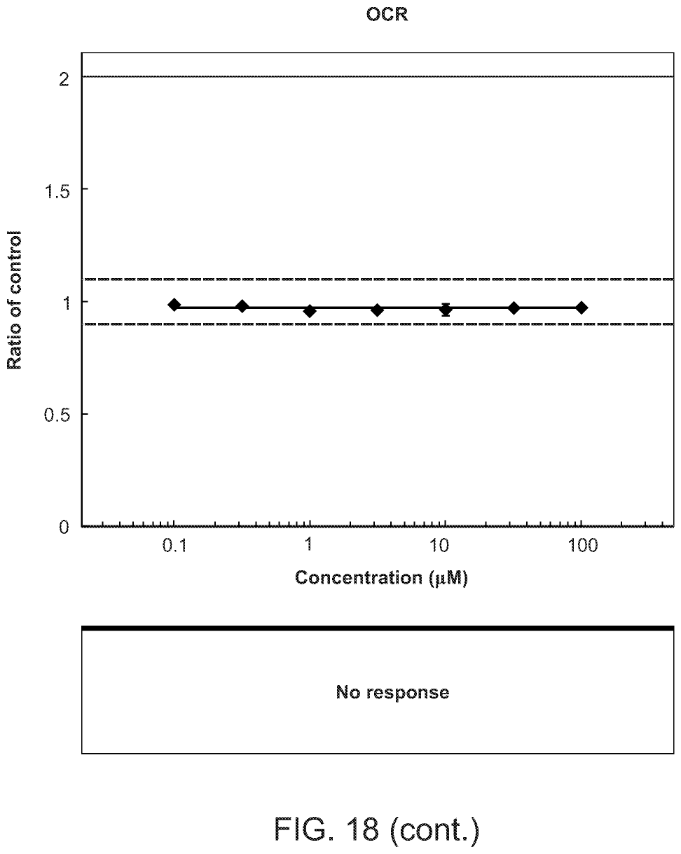

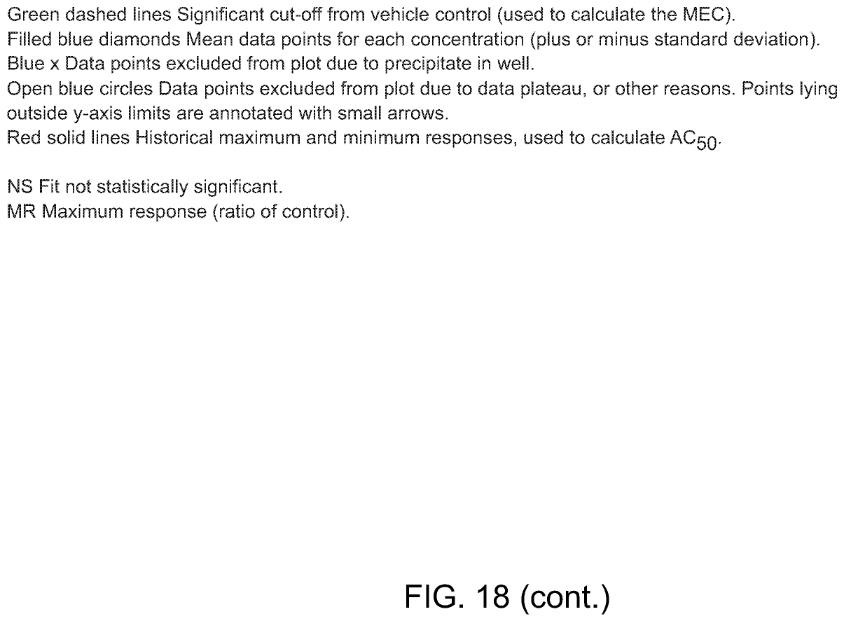

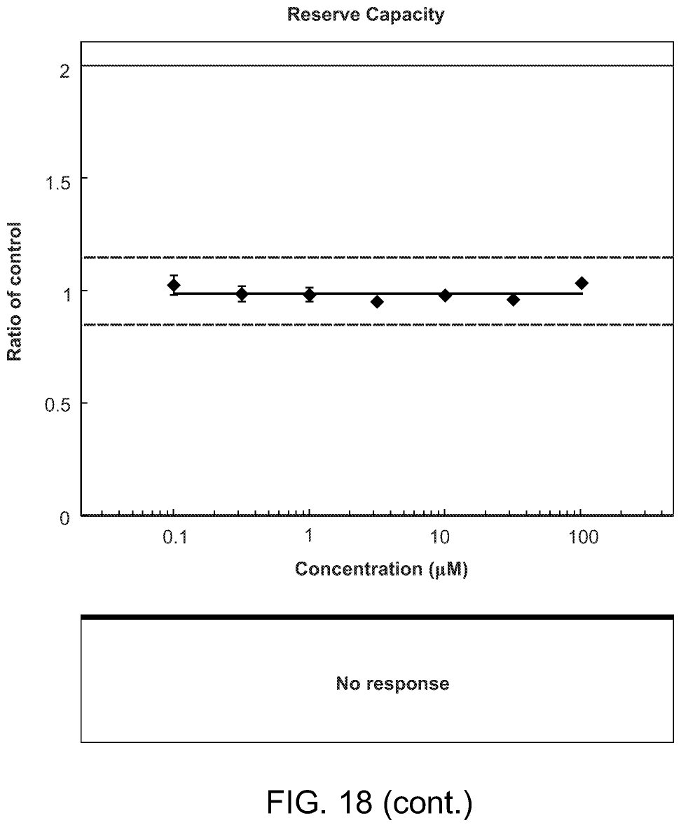

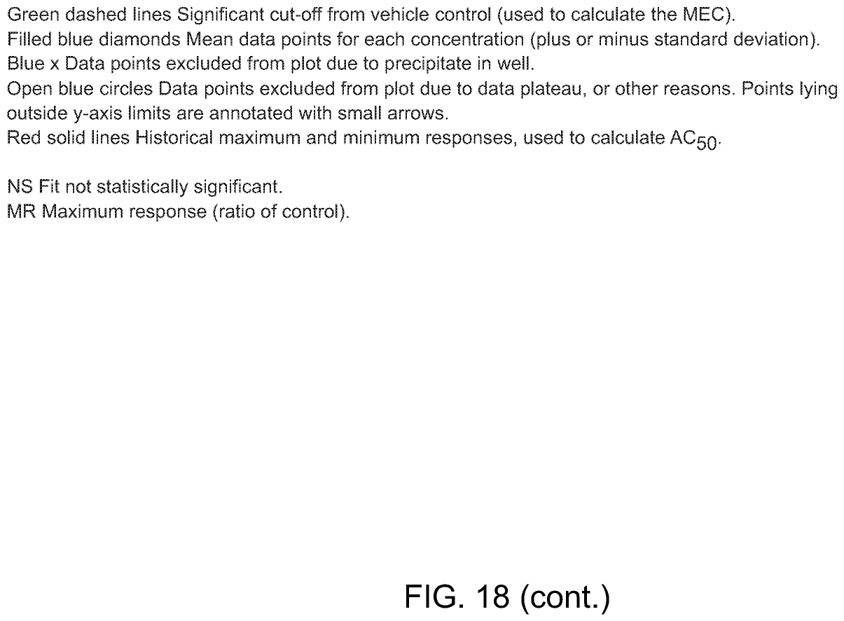

FIG. 18 is a series of graphs showing the effects of trimetazidine on oxygen consumption rate and reserve capacity.

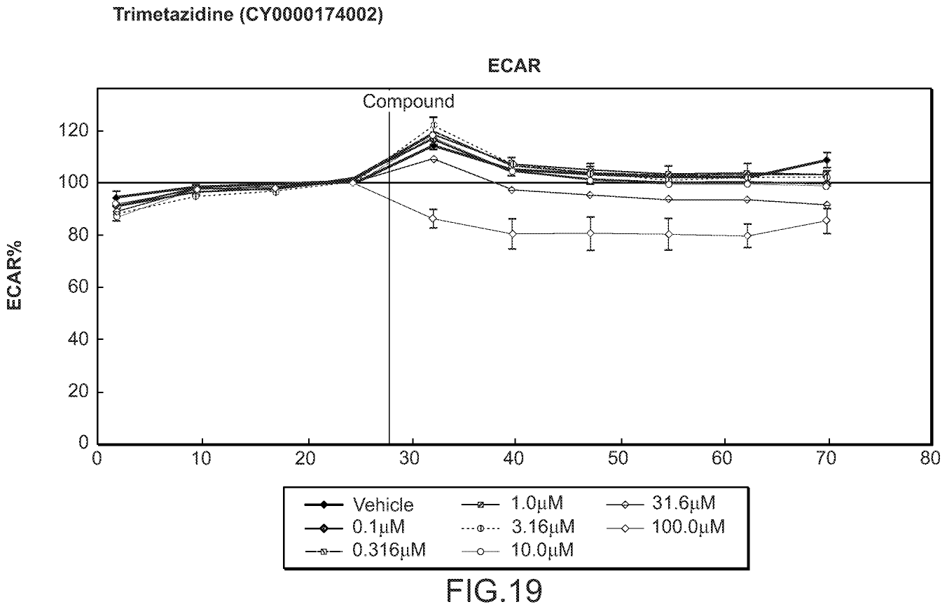

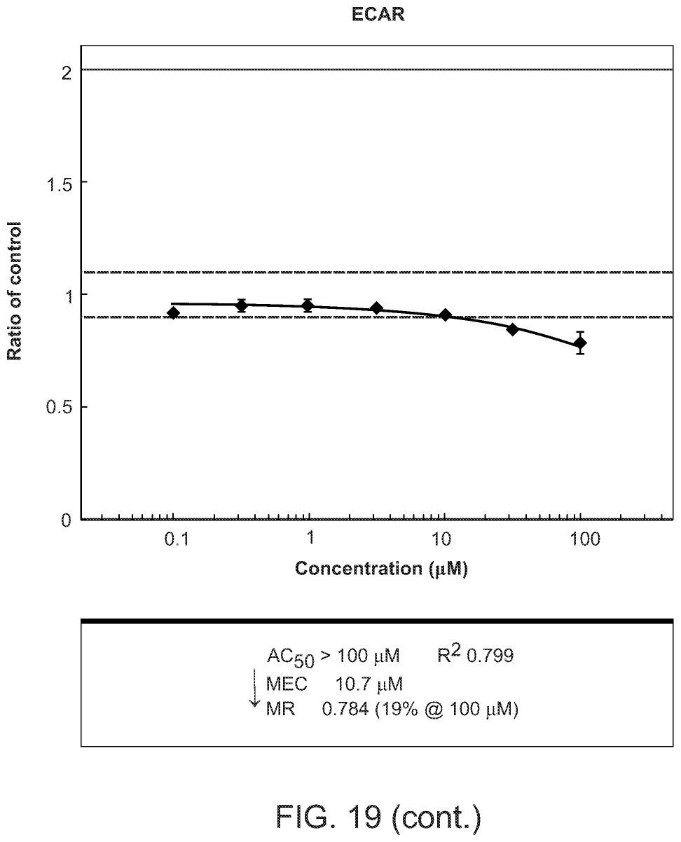

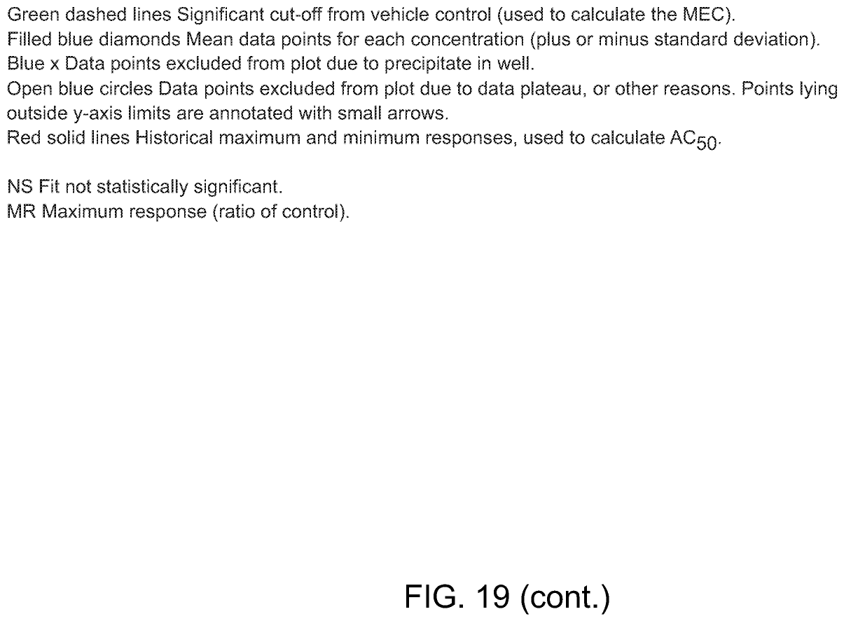

FIG. 19 is a series of graphs showing the effects of trimetazidine on extracellular acidification rate.

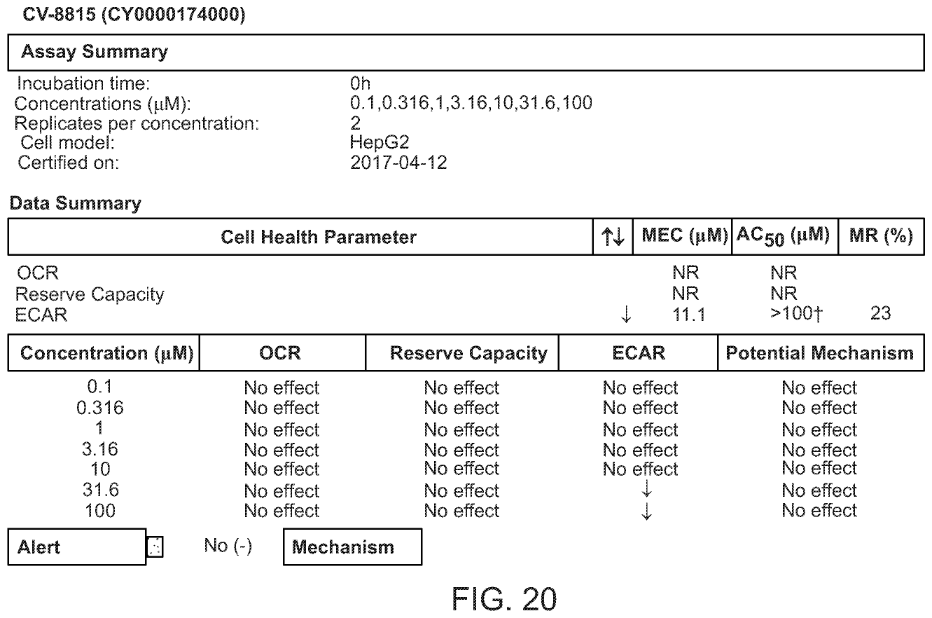

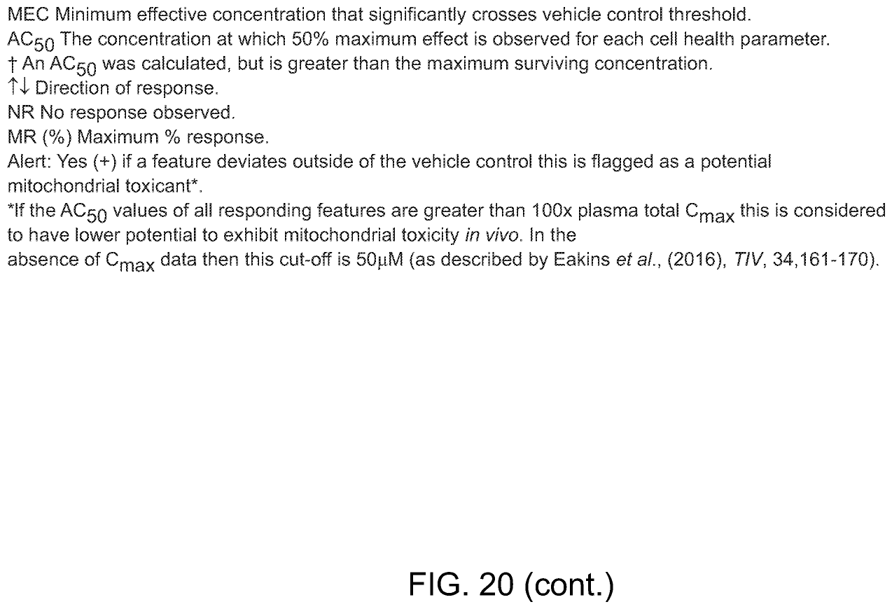

FIG. 20 is a table summarizing the effects of compound CV-8815 on various mitochondrial functional parameters.

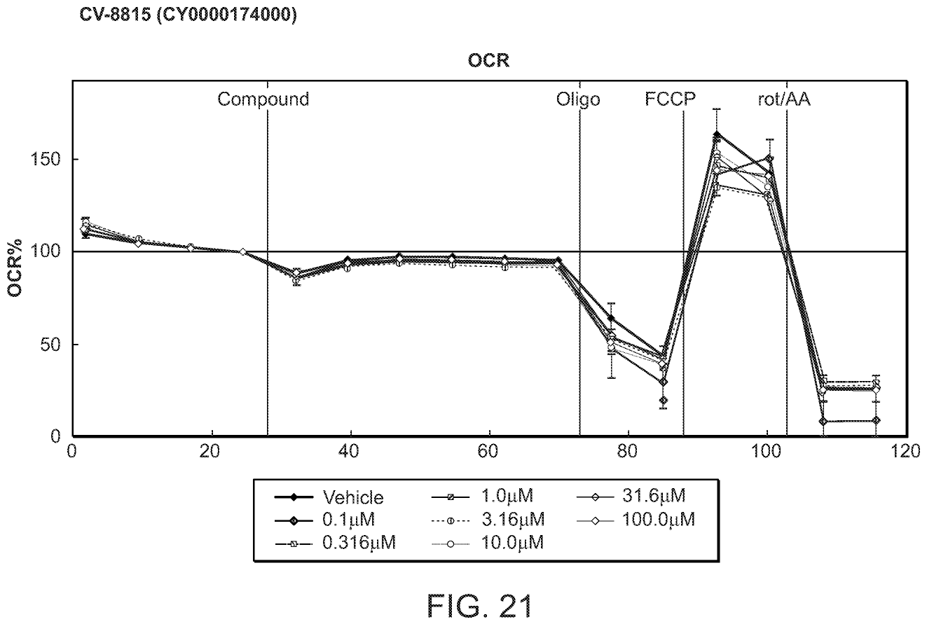

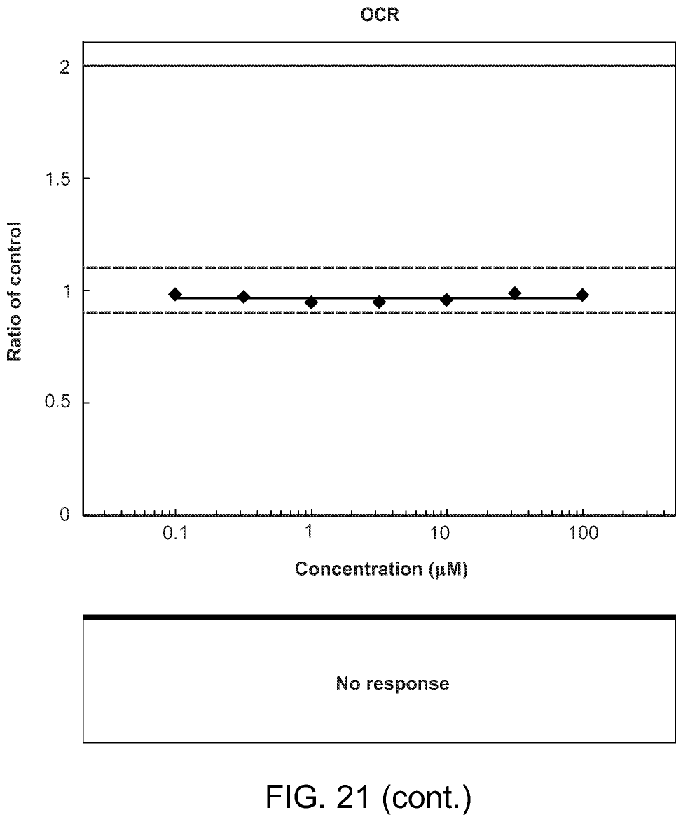

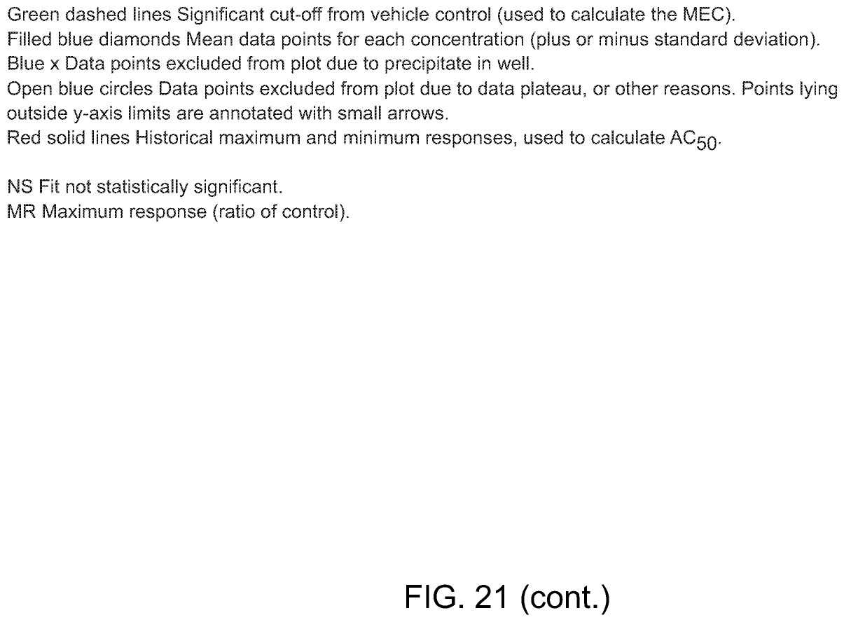

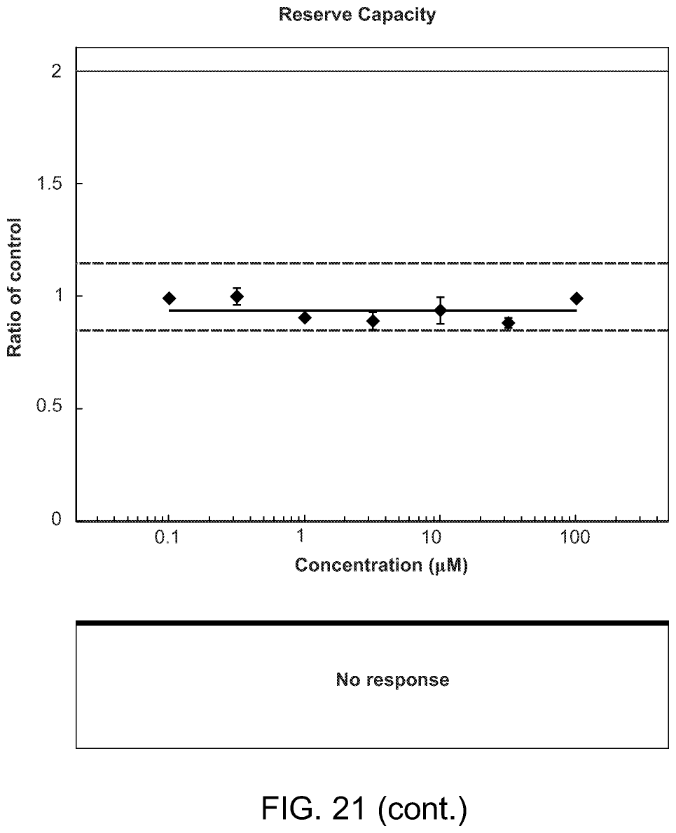

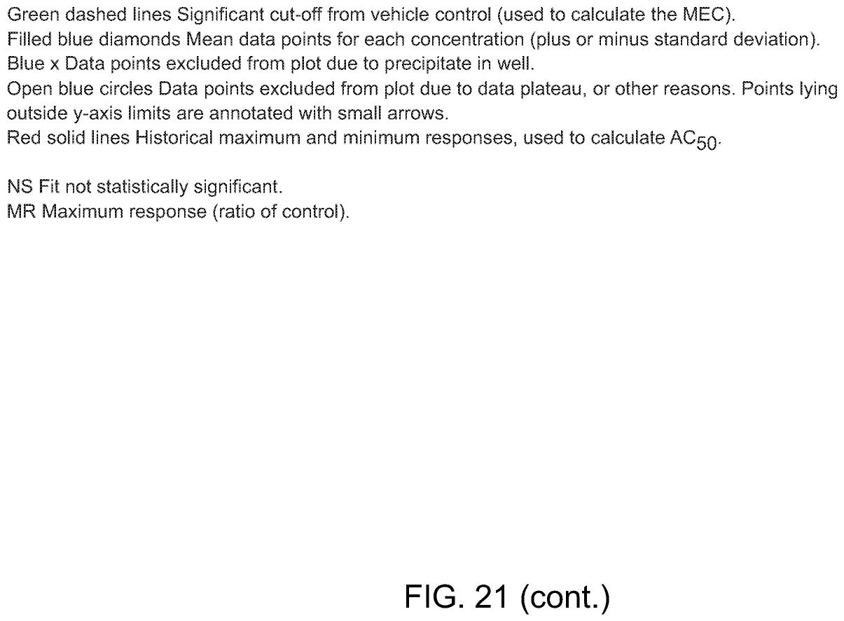

FIG. 21 is a series of graphs showing the effects of compound CV-8815 on oxygen consumption rate and reserve capacity.

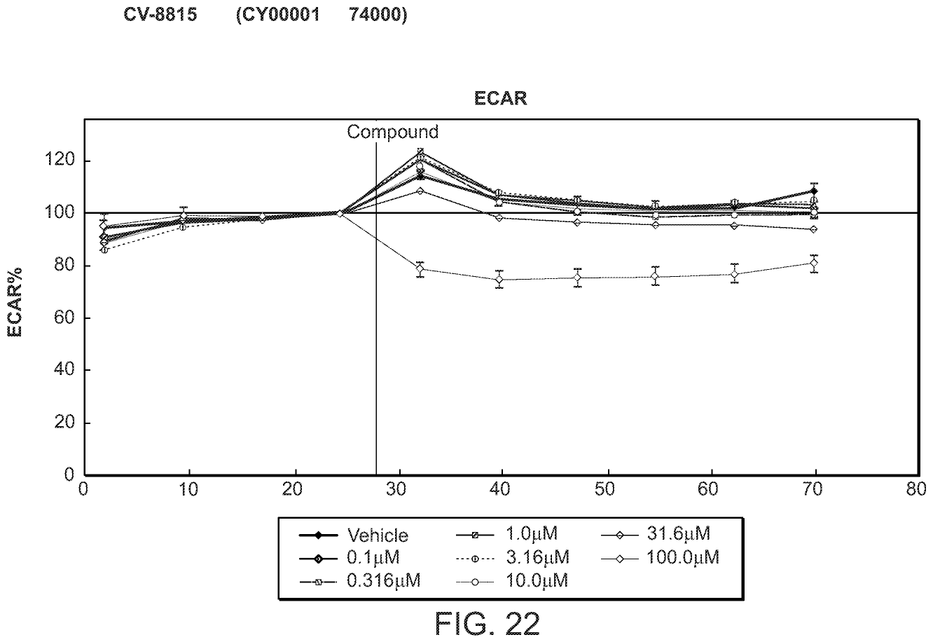

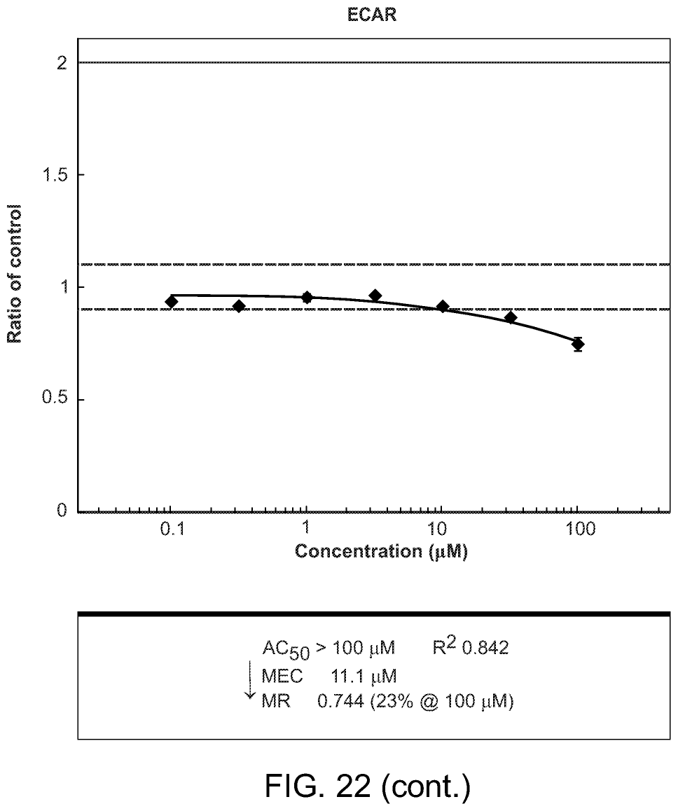

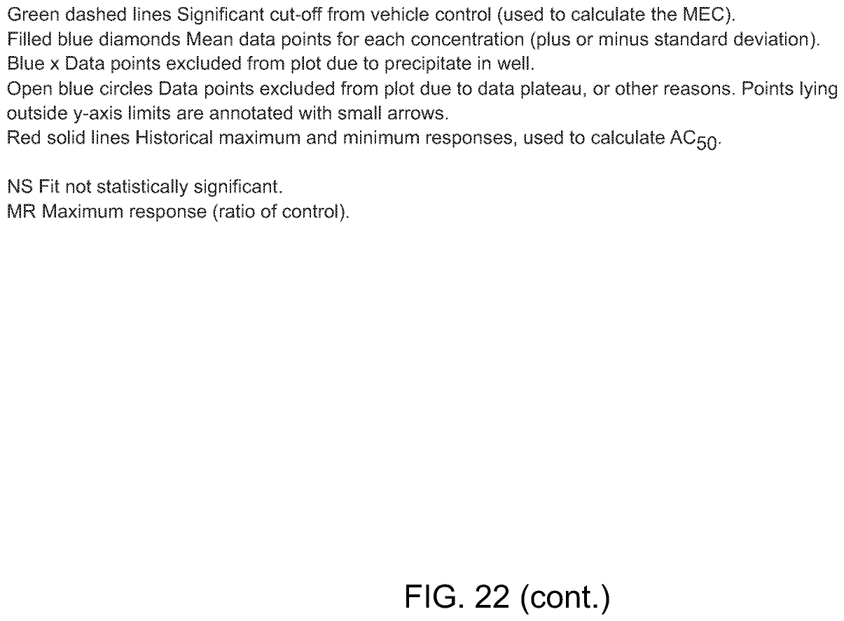

FIG. 22 is a series of graphs showing the effects of compound CV-8815 on extracellular acidification rate.

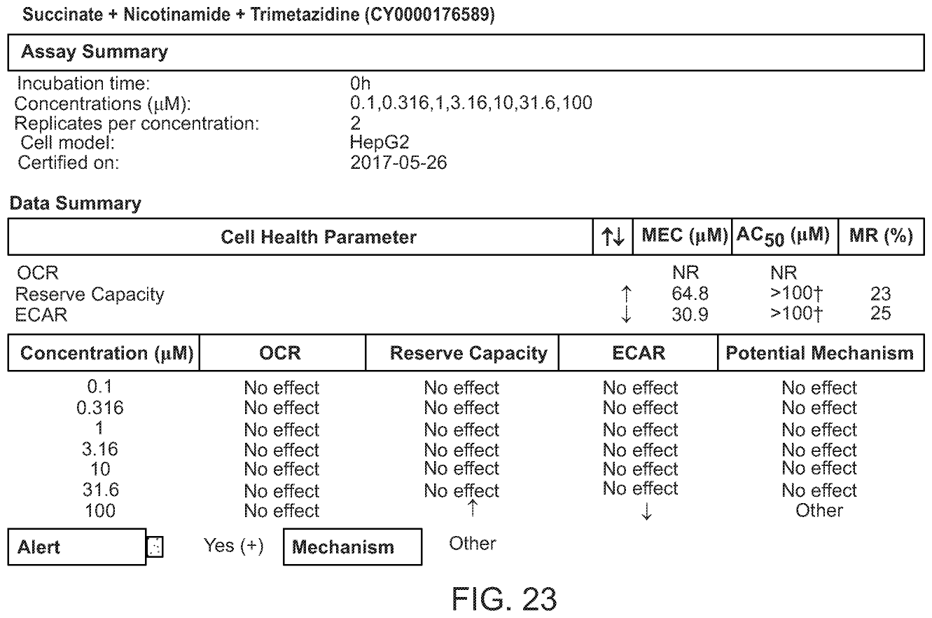

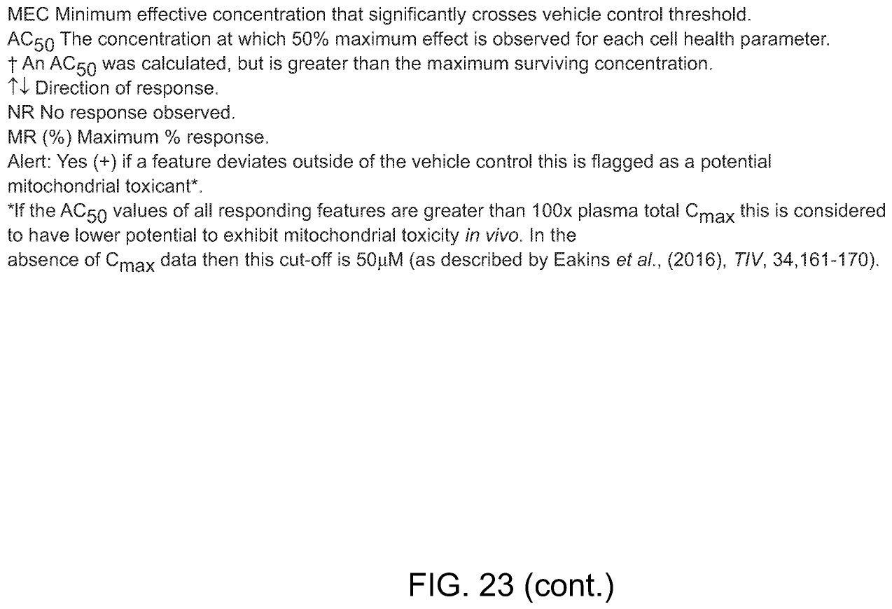

FIG. 23 is a table summarizing the effects of a combination of succinate, nicotinamide, and trimetazidine on various mitochondrial functional parameters.

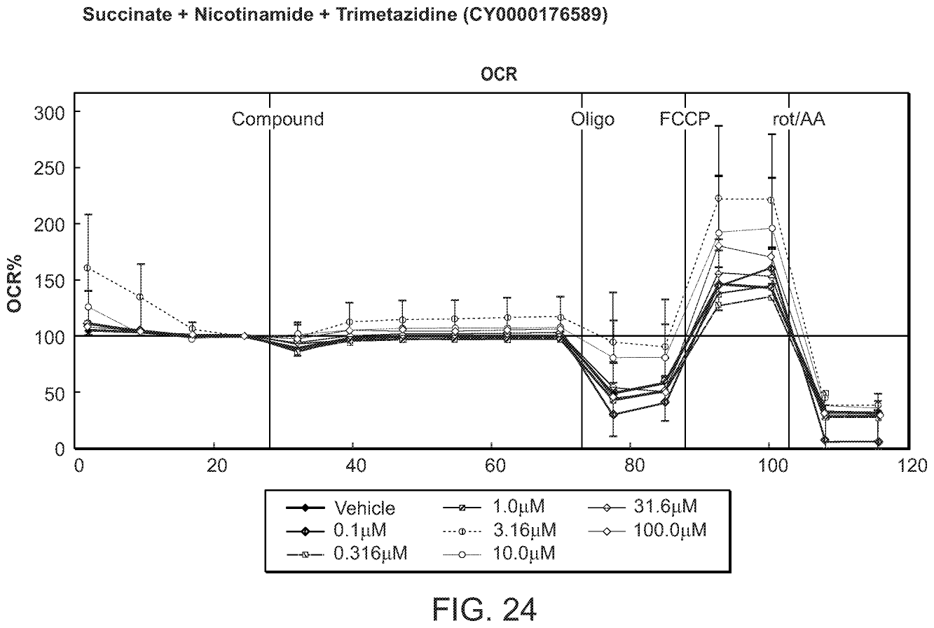

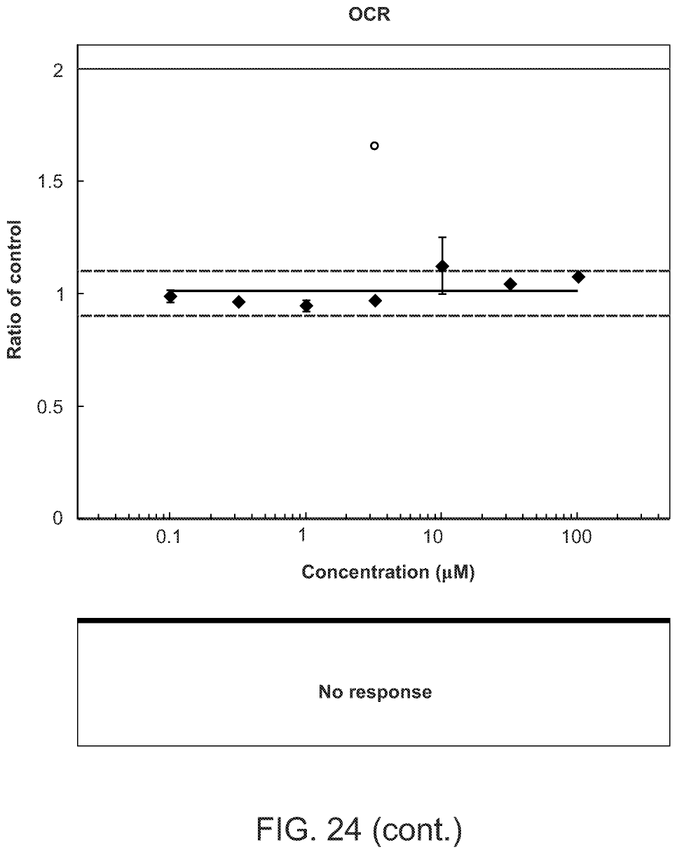

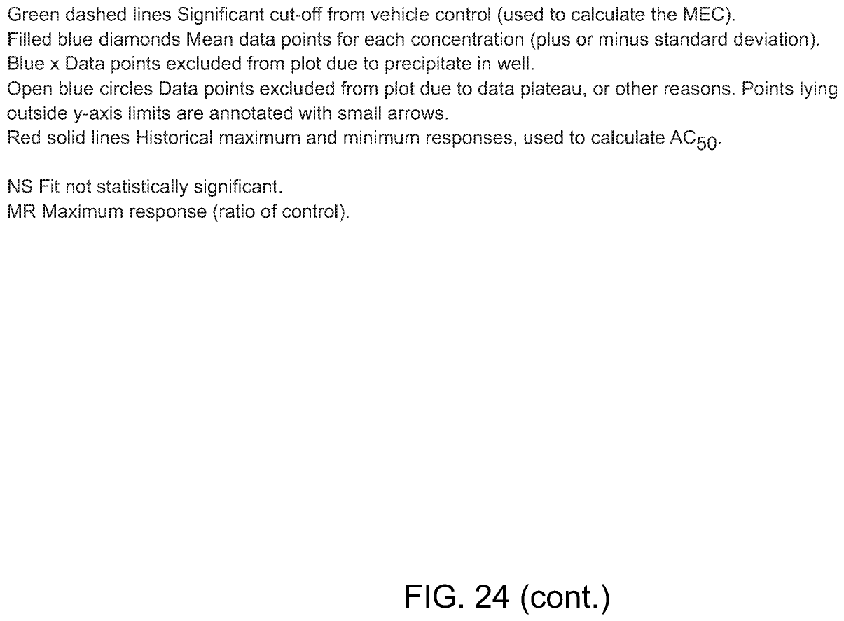

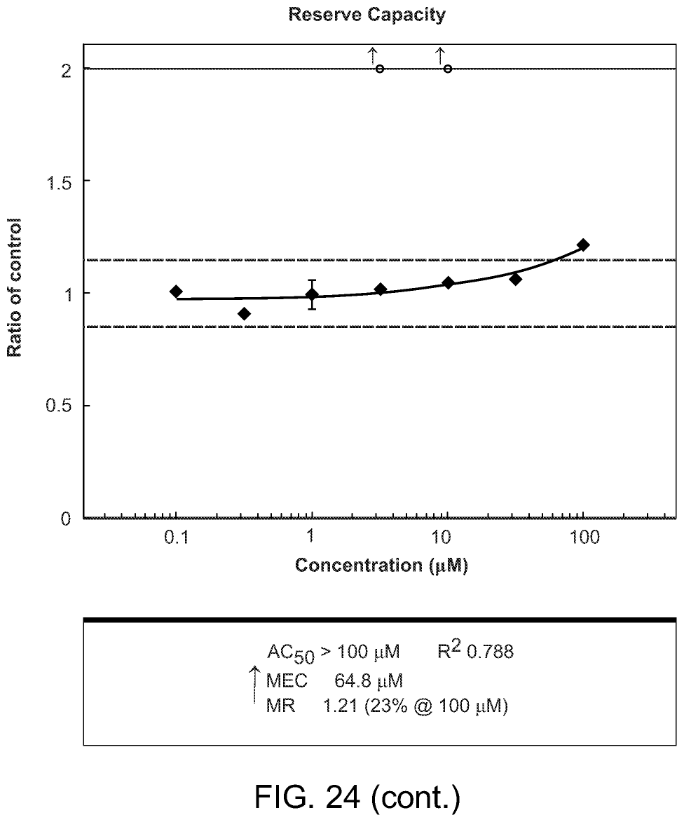

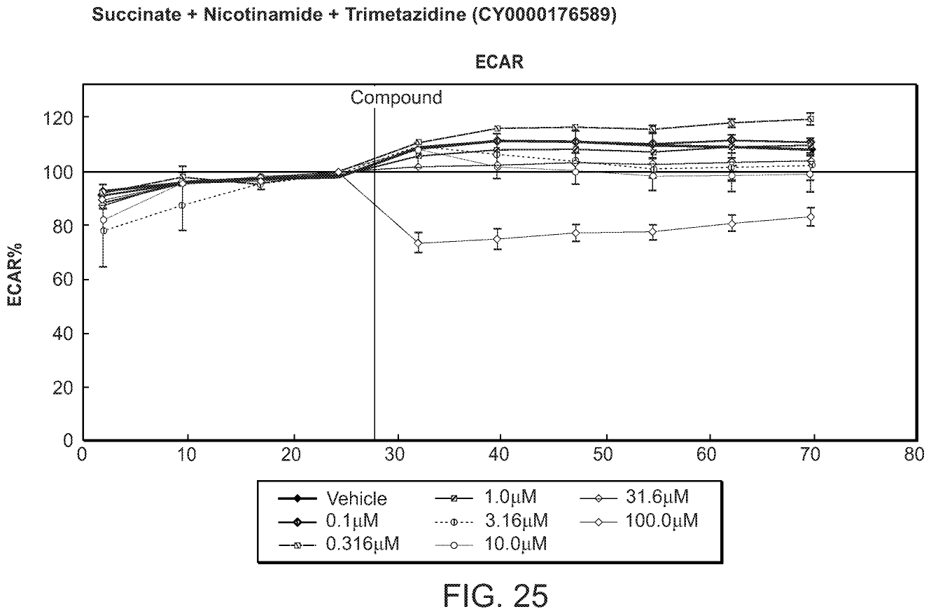

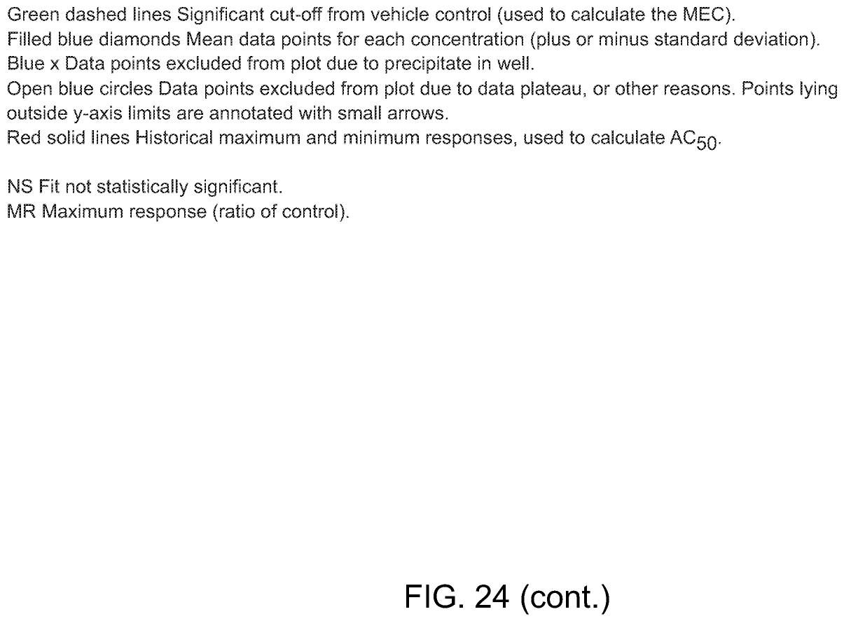

FIG. 24 is a series of graphs showing the effects of a combination of succinate, nicotinamide, and trimetazidine on oxygen consumption rate and reserve capacity.

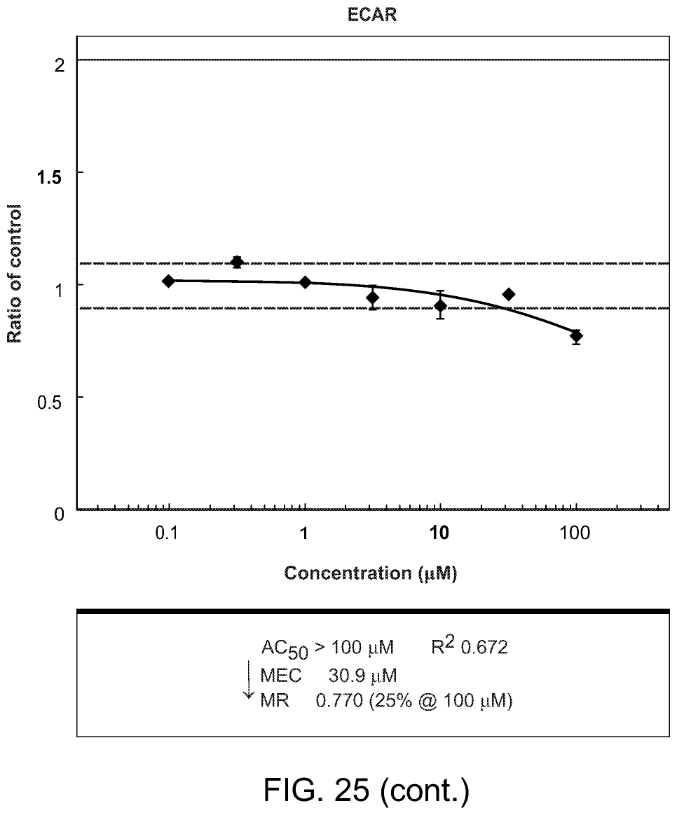

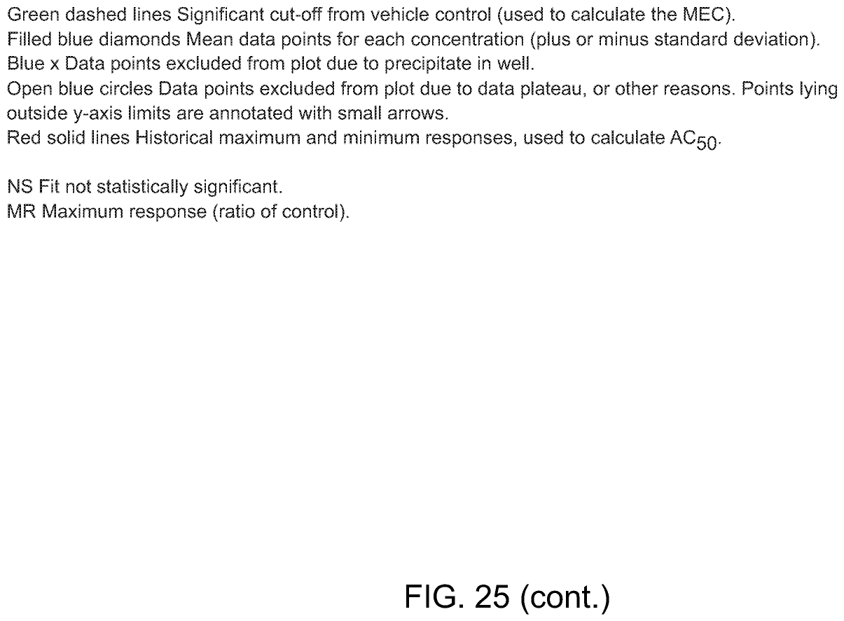

FIG. 25 is a series of graphs showing the effects of a combination of succinate, nicotinamide, and trimetazidine on extracellular acidification rate.

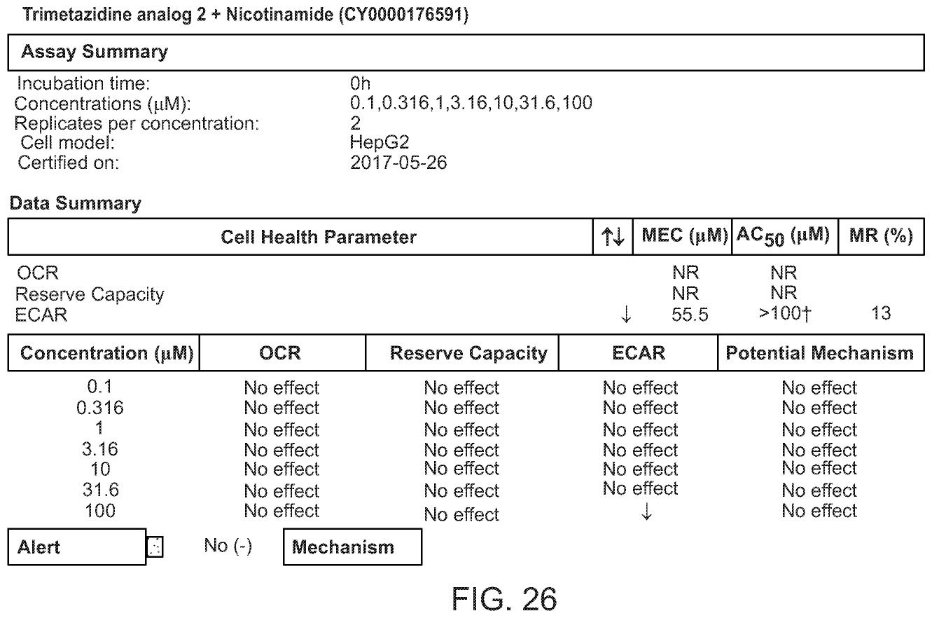

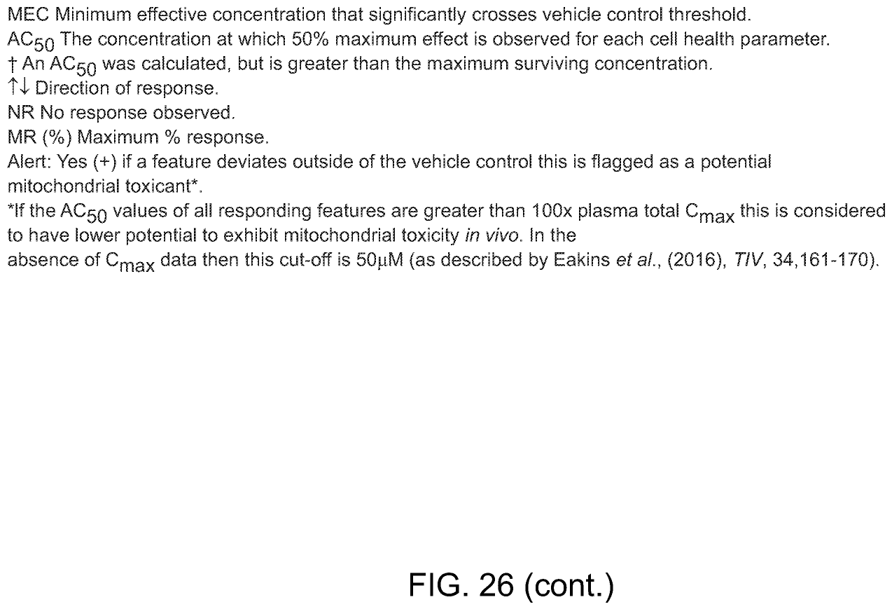

FIG. 26 is a table summarizing the effects of a combination of trimetazidine analog 2 and nicotinamide on various mitochondrial functional parameters.

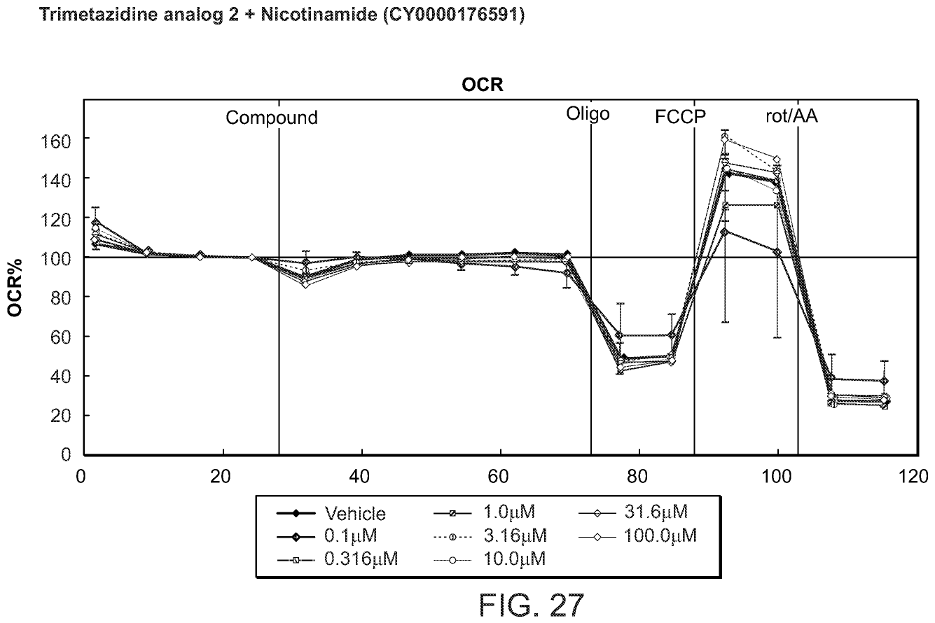

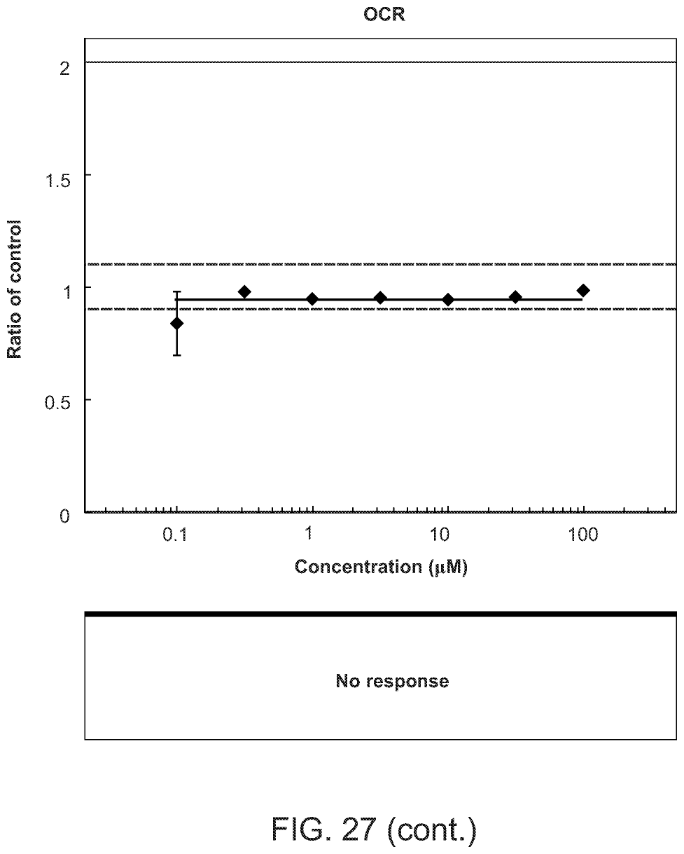

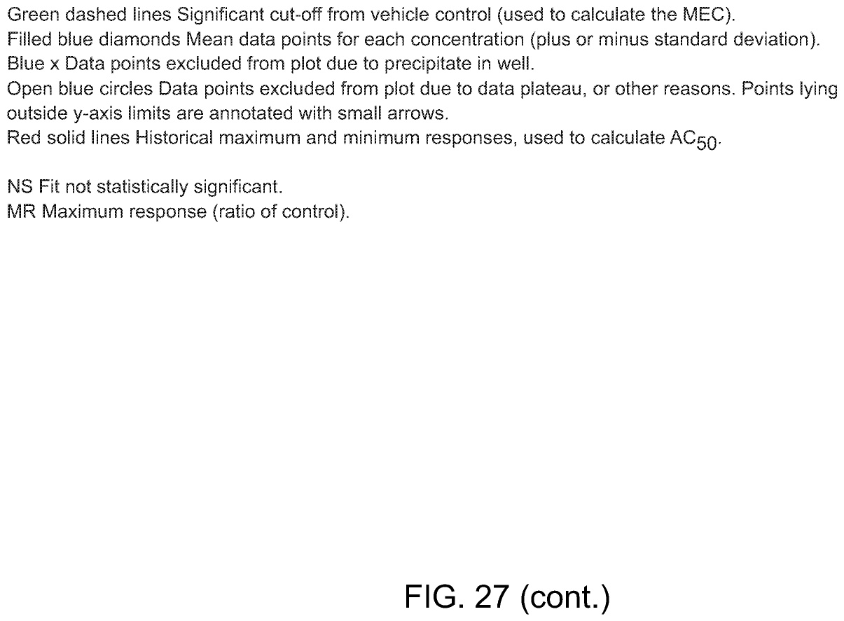

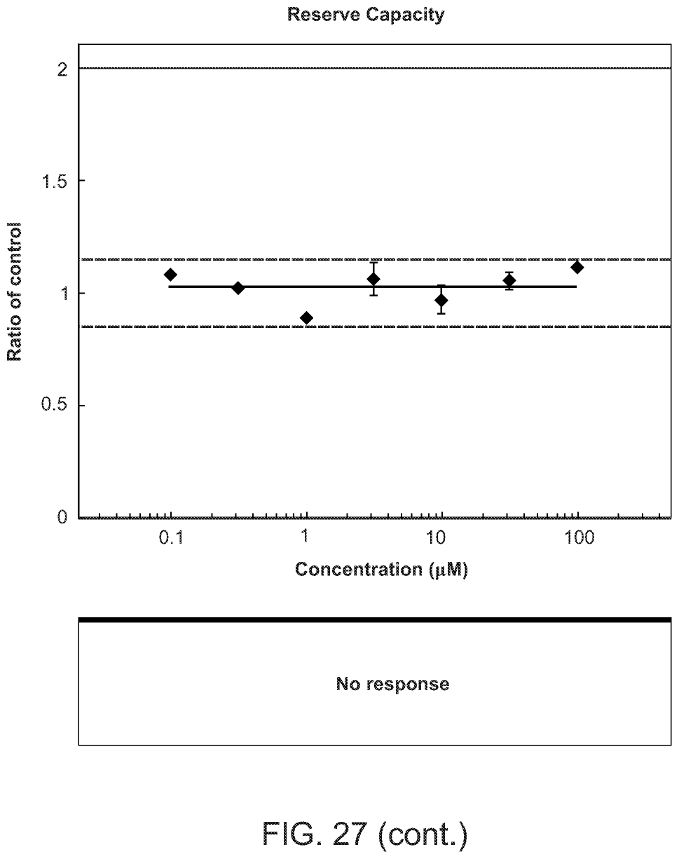

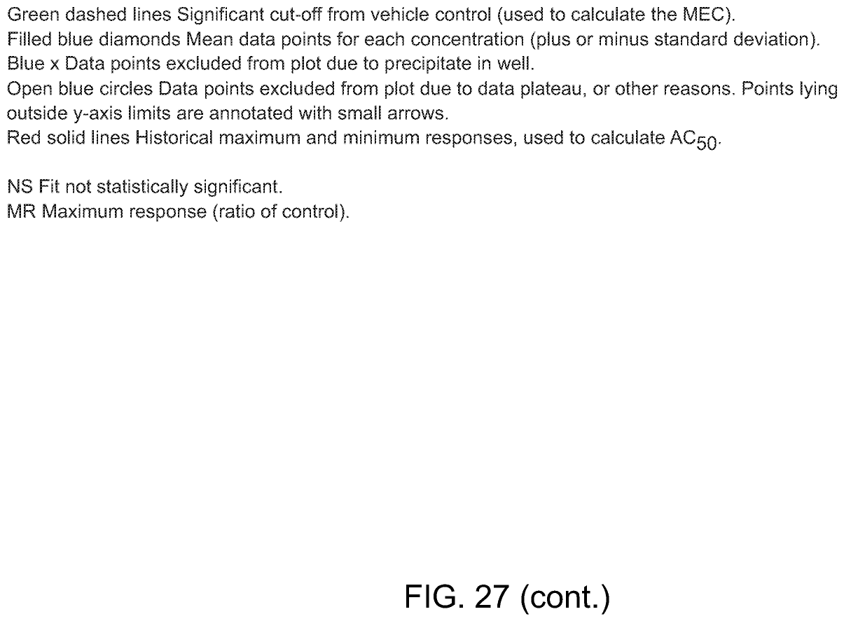

FIG. 27 is a series of graphs showing the effects of a combination of trimetazidine analog 2 and nicotinamide on oxygen consumption rate and reserve capacity.

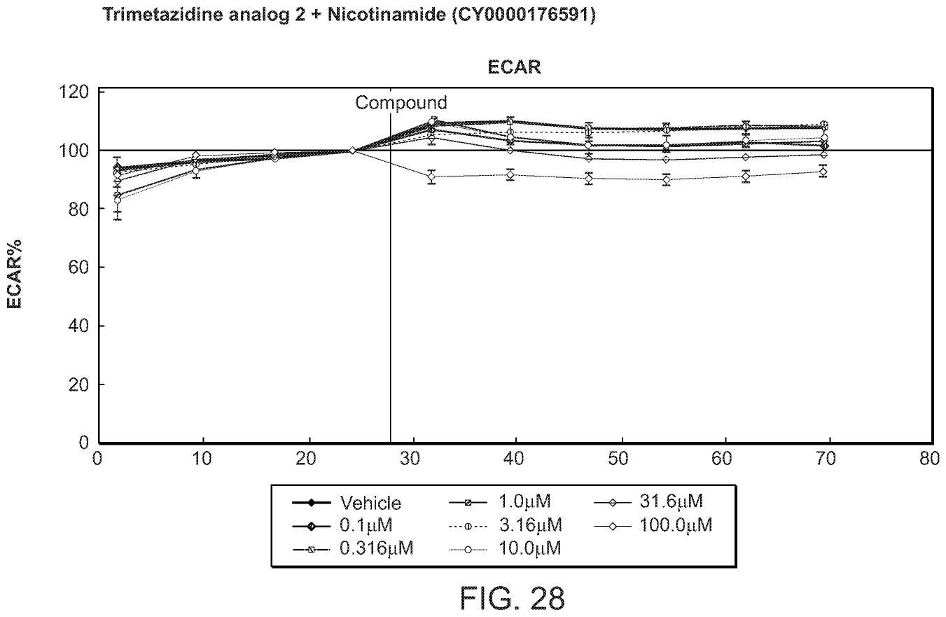

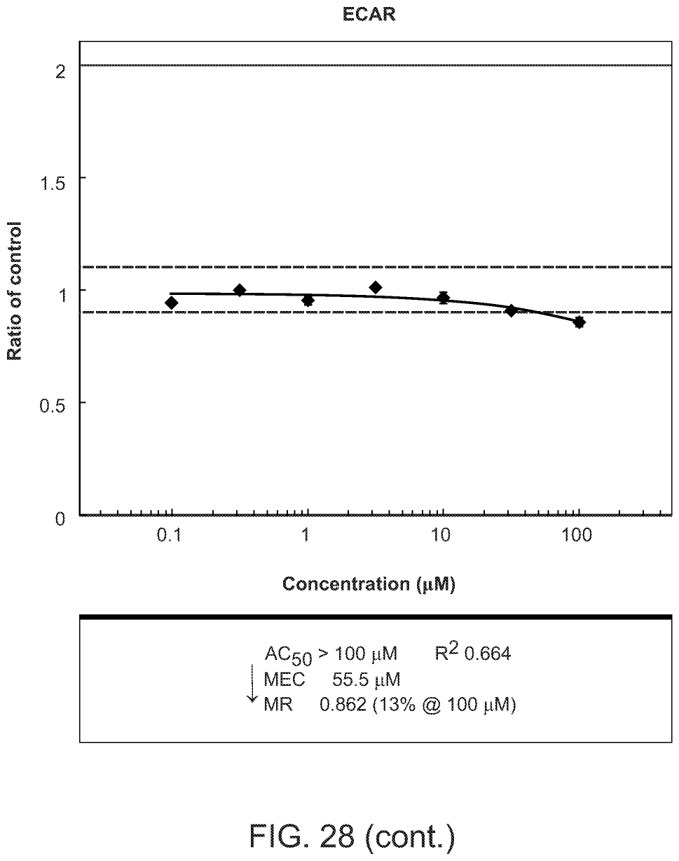

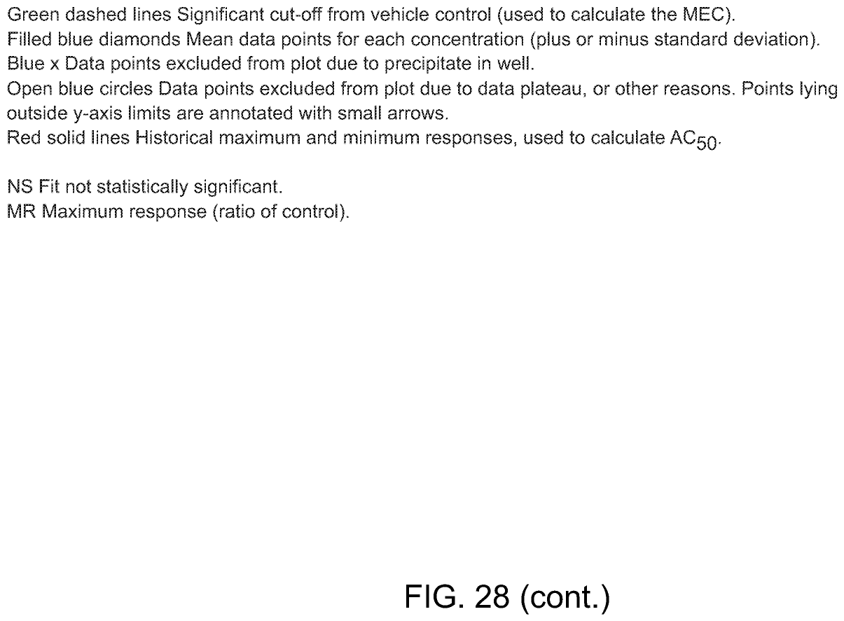

FIG. 28 is a series of graphs showing the effects a combination of trimetazidine analog 2 and nicotinamide on extracellular acidification rate.

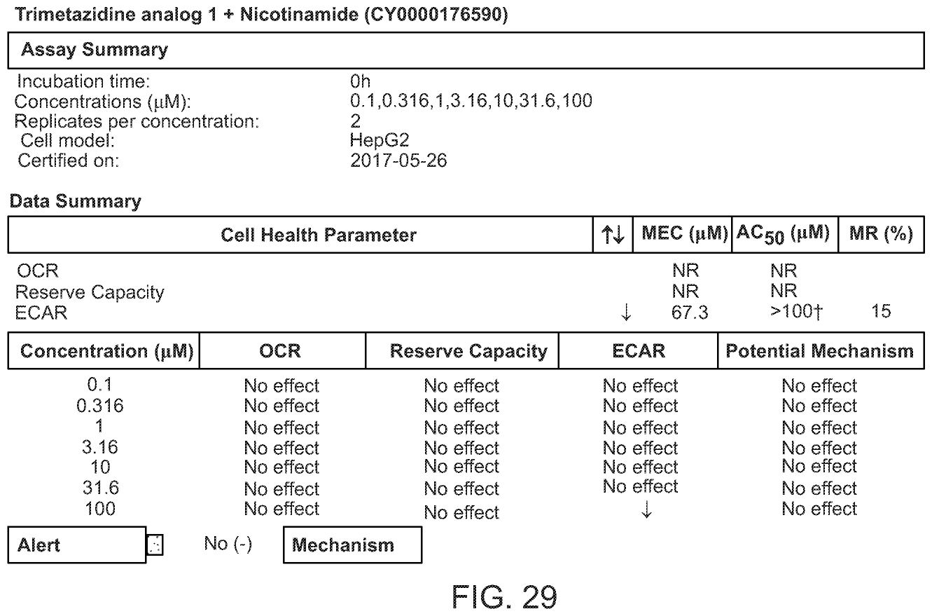

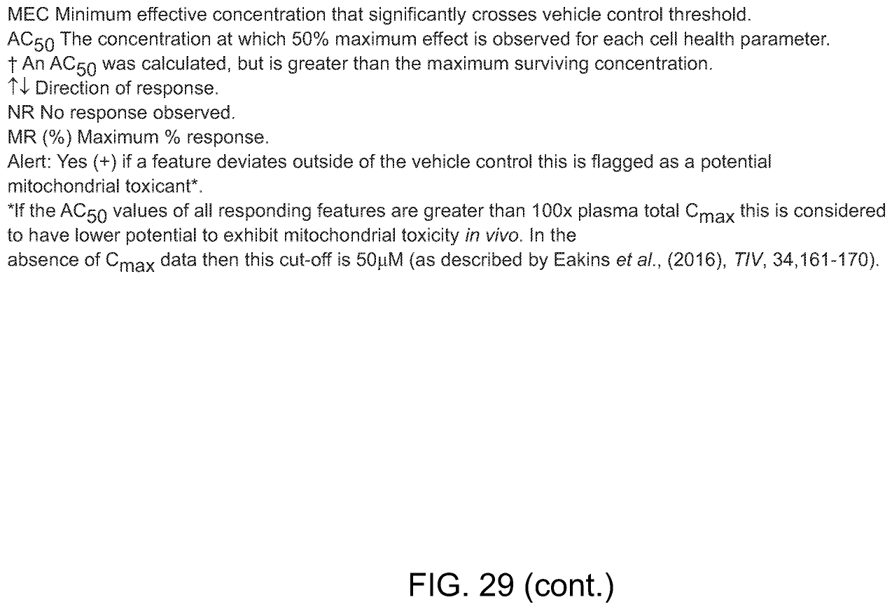

FIG. 29 is a table summarizing the effects of a combination of trimetazidine analog 1 and nicotinamide on various mitochondrial functional parameters.

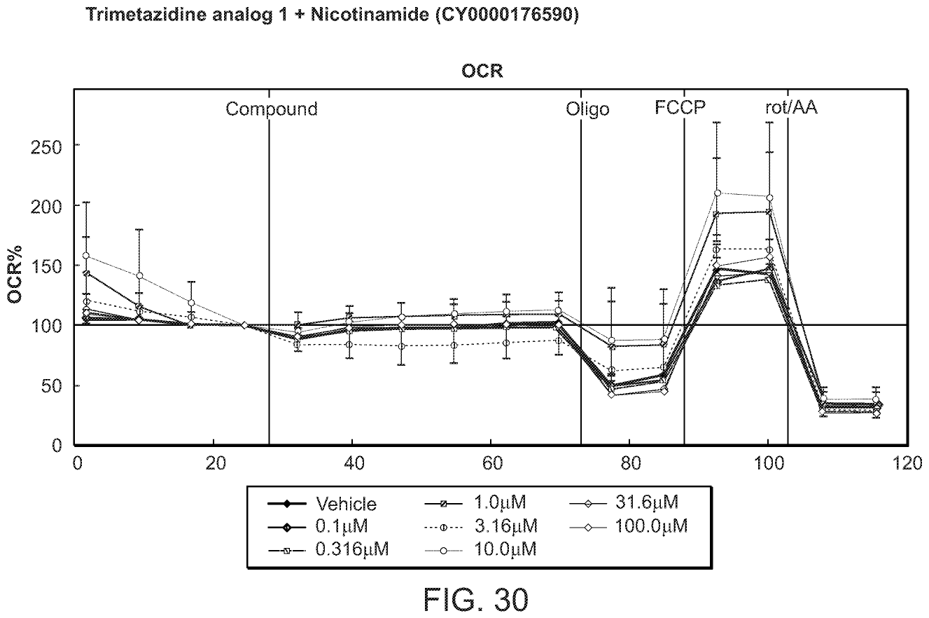

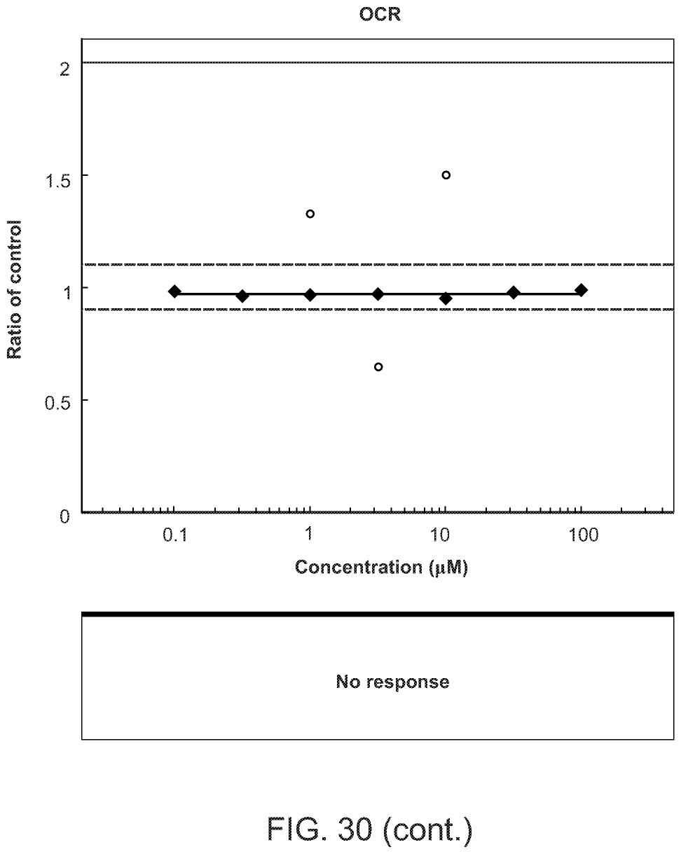

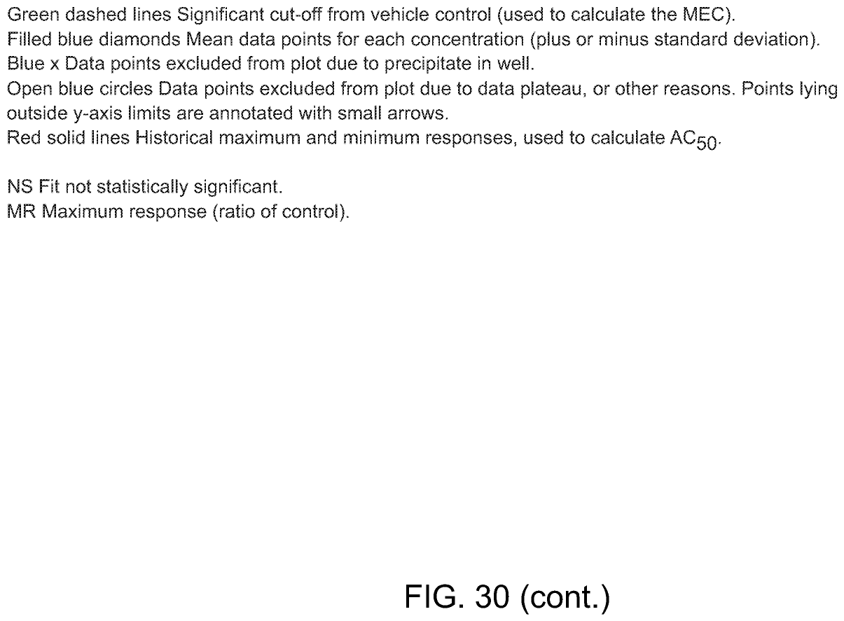

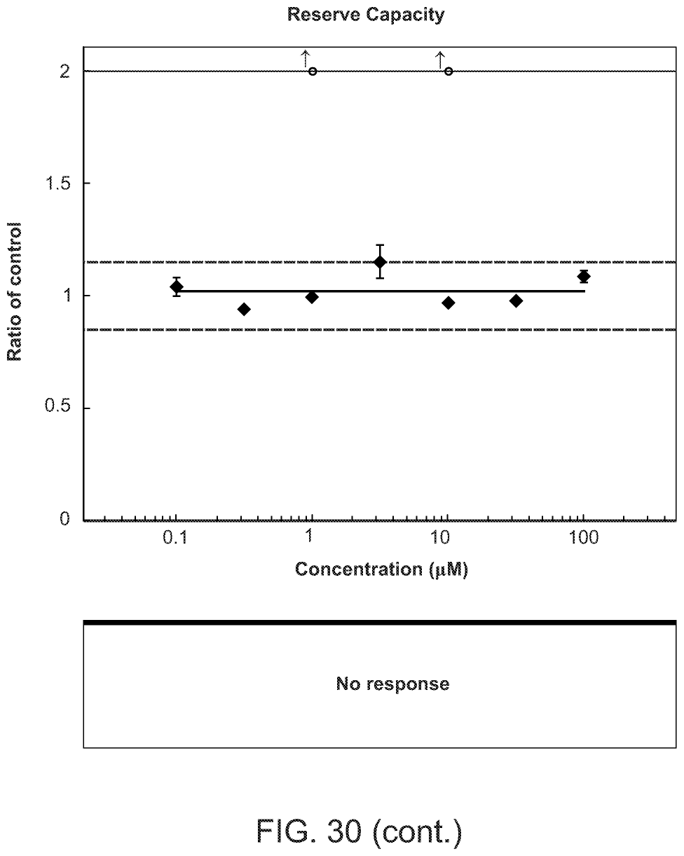

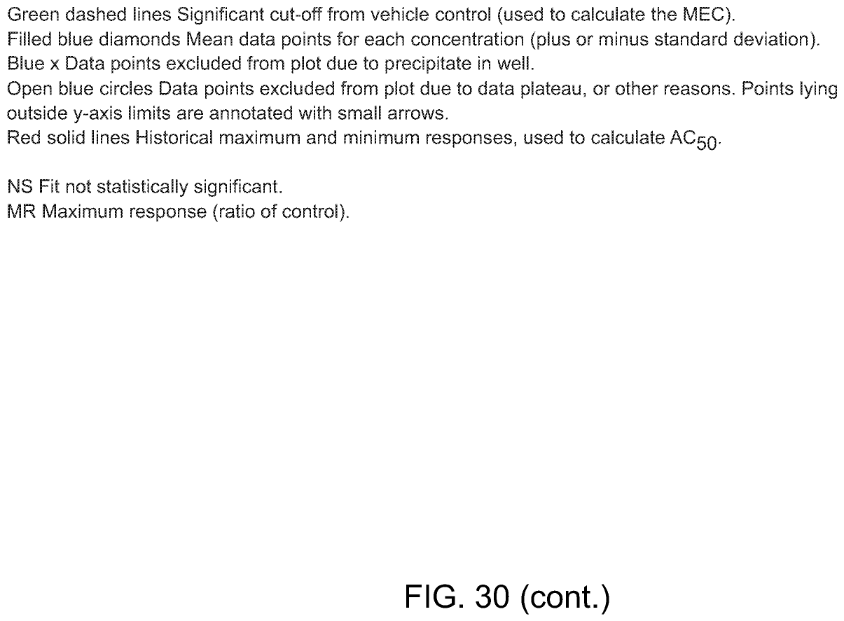

FIG. 30 is a series of graphs showing the effects of a combination of trimetazidine analog 1 and nicotinamide on oxygen consumption rate and reserve capacity.

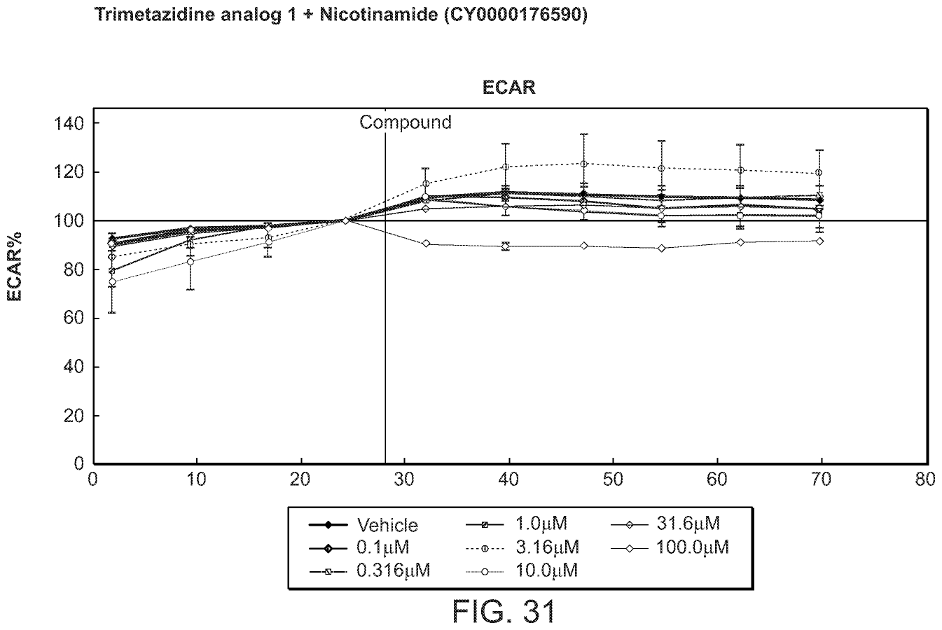

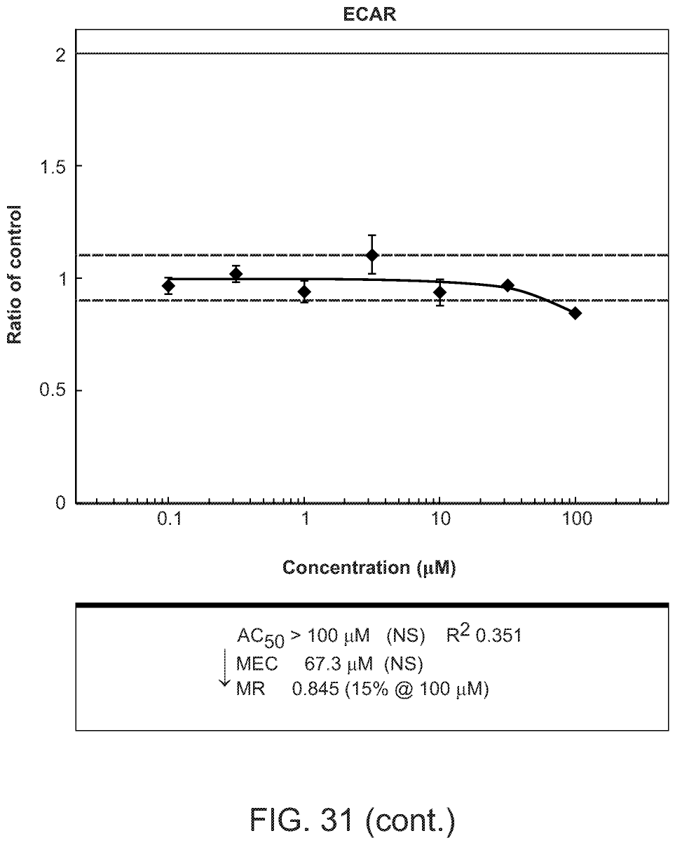

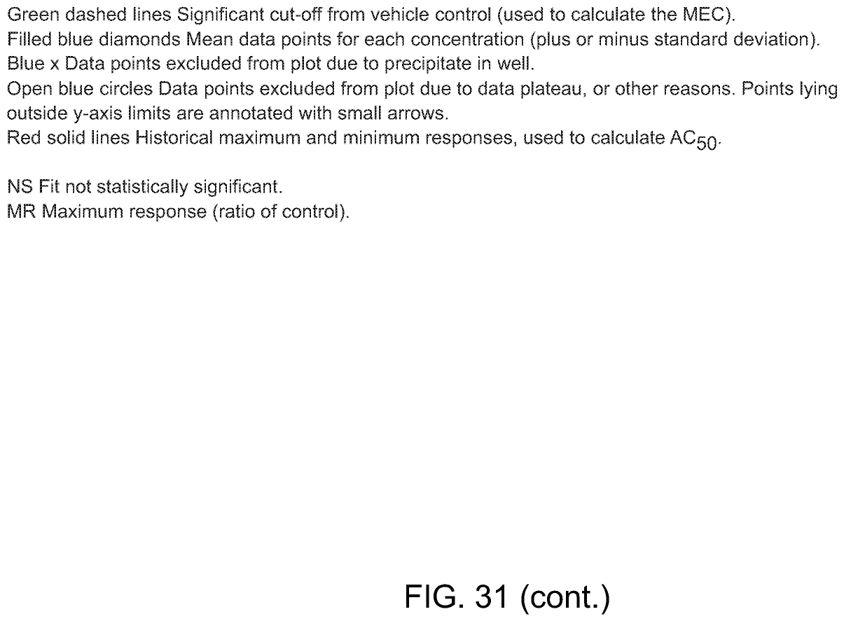

FIG. 31 is a series of graphs showing the effects of a combination of trimetazidine analog 1 and nicotinamide on extracellular acidification rate.

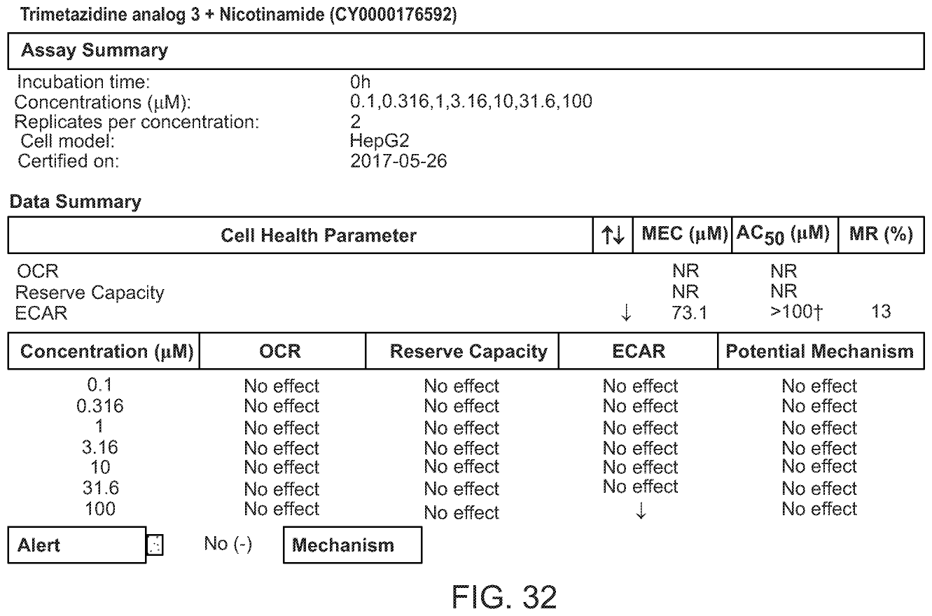

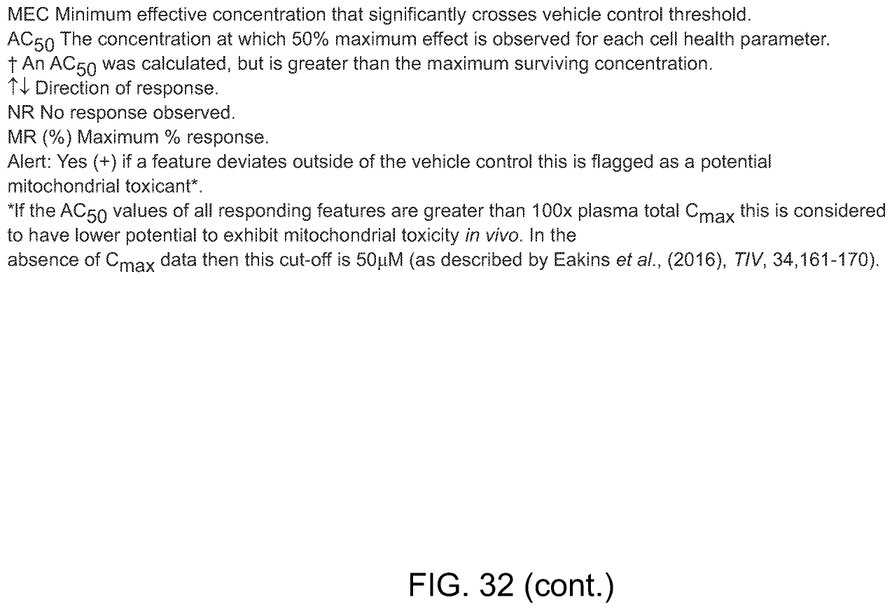

FIG. 32 is a table summarizing the effects of a combination of trimetazidine analog 3 and nicotinamide on various mitochondrial functional parameters.

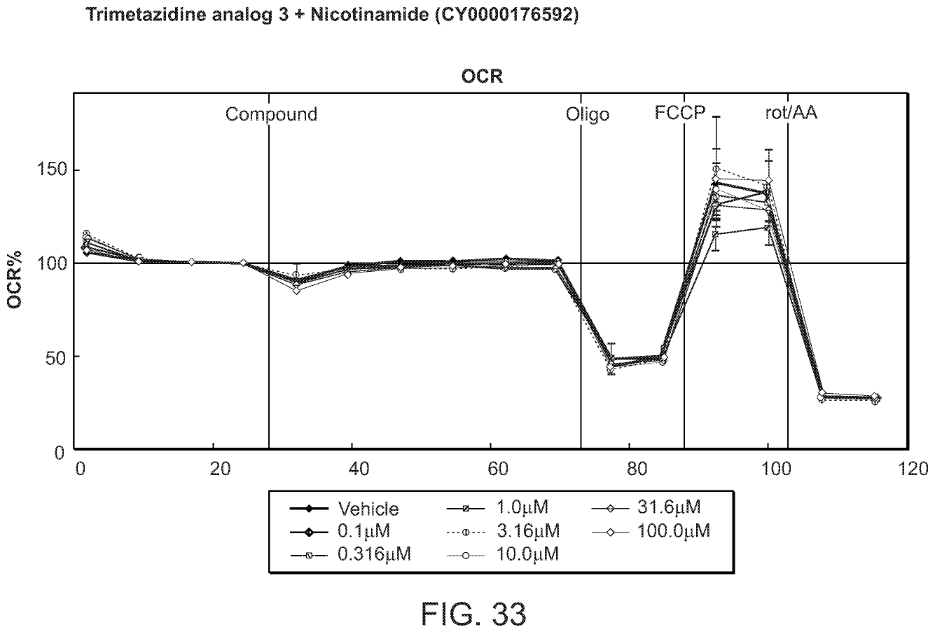

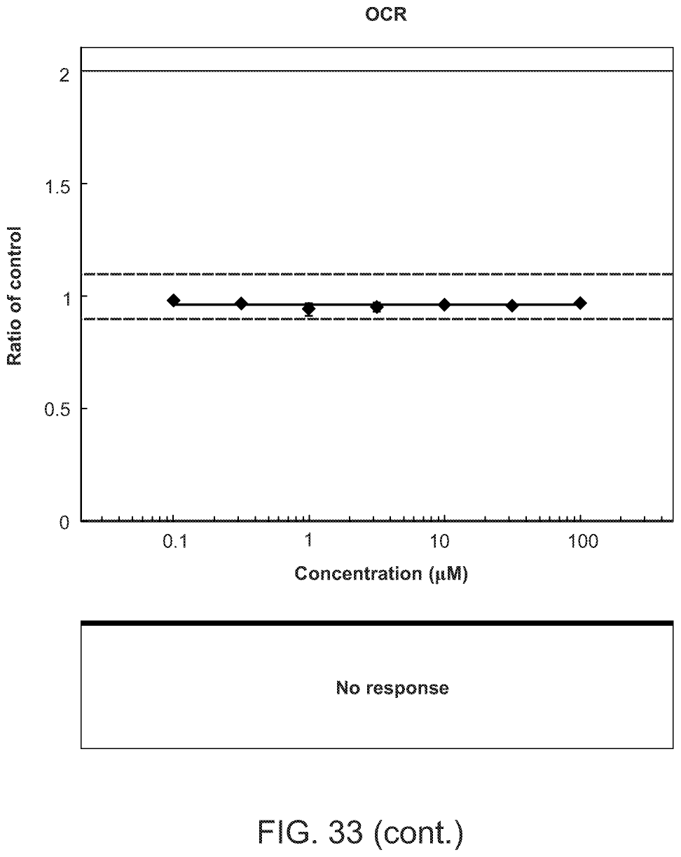

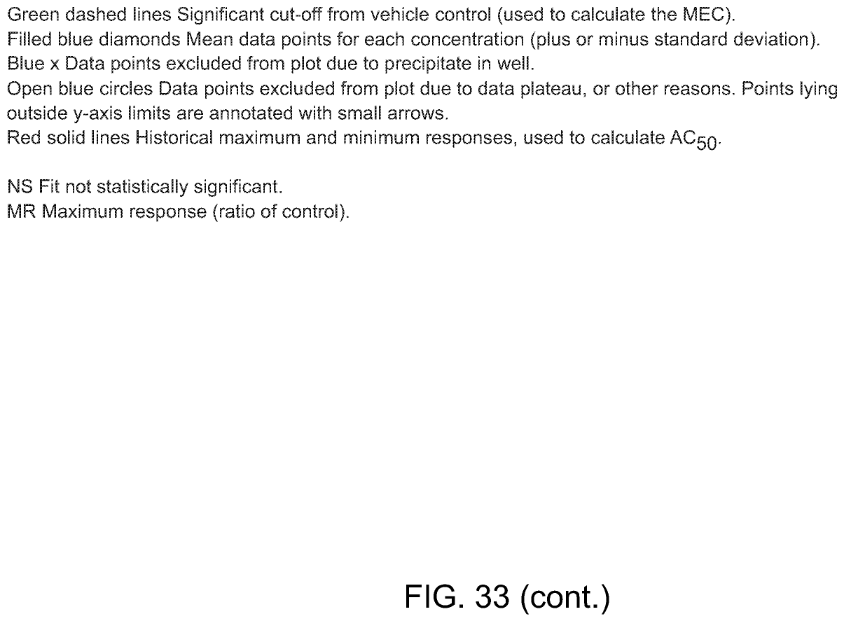

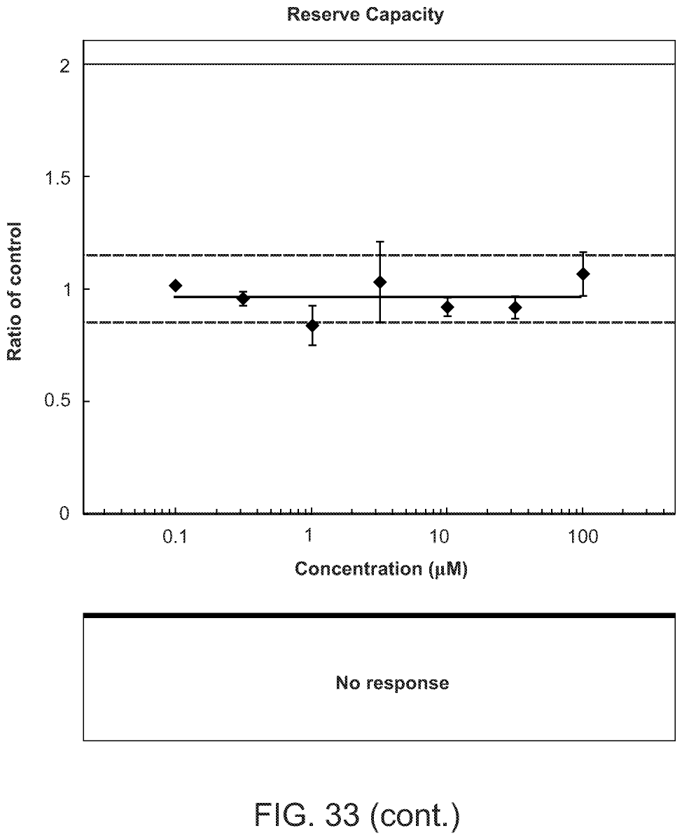

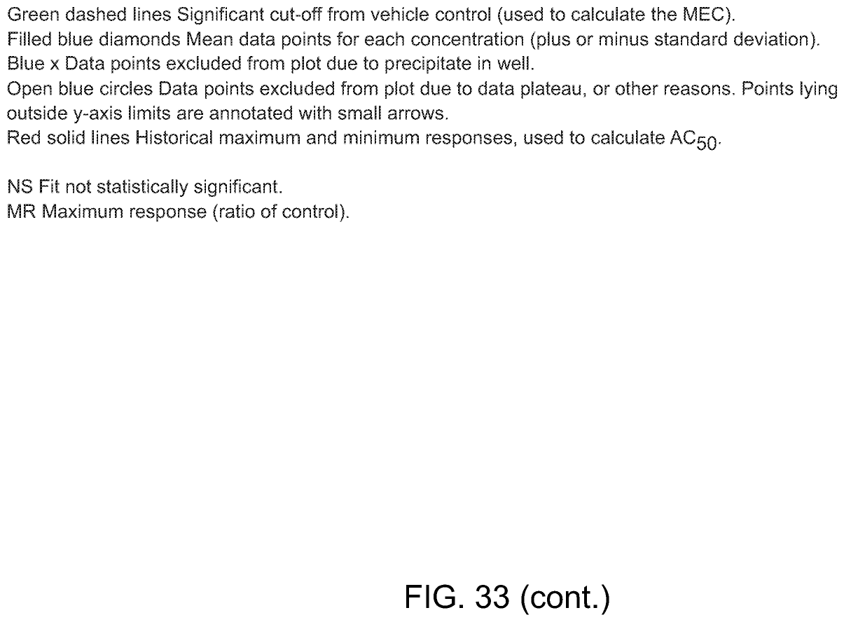

FIG. 33 is a series of graphs showing the effects of a combination of trimetazidine analog 3 and nicotinamide on oxygen consumption rate and reserve capacity.

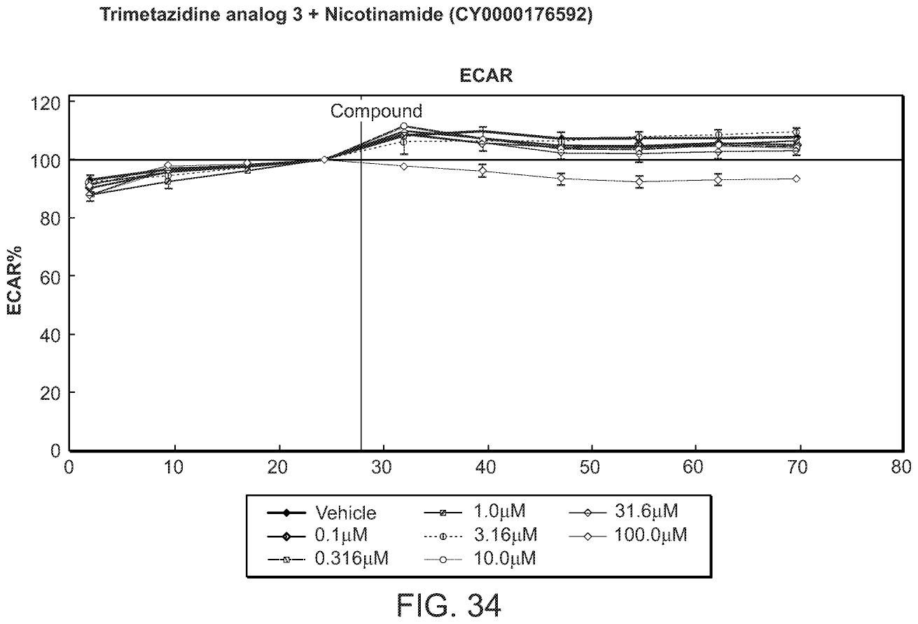

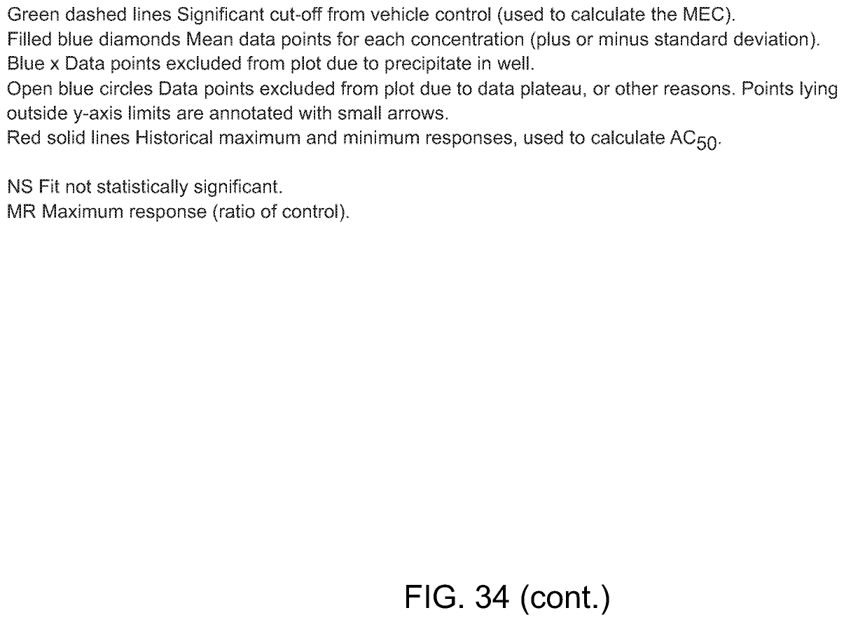

FIG. 34 is a series of graphs showing the effects of a combination of trimetazidine analog 3 and nicotinamide on extracellular acidification rate.

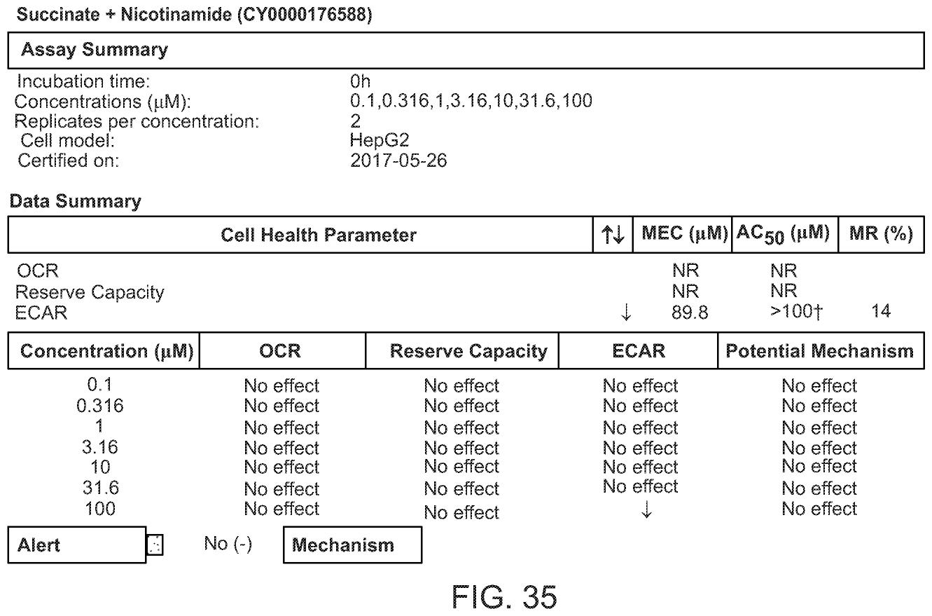

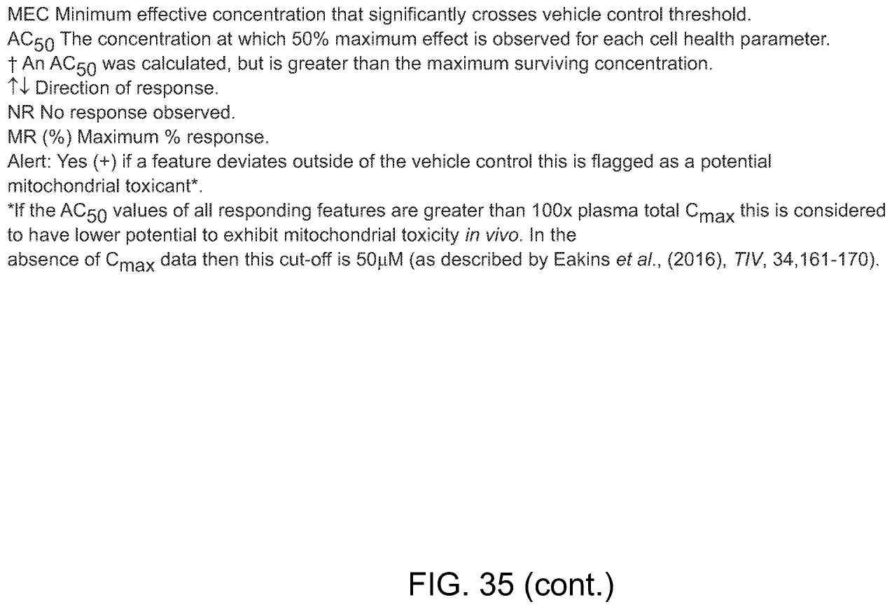

FIG. 35 is a table summarizing the effects of a combination of succinate and nicotinamide on various mitochondrial functional parameters.

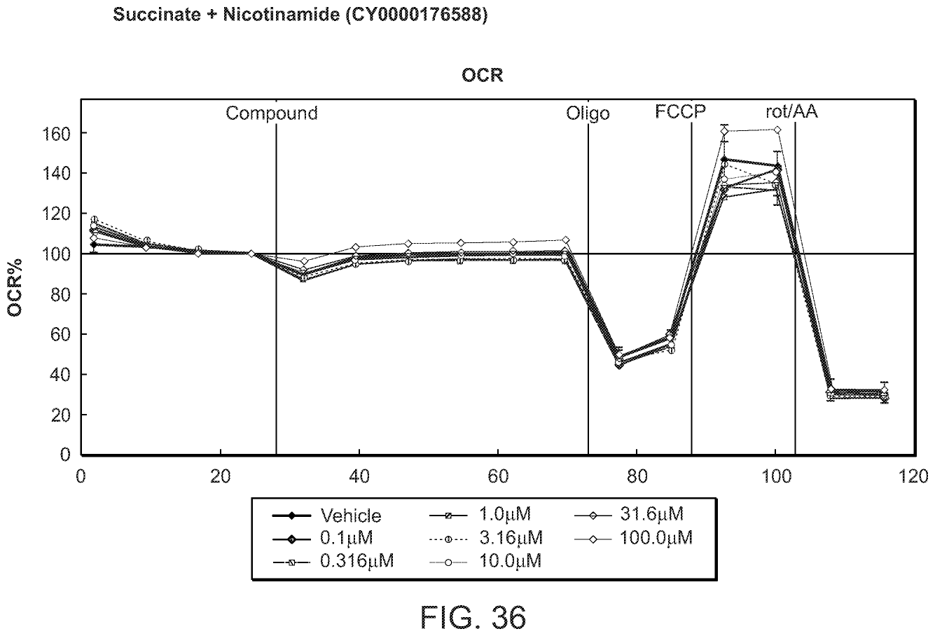

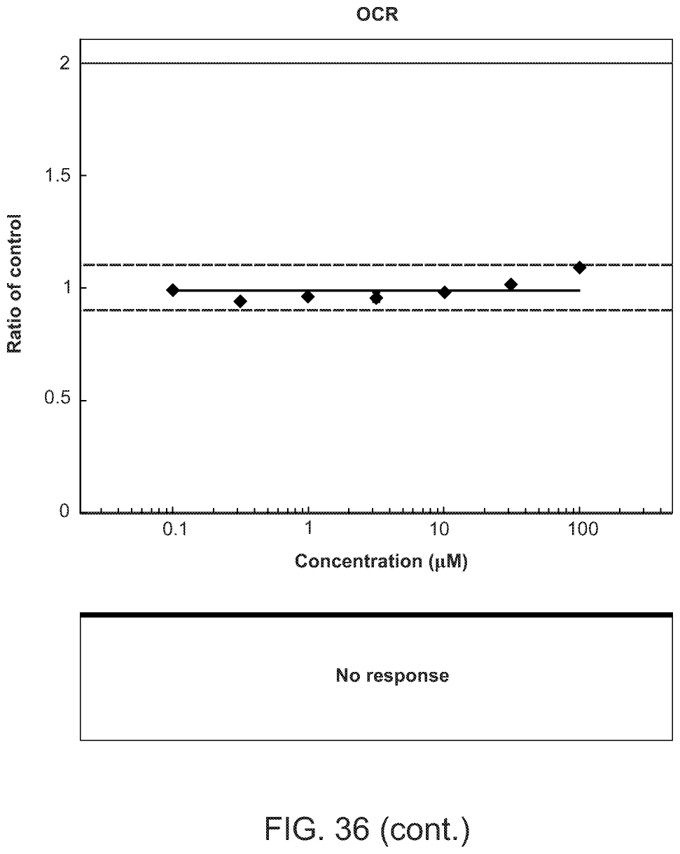

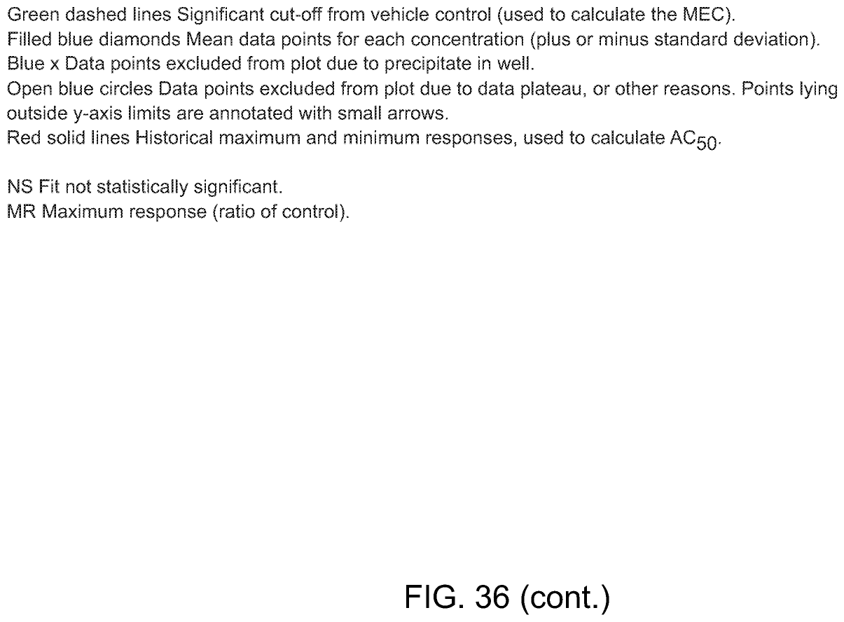

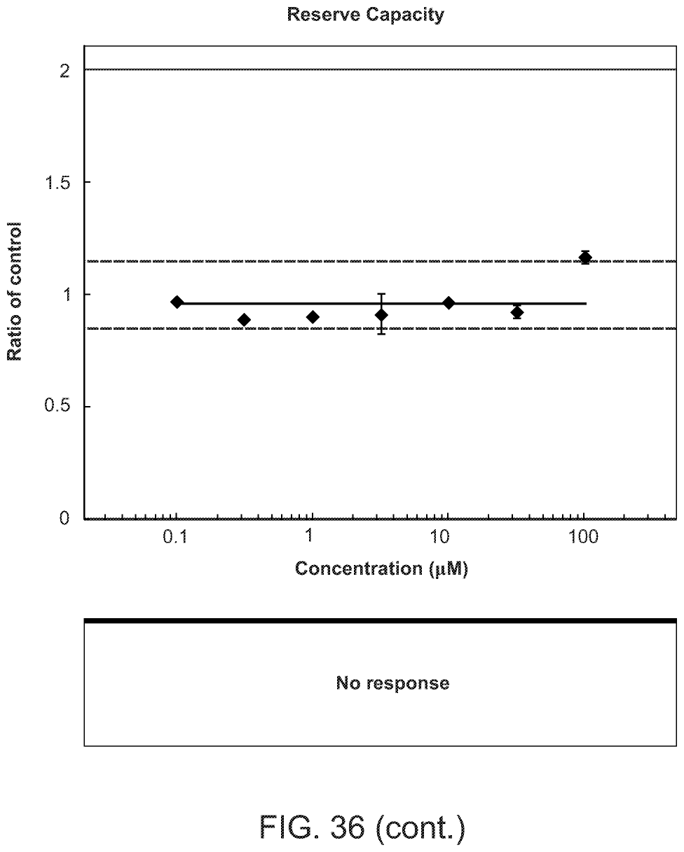

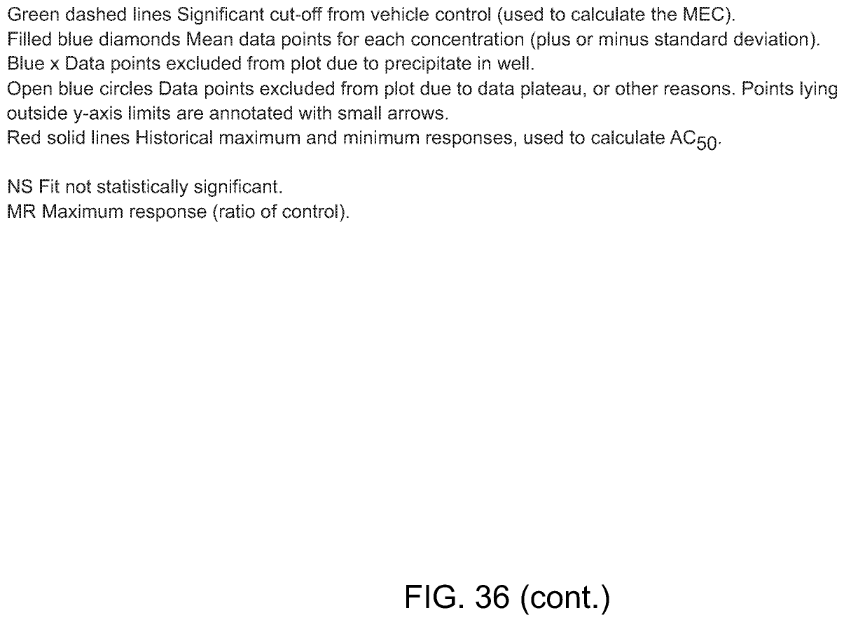

FIG. 36 is a series of graphs showing the effects of a combination of succinate and nicotinamide on oxygen consumption rate and reserve capacity.

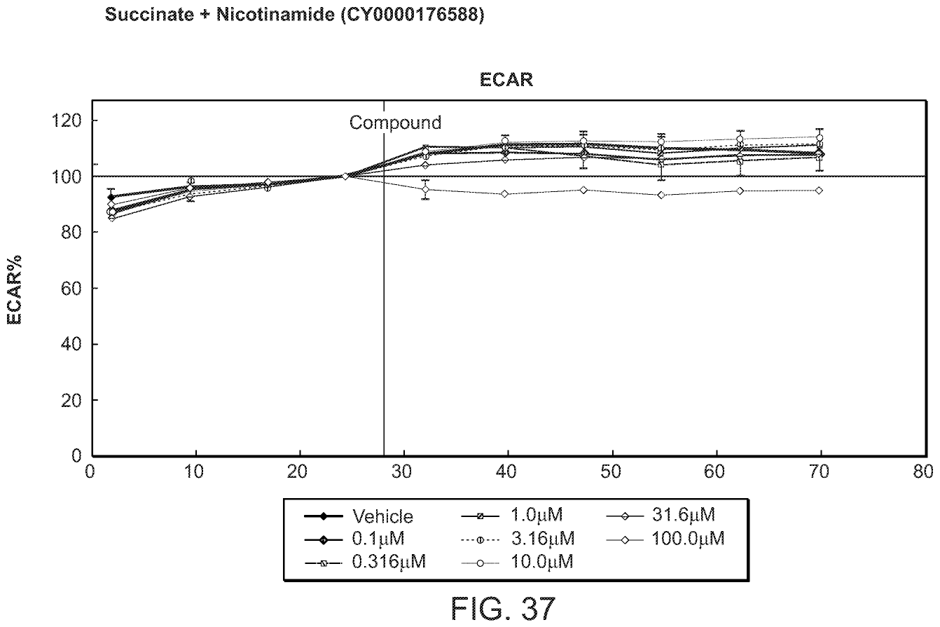

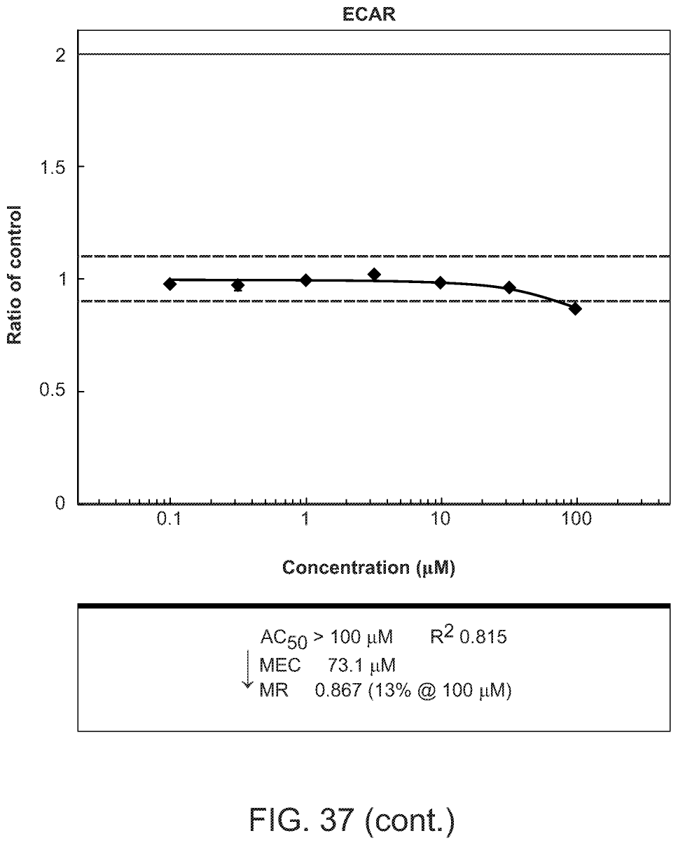

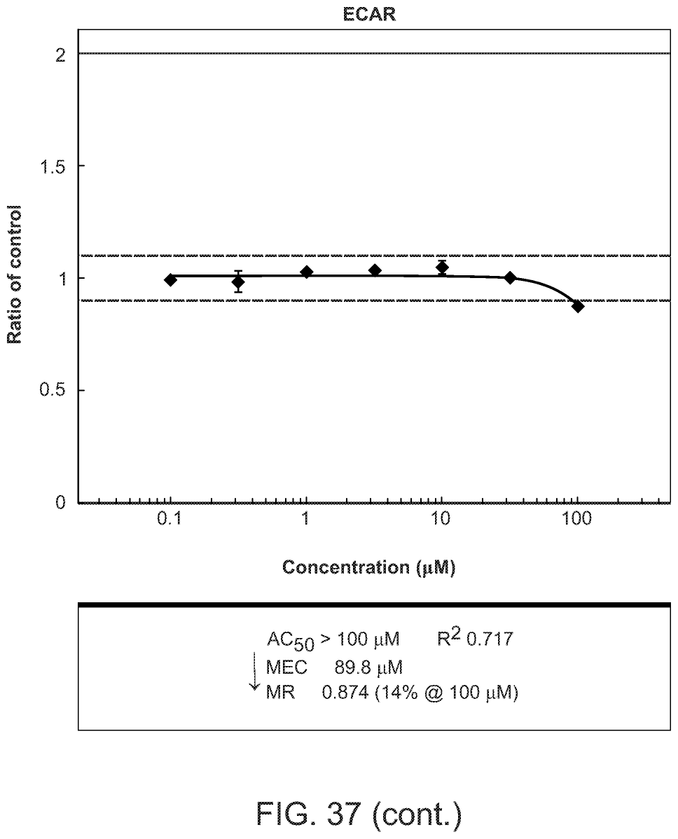

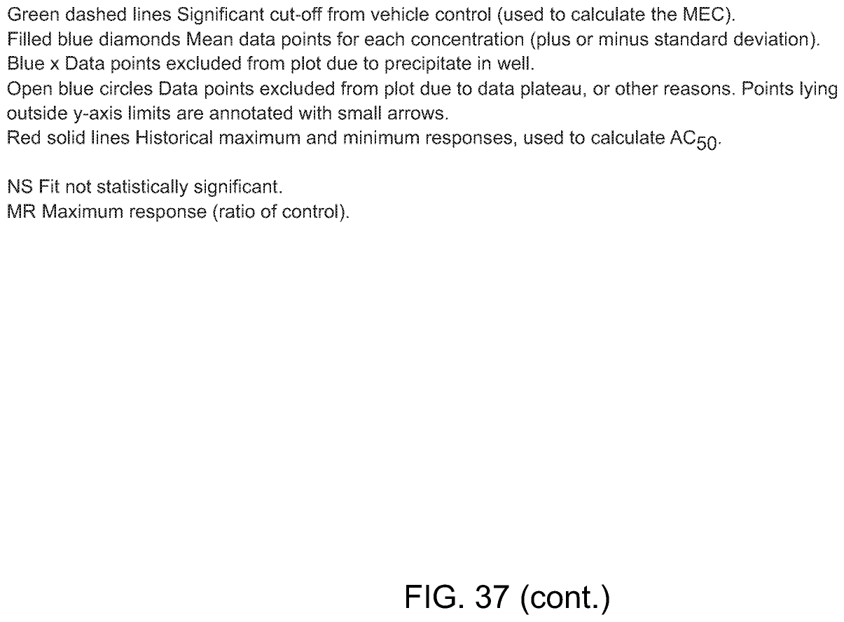

FIG. 37 is a series of graphs showing the effects of a combination of succinate and nicotinamide on extracellular acidification rate.

Effect of Compositions on Coronary Flow, Cardiac Function, and Infarct Size.

The effect of compositions on the coronary flow, cardiac function, and infarct size was analyzed.

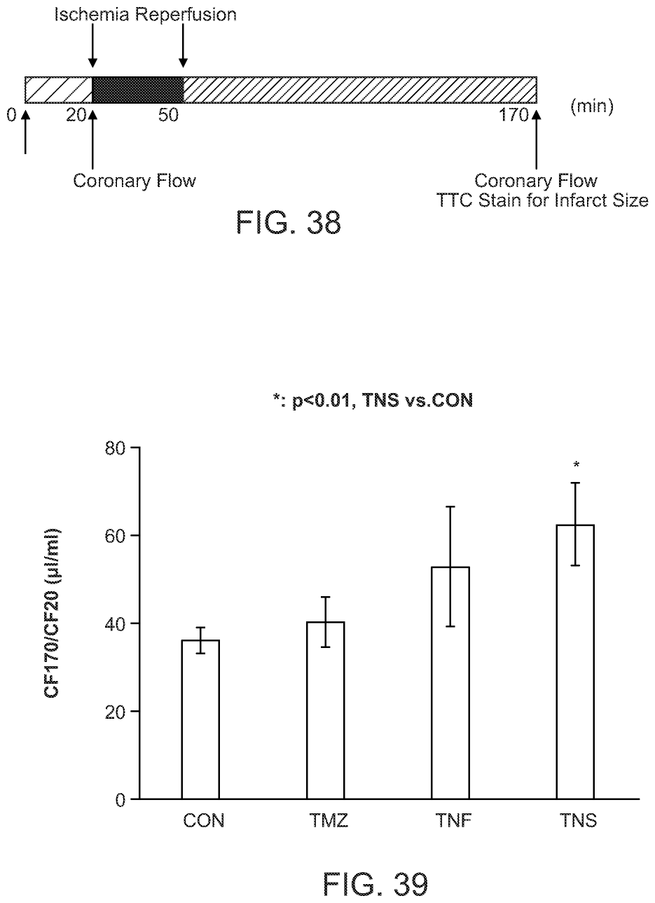

FIG. 38 is a schematic of the ischemia-reperfusion (IR) method used to analyze the effects of selected compositions on coronary flow, cardiac function, and infarct size. At time 0, mice were given (1) 20 μM trimetazidine (TMZ), (2) 2 μM each of trimetazidine, nicotinamide, and succinate (TNF), (3) 20 μM each of trimetazidine, nicotinamide, and succinate (TNS), or (4) the delivery vehicle (CON). At 20 minutes, ischemia was induced, and coronary flow was analyzed. At 50 minutes, reperfusion was initiated to restore blood flow. At 170 minutes, coronary flow and cardiac function was analyzed, and then the hearts were preserved, sectioned, and infarct size was measured by triphenyltetrazolium chloride (TTC) staining.

FIG. 39 is a graph of coronary flow of after IR. Data is expressed as ratio cardiac flow at 170 minutes to cardiac flow at 20 minutes. TNS treatment preserved coronary flow after IR. Raw data is provided in Tables 1-2.

TABLE 1

| | CF20 (ml/min) | CF170 (ml/min) | CF170/CF20 (μl/ml) |
|---|---|---|---|
| CON11 | 2.31E+00 | 1.11E−01 | 4.81E+01 |
| CON13 | 1.07E+00 | 4.80E−02 | 4.48E+01 |
| CON14 | 8.28E−01 | 4.50E−02 | 5.43E+01 |
| CON9 | 2.11E+00 | 6.96E−02 | 3.30E+01 |
| CON10 | 1.85E+00 | 4.92E−02 | 2.66E+01 |
| CON7 | 1.57E+00 | 5.40E−02 | 3.44E+01 |
| CON8 | 3.22E+00 | 6.78E−02 | 2.11E+01 |
| CON5 | 2.18E+00 | 6.60E−02 | 3.03E+01 |
| CON3 | 2.24E+00 | 7.92E−02 | 3.53E+01 |
| CON4 | 2.22E+00 | 7.84E−02 | 3.53E+01 |
| CON2 | 1.68E+00 | 5.12E−02 | 3.05E+01 |
| MEAN | 1.93E+00 | 6.54E−02 | 3.58E+01 |
| SD | 6.50E−01 | 1.94E−02 | 9.72E+00 |
| SE | 1.96E−01 | 5.86E−03 | 2.93E+00 |
| TTEST | | | |
| TMZ4 | 2.13E+00 | 5.16E−02 | 2.42E+01 |
| TMZ3 | 1.70E+00 | 1.00E−01 | 5.87E+01 |
| TMZ1 | 2.18E+00 | 7.78E−02 | 3.57E+01 |
| TMZ2 | 3.83E+00 | 1.29E−01 | 3.37E+01 |
| TMZ7 | 1.72E+00 | 8.98E−02 | 5.21E+01 |
| TMZ8 | 2.40E+00 | 6.56E−02 | 2.73E+01 |
| TMZ5 | 2.14E+00 | 5.56E−02 | 2.60E+01 |
| TMZ9 | 2.03E+00 | 1.30E−01 | 6.39E+01 |
| MEAN | 2.27E+00 | 8.74E−02 | 4.02E+01 |
| SD | 6.75E−01 | 3.06E−02 | 1.57E+01 |
| SE | 2.39E−01 | 1.08E−02 | 5.56E+00 |
| TTEST | | | |
| TNF1 | 2.24E+00 | 4.80E−02 | 2.14E+01 |
| TNF2 | 2.24E+00 | 3.80E−02 | 1.69E+01 |
| TNF3 | 7.32E−01 | 4.80E−02 | 6.56E+01 |
| TNF4 | 8.20E−01 | 4.90E−02 | 5.98E+01 |
| TNF5 | 1.09E+00 | 2.70E−02 | 2.48E+01 |
| TNF6 | 9.48E−01 | 1.50E−01 | 1.58E+02 |
| TNF7 | 8.08E−01 | 3.70E−02 | 4.58E+01 |
| TNF8 | 1.20E+00 | 4.60E−02 | 3.83E+01 |
| TNF9 | 1.45E+00 | 1.21E−01 | 8.33E+01 |
| TNF10 | 1.20E+00 | 1.52E−02 | 1.27E+01 |
| MEAN | 1.27E+00 | 5.79E−02 | 5.27E+01 |
| SD | 5.56E−01 | 4.28E−02 | 4.37E+01 |
| SE | 1.76E−01 | 1.35E−02 | 1.38E+01 |
| TTEST | 2.21E−02 | 6.06E−01 | 2.26E−01 |
| TNS1 | 1.52E+00 | 4.70E−02 | 3.08E+01 |
| TNS2 | 9.30E−01 | 2.90E−02 | 3.12E+01 |
| TNS3 | 2.24E+00 | 1.67E−01 | 7.46E+01 |
| TNS5 | 5.64E−01 | 5.00E−02 | 8.87E+01 |
| TNS6 | 6.28E−01 | 4.40E−02 | 7.01E+01 |
| TNS7 | 1.08E+00 | 6.40E−02 | 5.95E+01 |
| TNS8 | 8.72E−01 | 2.30E−02 | 2.64E+01 |
| TNS9 | 1.18E+00 | 8.50E−02 | 7.23E+01 |
| TNS10 | 1.70E+00 | 1.84E−01 | 1.08E+02 |
| MEAN | 1.19E+00 | 7.70E−02 | 6.24E+01 |
| SD | 5.43E−01 | 5.89E−02 | 2.82E+01 |
| SE | 1.81E−01 | 1.96E−02 | 9.42E+00 |
| TTEST | 1.35E−02 | 5.45E−01 | 8.80E−03 |
| vs TMZ | | | 6.82E−02 |

TABLE 2

| | CON | TMZ | TNF | TNS |
|---|---|---|---|---|
| MEAN | 36 | 40 | 53 | 62 |
| SD | 10 | 16 | 44 | 28 |
| SE | 3 | 6 | 14 | 9 |

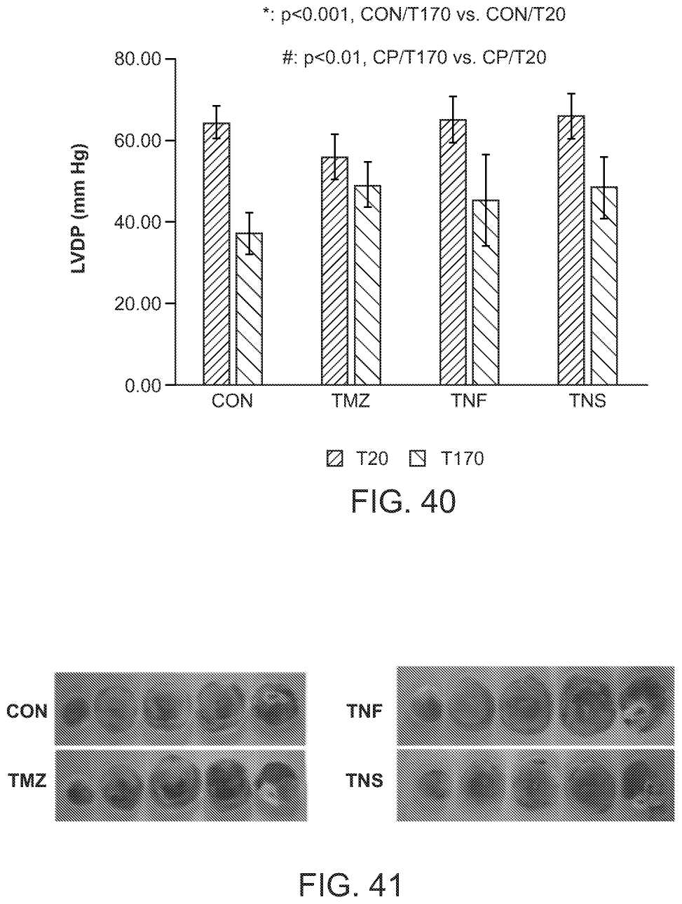

FIG. 40 is graph of left ventricular developed pressure (LVDP) after IR. Blue bars indicate LVDP at 20 minutes, and orange bars indicate LVDP at 170 minutes. TMZ, TNS, and TNF treatment prevented a decline in cardiac function after IR. Raw data is provided in Tables 3-6.

TABLE 3

| | pre- | | | | | |
|---|---|---|---|---|---|---|
| | ischemia | LVESP | LVEDP | HR | LVDP | LVDPxHR |
| 5-18-CN | CON11 | 6.61E+01 | 6.20E+00 | 3.28E+02 | 5.99E+01 | 1.97E+04 |
| | CON12 | 8.15E+01 | 3.73E+00 | 3.56E+02 | 7.78E+01 | 2.77E+04 |
| 6-10-CN | CON13 | 8.00E+01 | −3.74E+00 | 1.37E+02 | 8.37E+01 | 1.15E+04 |
| | CON14 | 7.28E+01 | 6.12E+00 | 4.54E+02 | 6.67E+01 | 3.03E+04 |
| 5-15-CN | CON9 | 8.07E+01 | 5.00E+00 | 1.42E+02 | 7.57E+01 | 1.08E+04 |
| | CON10 | 4.91E+01 | 1.15E+00 | 3.21E+02 | 4.80E+01 | 1.54E+04 |
| 5-12-CN | CON7 | 8.55E+01 | 6.35E+00 | 3.05E+02 | 7.91E+01 | 2.42E+04 |
| | CON8 | 5.06E+01 | 1.68E+00 | 3.04E+02 | 4.90E+01 | 1.49E+04 |
| 5-9-CN | CON5 | 5.45E+01 | 5.63E+00 | 2.75E+02 | 4.89E+01 | 1.35E+04 |
| | CON6 | 6.37E+01 | 4.31E+00 | 3.08E+02 | 5.94E+01 | 1.83E+04 |
| 5-7-CN | CON3 | 7.32E+01 | 2.70E+00 | 2.40E+02 | 7.05E+01 | 1.69E+04 |
| | CON4 | 4.91E+01 | 1.65E−01 | 3.14E+02 | 4.89E+01 | 1.54E+04 |
| 5-5-CN | CON1 | 9.48E+01 | 7.96E+00 | 3.04E+02 | 8.68E+01 | 2.64E+04 |
| | CON2 | 4.69E+01 | 1.64E−01 | 4.02E+02 | 4.67E+01 | 1.88E+04 |
| | MEAN | 6.77E+01 | 3.39E+00 | 2.99E+02 | 6.44E+01 | 1.88E+04 |
| | SD | 1.58E+01 | 3.21E+00 | 8.52E+01 | 1.46E+01 | 6.12E+03 |
| | SE | 4.21E+00 | 8.57E−01 | 2.28E+01 | 3.91E+00 | 1.63E+03 |
| | TTEST | | | | 2.42E−04 | |
| 5-14-TMZ | TMZ3 | 7.58E+01 | 6.53E+00 | 2.63E+02 | 6.93E+01 | 1.83E+04 |
| | TMZ4 | 8.44E+01 | 5.43E+00 | 2.93E+02 | 7.90E+01 | 2.31E+04 |
| 5-11-TMZ | TMZ1 | 7.15E+01 | 6.76E+00 | 1.66E+02 | 6.48E+01 | 1.08E+04 |
| | TMZ2 | 5.47E+01 | 1.74E+00 | 3.35E+02 | 5.30E+01 | 1.77E+04 |
| 5-8-TMZ | TMZ7 | 6.87E+01 | 3.58E+00 | 3.58E+02 | 6.51E+01 | 2.33E+04 |
| | TMZ8 | 4.27E+01 | 4.71E+00 | 3.33E+02 | 3.80E+01 | 1.26E+04 |
| 5-6-TMZ | TMZ5 | 3.30E+01 | 4.77E+00 | 3.48E+02 | 2.82E+01 | 9.82E+03 |
| | TMZ6 | 3.30E+01 | 1.46E+00 | 3.21E+02 | 3.15E+01 | 1.01E+04 |
| 5-4-TMZ | TMZ9 | 6.60E+01 | 7.25E+00 | 2.67E+02 | 5.87E+01 | 1.57E+04 |
| | TMZ10 | 7.38E+01 | 2.70E+00 | 3.32E+02 | 7.11E+01 | 2.36E+04 |
| | MEAN | 6.03E+01 | 4.49E+00 | 3.02E+02 | 5.59E+01 | 1.68E+04 |
| | SD | 1.85E+01 | 2.07E+00 | 5.75E+01 | 1.77E+01 | 5.56E+03 |
| | SE | 5.84E+00 | 6.56E−01 | 1.82E+01 | 5.58E+00 | 1.76E+03 |
| | | | | | 3.85E−01 | |
| 5-19-TNF | TNF1 | 5.02E+01 | 3.04E+00 | 4.09E+02 | 4.72E+01 | 1.93E+04 |
| | TNF2 | 4.65E+01 | 1.76E−01 | 2.76E+02 | 4.63E+01 | 1.28E+04 |
| 6-8-TNF | TNF3 | 7.13E+01 | 1.53E+00 | 6.48E+01 | 6.97E+01 | 4.52E+03 |
| | TNF4 | 9.97E+01 | 4.15E+00 | 1.54E+02 | 9.55E+01 | 1.47E+04 |
| 6-12-TNF | TNF5 | 7.14E+01 | −3.42E+00 | 2.77E+02 | 7.49E+01 | 2.07E+04 |
| | TNF6 | 8.98E+01 | 8.85E+00 | 3.10E+02 | 8.09E+01 | 2.51E+04 |
| 6-14-TNF | TNF7 | 6.58E+01 | 7.01E+00 | 3.98E+02 | 5.88E+01 | 2.34E+04 |
| | TNF8 | 5.99E+01 | 1.02E+00 | 2.28E+02 | 5.89E+01 | 1.34E+04 |
| 6-15-TNF | TNF9 | 7.89E+01 | 2.37E−01 | 2.71E+02 | 7.87E+01 | 2.13E+04 |
| | TNF10 | 4.01E+01 | 1.88E+00 | 3.14E+02 | 3.82E+01 | 1.20E+04 |
| | MEAN | 6.74E+01 | 2.45E+00 | 2.70E+02 | 6.49E+01 | 1.67E+04 |
| | SD | 1.90E+01 | 3.54E+00 | 1.04E+02 | 1.81E+01 | 6.32E+03 |
| | SE | 6.00E+00 | 1.12E+00 | 3.28E+01 | 5.73E+00 | 2.00E+03 |
| | | | | | 1.38E−01 | |
| 5-20-TNS | TNS1 | 5.59E+01 | 5.23E+00 | 3.33E+02 | 5.07E+01 | 1.69E+04 |
| | TNS2 | 5.54E+01 | −1.83E+00 | 1.24E+02 | 5.72E+01 | 7.09E+03 |
| 6-7-TNS | TNS3 | 8.78E+01 | 1.53E+00 | 1.64E+02 | 8.63E+01 | 1.42E+04 |
| | TNS4 | 1.07E+02 | 9.86E+00 | 2.41E+02 | 9.74E+01 | 2.35E+04 |
| 6-9-TNS | TNS5 | 8.97E+01 | 2.34E+00 | 8.35E+01 | 8.74E+01 | 7.29E+03 |
| | TNS6 | 6.17E+01 | 6.21E+00 | 1.85E+02 | 5.55E+01 | 1.03E+04 |
| 6-13-TNS | TNS7 | 6.62E+01 | 4.14E+00 | 3.36E+02 | 6.21E+01 | 2.09E+04 |
| | TNS8 | 6.54E+01 | 1.22E+01 | 1.22E+02 | 5.32E+01 | 6.47E+03 |
| 6-15-TNS | TNS9 | 6.16E+01 | 3.64E+00 | 3.45E+02 | 5.80E+01 | 2.00E+04 |
| | TNS10 | 5.44E+01 | 2.47E+00 | 4.12E+02 | 5.20E+01 | 2.14E+04 |
| | MEAN | 7.05E+01 | 4.58E+00 | 2.35E+02 | 6.60E+01 | 1.48E+04 |
| | SD | 1.80E+01 | 4.09E+00 | 1.15E+02 | 1.74E+01 | 6.61E+03 |
| | SE | 5.69E+00 | 1.29E+00 | 3.63E+01 | 5.49E+00 | 2.09E+03 |
| | | | | | 7.89E−02 | |

TABLE 4

| | after 2h | | | | | |
|---|---|---|---|---|---|---|
| | reperfusion | LVESP | LVEDP | HR | LVDP | LVDPxHR |
| 5-18-CN | CON11 | 7.78E+01 | 3.68E+01 | 1.18E+02 | 4.10E+01 | 4.82E+03 |
| | CON12 | 7.07E+01 | 2.23E+01 | 9.23E+01 | 4.84E+01 | 4.47E+03 |
| 6-10-CN | CON13 | 6.48E+01 | 5.54E+01 | 5.72E+02 | 9.39E+00 | 5.38E+03 |
| | CON14 | 9.54E+01 | 5.64E+01 | 2.08E+02 | 3.90E+01 | 8.12E+03 |
| 5-15-CN | CON9 | 5.18E+01 | 2.71E+01 | 1.75E+02 | 2.47E+01 | 4.33E+03 |
| | CON10 | 1.10E+02 | 3.13E+01 | 5.76E+01 | 7.84E+01 | 4.51E+03 |
| 5-12-CN | CON7 | 3.93E+01 | 1.42E+01 | 9.11E+01 | 2.51E+01 | 2.29E+03 |
| | CON8 | 5.29E+01 | 9.48E+00 | 6.07E+01 | 4.34E+01 | 2.64E+03 |
| 5-9-CN | CON5 | 6.56E+01 | 4.89E+01 | 6.50E+01 | 1.67E+01 | 1.09E+03 |
| | CON6 | 7.44E+01 | 6.56E+01 | 3.78E+01 | 8.81E+00 | 3.33E+02 |
| 5-7-CN | CON3 | 6.35E+01 | 9.99E+00 | 1.15E+02 | 5.35E+01 | 6.18E+03 |
| | CON4 | 8.76E+01 | 5.34E+01 | 1.06E+02 | 3.43E+01 | 3.65E+03 |
| 5-5-CN | CON1 | 9.29E+01 | 4.38E+01 | 2.61E+02 | 4.91E+01 | 1.28E+04 |
| | CON2 | 5.18E+01 | 4.43E+00 | 2.57E+02 | 4.74E+01 | 1.22E+04 |
| | MEAN | 7.13E+01 | 3.42E+01 | 1.58E+02 | 3.71E+01 | 5.20E+03 |
| | SD | 1.98E+01 | 2.02E+01 | 1.39E+02 | 1.90E+01 | 3.68E+03 |
| | SE | 5.29E+00 | 5.40E+00 | 3.72E+01 | 5.08E+00 | 9.83E+02 |
| | TTEST | | | | | |
| 5-14-TMZ | TMZ3 | 5.07E+01 | 2.93E+01 | 1.18E+02 | 2.14E+01 | 2.52E+03 |
| | TMZ4 | 7.66E+01 | 3.31E+01 | 1.19E+02 | 4.34E+01 | 5.15E+03 |
| 5-11-TMZ | TMZ1 | 9.19E+01 | 3.96E+01 | 1.01E+02 | 5.22E+01 | 5.28E+03 |
| | TMZ2 | 4.77E+01 | 1.80E+01 | 1.51E+02 | 2.97E+01 | 4.49E+03 |
| 5-8-TMZ | TMZ7 | 5.18E+01 | 3.36E+00 | 6.70E+01 | 4.84E+01 | 3.24E+03 |
| | TMZ8 | 4.86E+01 | 1.87E+00 | 9.22E+01 | 4.67E+01 | 4.31E+03 |
| 5-6-TMZ | TMZ5 | 6.09E+01 | 1.99E+01 | 2.22E+02 | 4.10E+01 | 9.11E+03 |
| | TMZ6 | 1.09E+02 | 3.21E+01 | 1.70E+02 | 7.65E+01 | 1.30E+04 |
| 5-4-TMZ | TMZ9 | 7.38E+01 | 1.84E+01 | 1.16E+02 | 5.53E+01 | 6.44E+03 |
| | TMZ10 | 7.61E+01 | 1.77E+00 | 2.38E+02 | 7.43E+01 | 1.77E+04 |
| | MEAN | 6.86E+01 | 1.97E+01 | 1.39E+02 | 4.89E+01 | 6.82E+03 |
| | SD | 2.05E+01 | 1.39E+01 | 5.58E+01 | 1.73E+01 | 4.82E+03 |
| | SE | 6.49E+00 | 4.39E+00 | 1.77E+01 | 5.46E+00 | 1.52E+03 |
| 5-19-TNF | TNF1 | 8.37E+01 | 6.66E+01 | 1.53E+02 | 1.71E+01 | 2.62E+03 |
| | TNF2 | 6.19E+00 | 5.54E+00 | 2.13E+03 | 6.48E−01 | 1.38E+03 |
| 6-8-TNF | TNF3 | 8.99E+01 | 1.88E+01 | 1.05E+01 | 7.11E+01 | 7.49E+02 |
| | TNF4 | 6.06E+01 | 1.34E+01 | 8.10E+01 | 4.72E+01 | 3.82E+03 |
| 6-12-TNF | TNF5 | 1.54E+02 | 4.15E+01 | 2.20E+01 | 1.13E+02 | 2.48E+03 |
| | TNF6 | 1.30E+02 | 4.25E+01 | 3.33E+01 | 8.77E+01 | 2.92E+03 |
| 6-14-TNF | TNF7 | 5.70E+01 | 4.00E+01 | 4.00E+01 | 1.70E+01 | 6.80E+02 |
| | TNF8 | 3.76E+01 | 1.87E+01 | 5.36E+01 | 1.88E+01 | 1.01E+03 |
| 6-15-TNF | TNF9 | 6.23E+01 | 3.38E+01 | 1.97E+02 | 2.85E+01 | 5.59E+03 |
| | TNF10 | 7.85E+01 | 2.75E+01 | 7.85E+01 | 5.10E+01 | 4.00E+03 |
| | MEAN | 7.60E+01 | 3.09E+01 | 2.80E+02 | 4.52E+01 | 2.53E+03 |
| | SD | 4.28E+01 | 1.79E+01 | 6.54E+02 | 3.59E+01 | 1.62E+03 |
| | SE | 1.35E+01 | 5.65E+00 | 2.07E+02 | 1.14E+01 | 5.12E+02 |
| 5-20-TNS | TNS1 | 6.47E+01 | 1.78E+01 | 1.04E+02 | 4.69E+01 | 4.88E+03 |
| | TNS2 | 8.95E+01 | 3.03E+01 | 5.55E+01 | 5.92E+01 | 3.29E+03 |
| 6-7-TNS | TNS3 | 7.79E+01 | 6.34E+01 | 1.28E+02 | 1.45E+01 | 1.85E+03 |
| | TNS4 | 7.74E+01 | 2.73E+01 | 1.02E+02 | 5.01E+01 | 5.09E+03 |
| 6-9-TNS | TNS5 | 1.37E+02 | 5.63E+01 | 1.63E+01 | 8.08E+01 | 1.32E+03 |
| | TNS6 | 8.59E+01 | 1.23E+01 | 1.06E+02 | 7.36E+01 | 7.79E+03 |
| 6-13-TNS | TNS7 | 5.76E+01 | 5.16E+01 | 1.35E+02 | 6.00E+00 | 8.07E+02 |
| | TNS8 | 4.96E+01 | 1.53E+01 | 1.22E+02 | 3.43E+01 | 4.20E+03 |
| 6-15-TNS | TNS9 | 9.97E+01 | 3.00E+01 | 7.46E+01 | 6.98E+01 | 5.21E+03 |
| | TNS10 | 4.32E+01 | −4.32E+00 | 7.20E+01 | 4.75E+01 | 3.42E+03 |
| | MEAN | 7.83E+01 | 3.00E+01 | 9.15E+01 | 4.83E+01 | 3.79E+03 |
| | SD | 2.74E+01 | 2.14E+01 | 3.69E+01 | 2.45E+01 | 2.11E+03 |
| | SE | 8.67E+00 | 6.78E+00 | 1.17E+01 | 7.75E+00 | 6.69E+02 |

TABLE 5

| | pre- | | | after 2 h | | |
|---|---|---|---|---|---|---|
| | ischemia | +dp/dtm | −dp/dtm | reperfusion | +dp/dtm | −dp/dtm |
| 5-18-CN | CON11 | 2.60E+03 | −1.82E+03 | CON11 | 1.44E+03 | −8.67E+02 |
| | CON12 | 2.95E+03 | −2.58E+03 | CON12 | 1.63E+03 | −1.07E+03 |
| 6-10-CN | CON13 | 3.10E+03 | −2.42E+03 | CON13 | 2.25E+02 | −2.22E+02 |
| | CON14 | 3.08E+03 | −2.10E+03 | CON14 | 3.44E+02 | −2.87E+02 |
| 5-15-CN | CON9 | 2.28E+03 | −1.38E+03 | CON9 | 9.45E+02 | −5.54E+02 |
| | CON10 | 2.06E+03 | −1.50E+03 | CON10 | 2.29E+03 | −1.75E+03 |
| 5-12-CN | CON7 | 2.71E+03 | −2.10E+03 | CON7 | 2.51E+02 | −2.55E+02 |
| | CON8 | 1.58E+03 | −1.10E+03 | CON8 | 3.63E+02 | −3.05E+02 |
| 5-9-CN | CONS | 2.17E+03 | −1.50E+03 | CONS | 2.39E+02 | −2.41E+02 |
| | CON6 | 2.25E+03 | −1.62E+03 | CON6 | 1.47E+02 | −1.49E+02 |
| 5-7-CN | CON3 | 2.63E+03 | −2.06E+03 | CON3 | 1.63E+03 | −1.06E+03 |
| | CON4 | 2.05E+03 | −1.38E+03 | CON4 | 1.10E+03 | −7.03E+02 |
| 5-5-CN | CON1 | 3.17E+03 | −2.37E+03 | CON1 | 1.03E+03 | −1.12E+03 |
| | CON2 | 2.10E+03 | −1.50E+03 | CON2 | 1.75E+03 | −1.27E+03 |
| | MEAN | 2.48E+03 | −1.82E+03 | MEAN | 9.56E+02 | −7.04E+02 |
| | SD | 4.84E+02 | 4.56E+02 | SD | 7.08E+02 | 4.95E+02 |
| | SE | 1.29E+02 | 1.22E+02 | SE | 1.89E+02 | 1.32E+02 |
| | TTEST | | | TTEST | | |
| 5-14-TMZ | TMZ3 | 2.41E+03 | −1.69E+03 | TMZ3 | 4.14E+02 | −3.57E+02 |
| | TMZ4 | 2.77E+03 | −2.26E+03 | TMZ4 | 1.48E+03 | −1.15E+03 |
| 5-11-TMZ | TMZ1 | 1.80E+03 | −1.59E+03 | TMZ1 | 1.38E+03 | −7.45E+02 |
| | TMZ2 | 2.15E+03 | −1.80E+03 | TMZ2 | 1.06E+03 | −6.85E+02 |
| 5-8-TMZ | TMZ7 | 3.40E+03 | −2.59E+03 | TMZ7 | 3.44E+02 | −3.39E+02 |
| | TMZ8 | 1.75E+03 | −1.20E+03 | TMZ8 | 7.36E+02 | −4.28E+02 |
| 5-6-TMZ | TMZ5 | 1.27E+03 | −8.82E+02 | TMZ5 | 1.28E+03 | −8.38E+02 |
| | TMZ6 | 1.24E+03 | −6.59E+02 | TMZ6 | 1.85E+03 | −1.06E+03 |
| 5-4-TMZ | TMZ9 | 1.98E+03 | −1.41E+03 | TMZ9 | 1.13E+03 | −6.38E+02 |
| | TMZ10 | 2.02E+03 | −1.56E+03 | TMZ10 | 1.62E+03 | −9.83E+02 |
| | MEAN | 2.08E+03 | −1.56E+03 | MEAN | 1.13E+03 | −7.22E+02 |
| | SD | 6.58E+02 | 5.81E+02 | SD | 5.01E+02 | 2.90E+02 |
| | SE | 2.08E+02 | 1.84E+02 | SE | 1.58E+02 | 9.16E+01 |
| | | | | | 5.16E−01 | 9.18E−01 |
| 5-19-TNF | TNF1 | 2.67E+03 | −1.49E+03 | TNF1 | 3.86E+02 | −3.75E+02 |
| | TNF2 | 2.85E+03 | −1.44E+03 | TNF2 | 1.46E+02 | −1.43E+02 |
| 6-8-TNF | TNF3 | 1.53E+03 | −7.24E+02 | TNF3 | 2.28E+02 | −2.34E+02 |
| | TNF4 | 3.86E+03 | −2.59E+03 | TNF4 | 2.84E+02 | −2.40E+02 |
| 6-12-TNF | TNF5 | 3.29E+03 | −2.34E+03 | TNF5 | 2.92E+03 | −2.08E+03 |
| | TNF6 | 3.03E+03 | −1.90E+03 | TNF6 | 2.48E+03 | −1.84E+03 |
| 6-14-TNF | TNF7 | 3.22E+03 | −1.62E+03 | TNF7 | 2.53E+02 | −2.48E+02 |
| | TNF8 | 1.74E+03 | −1.12E+03 | TNF8 | 1.53E+02 | −1.52E+02 |
| 6-15-TNF | TNF9 | 2.14E+03 | −2.33E+03 | TNF9 | 1.04E+03 | −6.31E+02 |
| | TNF10 | 1.86E+03 | −9.97E+02 | TNF10 | 2.04E+03 | −1.34E+03 |
| | MEAN | 2.62E+03 | −1.65E+03 | MEAN | 9.93E+02 | −7.29E+02 |
| | SD | 7.71E+02 | 6.26E+02 | SD | 1.08E+03 | 7.43E+02 |
| | SE | 2.44E+02 | 1.98E+02 | SE | 3.41E+02 | 2.35E+02 |
| | | | | | 1.09E−03 | 7.48E−03 |
| 5-20-TNS | TNS1 | 2.37E+03 | −1.60E+03 | TNS1 | 1.79E+03 | −1.12E+03 |
| | TNS2 | 2.87E+03 | −2.53E+03 | TNS2 | 1.84E+03 | −1.30E+03 |
| 6-7-TNS | TNS3 | 4.00E+03 | −2.67E+03 | TNS3 | 2.91E+02 | −3.02E+02 |
| | TNS4 | 3.32E+03 | −2.63E+03 | TNS4 | 1.62E+03 | −1.30E+03 |
| 6-9-TNS | TNS5 | 3.36E+03 | −2.21E+03 | TNS5 | 2.43E+02 | −2.46E+02 |
| | TNS6 | 2.53E+03 | −1.89E+03 | TNS6 | 2.36E+03 | −1.74E+03 |
| 6-13-TNS | TNS7 | 2.92E+03 | −1.75E+03 | TNS7 | 2.49E+02 | −2.47E+02 |
| | TNS8 | 1.12E+03 | −7.42E+02 | TNS8 | 1.29E+03 | −8.50E+02 |
| 6-15-TNS | TNS9 | 2.29E+03 | −1.75E+03 | TNS9 | 2.06E+03 | −1.59E+03 |
| | TNS10 | 2.11E+03 | −1.58E+03 | TNS10 | 1.26E+03 | −1.10E+03 |
| | MEAN | 2.69E+03 | −1.94E+03 | MEAN | 1.30E+03 | −9.80E+02 |
| | SD | 7.99E+02 | 5.95E+02 | SD | 7.86E+02 | 5.52E+02 |
| | SE | 2.53E+02 | 1.88E+02 | SE | 2.49E+02 | 1.75E+02 |
| | | 9.96E−04 | 1.55E−03 | | | |

TABLE 6

| | | CON | TMZ | TNF | TNS |
|---|---|---|---|---|---|
| T20 | Mean | 64.36 | 55.86 | 64.90 | 65.96 |
| T20 | SE | 3.91 | 5.58 | 5.73 | 5.49 |
| T170 | Mean | 37.09 | 48.91 | 45.16 | 48.27 |
| T170 | SE | 5.08 | 5.46 | 11.36 | 7.75 |

FIG. 41 shows images of TTC-stained heart slices after IR. TMZ and TNS treatment decreased infarct size after IR.

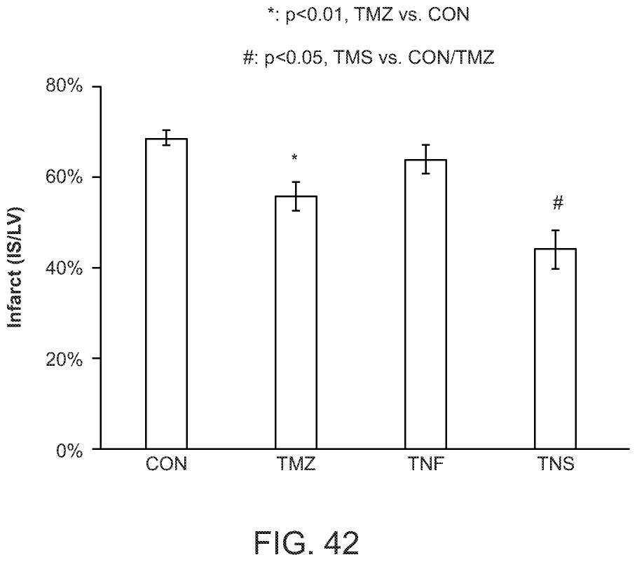

FIG. 42 is graph of infarct size after IR. TMZ and TNS treatment decreased infarct size after IR. Raw data is provided in Tables 7-55.

TABLE 7
CN11 raw values

| | | | |
|---|---|---|---|
| 1 | Slide11.jpg | 1649 | |
| 2 | Slide11.jpg | 10 | 0.06 |
| 3 | Slide11.jpg | 1385 | 8.40 |
| 4 | Slide11.jpg | 2808 | |
| 5 | Slide11.jpg | 104 | 0.81 |
| 6 | Slide11.jpg | 2525 | 19.78 |
| 7 | Slide11.jpg | 3807 | |
| 8 | Slide11.jpg | 1014 | 7.99 |
| 9 | Slide11.jpg | 2207 | 17.39 |
| 10 | Slide11.jpg | 3952 | |
| 11 | Slide11.jpg | 15 | 0.08 |
| 12 | Slide11.jpg | 3300 | 17.54 |
| 13 | Slide11.jpg | 3376 | |
| 14 | Slide11.jpg | 103 | 0.92 |
| 15 | Slide11.jpg | 2816 | 25.02 |
| 16 | Slide11.jpg | 1616 | |
| 17 | Slide11.jpg | 975 | 6.03 |
| 18 | Slide11.jpg | 409 | 2.53 |
| 19 | Slide11.jpg | 2805 | |
| 20 | Slide11.jpg | 819 | 6.42 |
| 21 | Slide11.jpg | 1496 | 11.73 |
| 22 | Slide11.jpg | 3973 | |
| 23 | Slide11.jpg | 1047 | 7.91 |
| 24 | Slide11.jpg | 2465 | 18.61 |
| 25 | Slide11.jpg | 3971 | |
| 26 | Slide11.jpg | 1102 | 5.83 |
| 27 | Slide11.jpg | 2430 | 12.85 |
| 28 | Slide11.jpg | 3516 | |
| 29 | Slide11.jpg | 1919 | 16.37 |
| 30 | Slide11.jpg | 920 | 7.85 |

TABLE 8
CN11 summary

| | |
|---|---|
| non-IS | 26.21 |
| IS | 70.86 |
| LV | 97.07 |
| IS/LV | 73% |

TABLE 9
CN12 raw values

| | | | |
|---|---|---|---|
| 1 | Slide12.jpg | 1562 | |
| 2 | Slide12.jpg | 1059 | 8.81 |
| 3 | Slide12.jpg | 485 | 4.04 |
| 4 | Slide12.jpg | 2925 | |
| 5 | Slide12.jpg | 260 | 1.78 |
| 6 | Slide12.jpg | 2159 | 14.76 |
| 7 | Slide12.jpg | 3492 | |
| 8 | Slide12.jpg | 263 | 1.88 |
| 9 | Slide12.jpg | 2886 | 20.66 |
| 10 | Slide12.jpg | 4855 | |
| 11 | Slide12.jpg | 1992 | 16.00 |
| 12 | Slide12.jpg | 2292 | 18.41 |
| 13 | Slide12.jpg | 2934 | |
| 14 | Slide12.jpg | 1405 | 6.70 |
| 15 | Slide12.jpg | 914 | 4.36 |
| 16 | Slide12.jpg | 2061 | |
| 17 | Slide12.jpg | 81 | 0.51 |
| 18 | Slide12.jpg | 1704 | 10.75 |
| 19 | Slide12.jpg | 2966 | |
| 20 | Slide12.jpg | 105 | 0.71 |
| 21 | Slide12.jpg | 2810 | 18.95 |
| 22 | Slide12.jpg | 4099 | |
| 23 | Slide12.jpg | 823 | 5.02 |
| 24 | Slide12.jpg | 2350 | 14.33 |
| 25 | Slide12.jpg | 3979 | |
| 26 | Slide12.jpg | 357 | 3.50 |
| 27 | Slide12.jpg | 2787 | 27.32 |
| 28 | Slide12.jpg | 2974 | |
| 29 | Slide12.jpg | 490 | 2.31 |
| 30 | Slide12.jpg | 2112 | 9.94 |

TABLE 10
CN12 summary

| | |
|---|---|
| non-IS | 23.61 |
| IS | 71.76 |
| LV | 95.37 |
| IS/LV | 75% |

TABLE 11
TNS1 raw values

| | | | |
|---|---|---|---|
| 1 | Slide15.jpg | 1857 | |
| 2 | Slide15.jpg | 58 | 0.28 |
| 3 | Slide15.jpg | 1672 | 8.10 |
| 4 | Slide15.jpg | 3383 | |
| 5 | Slide15.jpg | 901 | 4.53 |
| 6 | Slide15.jpg | 1873 | 9.41 |
| 7 | Slide15.jpg | 3460 | |
| 8 | Slide15.jpg | 1452 | 13.43 |
| 9 | Slide15.jpg | 2272 | 21.01 |
| 10 | Slide15.jpg | 3712 | |
| 11 | Slide15.jpg | 772 | 8.32 |
| 12 | Slide15.jpg | 2422 | 26.10 |
| 13 | Slide15.jpg | 3088 | |
| 14 | Slide15.jpg | 498 | 3.87 |
| 15 | Slide15.jpg | 1733 | 13.47 |
| 16 | Slide15.jpg | 1762 | |
| 17 | Slide15.jpg | 65 | 0.33 |
| 18 | Slide15.jpg | 1626 | 8.31 |
| 19 | Slide15.jpg | 3532 | |
| 20 | Slide15.jpg | 2034 | 9.79 |
| 21 | Slide15.jpg | 1206 | 5.80 |
| 22 | Slide15.jpg | 3411 | |
| 23 | Slide15.jpg | 1752 | 16.44 |
| 24 | Slide15.jpg | 1006 | 9.44 |
| 25 | Slide15.jpg | 4241 | |
| 26 | Slide15.jpg | 2148 | 20.26 |
| 27 | Slide15.jpg | 1101 | 10.38 |
| 28 | Slide15.jpg | 3440 | |
| 29 | Slide15.jpg | 2307 | 16.10 |
| 30 | Slide15.jpg | 165 | 1.15 |

TABLE 12
TNS1 summary

| | |
|---|---|
| non-IS | 46.67 |
| IS | 56.59 |
| LV | 103.26 |
| IS/LV | 55% |

TABLE 13
TNS2 raw values

| | | | |
|---|---|---|---|
| 1 | Slide16.jpg | 1565 | |
| 2 | Slide16.jpg | 1058 | 7.44 |
| 3 | Slide16.jpg | 145 | 1.02 |
| 4 | Slide16.jpg | 2654 | |
| 5 | Slide16.jpg | 431 | 3.90 |
| 6 | Slide16.jpg | 2043 | 18.47 |
| 7 | Slide16.jpg | 3247 | |
| 8 | Slide16.jpg | 1053 | 8.43 |
| 9 | Slide16.jpg | 1584 | 12.68 |
| 10 | Slide16.jpg | 3892 | |
| 11 | Slide16.jpg | 2391 | 22.73 |
| 12 | Slide16.jpg | 863 | 8.20 |
| 13 | Slide16.jpg | 2505 | |
| 14 | Slide16.jpg | 1488 | 14.85 |
| 15 | Slide16.jpg | 363 | 3.62 |
| 16 | Slide16.jpg | 1526 | |
| 17 | Slide16.jpg | 9 | 0.06 |
| 18 | Slide16.jpg | 1357 | 9.78 |
| 19 | Slide16.jpg | 2337 | |
| 20 | Slide16.jpg | 16 | 0.16 |
| 21 | Slide16.jpg | 1899 | 19.50 |
| 22 | Slide16.jpg | 3558 | |
| 23 | Slide16.jpg | 1453 | 10.62 |
| 24 | Slide16.jpg | 1504 | 10.99 |
| 25 | Slide16.jpg | 4041 | |
| 26 | Slide16.jpg | 517 | 4.73 |
| 27 | Slide16.jpg | 2763 | 25.30 |
| 28 | Slide16.jpg | 2946 | |
| 29 | Slide16.jpg | 631 | 5.35 |
| 30 | Slide16.jpg | 1326 | 11.25 |

TABLE 14
TNS2 summary

| | |
|---|---|
| non-IS | 39.14 |
| IS | 60.41 |
| LV | 99.56 |
| IS/LV | 61% |

TABLE 15
TNF1 raw values

| | | | |
|---|---|---|---|
| 1 | Slide17.jpg | 1326 | |
| 2 | Slide17.jpg | 63 | 0.24 |
| 3 | Slide17.jpg | 1183 | 4.46 |
| 4 | Slide17.jpg | 3158 | |
| 5 | Slide17.jpg | 825 | 5.49 |
| 6 | Slide17.jpg | 2014 | 13.39 |
| 7 | Slide17.jpg | 4805 | |
| 8 | Slide17.jpg | 1774 | 12.92 |
| 9 | Slide17.jpg | 1722 | 12.54 |
| 10 | Slide17.jpg | 4675 | |
| 11 | Slide17.jpg | 1984 | 15.28 |
| 12 | Slide17.jpg | 2470 | 19.02 |
| 13 | Slide17.jpg | 2754 | |
| 14 | Slide17.jpg | 269 | 2.05 |
| 15 | Slide17.jpg | 1377 | 10.50 |
| 16 | Slide17.jpg | 1373 | |
| 17 | Slide17.jpg | 1067 | 3.89 |
| 18 | Slide17.jpg | 43 | 0.16 |
| 19 | Slide17.jpg | 3113 | |
| 20 | Slide17.jpg | 803 | 5.42 |
| 21 | Slide17.jpg | 2008 | 13.55 |
| 22 | Slide17.jpg | 4657 | |
| 23 | Slide17.jpg | 1189 | 8.94 |
| 24 | Slide17.jpg | 2398 | 18.02 |
| 25 | Slide17.jpg | 4607 | |
| 26 | Slide17.jpg | 1256 | 9.81 |
| 27 | Slide17.jpg | 1978 | 15.46 |
| 28 | Slide17.jpg | 2769 | |
| 29 | Slide17.jpg | 2115 | 16.04 |
| 30 | Slide17.jpg | 72 | 0.55 |

TABLE 16
TNF1 summary

| | |
|---|---|
| non-IS | 40.03 |
| IS | 53.82 |
| LV | 93.86 |
| IS/LV | 57% |

TABLE 17
TNF2 raw values

| | | | |
|---|---|---|---|
| 1 | Slide18.jpg | 2133 | |
| 2 | Slide18.jpg | 1861 | 12.21 |
| 3 | Slide18.jpg | 239 | 1.57 |
| 4 | Slide18.jpg | 4037 | |
| 5 | Slide18.jpg | 753 | 5.60 |
| 6 | Slide18.jpg | 2304 | 17.12 |
| 7 | Slide18.jpg | 4663 | |
| 8 | Slide18.jpg | 1548 | 10.62 |
| 9 | Slide18.jpg | 2917 | 20.02 |
| 10 | Slide18.jpg | 5017 | |
| 11 | Slide18.jpg | 2648 | 20.06 |
| 12 | Slide18.jpg | 2480 | 18.78 |
| 13 | Slide18.jpg | 3629 | |
| 14 | Slide18.jpg | 1698 | 13.10 |
| 15 | Slide18.jpg | 348 | 2.69 |
| 16 | Slide18.jpg | 2130 | |
| 17 | Slide18.jpg | 4 | 0.03 |
| 18 | Slide18.jpg | 1988 | 13.07 |
| 19 | Slide18.jpg | 4108 | |
| 20 | Slide18.jpg | 253 | 1.85 |
| 21 | Slide18.jpg | 3796 | 27.72 |
| 22 | Slide18.jpg | 4612 | |
| 23 | Slide18.jpg | 815 | 5.65 |
| 24 | Slide18.jpg | 2427 | 16.84 |
| 25 | Slide18.jpg | 4880 | |
| 26 | Slide18.jpg | 562 | 4.38 |
| 27 | Slide18.jpg | 3535 | 27.53 |
| 28 | Slide18.jpg | 3507 | |
| 29 | Slide18.jpg | 497 | 3.97 |
| 30 | Slide18.jpg | 1837 | 14.67 |

TABLE 18
TNF2 summary

| | |
|---|---|
| non-IS | 38.73 |
| IS | 80.00 |
| LV | 118.73 |
| IS/LV | 73% |

TABLE 19
TNS3 raw values

| | | | |
|---|---|---|---|
| 1 | Slide19.jpg | 1484 | |
| 2 | Slide19.jpg | 923 | 4.98 |
| 3 | Slide19.jpg | 714 | 3.85 |
| 4 | Slide19.jpg | 3124 | |
| 5 | Slide19.jpg | 990 | 6.65 |
| 6 | Slide19.jpg | 1845 | 12.40 |
| 7 | Slide19.jpg | 3414 | |
| 8 | Slide19.jpg | 1282 | 13.89 |
| 9 | Slide19.jpg | 1833 | 19.87 |
| 10 | Slide19.jpg | 3380 | |
| 11 | Slide19.jpg | 2123 | 16.33 |
| 12 | Slide19.jpg | 1042 | 8.02 |
| 13 | Slide19.jpg | 2105 | |
| 14 | Slide19.jpg | 957 | 7.73 |
| 15 | Slide19.jpg | 308 | 2.49 |
| 16 | Slide19.jpg | 1524 | |
| 17 | Slide19.jpg | 10 | 0.05 |
| 18 | Slide19.jpg | 1530 | 8.03 |
| 19 | Slide19.jpg | 2860 | |
| 20 | Slide19.jpg | 13 | 0.10 |
| 21 | Slide19.jpg | 2293 | 16.84 |
| 22 | Slide19.jpg | 3358 | |
| 23 | Slide19.jpg | 960 | 10.58 |
| 24 | Slide19.jpg | 2639 | 29.08 |
| 25 | Slide19.jpg | 2538 | |
| 26 | Slide19.jpg | 296 | 3.03 |
| 27 | Slide19.jpg | 1797 | 18.41 |
| 28 | Slide19.jpg | 1992 | |
| 29 | Slide19.jpg | 1105 | 9.43 |
| 30 | Slide19.jpg | 401 | 3.42 |

TABLE 20
TNS3 summary

| | |
|---|---|
| non-IS | 36.39 |
| IS | 61.20 |
| LV | 97.58 |
| IS/LV | 63% |

TABLE 21
TNS4 raw values

| | | | |
|---|---|---|---|
| 1 | Slide20.jpg | 1524 | |
| 2 | Slide20.jpg | 47 | 0.28 |
| 3 | Slide20.jpg | 1417 | 8.37 |
| 4 | Slide20.jpg | 2478 | |
| 5 | Slide20.jpg | 582 | 5.17 |
| 6 | Slide20.jpg | 1617 | 14.36 |
| 7 | Slide20.jpg | 3284 | |
| 8 | Slide20.jpg | 1226 | 11.20 |
| 9 | Slide20.jpg | 2072 | 18.93 |
| 10 | Slide20.jpg | 3639 | |
| 11 | Slide20.jpg | 771 | 7.20 |
| 12 | Slide20.jpg | 2177 | 20.34 |
| 13 | Slide20.jpg | 3114 | |
| 14 | Slide20.jpg | 491 | 5.36 |
| 15 | Slide20.jpg | 2189 | 23.90 |
| 16 | Slide20.jpg | 1648 | |
| 17 | Slide20.jpg | 1244 | 6.79 |
| 18 | Slide20.jpg | 94 | 0.51 |
| 19 | Slide20.jpg | 2912 | |
| 20 | Slide20.jpg | 1446 | 10.92 |
| 21 | Slide20.jpg | 1262 | 9.53 |
| 22 | Slide20.jpg | 4073 | |
| 23 | Slide20.jpg | 2350 | 17.31 |
| 24 | Slide20.jpg | 1049 | 7.73 |
| 25 | Slide20.jpg | 3470 | |
| 26 | Slide20.jpg | 2445 | 23.96 |
| 27 | Slide20.jpg | 1052 | 10.31 |
| 28 | Slide20.jpg | 3219 | |
| 29 | Slide20.jpg | 2120 | 22.39 |
| 30 | Slide20.jpg | 32 | 0.34 |

TABLE 22
TNS4 summary

| | |
|---|---|
| non-IS | 55.29 |
| IS | 57.16 |
| LV | 112.45 |
| IS/LV | 51% |

TABLE 23
TNF3 raw values

| | | | |
|---|---|---|---|
| 1 | Slide21.jpg | 1551 | |
| 2 | Slide21.jpg | 3 | 0.02 |
| 3 | Slide21.jpg | 1502 | 10.65 |
| 4 | Slide21.jpg | 3054 | |
| 5 | Slide21.jpg | 922 | 6.34 |
| 6 | Slide21.jpg | 2049 | 14.09 |
| 7 | Slide21.jpg | 3374 | |
| 8 | Slide21.jpg | 1280 | 12.52 |
| 9 | Slide21.jpg | 1566 | 15.32 |
| 10 | Slide21.jpg | 2799 | |
| 11 | Slide21.jpg | 1476 | 14.77 |
| 12 | Slide21.jpg | 1061 | 10.61 |
| 13 | Slide21.jpg | 2330 | |
| 14 | Slide21.jpg | 398 | 3.25 |
| 15 | Slide21.jpg | 1012 | 8.25 |
| 16 | Slide21.jpg | 1689 | |
| 17 | Slide21.jpg | 7 | 0.05 |
| 18 | Slide21.jpg | 1544 | 10.06 |
| 19 | Slide21.jpg | 2894 | |
| 20 | Slide21.jpg | 361 | 2.62 |
| 21 | Slide21.jpg | 1925 | 13.97 |
| 22 | Slide21.jpg | 3254 | |
| 23 | Slide21.jpg | 1137 | 11.53 |
| 24 | Slide21.jpg | 1267 | 12.85 |
| 25 | Slide21.jpg | 2814 | |
| 26 | Slide21.jpg | 1272 | 12.66 |
| 27 | Slide21.jpg | 1113 | 11.07 |
| 28 | Slide21.jpg | 2821 | |
| 29 | Slide21.jpg | 1438 | 9.69 |
| 30 | Slide21.jpg | 174 | 1.17 |

TABLE 24
TNF3 summary

| | |
|---|---|
| non-IS | 36.71 |
| IS | 54.02 |
| LV | 90.74 |
| IS/LV | 60% |

TABLE 25
TNF4 raw values

| | | | |
|---|---|---|---|
| 1 | Slide22.jpg | 1354 | |
| 2 | Slide22.jpg | 72 | 0.37 |
| 3 | Slide22.jpg | 1335 | 6.90 |
| 4 | Slide22.jpg | 2892 | |
| 5 | Slide22.jpg | 672 | 3.95 |
| 6 | Slide22.jpg | 2093 | 12.30 |
| 7 | Slide22.jpg | 3414 | |
| 8 | Slide22.jpg | 1342 | 9.83 |
| 9 | Slide22.jpg | 2213 | 16.21 |
| 10 | Slide22.jpg | 3698 | |
| 11 | Slide22.jpg | 1168 | 10.11 |
| 12 | Slide22.jpg | 2317 | 20.05 |
| 13 | Slide22.jpg | 2565 | |
| 14 | Slide22.jpg | 243 | 2.94 |
| 15 | Slide22.jpg | 1398 | 16.90 |
| 16 | Slide22.jpg | 1486 | |
| 17 | Slide22.jpg | 638 | 3.01 |
| 18 | Slide22.jpg | 583 | 2.75 |
| 19 | Slide22.jpg | 2719 | |
| 20 | Slide22.jpg | 26 | 0.16 |
| 21 | Slide22.jpg | 2164 | 13.53 |
| 22 | Slide22.jpg | 3514 | |
| 23 | Slide22.jpg | 568 | 4.04 |
| 24 | Slide22.jpg | 2361 | 16.80 |
| 25 | Slide22.jpg | 3908 | |
| 26 | Slide22.jpg | 1498 | 12.27 |
| 27 | Slide22.jpg | 1805 | 14.78 |
| 28 | Slide22.jpg | 2946 | |
| 29 | Slide22.jpg | 16 | 0.17 |
| 30 | Slide22.jpg | 1969 | 20.72 |

TABLE 26
TNF4 summary

| | |
|---|---|
| non-IS | 23.42 |
| IS | 70.46 |
| LV | 93.88 |
| IS/LV | 75% |

TABLE 27
TNS5 raw values

| | | | |
|---|---|---|---|
| 1 | Slide23.jpg | 1615 | |
| 2 | Slide23.jpg | 8 | 0.04 |
| 3 | Slide23.jpg | 1571 | 8.75 |
| 4 | Slide23.jpg | 2789 | |
| 5 | Slide23.jpg | 1477 | 11.65 |
| 6 | Slide23.jpg | 1042 | 8.22 |
| 7 | Slide23.jpg | 3558 | |
| 8 | Slide23.jpg | 2026 | 22.21 |
| 9 | Slide23.jpg | 1327 | 14.55 |
| 10 | Slide23.jpg | 3822 | |
| 11 | Slide23.jpg | 1044 | 8.74 |
| 12 | Slide23.jpg | 1590 | 13.31 |
| 13 | Slide23.jpg | 3246 | |
| 14 | Slide23.jpg | 1224 | 8.67 |
| 15 | Slide23.jpg | 705 | 5.00 |
| 16 | Slide23.jpg | 1445 | |
| 17 | Slide23.jpg | 1228 | 7.65 |
| 18 | Slide23.jpg | 200 | 1.25 |
| 19 | Slide23.jpg | 2732 | |
| 20 | Slide23.jpg | 1951 | 15.71 |
| 21 | Slide23.jpg | 782 | 6.30 |
| 22 | Slide23.jpg | 3858 | |
| 23 | Slide23.jpg | 3039 | 30.72 |
| 24 | Slide23.jpg | 400 | 4.04 |
| 25 | Slide23.jpg | 3697 | |
| 26 | Slide23.jpg | 2609 | 22.58 |
| 27 | Slide23.jpg | 943 | 8.16 |
| 28 | Slide23.jpg | 3358 | |
| 29 | Slide23.jpg | 1492 | 10.22 |
| 30 | Slide23.jpg | 583 | 3.99 |

TABLE 28
TNS5 summary

| | |
|---|---|
| non-IS | 69.10 |
| IS | 36.78 |
| LV | 105.88 |
| IS/LV | 35% |

TABLE 29
TNS6 raw values

| | | | |
|---|---|---|---|
| 1 | Slide24.jpg | 1216 | |
| 2 | Slide24.jpg | 258 | 1.49 |
| 3 | Slide24.jpg | 770 | 4.43 |
| 4 | Slide24.jpg | 3079 | |
| 5 | Slide24.jpg | 1436 | 10.26 |
| 6 | Slide24.jpg | 1417 | 10.12 |
| 7 | Slide24.jpg | 3677 | |
| 8 | Slide24.jpg | 2085 | 11.34 |
| 9 | Slide24.jpg | 1122 | 6.10 |
| 10 | Slide24.jpg | 3908 | |
| 11 | Slide24.jpg | 2151 | 15.96 |
| 12 | Slide24.jpg | 1415 | 10.50 |
| 13 | Slide24.jpg | 2371 | |
| 14 | Slide24.jpg | 1651 | 14.62 |
| 15 | Slide24.jpg | 495 | 4.38 |
| 16 | Slide24.jpg | 1123 | |
| 17 | Slide24.jpg | 879 | 5.48 |
| 18 | Slide24.jpg | 262 | 1.63 |
| 19 | Slide24.jpg | 3090 | |
| 20 | Slide24.jpg | 1775 | 12.64 |
| 21 | Slide24.jpg | 1121 | 7.98 |
| 22 | Slide24.jpg | 3470 | |
| 23 | Slide24.jpg | 2215 | 12.77 |
| 24 | Slide24.jpg | 1219 | 7.03 |
| 25 | Slide24.jpg | 3666 | |
| 26 | Slide24.jpg | 2524 | 19.97 |
| 27 | Slide24.jpg | 1411 | 11.16 |
| 28 | Slide24.jpg | 2470 | |
| 29 | Slide24.jpg | 1397 | 11.88 |
| 30 | Slide24.jpg | 140 | 1.19 |

TABLE 30
TNS6 summary

| | |
|---|---|
| non-IS | 58.20 |
| IS | 32.27 |
| LV | 90.47 |
| IS/LV | 36% |

TABLE 31
CN13 raw values

| | | | |
|---|---|---|---|
| 1 | Slide25.jpg | 1010 | |
| 2 | Slide25.jpg | 4 | 0.04 |
| 3 | Slide25.jpg | 1006 | 8.96 |
| 4 | Slide25.jpg | 2216 | |
| 5 | Slide25.jpg | 756 | 5.80 |
| 6 | Slide25.jpg | 1708 | 13.10 |
| 7 | Slide25.jpg | 3122 | |
| 8 | Slide25.jpg | 744 | 5.72 |
| 9 | Slide25.jpg | 1674 | 12.87 |
| 10 | Slide25.jpg | 3214 | |
| 11 | Slide25.jpg | 177 | 1.87 |
| 12 | Slide25.jpg | 1678 | 17.75 |
| 13 | Slide25.jpg | 2504 | |
| 14 | Slide25.jpg | 371 | 3.41 |
| 15 | Slide25.jpg | 770 | 7.07 |
| 16 | Slide25.jpg | 940 | |
| 17 | Slide25.jpg | 3 | 0.03 |
| 18 | Slide25.jpg | 902 | 8.64 |
| 19 | Slide25.jpg | 1907 | |
| 20 | Slide25.jpg | 266 | 2.37 |
| 21 | Slide25.jpg | 1439 | 12.83 |
| 22 | Slide25.jpg | 2763 | |
| 23 | Slide25.jpg | 1036 | 9.00 |
| 24 | Slide25.jpg | 1855 | 16.11 |
| 25 | Slide25.jpg | 2930 | |
| 26 | Slide25.jpg | 988 | 11.46 |
| 27 | Slide25.jpg | 1618 | 18.78 |
| 28 | Slide25.jpg | 2498 | |
| 29 | Slide25.jpg | 280 | 2.58 |
| 30 | Slide25.jpg | 1839 | 16.93 |

TABLE 32
CN13 summary

| | |
|---|---|
| non-IS | 21.14 |
| IS | 66.52 |
| LV | 87.66 |
| IS/LV | 76% |

TABLE 33
CN14 raw values

| | | | |
|---|---|---|---|
| 1 | Slide26.jpg | 1387 | |
| 2 | Slide26.jpg | 40 | 0.23 |
| 3 | Slide26.jpg | 1356 | 7.82 |
| 4 | Slide26.jpg | 2994 | |
| 5 | Slide26.jpg | 699 | 4.67 |
| 6 | Slide26.jpg | 1620 | 10.82 |
| 7 | Slide26.jpg | 3017 | |
| 8 | Slide26.jpg | 1087 | 11.89 |
| 9 | Slide26.jpg | 1443 | 15.78 |
| 10 | Slide26.jpg | 2871 | |
| 11 | Slide26.jpg | 2644 | 29.47 |
| 12 | Slide26.jpg | 188 | 2.10 |
| 13 | Slide26.jpg | 2504 | |
| 14 | Slide26.jpg | 7 | 0.05 |
| 15 | Slide26.jpg | 1996 | 13.55 |
| 16 | Slide26.jpg | 1424 | |
| 17 | Slide26.jpg | 490 | 2.75 |
| 18 | Slide26.jpg | 931 | 5.23 |
| 19 | Slide26.jpg | 2926 | |
| 20 | Slide26.jpg | 40 | 0.27 |
| 21 | Slide26.jpg | 2231 | 15.25 |
| 22 | Slide26.jpg | 3248 | |
| 23 | Slide26.jpg | 782 | 7.95 |
| 24 | Slide26.jpg | 2137 | 21.71 |
| 25 | Slide26.jpg | 3401 | |
| 26 | Slide26.jpg | 348 | 3.27 |
| 27 | Slide26.jpg | 2624 | 24.69 |
| 28 | Slide26.jpg | 2079 | |
| 29 | Slide26.jpg | 573 | 4.69 |
| 30 | Slide26.jpg | 1042 | 8.52 |

TABLE 34
CN14 summary

| | |
|---|---|
| non-IS | 32.62 |
| IS | 62.74 |
| LV | 95.36 |
| IS/LV | 66% |

TABLE 35
TNF5 raw values

| | | | |
|---|---|---|---|
| 1 | Slide27.jpg | 1504 | |
| 2 | Slide27.jpg | 22 | 0.13 |
| 3 | Slide27.jpg | 1336 | 7.99 |
| 4 | Slide27.jpg | 2786 | |
| 5 | Slide27.jpg | 390 | 3.22 |
| 6 | Slide27.jpg | 1956 | 16.15 |
| 7 | Slide27.jpg | 3792 | |
| 8 | Slide27.jpg | 1444 | 10.66 |
| 9 | Slide27.jpg | 2232 | 16.48 |
| 10 | Slide27.jpg | 3470 | |
| 11 | Slide27.jpg | 587 | 5.41 |
| 12 | Slide27.jpg | 2824 | 26.04 |
| 13 | Slide27.jpg | 3002 | |
| 14 | Slide27.jpg | 2361 | 16.52 |
| 15 | Slide27.jpg | 1329 | 9.30 |
| 16 | Slide27.jpg | 1666 | |
| 17 | Slide27.jpg | 274 | 1.48 |
| 18 | Slide27.jpg | 1024 | 5.53 |
| 19 | Slide27.jpg | 2735 | |
| 20 | Slide27.jpg | 9 | 0.08 |
| 21 | Slide27.jpg | 2897 | 24.36 |
| 22 | Slide27.jpg | 3575 | |
| 23 | Slide27.jpg | 1217 | 9.53 |
| 24 | Slide27.jpg | 2163 | 16.94 |
| 25 | Slide27.jpg | 3350 | |
| 26 | Slide27.jpg | 997 | 9.52 |
| 27 | Slide27.jpg | 1812 | 17.31 |
| 28 | Slide27.jpg | 3022 | |
| 29 | Slide27.jpg | 12 | 0.08 |
| 30 | Slide27.jpg | 1778 | 12.36 |

TABLE 36
TNF5 summary

| | |
|---|---|
| non-IS | 28.32 |
| IS | 76.23 |
| LV | 104.55 |
| IS/LV | 73% |

TABLE 37
TNF6 raw values

| | | | |
|---|---|---|---|
| 1 | Slide28.jpg | 1114 | |
| 2 | Slide28.jpg | 62 | 0.45 |
| 3 | Slide28.jpg | 879 | 6.31 |
| 4 | Slide28.jpg | 2858 | |
| 5 | Slide28.jpg | 459 | 3.85 |
| 6 | Slide28.jpg | 1713 | 14.38 |
| 7 | Slide28.jpg | 3625 | |
| 8 | Slide28.jpg | 369 | 3.56 |
| 9 | Slide28.jpg | 2924 | 28.23 |
| 10 | Slide28.jpg | 3948 | |
| 11 | Slide28.jpg | 511 | 4.27 |
| 12 | Slide28.jpg | 2866 | 23.96 |
| 13 | Slide28.jpg | 3135 | |
| 14 | Slide28.jpg | 386 | 3.08 |
| 15 | Slide28.jpg | 1447 | 11.54 |
| 16 | Slide28.jpg | 1126 | |
| 17 | Slide28.jpg | 10 | 0.07 |
| 18 | Slide28.jpg | 1043 | 7.41 |
| 19 | Slide28.jpg | 3156 | |
| 20 | Slide28.jpg | 160 | 1.22 |
| 21 | Slide28.jpg | 3062 | 23.29 |
| 22 | Slide28.jpg | 3790 | |
| 23 | Slide28.jpg | 827 | 7.64 |
| 24 | Slide28.jpg | 2644 | 24.42 |
| 25 | Slide28.jpg | 3618 | |
| 26 | Slide28.jpg | 1607 | 14.66 |
| 27 | Slide28.jpg | 2452 | 22.36 |
| 28 | Slide28.jpg | 3440 | |
| 29 | Slide28.jpg | 1023 | 7.43 |
| 30 | Slide28.jpg | 1770 | 12.86 |

TABLE 38
TNF6 summary

| | |
|---|---|
| non-IS | 23.11 |
| IS | 87.38 |
| LV | 110.50 |
| IS/LV | 79% |

TABLE 39
TNS7 raw values

| | | | |
|---|---|---|---|
| 1 | Slide29.jpg | 1713 | |
| 2 | Slide29.jpg | 607 | 4.61 |
| 3 | Slide29.jpg | 782 | 5.93 |
| 4 | Slide29.jpg | 2484 | |
| 5 | Slide29.jpg | 195 | 1.88 |
| 6 | Slide29.jpg | 1842 | 17.80 |
| 7 | Slide29.jpg | 2807 | |
| 8 | Slide29.jpg | 1568 | 12.29 |
| 9 | Slide29.jpg | 380 | 2.98 |
| 10 | Slide29.jpg | 3271 | |
| 11 | Slide29.jpg | 2187 | 20.06 |
| 12 | Slide29.jpg | 350 | 3.21 |
| 13 | Slide29.jpg | 2309 | |
| 14 | Slide29.jpg | 610 | 5.55 |
| 15 | Slide29.jpg | 1008 | 9.17 |
| 16 | Slide29.jpg | 1923 | |
| 17 | Slide29.jpg | 865 | 5.85 |
| 18 | Slide29.jpg | 631 | 4.27 |
| 19 | Slide29.jpg | 3033 | |
| 20 | Slide29.jpg | 1501 | 11.88 |
| 21 | Slide29.jpg | 780 | 6.17 |
| 22 | Slide29.jpg | 3287 | |
| 23 | Slide29.jpg | 2214 | 14.82 |
| 24 | Slide29.jpg | 456 | 3.05 |
| 25 | Slide29.jpg | 3395 | |
| 26 | Slide29.jpg | 2398 | 21.19 |
| 27 | Slide29.jpg | 287 | 2.54 |
| 28 | Slide29.jpg | 2969 | |
| 29 | Slide29.jpg | 1647 | 11.65 |
| 30 | Slide29.jpg | 67 | 0.47 |

TABLE 40
TNS7 summary

| | |
|---|---|
| non-IS | 54.88 |
| IS | 27.79 |
| LV | 82.68 |
| IS/LV | 34% |

TABLE 41
TNS8 raw values

| | | | |
|---|---|---|---|
| 1 | Slide30.jpg | 1123 | |
| 2 | Slide30.jpg | 11 | 0.05 |
| 3 | Slide30.jpg | 988 | 4.40 |
| 4 | Slide30.jpg | 2352 | |
| 5 | Slide30.jpg | 279 | 2.25 |
| 6 | Slide30.jpg | 2001 | 16.16 |
| 7 | Slide30.jpg | 3274 | |
| 8 | Slide30.jpg | 1085 | 7.29 |
| 9 | Slide30.jpg | 1821 | 12.24 |
| 10 | Slide30.jpg | 3333 | |
| 11 | Slide30.jpg | 2048 | 17.20 |
| 12 | Slide30.jpg | 838 | 7.04 |
| 13 | Slide30.jpg | 2240 | |
| 14 | Slide30.jpg | 793 | 7.08 |
| 15 | Slide30.jpg | 840 | 7.50 |
| 16 | Slide30.jpg | 914 | |
| 17 | Slide30.jpg | 866 | 4.74 |
| 18 | Slide30.jpg | 64 | 0.35 |
| 19 | Slide30.jpg | 2811 | |
| 20 | Slide30.jpg | 397 | 2.68 |
| 21 | Slide30.jpg | 2135 | 14.43 |
| 22 | Slide30.jpg | 3378 | |
| 23 | Slide30.jpg | 588 | 3.83 |
| 24 | Slide30.jpg | 2250 | 14.65 |
| 25 | Slide30.jpg | 3241 | |
| 26 | Slide30.jpg | 2671 | 23.08 |
| 27 | Slide30.jpg | 287 | 2.48 |
| 28 | Slide30.jpg | 2697 | |
| 29 | Slide30.jpg | 1247 | 9.25 |
| 30 | Slide30.jpg | 23 | 0.17 |

TABLE 42
TNS8 summary

| | |
|---|---|
| non-IS | 38.73 |
| IS | 39.71 |
| LV | 78.44 |
| IS/LV | 51% |

TABLE 43
TNF7 raw values

| | | | |
|---|---|---|---|
| 1 | Slide31.jpg | 1733 | |
| 2 | Slide31.jpg | 15 | 0.06 |
| 3 | Slide31.jpg | 1704 | 6.88 |
| 4 | Slide31.jpg | 3401 | |
| 5 | Slide31.jpg | 719 | 3.38 |
| 6 | Slide31.jpg | 2216 | 10.43 |
| 7 | Slide31.jpg | 3789 | |
| 8 | Slide31.jpg | 917 | 7.02 |
| 9 | Slide31.jpg | 2163 | 16.56 |
| 10 | Slide31.jpg | 4149 | |
| 11 | Slide31.jpg | 719 | 5.03 |
| 12 | Slide31.jpg | 3423 | 23.93 |
| 13 | Slide31.jpg | 3309 | |
| 14 | Slide31.jpg | 1479 | 8.49 |
| 15 | Slide31.jpg | 1771 | 10.17 |
| 16 | Slide31.jpg | 1777 | |
| 17 | Slide31.jpg | 1049 | 4.13 |
| 18 | Slide31.jpg | 678 | 2.67 |
| 19 | Slide31.jpg | 3117 | |
| 20 | Slide31.jpg | 221 | 1.13 |
| 21 | Slide31.jpg | 2281 | 11.71 |
| 22 | Slide31.jpg | 3970 | |
| 23 | Slide31.jpg | 2416 | 17.65 |
| 24 | Slide31.jpg | 796 | 5.81 |
| 25 | Slide31.jpg | 4354 | |
| 26 | Slide31.jpg | 3291 | 21.92 |
| 27 | Slide31.jpg | 697 | 4.64 |
| 28 | Slide31.jpg | 3316 | |
| 29 | Slide31.jpg | 2414 | 13.83 |
| 30 | Slide31.jpg | 62 | 0.36 |

TABLE 44
TNF7 summary

| | |
|---|---|
| non-IS | 41.32 |
| IS | 46.57 |
| LV | 87.90 |
| IS/LV | 53% |

TABLE 45
TNF8 raw values

| | | | |
|---|---|---|---|
| 1 | Slide32.jpg | 1553 | |
| 2 | Slide32.jpg | 572 | 2.58 |
| 3 | Slide32.jpg | 873 | 3.93 |
| 4 | Slide32.jpg | 3334 | |
| 5 | Slide32.jpg | 1084 | 5.53 |
| 6 | Slide32.jpg | 1525 | 7.78 |
| 7 | Slide32.jpg | 4166 | |
| 8 | Slide32.jpg | 2437 | 12.87 |
| 9 | Slide32.jpg | 1557 | 8.22 |
| 10 | Slide32.jpg | 4558 | |
| 11 | Slide32.jpg | 2698 | 20.13 |
| 12 | Slide32.jpg | 1306 | 9.74 |
| 13 | Slide32.jpg | 3405 | |
| 14 | Slide32.jpg | 2991 | 25.47 |
| 15 | Slide32.jpg | 51 | 0.43 |
| 16 | Slide32.jpg | 1543 | |
| 17 | Slide32.jpg | 3 | 0.01 |
| 18 | Slide32.jpg | 1407 | 6.38 |
| 19 | Slide32.jpg | 3359 | |
| 20 | Slide32.jpg | 581 | 2.94 |
| 21 | Slide32.jpg | 2011 | 10.18 |
| 22 | Slide32.jpg | 3986 | |
| 23 | Slide32.jpg | 202 | 1.11 |
| 24 | Slide32.jpg | 3788 | 20.91 |
| 25 | Slide32.jpg | 4684 | |
| 26 | Slide32.jpg | 425 | 3.08 |
| 27 | Slide32.jpg | 3308 | 24.01 |
| 28 | Slide32.jpg | 3498 | |
| 29 | Slide32.jpg | 920 | 7.63 |
| 30 | Slide32.jpg | 1731 | 14.35 |

TABLE 46
TNF8 summary

| | |
|---|---|
| non-IS | 40.68 |
| IS | 52.97 |
| LV | 93.65 |
| IS/LV | 57% |

TABLE 47
TNS9 raw values

| | | | |
|---|---|---|---|
| 1 | Slide33.jpg | 2637 | |
| 2 | Slide33.jpg | 14 | 0.06 |
| 3 | Slide33.jpg | 2081 | 9.47 |
| 4 | Slide33.jpg | 4101 | |
| 5 | Slide33.jpg | 1571 | 7.28 |
| 6 | Slide33.jpg | 1516 | 7.02 |
| 7 | Slide33.jpg | 4527 | |
| 8 | Slide33.jpg | 2519 | 18.36 |
| 9 | Slide33.jpg | 1555 | 11.34 |
| 10 | Slide33.jpg | 3326 | |
| 11 | Slide33.jpg | 3188 | 19.17 |
| 12 | Slide33.jpg | 27 | 0.16 |
| 13 | Slide33.jpg | 2336 | |
| 14 | Slide33.jpg | 1885 | 9.68 |
| 15 | Slide33.jpg | 240 | 1.23 |
| 16 | Slide33.jpg | 2343 | |
| 17 | Slide33.jpg | 2027 | 10.38 |
| 18 | Slide33.jpg | 21 | 0.11 |
| 19 | Slide33.jpg | 3393 | |
| 20 | Slide33.jpg | 1928 | 10.80 |
| 21 | Slide33.jpg | 945 | 5.29 |
| 22 | Slide33.jpg | 4425 | |
| 23 | Slide33.jpg | 2984 | 22.25 |
| 24 | Slide33.jpg | 637 | 4.75 |
| 25 | Slide33.jpg | 3063 | |
| 26 | Slide33.jpg | 773 | 5.05 |
| 27 | Slide33.jpg | 1885 | 12.31 |
| 28 | Slide33.jpg | 2324 | |
| 29 | Slide33.jpg | 1390 | 7.18 |
| 30 | Slide33.jpg | 9 | 0.05 |

TABLE 48
TNS9 summary

| | |
|---|---|
| non-IS | 55.11 |
| IS | 25.86 |
| LV | 80.97 |
| IS/LV | 32% |

TABLE 49
TNS10 raw values

| | | | |
|---|---|---|---|
| 1 | Slide34.jpg | 1775 | |
| 2 | Slide34.jpg | 1082 | 4.88 |
| 3 | Slide34.jpg | 348 | 1.57 |
| 4 | Slide34.jpg | 3607 | |
| 5 | Slide34.jpg | 1823 | 11.12 |
| 6 | Slide34.jpg | 1483 | 9.05 |
| 7 | Slide34.jpg | 4313 | |
| 8 | Slide34.jpg | 1087 | 6.80 |
| 9 | Slide34.jpg | 2173 | 13.60 |
| 10 | Slide34.jpg | 4275 | |
| 11 | Slide34.jpg | 2471 | 15.03 |
| 12 | Slide34.jpg | 1734 | 10.55 |
| 13 | Slide34.jpg | 2864 | |
| 14 | Slide34.jpg | 2424 | 18.62 |
| 15 | Slide34.jpg | 43 | 0.33 |
| 16 | Slide34.jpg | 1601 | |
| 17 | Slide34.jpg | 1600 | 8.00 |
| 18 | Slide34.jpg | 16 | 0.08 |
| 19 | Slide34.jpg | 3486 | |
| 20 | Slide34.jpg | 933 | 5.89 |
| 21 | Slide34.jpg | 935 | 5.90 |
| 22 | Slide34.jpg | 4312 | |
| 23 | Slide34.jpg | 3250 | 20.35 |
| 24 | Slide34.jpg | 722 | 4.52 |
| 25 | Slide34.jpg | 4178 | |
| 26 | Slide34.jpg | 3996 | 24.87 |
| 27 | Slide34.jpg | 231 | 1.44 |
| 28 | Slide34.jpg | 3046 | |
| 29 | Slide34.jpg | 2854 | 20.61 |
| 30 | Slide34.jpg | 39 | 0.28 |

TABLE 50
TNS10 summary

| | |
|---|---|
| non-IS | 68.08 |
| IS | 23.66 |
| LV | 91.74 |
| IS/LV | 26% |

TABLE 51
TNF9 raw values

| | | | |
|---|---|---|---|
| 1 | Slide35.jpg | 1737 | |
| 2 | Slide35.jpg | 841 | 2.91 |
| 3 | Slide35.jpg | 788 | 2.72 |
| 4 | Slide35.jpg | 3368 | |
| 5 | Slide35.jpg | 1416 | 7.99 |
| 6 | Slide35.jpg | 1230 | 6.94 |
| 7 | Slide35.jpg | 4474 | |
| 8 | Slide35.jpg | 1046 | 8.18 |
| 9 | Slide35.jpg | 3356 | 26.25 |
| 10 | Slide35.jpg | 4877 | |
| 11 | Slide35.jpg | 1303 | 6.68 |
| 12 | Slide35.jpg | 3142 | 16.11 |
| 13 | Slide35.jpg | 3803 | |
| 14 | Slide35.jpg | 2906 | 16.81 |
| 15 | Slide35.jpg | 15 | 0.09 |
| 16 | Slide35.jpg | 1719 | |
| 17 | Slide35.jpg | 8 | 0.03 |
| 18 | Slide35.jpg | 1545 | 5.39 |
| 19 | Slide35.jpg | 3500 | |
| 20 | Slide35.jpg | 9 | 0.05 |
| 21 | Slide35.jpg | 3382 | 18.36 |
| 22 | Slide35.jpg | 4790 | |
| 23 | Slide35.jpg | 9 | 0.07 |
| 24 | Slide35.jpg | 4476 | 32.71 |
| 25 | Slide35.jpg | 4213 | |
| 26 | Slide35.jpg | 1798 | 10.67 |
| 27 | Slide35.jpg | 2840 | 16.85 |
| 28 | Slide35.jpg | 3714 | |
| 29 | Slide35.jpg | 2917 | 17.28 |
| 30 | Slide35.jpg | 342 | 2.03 |

TABLE 52
TNF9 summary

| | |
|---|---|
| non-IS | 35.33 |
| IS | 63.72 |
| LV | 99.05 |
| IS/LV | 64% |

TABLE 53
TNF10 raw values

| | | | |
|---|---|---|---|
| 1 | Slide36.jpg | 2294 | |
| 2 | Slide36.jpg | 14 | 0.08 |
| 3 | Slide36.jpg | 2183 | 12.37 |
| 4 | Slide36.jpg | 4093 | |
| 5 | Slide36.jpg | 189 | 1.34 |
| 6 | Slide36.jpg | 3572 | 25.31 |
| 7 | Slide36.jpg | 4330 | |
| 8 | Slide36.jpg | 829 | 9.38 |
| 9 | Slide36.jpg | 2710 | 30.67 |
| 10 | Slide36.jpg | 2189 | |
| 11 | Slide36.jpg | 185 | 1.18 |
| 12 | Slide36.jpg | 1581 | 10.11 |
| 13 | Slide36.jpg | 1961 | |
| 14 | Slide36.jpg | 344 | 1.40 |
| 15 | Slide36.jpg | 1293 | 5.27 |
| 16 | Slide36.jpg | 2188 | |
| 17 | Slide36.jpg | 1766 | 10.49 |
| 18 | Slide36.jpg | 382 | 2.27 |
| 19 | Slide36.jpg | 4243 | |
| 20 | Slide36.jpg | 2206 | 15.08 |
| 21 | Slide36.jpg | 1246 | 8.52 |
| 22 | Slide36.jpg | 4883 | |
| 23 | Slide36.jpg | 3763 | 37.76 |
| 24 | Slide36.jpg | 583 | 5.85 |
| 25 | Slide36.jpg | 2162 | |
| 26 | Slide36.jpg | 2025 | 13.11 |
| 27 | Slide36.jpg | 18 | 0.12 |
| 28 | Slide36.jpg | 2558 | |
| 29 | Slide36.jpg | 1179 | 3.69 |
| 30 | Slide36.jpg | 615 | 1.92 |

TABLE 54
TNF10 summary

| | |
|---|---|
| non-IS | 46.76 |
| IS | 51.20 |
| LV | 97.96 |
| IS/LV | 52% |

TABLE 55
Composite image data

| | IS/LV | | IS/LV | | IS/LV | | IS/LV |
|---|---|---|---|---|---|---|---|
| CON7 | 70% | TMZ3 | 64% | TNF1 | 57% | TNS1 | 55% |
| CON5 | 65% | TMZ1 | 68% | TNF2 | 67% | TNS2 | 61% |
| CON6 | 75% | TMZ2 | 60% | TNF3 | 60% | TNS3 | 63% |
| CON4 | 65% | TMZ7 | 43% | TNF4 | 75% | TNS4 | 51% |
| CON3 | 64% | TMZ8 | 51% | TNF5 | 73% | TNS5 | 35% |
| CON1 | 77% | TMZ5 | 58% | TNF6 | 79% | TNS6 | 36% |
| CON2 | 55% | TMZ6 | 49% | TNF7 | 53% | TNS7 | 34% |
| CON8 | 68% | TMZ9 | 44% | TNF8 | 57% | TNS8 | 51% |
| CON9 | 67% | TMZ10 | 49% | TNF9 | 64% | TNS9 | 31% |
| CON10 | 62% | TMZ4 | 71% | TNF10 | 52% | TNS10 | 26% |
| CON11 | 73% | | | | | | |
| CON12 | 75% | | | | | | |
| CON13 | 76% | | | | | | |
| CON14 | 66% | | | | | | |
| Mean | 68% | Mean | 56% | Mean | 64% | Mean | 44% |
| SD | 6% | SD | 10% | SD | 10% | SD | 13% |
| SE | 2% | SE | 3% | SE | 3% | SE | 4% |
| TTEST | | | 8.77E−04 | | 1.61E−01 | | 4.79E−06 |
| | | | | | | TMZ/TNS | 4.00E−02 |

The results show that a combination of trimetazidine, nicotinamide, and succinate at 20 μM preserved coronary flow and cardiac functional recovery and decreased infarct size in isolated hearts after ischemia-reperfusion. This combination was more effective in decreasing infarct size than TMZ alone. A combination of trimetazidine, nicotinamide, and succinate at 2 μM did not appear to decrease myocardial ischemia-reperfusion injury.

This study suggested that the combination of trimetazidine, nicotinamide, and succinate at 20 μM generated better protection against ischemia-reperfusion injury in Langendorff system.

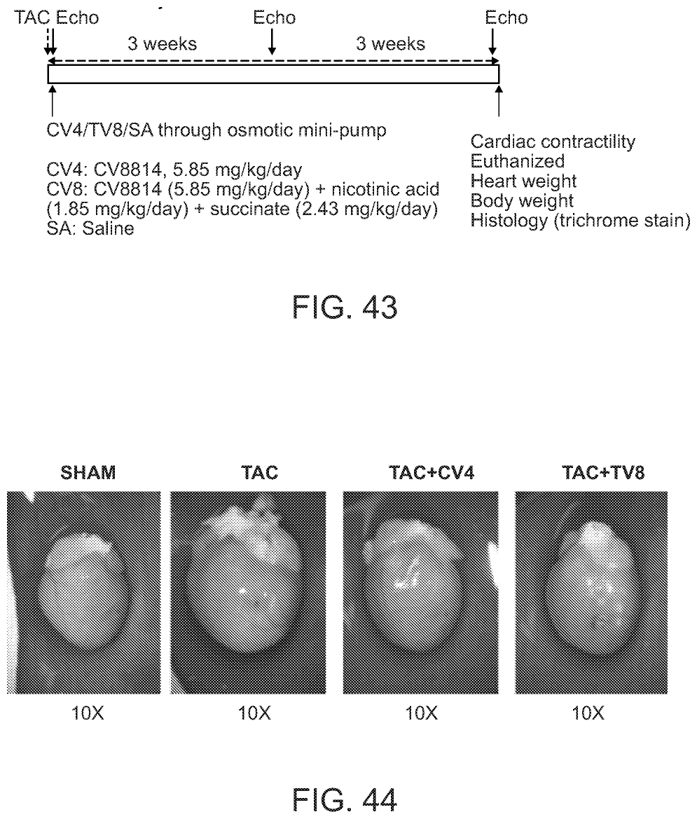

FIG. 43 is a schematic of the method used to analyze the effects of selected compositions on cardiac function. Following transverse aortic constriction (TAC) or a sham procedure, mice were given one of the following via an osmotic mini-pump: CV8814 at 5.85 mg/kg/day (CV4); CV8814 at 5.85 mg/kg/day, nicotinic acid at 1.85 mg/kg/day, and succinate at 2.43 mg/kg/day (TV8); or saline (SA). Echocardiograms were measured 24 hours following TAC, three weeks after TAC, and 6 weeks after TAC. Mice were sacrificed at 6 weeks, and tissues were analyzed.

FIG. 44 shows hearts from mice six weeks after a sham procedure (SHAM), TAC followed by saline administration (TAC), TAC followed by CV4 administration (CV4), or TAC followed by TV8 administration.

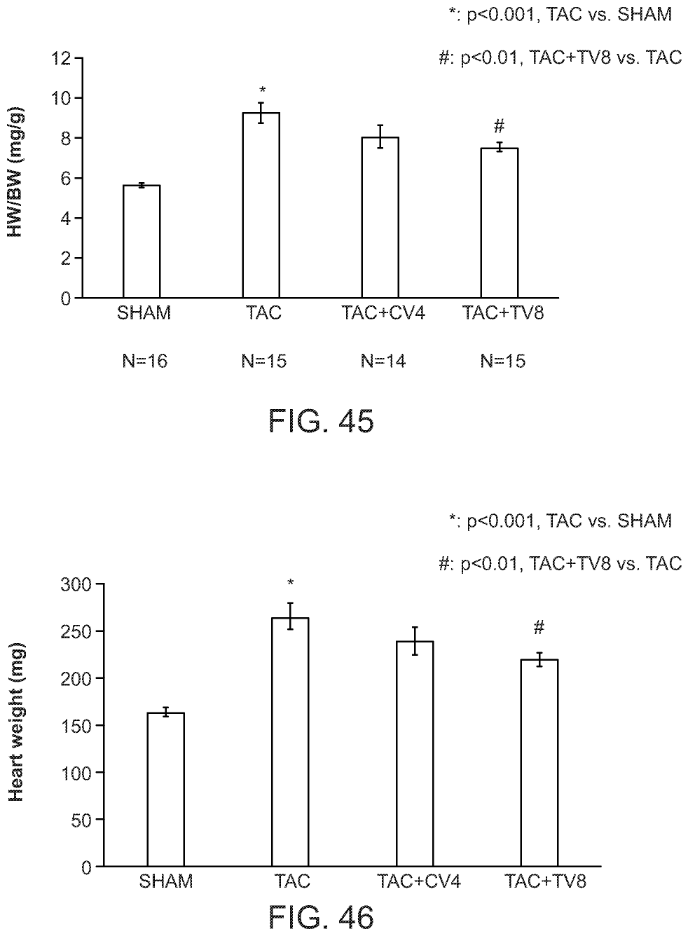

FIG. 45 is of graph of heart weight relative to body weight six weeks after transverse aortic constriction. Treatments are as indicated in relation to FIG. 44.

FIG. 46 is graph of heart weight six weeks after transverse aortic constriction. Treatments are as indicated in relation to FIG. 44.

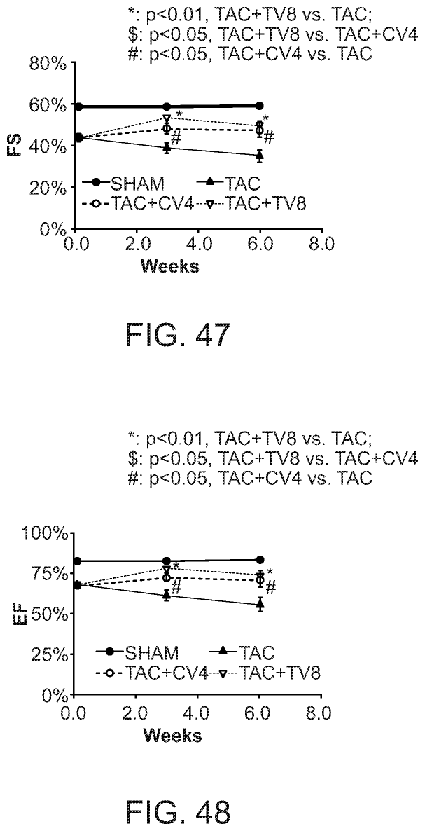

FIG. 47 is a graph of fractional shortening (FS) at indicated time points after transverse aortic constriction. Treatments are as indicated in relation to FIG. 44.

FIG. 48 is a graph of ejection fraction (EF) at indicated time points after transverse aortic constriction. Treatments are as indicated in relation to FIG. 44.

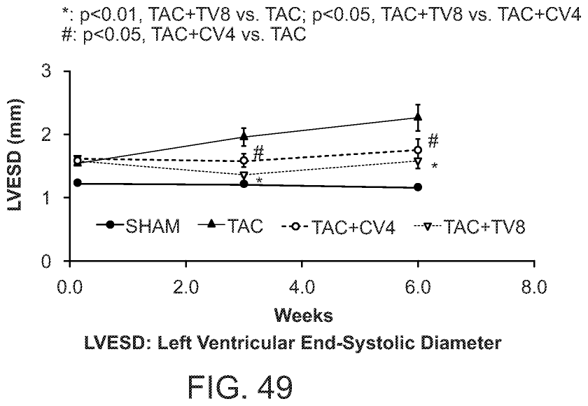

FIG. 49 is a graph of left ventricular end-systolic diameter at indicated time points after transverse aortic constriction. Treatments are as indicated in relation to FIG. 44.

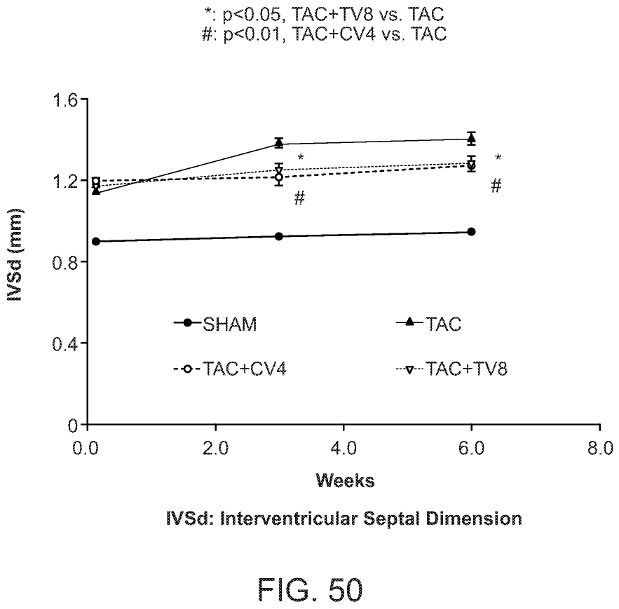

FIG. 50 is a graph of intraventricular septal dimension at indicated time points after transverse aortic constriction. Treatments are as indicated in relation to FIG. 44.

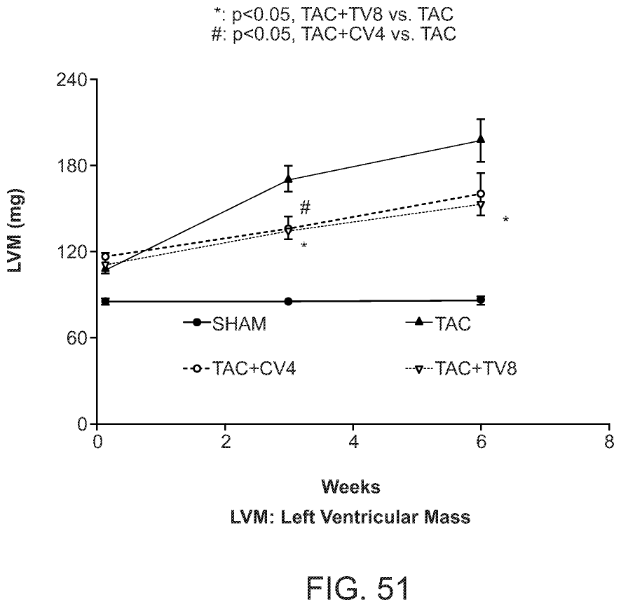

FIG. 51 is a graph of left ventricular mass at indicated time points after transverse aortic constriction. Treatments are as indicated in relation to FIG. 44.

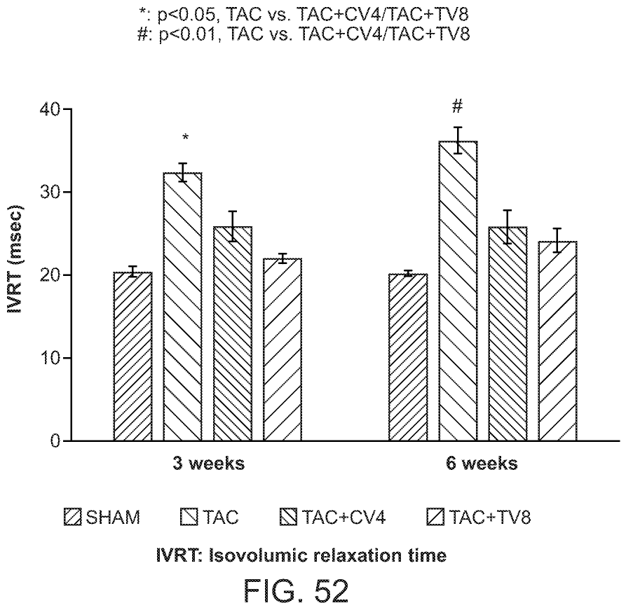

FIG. 52 is a graph of isovolumic relaxation time at indicated time points after transverse aortic constriction. Treatments are as indicated in relation to FIG. 44.

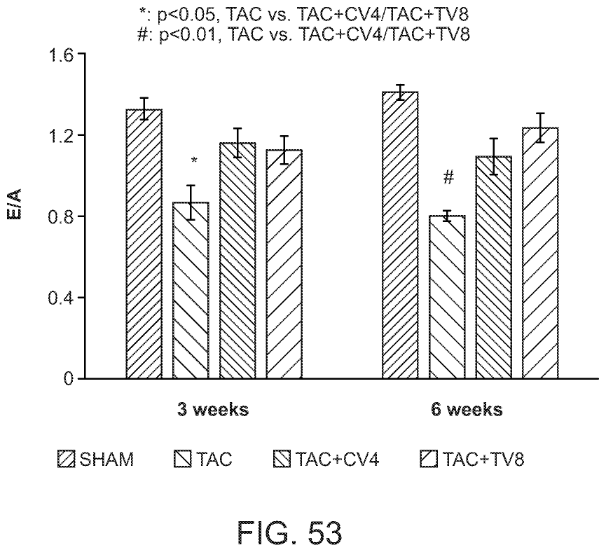

FIG. 53 is a graph of the ratio peak velocity flow in early diastole vs. late diastole at indicated time points after transverse aortic constriction. Treatments are as indicated in relation to FIG. 44.

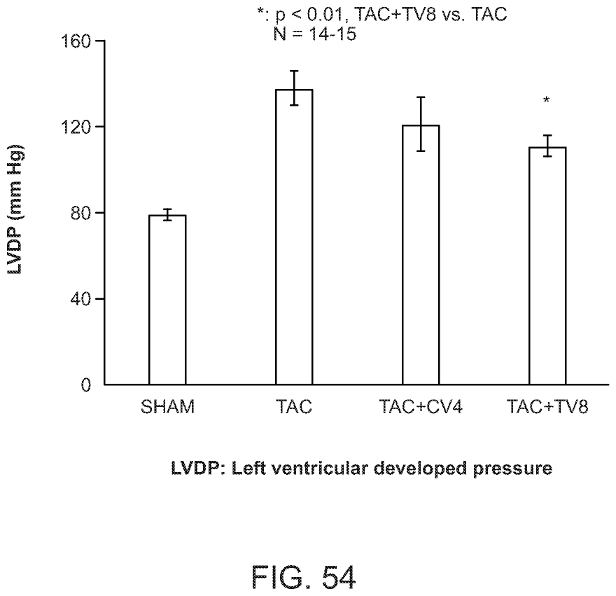

FIG. 54 is a graph of left ventricular developed pressure at six weeks after transverse aortic constriction. Treatments are as indicated in relation to FIG. 44.

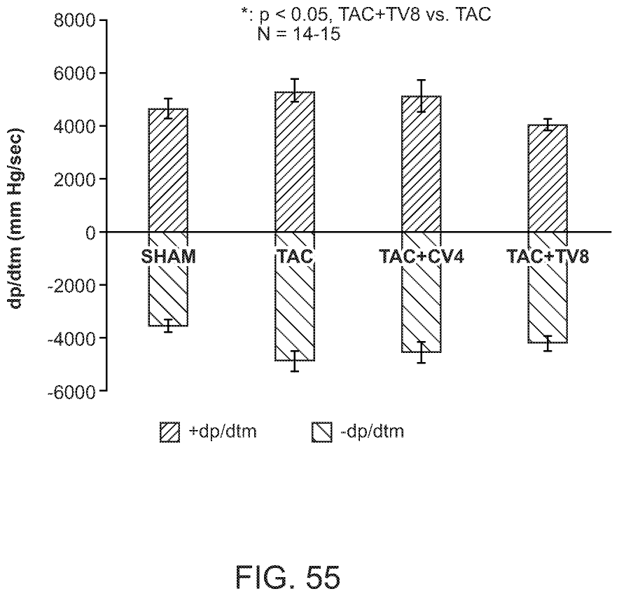

FIG. 55 is a graph of the rate of left ventricle pressure rise at six weeks after transverse aortic constriction. Treatments are as indicated in relation to FIG. 44.

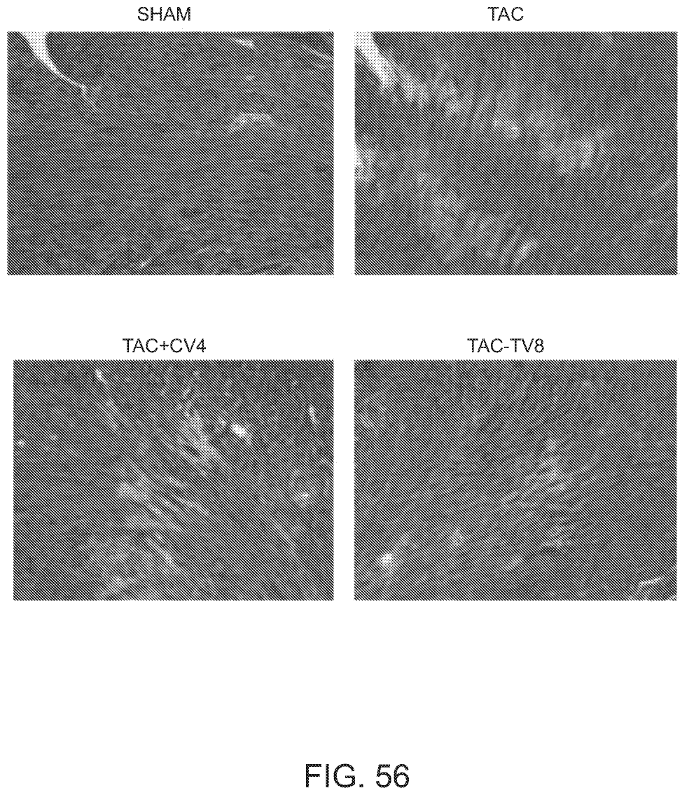

FIG. 56 shows microscopic images of cardiac tissue t six weeks after transverse aortic constriction. Upper left panel, sham TAC procedure; upper right panel, TAC; lower left panel, TAC followed by CV4 administration; and lower right panel, TAC followed by TV8 administration.

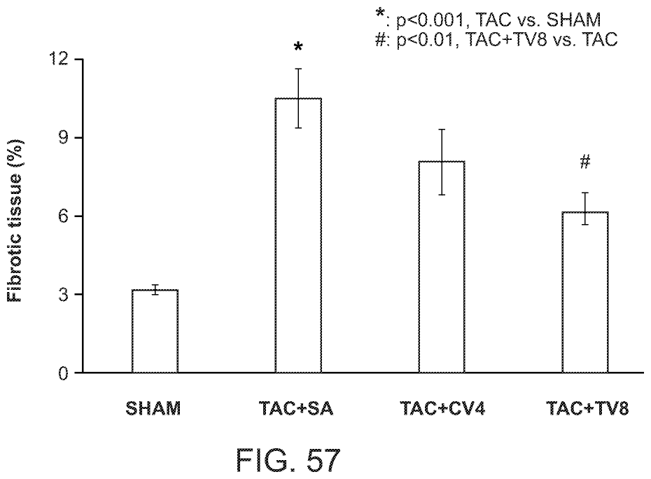

FIG. 57 is a graph showing the level of cardiac fibrosis at six weeks after transverse aortic constriction. Treatments are as indicated in relation to FIG. 44.

Chemical Synthesis Schemes.

Compounds that shift cellular metabolism from fatty acid oxidation to glucose oxidation include 2-(4-(2,3,4-trimethoxybenzyl)piperazin-1-yl)ethan-1-ol (referred to herein as CV8814) and 2-(4-(2,3,4-trimethoxybenzyl)piperazin-1-yl)ethyl nicotinate (referred to herein as CV-8972). These compounds may be synthesized according to the following scheme:

### Stage 1:

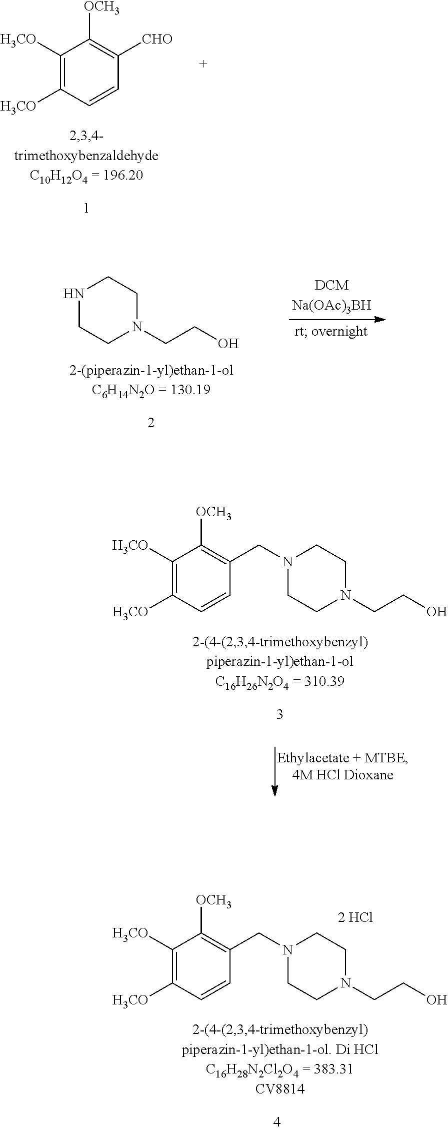 [SMILES: COc1ccc(C=O)c(OC)c1OC ; OCCN1CCNCC1 ; COc1ccc(CN2CCN(CCO)CC2)c(OC)c1OC ; COc1ccc(CN2CCN(CCO)CC2)c(OC)c1OC]

### Stage 2:

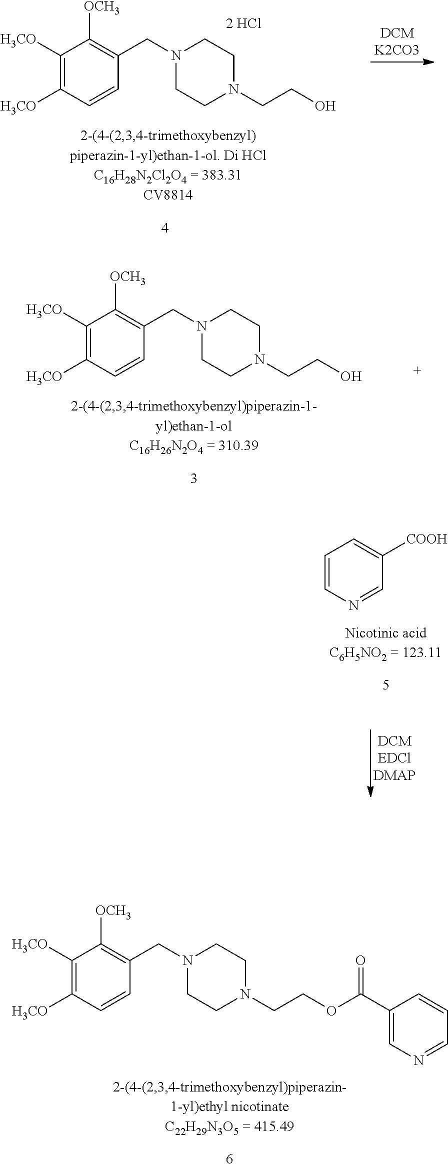 [SMILES: COc1ccc(CN2CCN(CCO)CC2)c(OC)c1OC ; COc1ccc(CN2CCN(CCO)CC2)c(OC)c1OC ; O=C(O)c1cccnc1 ; COc1ccc(CN2CCN(CCOC(=O)c3cccnc3)CC2)c(OC)c1OC]

### Stage 3:

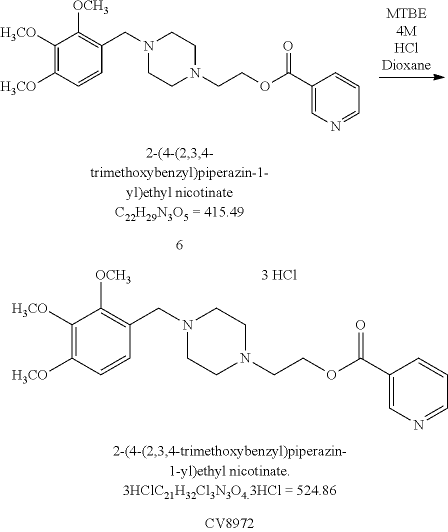 [SMILES: COc1ccc(CN2CCN(CCOC(=O)c3cccnc3)CC2)c(OC)c1OC ; COc1ccc(CN2CCN(CCOC(=O)c3cccnc3)CC2)c(OC)c1OC]

The product was converted to the desired polymorph by recrystallization. The percentage of water and the ratio of methanol:methyl ethyl ketone (MEK) were varied in different batches using 2.5 g of product.

In batch MBA 25, 5% water w/r/t total volume of solvent (23 volumes) containing 30% methanol:70% MEK was used for precipitation. The yield was 67% of monohydrate of CV-8972. Water content was determined by KF to be 3.46%.

In batch MBA 26, 1.33% water w/r/t total volume of solvent (30 volumes) containing 20% methanol:80% MEK was used for precipitation. The yield was 86.5% of monohydrate of CV-8972. Water content was determined by KF to be 4.0%. The product was dried under vacuum at 40° C. for 24 hours to decrease water content to 3.75%.

In batch MBA 27, 3% water w/r/t total volume of solvent (32 volumes) containing 22% methanol:78% MEK was used for precipitation. The yield was 87.22% of monohydrate of CV-8972. Water content was determined by KF to be 3.93% after 18 hours of drying at room temperature under vacuum. The product was further dried under vacuum at 40° C. for 24 hours to decrease water content to 3.54%.

In other batches, the ratio and total volume of solvent were held constant at 20% methanol:80% MEK and 30 volumes in batches using 2.5 g of product, and only the percentage of water was varied.

In batch MBA 29, 1.0 equivalent of water was added. Material was isolated and dried under vacuum at 40° C. for 24 hours. Water content was determined by KF to be 0.89%, showing that the monohydrate form was not forming stoichiometrically.

In batch MBA 30, 3% water was added. Material was isolated and dried under vacuum at 40° C. for 24 hours. Water content was determined by KF to be 3.51%, showing that monohydrate is forming with addition of excess water.

In batch MBA 31, 5% water was added. Material was isolated and dried under vacuum at 40° C. for 24 hours. Water content was determined by KF to be 3.30%, showing that monohydrate is forming with addition of excess water.

Results are summarized in Table 56.

TABLE 56

| | Water | | | Amount of | | | | | |
|---|---|---|---|---|---|---|---|---|---|
| | percentage | | KF result | Water used | | | | | |
| | theoretical | KF | (Sample | for reaction | | | | | Drying |
| | (for | result | after drying | (based | Ratio of | | Yield | Drying | temper- |
| | monohydrate | (% of | at 40° C. for | on total | MeOH: | Total | obtained | Time | ature |
| Sample | preparation) | water) | 24 hours) | volume) | MEK | Volume | (%) | (hr) | (° C.) |
| 289-MBA-25 | 3.32% | 3.46 | — | 5% | 30-70 | 23 vol | 67.6 | 24 | 22 |
| 289-MBA-26 | 3.32% | 4.00 | 3.75 | 1.33% | 20-80 | 30 vol | 86.5 | 19 | 23 |
| 289-MBA-27 | 3.32% | 3.93 | 3.54 | 3% | 22-78 | 32 vol | 87.22 | 18 | 23 |
| 289-MBA-29 | 3.32% | — | 0.89 | 1.0 eq based | 20-80 | 30 vol | 84 | 24 | 40 |
| | | | | on input | | | | | |
| | | | | weight | | | | | |
| 289-MBA-30 | 3.32% | — | 3.51 | 3% | 20-80 | 30 vol | 90 | 24 | 40 |
| 289-MBA-31 | 3.32% | — | 3.30 | 5% | 20-80 | 30 vol | 81 | 24 | 40 |

Metabolism of Compounds in Dogs

The metabolism of various compounds was analyzed in dogs.

FIG. 58 is a graph showing levels of CV-8814 (solid triangles, solid lines) and trimetazidine (open triangles, dashed lines) after intravenous administration of CV-8834 at 2.34 mg/kg. CV-8834 is a compound of formula (II) in which y=1.

FIG. 59 is a graph showing levels of CV-8814 (solid triangles, solid lines) and trimetazidine (open triangles, dashed lines) after oral administration of CV-8834 at 77.4 mg/kg.

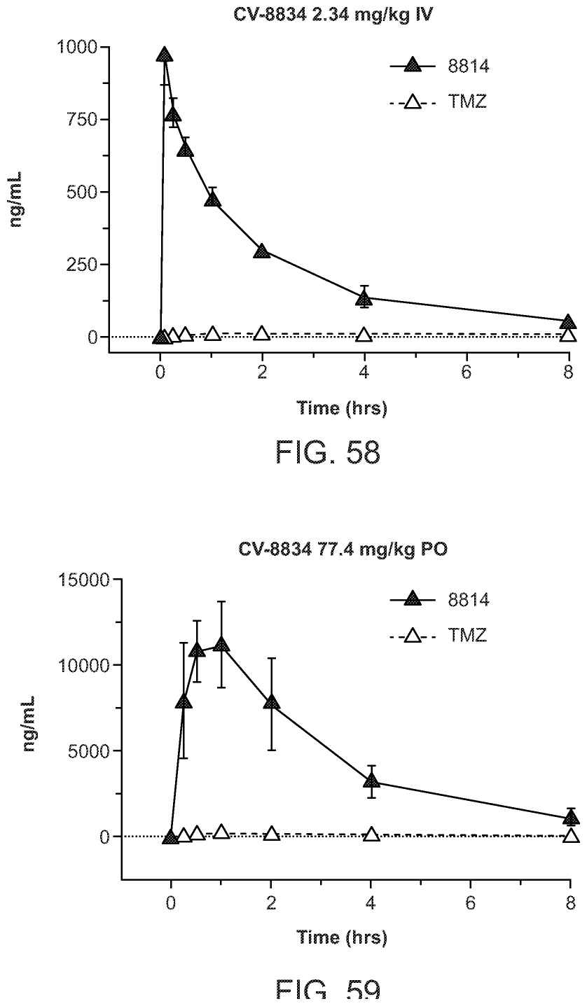

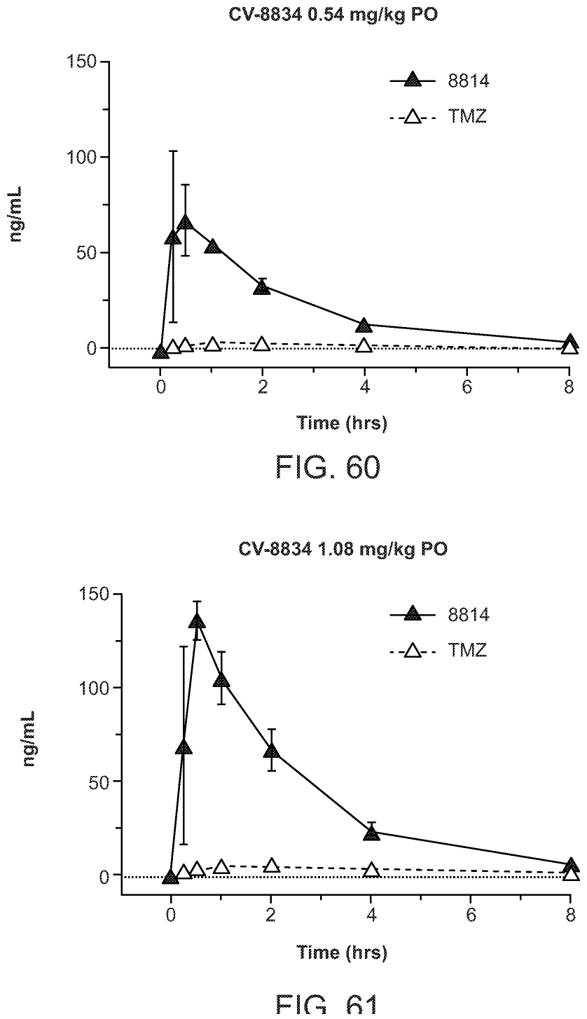

FIG. 60 is a graph showing levels of CV-8814 (solid triangles, solid lines) and trimetazidine (open triangles, dashed lines) after oral administration of CV-8834 at 0.54 mg/kg.

FIG. 61 is a graph showing levels of CV-8814 (solid triangles, solid lines) and trimetazidine (open triangles, dashed lines) after oral administration of CV-8834 at 1.08 mg/kg.

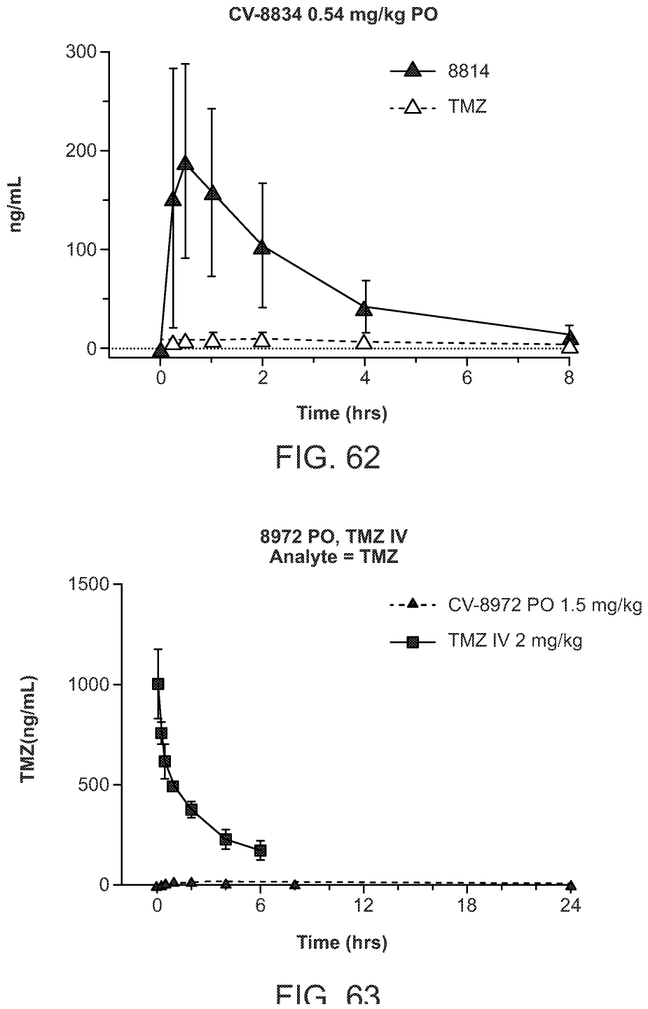

FIG. 62 is a graph showing levels of CV-8814 (solid triangles, solid lines) and trimetazidine (open triangles, dashed lines) after oral administration of CV-8834 at 2.15 mg/kg.

Data from FIGS. 55-59 is summarized in Table 57.

TABLE 57

| | Route | Dose | | | Cₘₐₓ | AUC₀₋₈ | |
|---|---|---|---|---|---|---|---|
| Com- | of | (mg/ | | Tₘₐₓ | (ng/ | (ng × | |
| pound | admin. | kg) | Analyte | (hours) | mL) | hr/mL) | % F |
| CV-8834 | PO | 77.4 | 8814 | 0.75 | 12100 | 38050 | 69 |
| CV-8834 | PO | 77.4 | TMZ | 1.67 | 288 | 1600 | 72 |
| CV-8834 | IV | 2.34 | 8814 | 0.083 | 974 | 1668 | — |
| CV-8834 | IV | 2.34 | TMZ | 2.67 | 13.4 | 66.7 | — |
| CV-8834 | PO | 0.54 | 8814 | 0.5 | 74.0 | 175 | 45 |
| CV-8834 | PO | 0.54 | TMZ | 1.17 | 3.63 | 17.6 | >100 |
| CV-8834 | PO | 1.08 | 8814 | 0.5 | 136 | 335 | 44 |
| CV-8834 | PO | 1.08 | TMZ | 0.866 | 6.19 | 30.4 | 99 |
| CV-8834 | PO | 2.15 | 8814 | 0.583 | 199 | 536 | 35 |
| CV-8834 | PO | 2.15 | TMZ | 1.17 | 9.80 | 51.6 | 84 |

FIG. 63 is a graph showing levels of trimetazidine after oral administration of CV-8972 at 1.5 mg/kg (triangles) or intravenous administration of trimetazidine at 2 mg/kg (squares).

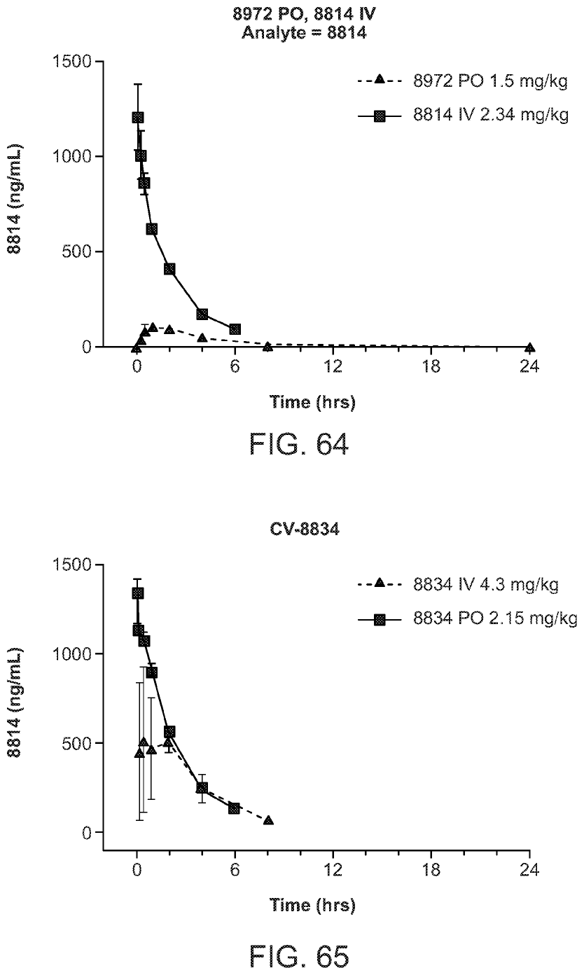

FIG. 64 is a graph showing levels of CV-8814 after oral administration of CV-8972 at 1.5 mg/kg (triangles) or intravenous administration of CV-8814 at 2.34 mg/kg (squares).

FIG. 65 is a graph showing levels of CV-8814 after intravenous administration of CV-8834 at 4.3 mg/kg (squares) or oral administration of CV-8834 at 2.15 mg/kg (triangles).

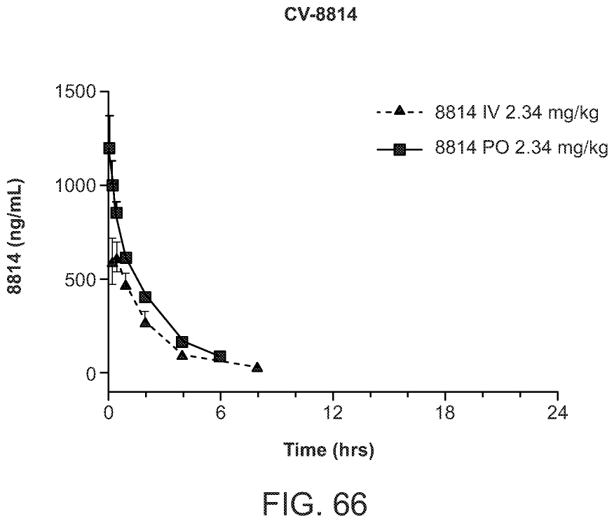

FIG. 66 is a graph showing levels of CV-8814 after intravenous administration of CV-8814 at 2.34 mg/kg (squares) or oral administration of CV-8814 at 2.34 mg/kg (triangles).

Data from FIGS. 60-63 is summarized in Table 58.

TABLE 58

| | Route of | Dose | | | | Tₘₐₓ | Cₘₐₓ | AUC₀₋₂₄ | |
|---|---|---|---|---|---|---|---|---|---|
| Compound | admin. | (mg/kg) | Vehicle | Fasted | Analyte | (hours) | (ng/mL) | (ng × hr/mL) | % F |
| CV-8972 | PO | 1.5 | — | — | TMZ | 2.0 | 17.0 | 117 | 4.3% |
| TMZ | IV | 2 | 0.9% NaCl | 8 hrs | TMZ | 0.083 | 1002 | 3612 | — |
| CV-8972 | PO | 1.5 | — | — | 8814 | 1.125 | 108 | 534 | 27% |
| CV-8814 | IV | 2.34 | 0.9% NaCl | 8 hrs | 8814 | 0.083 | 1200 | 3059 | — |
| CV-8834 | PO | 4.3 | 0.9% NaCl | 8 hrs | 8814 | 1.0 | 692 | 2871 | 69% |
| CV-8834 | IV | 4.3 | 0.9% NaCl | 8 hrs | 8814 | 0.083 | 1333 | 4154 | — |
| CV-8834 | PO | 4.3 | 0.9% NaCl | 8 hrs | 8814 | 1.0 | 692 | 2871 | 51% |
| CV-8814 | IV | 2.34 | 0.9% NaCl | 8 hrs | 8814 | 0.083 | 1200 | 3059 | — |
| CV-8814 | PO | 2.34 | 0.9% NaCl | 8 hrs | 8814 | 0.333 | 672 | 1919 | 63% |
| CV-8814 | IV | 2.34 | 0.9% NaCl | 8 hrs | 8814 | 0.083 | 1200 | 3059 | — |

Effect of CV-8814 on Enzyme Activity

The effect of CV-8814 on the activity of various enzymes was analyzed in in vitro assays. Enzyme activity was assayed in the presence of 10 μM CV-8814 using conditions of time, temperature, substrate, and buffer that were optimized for each enzyme based on published literature. Inhibition of 50% or greater was not observed for any of the following enzymes: ATPase, Na⁺/K⁺, pig heart; Cholinesterase, Acetyl, ACES, human; Cyclooxygenase COX-1, human; Cyclooxygenase COX-2, human; Monoamine Oxidase MAO-A, human; Monoamine Oxidase MAO-B, human; Peptidase, Angiotensin Converting Enzyme, rabbit; Peptidase, CTSG (Cathepsin G), human; Phosphodiesterase PDE3, human; Phosphodiesterase PDE4, human; Protein Serine/Threonine Kinase, PKC, Non-selective, rat; Protein Tyrosine Kinase, Insulin Receptor, human; Protein Tyrosine Kinase, LCK, human; Adenosine A1, human; Adenosine A_{2A}, human; Adrenergic α_{1A}, rat; Adrenergic α_{1B}, rat; Adrenergic α_{1D}, human; Adrenergic α_{2A}, human; Adrenergic α_{2B}, human; Adrenergic β₁, human; Adrenergic β₂, human; Androgen (Testosterone), human; Angiotensin AT₁, human; Bradykinin B₂, human; Calcium Channel L-Type, Benzothiazepine, rat; Calcium Channel L-Type, Dihydropyridine, rat; Calcium Channel L-Type, Phenylalkylamine, rat; Calcium Channel N-Type, rat; Cannabinoid CB₁, human; Cannabinoid CB₂, human; Chemokine CCR1, human; Chemokine CXCR2 (IL-8R_{B}), human; Cholecystokinin CCK₁ (CCK_{A}), human; Cholecystokinin CCK₂ (CCK_{B}), human; Dopamine D₁, human; Dopamine D_{2L}, human; Dopamine D_{2S}, human; Endothelin ET_{A}, human; Estrogen ERα, human; GABA_{A}, Chloride Channel, TBOB, rat; GABA_{A}, Flunitrazepam, Central, rat; GABA_{A}, Ro-15-1788, Hippocampus, rat; GABA_{B1A}, human; Glucocorticoid, human; Glutamate, AMPA, rat; Glutamate, Kainate, rat; Glutamate, Metabotropic, mGlu5, human; Glutamate, NMDA, Agonism, rat; Glutamate, NMDA, Glycine, rat; Glutamate, NMDA, Phencyclidine, rat; Glutamate, NMDA, Polyamine, rat; Glycine, Strychnine-Sensitive, rat; Histamine H₁, human; Histamine H₂, human; Melanocortin MC₁, human; Melanocortin MC₄, human; Muscarinic M₁, human; Muscarinic M₂, human; Muscarinic M₃, human; Muscarinic M₄, human; Neuropeptide Y Y₁, human; Nicotinic Acetylcholine, human; Nicotinic Acetylcholine al, Bungarotoxin, human; Opiate δ₁ (OP1, DOP), human; Opiate κ (OP2, KOP), human; Opiate μ (OP3, MOP), human; Platelet Activating Factor (PAF), human; Potassium Channel [KATP], hamster; Potassium Channel hERG, human; PPARγ, human; Progesterone PR-B, human; Serotonin (5-Hydroxytryptamine) 5-HT_{1A}, human; Serotonin (5-Hydroxytryptamine) 5-HT_{1B}, human; Serotonin (5-Hydroxytryptamine) 5-HT_{2A}, human; Serotonin (5-Hydroxytryptamine) 5-HT_{2B}, human; Serotonin (5-Hydroxytryptamine) 5-HT_{2C}, human; Serotonin (5-Hydroxytryptamine) 5-HT₃, human; Sodium Channel, Site 2, rat; Tachykinin NK₁, human; Transporter, Adenosine, guinea pig; Transporter, Dopamine (DAT), human; Transporter, GABA, rat; Transporter, Norepinephrine (NET), human; Transporter, Serotonin (5-Hydroxytryptamine) (SERT), human; and Vasopressin V_{1A}, human.

Analysis of CV-8972 Batch Properties

CV-8972 (2-(4-(2,3,4-trimethoxybenzyl)piperazin-1-yl)ethyl nicotinate, HCl salt, monohydrate) was prepared and analyzed. The batch was determined to be 99.62% pure by HPLC.

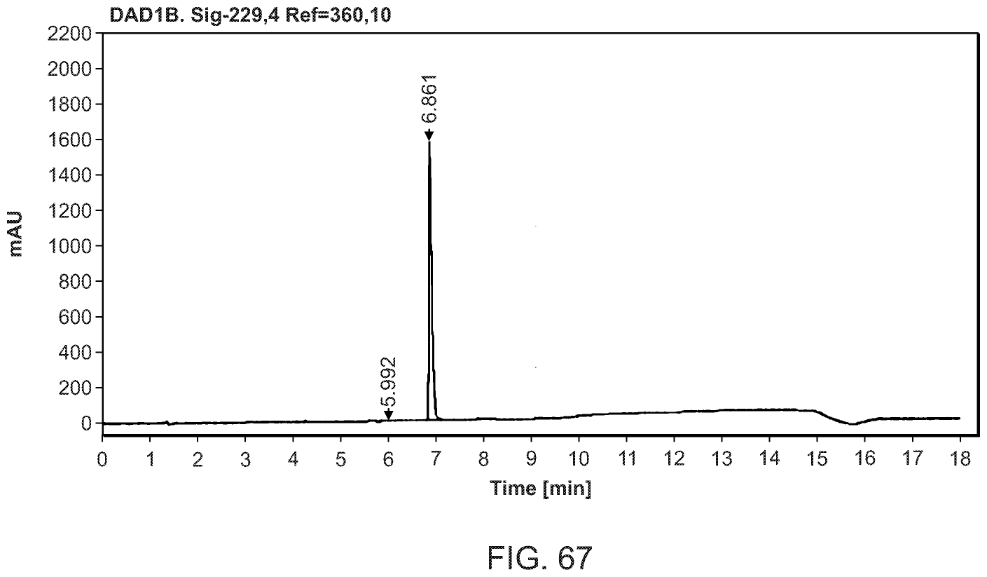

FIG. 67 is a graph showing the HPLC elution profile of a batch of CV-8972.

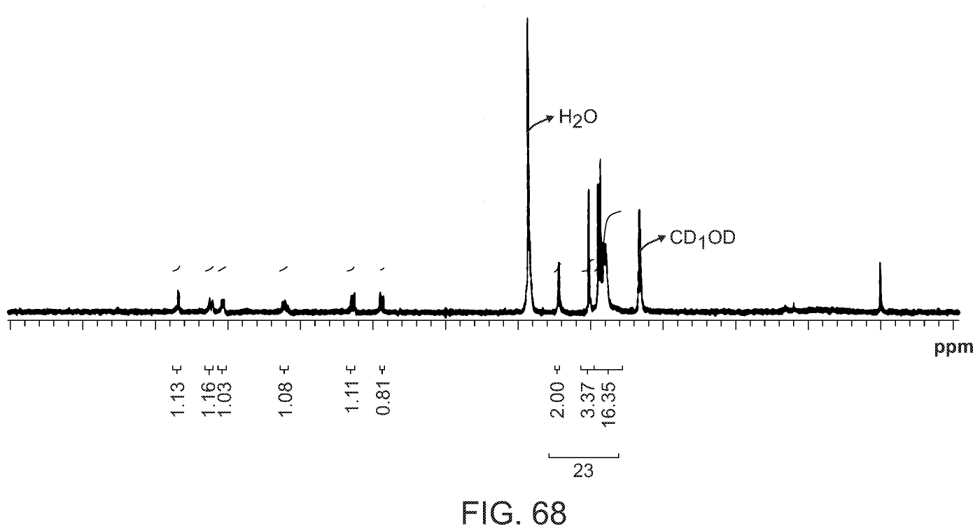

FIG. 68 is a graph showing analysis of molecular species present in a batch of CV-8972.

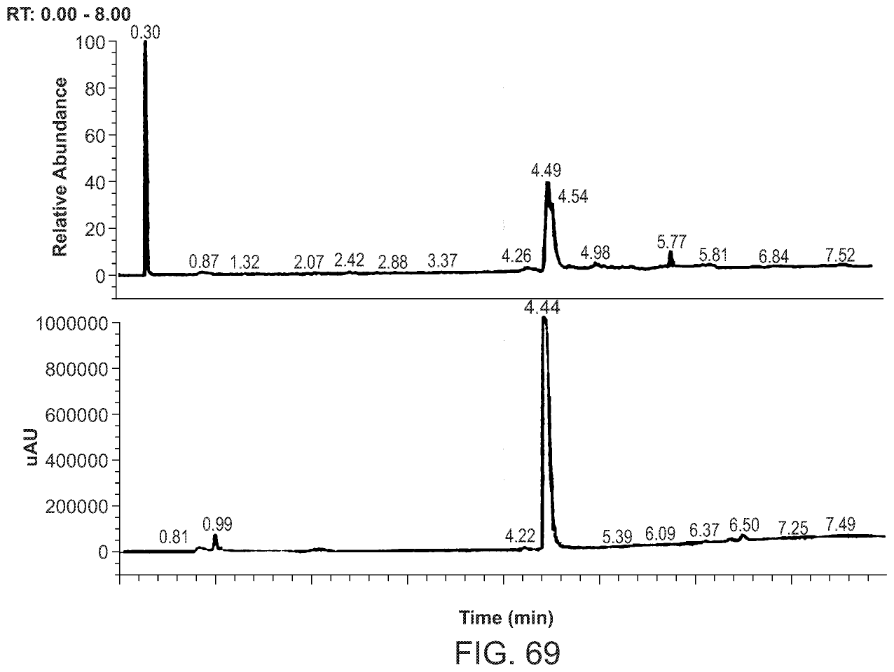

FIG. 69 is a pair of graphs showing HPLC elution profiles of molecular species present in a batch of CV-8972.

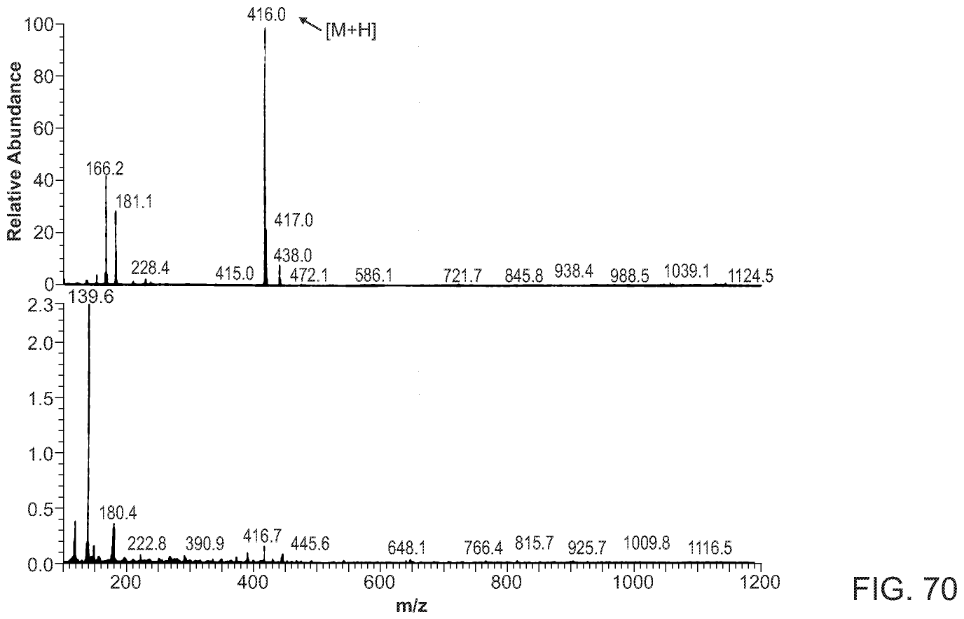

FIG. 70 is a pair of graphs showing HPLC elution profiles of molecular species present in a batch of CV-8972.

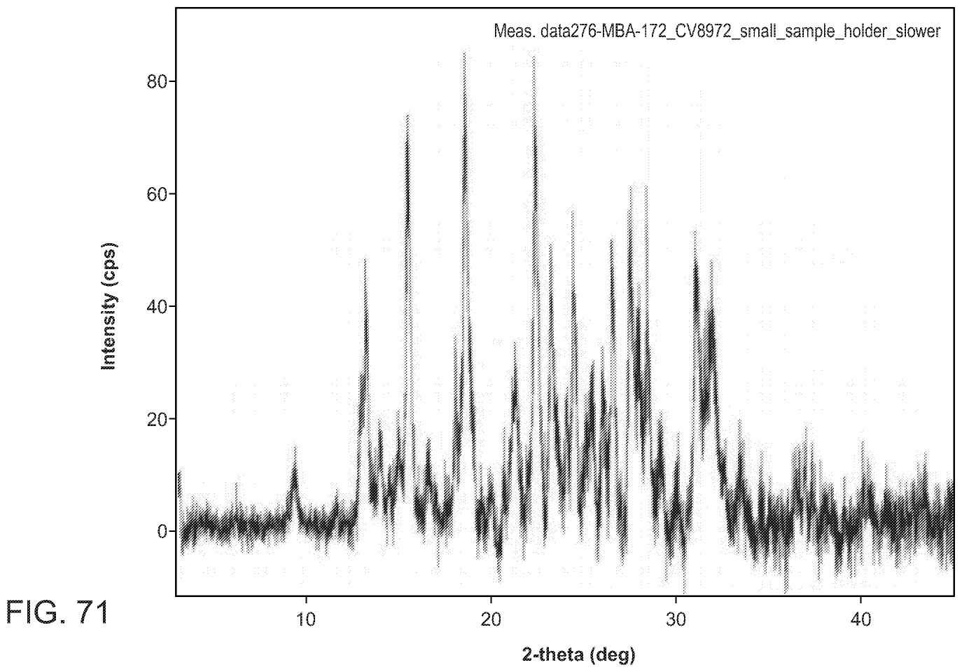

FIG. 71 is a graph showing X-ray powder diffraction analysis of a batch of CV-8972.

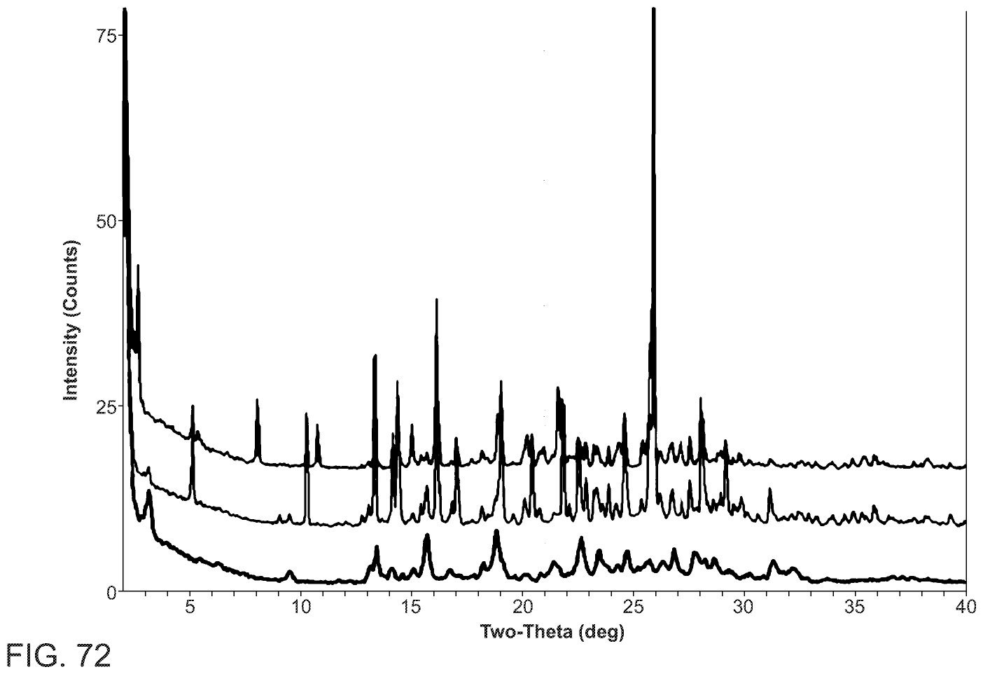

FIG. 72 is a graph showing X-ray powder diffraction analysis of batches of CV-8972. Batch 289-MBA-15-A, shown in blue, contains form B of CV-8972, batch 276-MBA-172, shown in black contains form A of CV-8972, and batch 289-MBA-16, shown in red, contains a mixture of forms A and B.

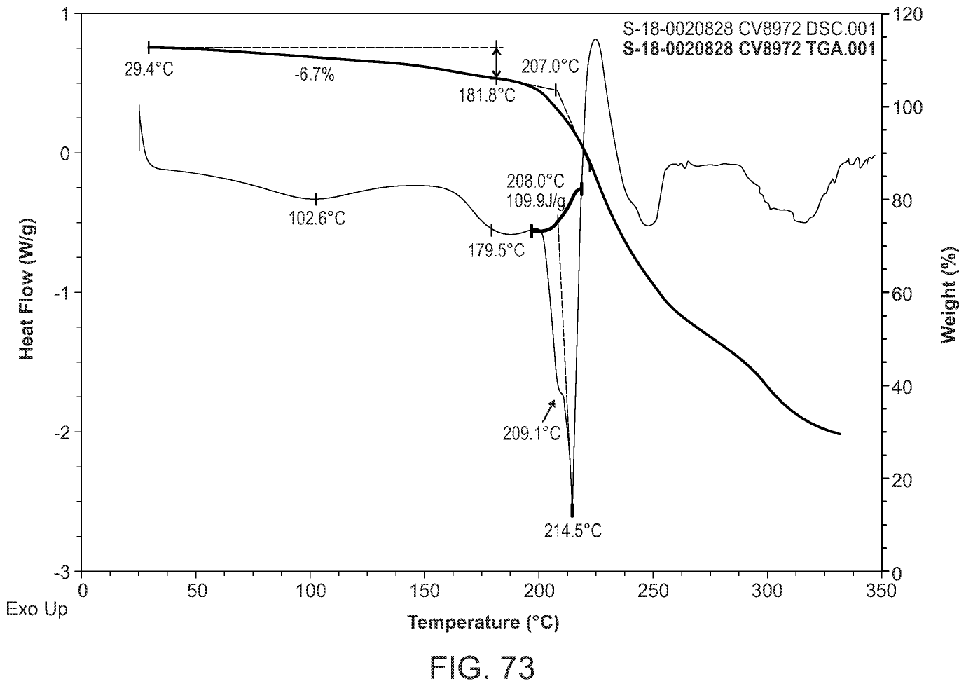

FIG. 73 is a graph showing differential scanning calorimetry and thermal gravimetric analysis of batch 276-MBA-172 of CV-8972.

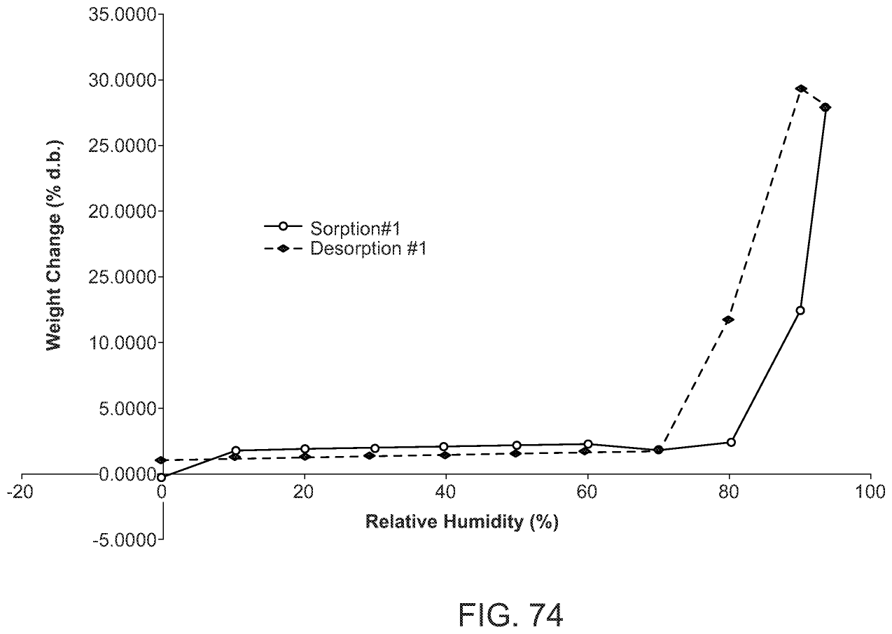

FIG. 74 is a graph showing dynamic vapor sorption (DVS) of batch 276-MBA-172 of CV-8972.

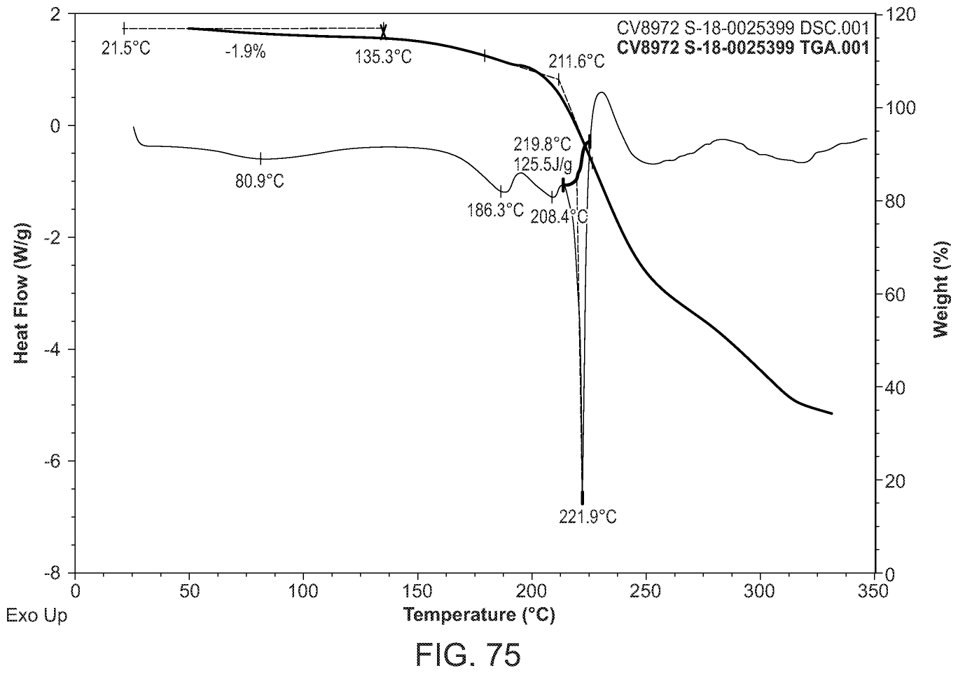

FIG. 75 is a graph showing differential scanning calorimetry and thermal gravimetric analysis of batch 289-MBA-15-A of CV-8972.

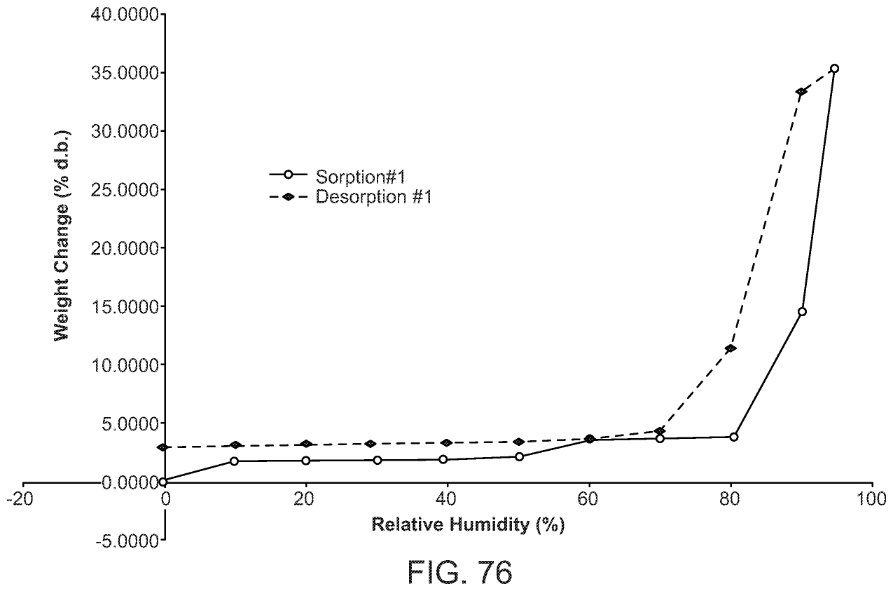

FIG. 76 is a graph showing dynamic vapor sorption (DVS) of batch 289-MBA-15-A of CV-8972.

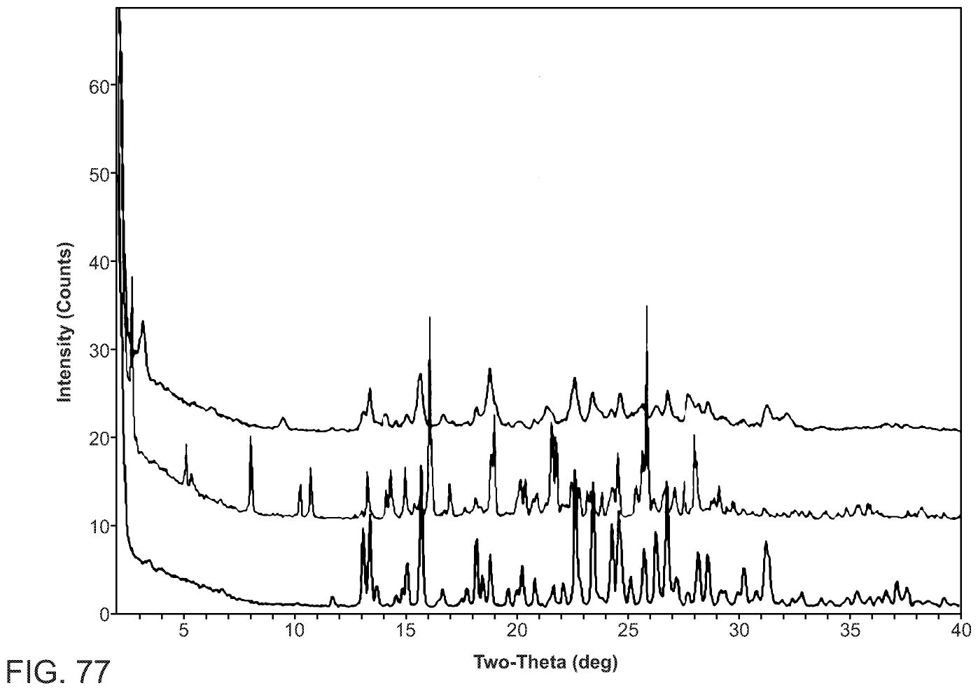

FIG. 77 is a graph showing X-ray powder diffraction analysis of samples of CV-8972. A pre-DVS sample from batch 276-MBA-172 is shown in blue, a pre-DVS sample from batch 289-MBA-15-A is shown in red, and a post-DVS sample from batch 289-MBA-15-A is shown in black.

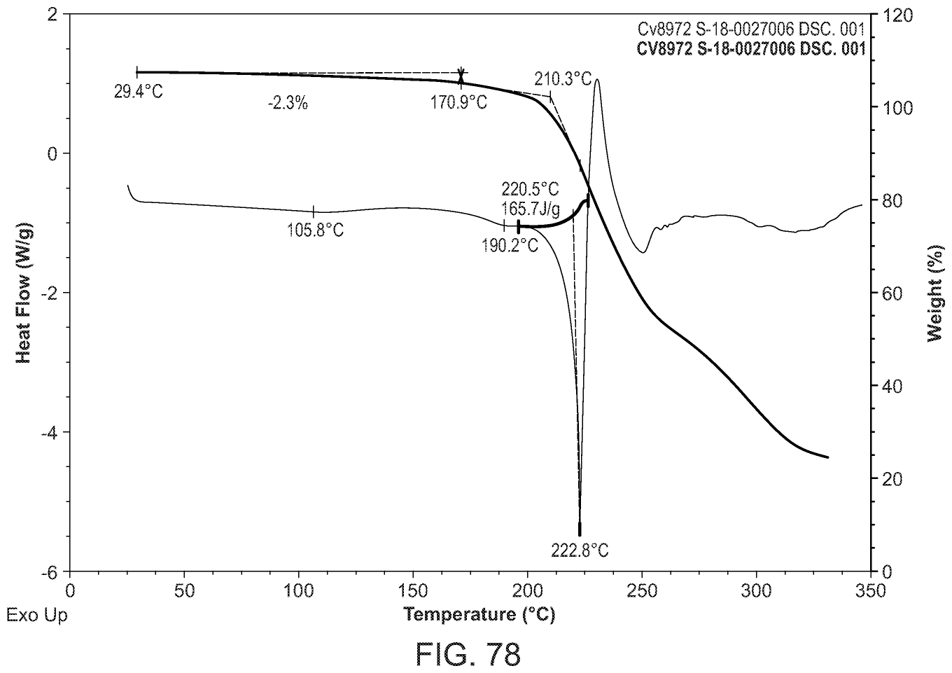

FIG. 78 is a graph showing differential scanning calorimetry and thermal gravimetric analysis of batch 289-MBA-16 of CV-8972.

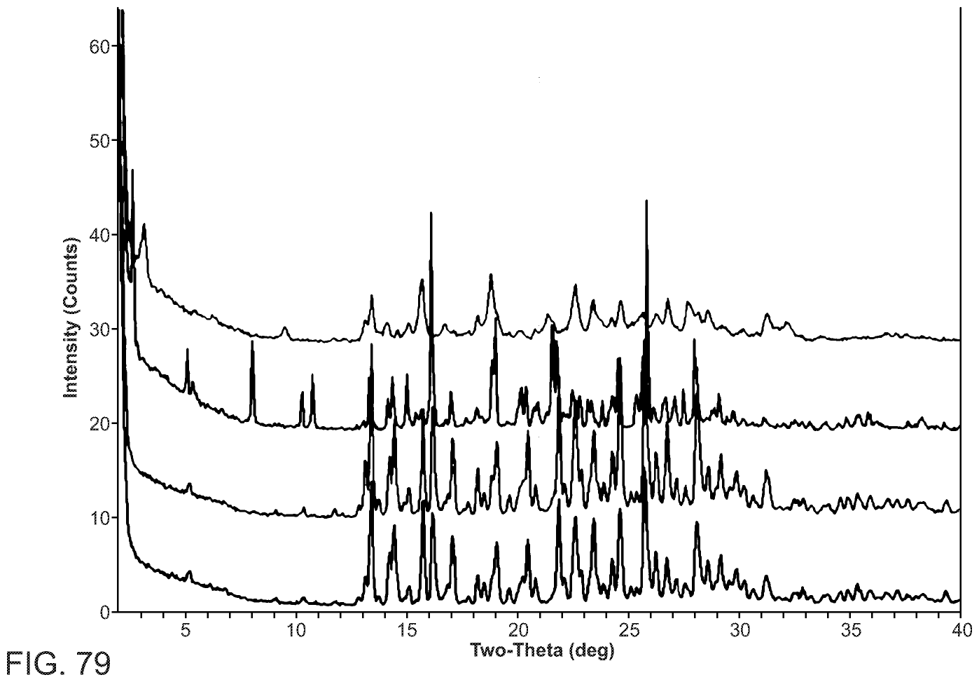

FIG. 79 is a graph showing X-ray powder diffraction analysis of samples of CV-8972. Form B is shown in green, form A is shown in blue, a sample from an ethanol slurry of batch 289-MBA-15-A is shown in red, and a sample from an ethanol slurry of batch 289-MBA-16 is shown in black.

The stability of CV-8972 was analyzed.

Samples from batch 289-MBA-15-A (containing form B) were added to various solvents, incubated under various conditions, and analyzed by X-ray powder diffraction. Results are summarized in Table 59.

TABLE 59

| Solvent | Conditions | XRPD results |
|---|---|---|
| EtOH | Slurry, RT, 3 d | Form A + Form B |
| MeOH/H2O (95:5) | Slurry, RT, 5 d | Form A |
| A_{w} = 0.16 | | |
| IPA/H2O (98:2) | Slurry, RT, 5 d | Form A |
| A_{w} = 0.26 | | |
| MeOH/H2O (80:20) | Slurry, RT, 5 d | Form A |
| A_{w} = 0.48 | | |
| EtOH/H2O (90:10) | Slurry, RT, 5 d | Form A |
| A_{w} = 0.52 | | |
| IPA/H2O (90:10) | Slurry, RT, 5 d | Form A |
| A_{w} = 0.67 | | |
| Acetone/H2O (90:10) | Slurry, RT, 5 d | Form A |
| A_{w} = 0.72 | | |
| ACN/H2O (90:10) | Slurry, RT, 5 d | Form A |
| A_{w} = 0.83 | | |
| EtOAc/H2O (97:3) | Slurry, RT, 5 d | Form A |
| A_{w} = 0.94) | | |
| MeOH | Slurry, RT, 5 d | Form A + Form B |
| EtOAc | Slurry, RT, 5 d | Form A + Form B |
| MEK | Slurry, RT, 5 d | Form A |
| — | 100° C., 20 minutes | Form B, |
| | | shifted with minor |
| | | Form A |
| EtOH | CC from 60° C. | Form C + minor Form A |

Samples from batch 289-MBA-16 (containing forms A and B) were added to various solvents, incubated under various conditions, and analyzed by X-ray powder diffraction. Results are summarized in Table 60.

TABLE 60

| Solvent | Conditions | XRPD results |
|---|---|---|
| EtOH | Slurry, RT, 3 d | Form A + Form B |
| MeOH | Vapor diffusion w/ MTBE | Form A |
| EtOAc | Attempted to dissolve at ~60° C., | Form A + Form B |
| | solids remained, cooled slowly | |
| | to RT, let stir at RT from 60° C. | |

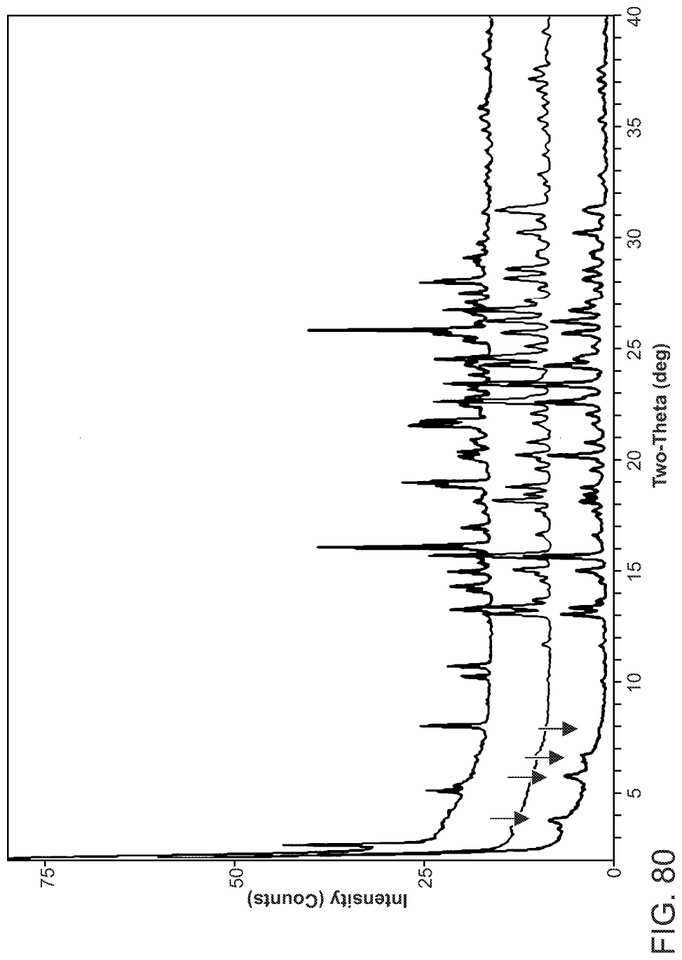

FIG. 80 is a graph showing X-ray powder diffraction analysis of samples of CV-8972. A sample containing form B is shown in blue, a sample containing form A is shown in red, and a sample containing a mixture of forms A and C is shown in black.

The stability of CV-8972 was analyzed. Aqueous samples containing CV-8972 at different concentrations and pH were incubated for various periods and analyzed. Results are shown in Table 61.

| | | | | | | | | Decrease in purity |
|---|---|---|---|---|---|---|---|---|
| | | | | | | | | of CV-8972 |
| | Time | | Retention Time | | | | | between time |
| Sample | (hrs) | pH | 2.2 | 2.6 | 4.2 | 4.7 | 5.6 | points |
| 276-MBA-172 | 0 | 6.6 | 3.39 | 0.6 | 0.23 | 0.54 | 95.24 | |
| 10 mg/mL pH 6 | 1 | 6.8 | 4.81 | 0.81 | 0.23 | 0.73 | 93.43 | 1.81 |
| (Form A) | 4 | 6.8 | 5.72 | 0.9 | 0.21 | 0.83 | 91.82 | 1.61 |
| | 6 | 6.7 | 6.45 | 0.81 | ND | 0.93 | 91.8 | 0.02 |
| | 22 | 6.7 | 7.38 | 1.54 | 0.13 | 1.11 | 89.66 | 2.14 |
| 276-MBA-172 | 0 | 6.1 | ND | ND | 1.29 | ND | 98.01 | |
| 2 mg/mL pH 6 | 1 | 6.1 | 1.5 | ND | 1.28 | ND | 97.22 | 0.79 |
| (Form A) | 4 | 6.1 | 2.03 | ND | 0.95 | ND | 97.01 | 0.21 |
| | 6 | 6.1 | 2.47 | ND | 1.02 | ND | 96.51 | 0.5 |
| | 22 | 6.1 | | | | | | |
| 289-MBA-15-A 10 | 0 | 6 | 3.3 | 0.6 | 0.26 | 0.48 | 95.36 | |
| mg/mL pH 6 (Form | 1 | 6.1 | 3.76 | 0.65 | 0.25 | 0.53 | 94.81 | 0.55 |
| B) | 4 | 6 | 3.97 | 0.59 | 0.19 | 0.56 | 94.69 | 0.12 |
| | 6 | 5.9 | 4.3 | 0.54 | 0.17 | 0.6 | 94.39 | 0.3 |
| | 22 | 5.9 | 4.53 | 0.69 | 0.19 | 0.65 | 93.93 | 0.46 |
| 289-MBA-15-A 2 | 0 | 6.9 | 1.33 | ND | 1.19 | ND | 97.48 | |
| mg/mL pH 6 (Form 1 | 6.9 | 3.73 | ND | 1.17 | ND | 95.1 | 2.38 | |
| B) | 4 | 6.8 | 5.25 | 0.67 | 0.84 | 0.79 | 92.45 | 2.65 |
| | 6 | 6.8 | 6.63 | 0.9 | 0.83 | 0.99 | 90.65 | 1.8 |
| | 22 | 6.7 | 7.72 | 1.13 | 0.86 | 1.14 | 89.15 | 1.5 |
| 276-MBA-172 10 | 0 | 7.1 | 5.9 | 0.94 | 0.22 | 0.78 | 92.85 | |
| mg/mL pH 7 (Form 1 | 7.2 | 8.12 | 1.45 | 0.21 | 1.17 | 89.05 | 3.8 | |
| A) | 4 | 7.1 | 10.14 | 1.48 | 0.13 | 1.46 | 86.8 | 2.25 |
| | 6 | 7.1 | 11.63 | 1.78 | 0.13 | 1.67 | 84.79 | 2.01 |
| | 22 | 7 | | | | | | |
| 276-MBA-172 2 | 0 | 6.7 | 1.42 | ND | 1.05 | ND | 97.53 | |
| mg/mL pH 7 (Form 1 | 6.8 | 3.31 | ND | 1.06 | 0.57 | 95.06 | 2.47 | |
| A) | 4 | 6.7 | 4.21 | 0.58 | 0.82 | 0.69 | 93.7 | 1.36 |
| | 6 | 6.7 | 5.63 | 0.67 | 0.74 | 0.85 | 92.12 | 1.58 |
| | 22 | 6.8 | 6.26 | 0.85 | 0.85 | 0.98 | 91.07 | 1.05 |
| 289-MBA-15-A 10 | 0 | 7.4 | 6.2 | 1.16 | 0.27 | 0.87 | 91.5 | |
| mg/mL pH 7 (Form 1 | 7.4 | 10.47 | 1.65 | 0.25 | 1.44 | 86.18 | 5.32 | |
| B) | 4 | 7.4 | 13.64 | 1.93 | 0.19 | 1.89 | 82.36 | 3.82 |
| | 6 | 7.3 | 15.66 | 2.57 | 0.2 | 0.2 | 79.37 | 2.99 |
| | 22 | 7.1 | | | | | | |
| 289-MBA-15-A 2 | 0 | 6.5 | 1.62 | ND | 0.9 | ND | 97.48 | |
| mg/mL pH 7 (Form 1 | 6.6 | 3.16 | ND | 0.89 | 0.49 | 95.46 | 2.02 | |
| B) | 4 | 6.5 | 4.27 | 0.53 | 0.66 | 0.62 | 93.92 | 1.54 |
| | 6 | 6.5 | | | | | | |
| | 22 | 6.5 | | | | | | |

Samples from batch S-18-0030513 (containing form A) were added to various solvents, incubated under various conditions, and analyzed by X-ray powder diffraction. Results are summarized in Table 62.

TABLE 62

| Solvent | Conditions | XRPD results |
|---|---|---|
| CHCl3 | Slurry, RT | Form A |
| EtOAc | Slurry, RT | Form A |
| THF | Slurry, RT | Form A |
| — | VO, RT | Form A |
| — | 80° C., 20 minutes | Form A with slight peak shifting |
| — | 100° C., 20 minutes | Form B + Form A, shifted |
| — | 97% RH Stress of | Form A |
| | Form A dried at | |
| | 80° C. for 20 min | |
| EtOH | Crash cool from | Form A + Form C |
| | 70° C. | |
| MEK/H2O | Slow cool from | Form A |
| 99:1 | 70° C. | |

Samples from batch 289-MBA-16 (containing forms A and B) were added to various solvents, incubated under various conditions, and analyzed by X-ray powder diffraction. Results are summarized in Table 63.

TABLE 63

| Solvent | Conditions | XRPD results |
|---|---|---|
| EtOH | Slurry, RT, 3 d | Form A + Form B |
| MeOH | VD w/ MTBE | Form A |
| EtOAc | SC from 60° C. | Form A + Form B |
| THF | SC from 60° C. | Form B |
| EtOH | SC from 60° C. | Form A + Form C |
| MeOH/H2O (95:5) | Slurry, overnight, 1 g scale | Form A |

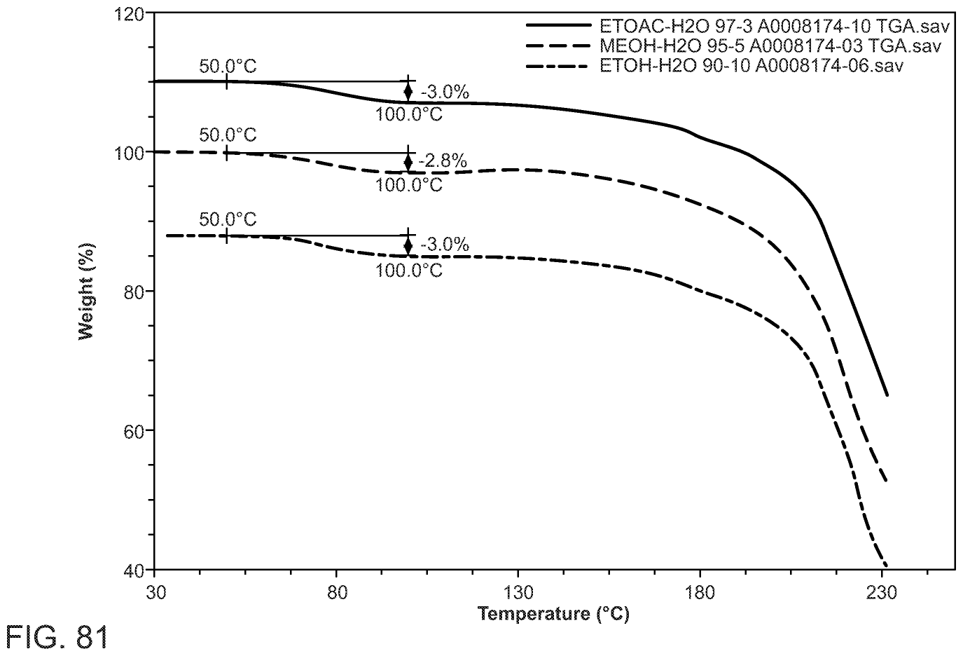

FIG. 81 is a graph showing differential scanning calorimetry and thermal gravimetric analysis of samples containing form A of CV-8972. A sample from an ethanol acetate-water slurry is shown with solid lines, a sample from a methanol-water slurry is shown with regularly-dashed lines, and a sample from an ethanol-water slurry is shown with dashed-dotted lines.

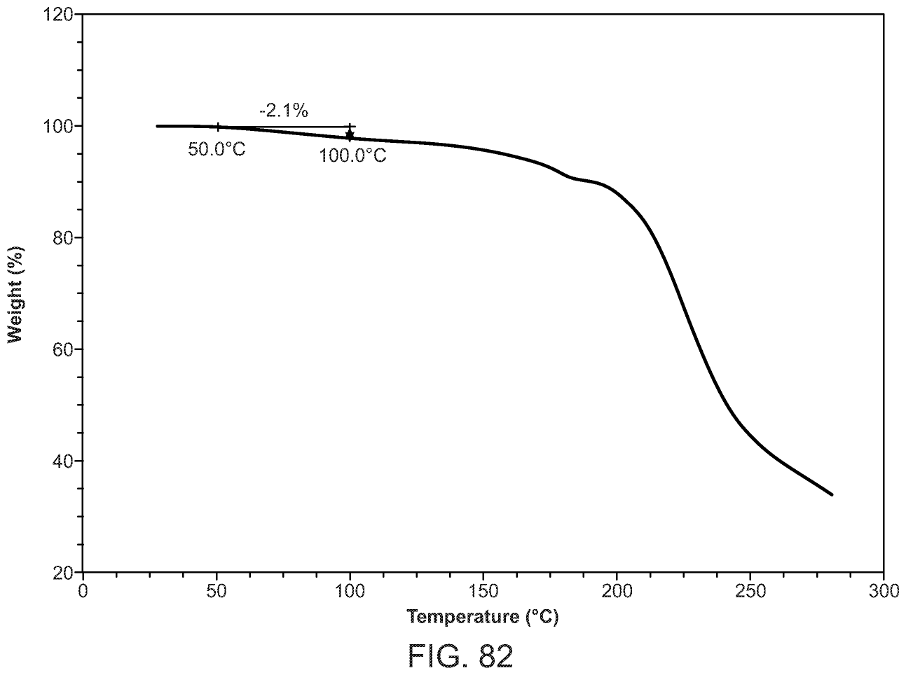

FIG. 82 is a graph showing differential scanning calorimetry and thermal gravimetric analysis of a sample containing form A of CV-8972. Prior to analysis, the sample was dried at 100° C. for 20 minutes.

Samples containing form A of CV-8972 were analyzed for stability in response to humidity. Samples were incubated at 40 ° C., 75% relative humidity for various periods and analyzed. Results are shown in Table 64.

TABLE 64

| | Retention Time | | | |
|---|---|---|---|---|
| Time (days) | 1.9 | 3.9 | 4.5 | 5.4 |
| 0 | ND | 1.16 | ND | 98.84 |
| 1 | ND | 0.68 | ND | 99.32 |
| 7 | 0.63 | 0.14 | 0.12 | 99.12 |

Form A of CV-8972 were analyzed for stability in aqueous solution. Aqueous samples containing CV-8972 at different concentrations and pH were incubated for various periods and analyzed. Results are shown in Table 65.

TABLE 65

| Concentration | | | | | | | % change |
|---|---|---|---|---|---|---|---|
| of | Time | Retention Time | | | | | from t0 of |
| CV-8972 | (hrs) | 1.9 | 2.2 | 3.9 | 4.5 | 5.4 | RT 5.4 |
| 21 mg/mL, | 0 | ND | ND | 1.12 | ND | 98.88 | — |
| Initial | 1 | 1.03 | ND | 0.94 | ND | 98.03 | −0.86 |
| pH = 2.0 | 2 | 1.9 | ND | 1 | ND | 97.11 | −1.79 |
| | 6 | 5.25 | 0.83 | 0.96 | 0.78 | 92.18 | −6.78 |
| 12.5 mg/mL, | 0 | ND | ND | 1.79 | ND | 98.21 | — |
| Initial | 1 | 1.38 | ND | 1.41 | ND | 97.21 | −1.02 |
| pH = 2.1 | 2 | 2.43 | ND | 1.67 | ND | 95.9 | −2.35 |
| | 6 | 6.59 | 1.04 | 1.74 | 1.04 | 89.58 | −8.79 |
| 4.2 mg/mL, | 0 | ND | ND | 5.35 | ND | 94.65 | — |
| Initial | 1 | ND | ND | 4.02 | ND | 95.98 | 1.41 |
| pH = 2.3 | 2 | 3.72 | ND | 5.09 | ND | 91.19 | −3.66 |
| | 6 | 9.71 | ND | 5.3 | ND | 84.99 | −10.21 |

The amount of CV-8972 present in various dosing compositions was analyzed. Results are shown in Table 66.

TABLE 66

| | | | Initial | | Total vol. | | | |
|---|---|---|---|---|---|---|---|---|
| Target | Vol. API | Mass | pH | Vol. 1N | base | pH after | Vol. addl. | |
| Dose | soln. | CV8972 | API | NaOH | soln. | base soln. | 1N NaOH | Final Dose |
| (mg/mL) | (mL) | (mg) | soln. | (mL) | (mL) | addn. | added (mL) | (mg/mL) |
| 10 | 30 | 779.06 | 2.0 | 2.07 | 30 | 3.6 | 0.7 | 9.92 |
| 2 | 30 | 157.38 | 2.4 | 0.19 | 30 | 2.8 | 0.35 | 2.02 |
| 10 | 50 | 777.05 | 2.1 | 2.77 | 10 | 6.2 | — | 10.01 |
| 2 | 50 | 142.08 | 2.5 | 0.99 | 10 | 3.0 | 0.3 | 1.82 |

Brain-to-Plasma Ratio of Compounds In Vivo

The brain-to-plasma ratio of trimetazidine and CV-8814 was analyzed after intravenous administration of the compounds to rats. Dosing solutions were analyzed by liquid chromatography tandem mass spectrometry (LC-MS/MS). Results are shown in Table 67.

TABLE 67

| | | | | Measured | |
|---|---|---|---|---|---|
| | | | Nominal | Dosing | |
| | Route of | | Dosing | Solution | |
| Test | Admin- | | Conc. | Conc. | % of |
| Article | istration | Vehicle | (mg/mL) | (mg/mL) | Nominal |
| TMZ | IV | Normal | 1.0 | 1.14 | 114 |
| | | Saline* | | | |
| CV- | IV | Normal | 0.585 | 0.668 | 114 |
| 8814 | | Saline* | | | |

The concentrations of compounds in the brain and plasma were analyzed 2 hours after administering compounds at 1 mg/kg to rats. Results from trimetazidine-treated rats are shown in Table 68. Results from CV-8814-treated rats are shown in Table 69.

TABLE 68
TMZ-treated rats

| Rat# | 11 | 12 | 13 |
|---|---|---|---|
| Brain Weight (g) | 1.781 | 1.775 | 1.883 |
| Brain Homogenate Volume (mL) | 8.91 | 8.88 | 9.42 |
| Brain Homogenate Conc. | 7.08 | 7.35 | 7.90 |
| (ng/mL) | | | |
| Brain Tissue Conc. (ng/g) | 35.4 | 36.8 | 39.5 |
| Plasma Conc. (ng/g)¹ | 22.7 | 14.0 | 14.1 |
| B:P Ratio | 1.56 | 2.63 | 2.80 |

TABLE 69
CV-8814-treated rats

| Rat# | 14 | 15 | 16 |
|---|---|---|---|
| Brain Weight (g) | 1.857 | 1.902 | 2.026 |
| Brain Homogenate Volume (mL) | 9.29 | 9.51 | 10.1 |
| Brain Homogenate Conc. | 4.01 | 4.21 | 4.74 |
| (ng/mL) | | | |
| Brain Tissue Conc. (ng/g) | 20.1 | 21.1 | 24 |
| Plasma Conc. (ng/g)¹ | 19.3 | 17.0 | 14.0 |
| B:P Ratio | 1.04 | 1.24 | 1.693 |

The average B:P ratio for trimetazidine-treated rats was 2.33±0.672. The average B:P ratio for trimetazidine-treated rats was 1.32±0.335.

### Example 2

Compounds were tested for the ability to protect the heart against ventricular remodeling.

Experimental Methods

One hundred-seven mice were divided into six groups: (1) sham, (2) TAC treated with saline vehicle, (3) TAC treated with trimetazidine (TMZ), (4) TAC treated with nicotinic acid (NA), (5) TAC treated with CV-8814, and (6) TAC treated with CV-8972. Each mouse was labeled with one specific ear tag. Mice were subjected to sham or TAC surgery. After echocardiography evaluation at 24 hr post-surgery, if TAC mice had low cardiac function (FS<10%) or high cardiac function (FS>50%) with no increase in left ventricular wall thickness (<1.0 mm), they were excluded from the study. The remaining TAC mice were treated with saline as a control, TMZ (6 mg/kg/day), NA (2.4 mg/kg/day), CV-8814 (7.5 mg/kg/day), CV-8972 (10 mg/kg/day) for six weeks through a subcutaneous osmotic minipump (Alzet Model 2006). Left ventricular remodeling and functional changes were measured and recorded at 3-weeks and 6-weeks post-surgery. Fourteen TAC mice died during the study, and 93 mice survived to the end of the 6-week experiment. After week-6 echocardiography, all mice were euthanized. Mouse body weights and the heart weights were recorded, and heart weight/body weight ratios were calculated. The residual volume in each mini-pump was measured to verify drug delivery. The hearts were fixed with 10% formalin, sectioned and stained with Masson's trichrome for analysis of cardiac fibrosis.

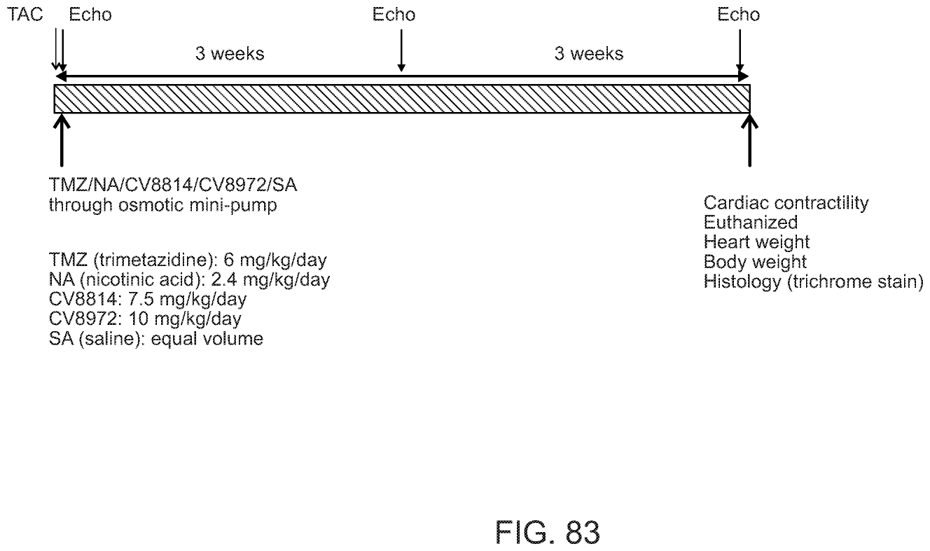

FIG. 83 is a schematic of the method used to analyze the effects of selected compositions on cardiac function. Following transverse aortic constriction (TAC) or a sham procedure, mice were given one of the following via an osmotic mini-pump: trimetazidine (TMZ) at 6 mg/kg/day; nicotinic acid (NA) at 2.4 mg/kg/day; CV-8814 at 7.5 mg/kg/day; CV-8972 at 10 mg/kg/day; or saline (SA). Echocardiograms were measured 24 hours following TAC, three weeks after TAC, and 6 weeks after TAC. Mice were sacrificed at 6 weeks, and tissues were analyzed.

Animals

Male C57BL6 mice were purchased from The Jackson Laboratory. Mice were housed in groups of four to five per cage in a room maintained at 23±1° C. and 55±5% humidity with a 12-h light/dark cycle and were given ad libitum access to food and water. At the beginning of experiments, mice were 11-12 weeks old.

Preparation of TAC Mice

Mice were anesthetized with ketamine (100 mg/kg) and xylazine (10 mg/kg). Endotracheal intubation was performed. The endotracheal tube was connected to a small animal ventilator at 100 breaths/min and a tidal volume of 0.2 ml. Animals were placed in the supine position. A midline incision was made, and the chest cavity was entered at the second intercostal space to expose the aortic arch. A 27 gauge blunt needle was tied against the transverse aorta; then the needle was promptly removed. The wound was closed in two layers.

Echocardiography

In vivo cardiac function was assessed by transthoracic echocardiography (Acuson P300, 18 MHz transducer; Siemens) in conscious mice, as described previously (Reference 2). From the left ventricle short axis view, M-mode echocardiogram was acquired to measure interventricular septal thickness at end diastole (IVSd), left ventricular posterior wall thickness at end diastole (LVPWd), left ventricular end diastolic diameter (LVEDD), and left ventricular end systolic diameter (LVESD). Fractional shortening (FS) and ejection fraction (EF) were calculated through LVEDD and LVESD [FS=(LVEDD−LVESD)/LVEDD %, EF=(LVEDD2−LVESD2)/LVEDD2%]. Left ventricular mass (LVM) was assessed by the equation: 1.05 [(LVEDD+LVPTD+IVSd)3−LVEDD3]. Early diastolic filling peak velocity (E), late filling peak velocity (A), and isovolumetric relaxation time (IVRT) were measured from the medial or septal wall at the mitral valve level from tissue Doppler images. LV diastolic function was assessed by measuring the E/A ratio and IVRT. Three to five beats were averaged for each mouse. Studies and analyses were performed by investigators blinded to treatments.

Measurement of Cardiac Fibrosis

Hearts were fixed with 10% buffered formalin, embedded in paraffin, and sectioned at 6 μm, as described previously (Reference 3). One middle section per heart was stained with Masson's trichrome. Fibrotic blue and whole heart tissue areas were measured using computerized planimetry (Image J, NIH). The fibrotic area was presented as a percentage of the fibrotic area to the whole heart tissue area. Five random fields per heart were counted and averaged. Thus, a total 65-75 fields per treatment group were measured. The observer was blinded to the origin of the cardiac sections.

Statistical Analysis

Data are presented as the mean±standard error of the mean. The difference between two groups was analyzed by using Student's t test. Differences were considered significant if p<0.05.

Survival in Treated TAC Mice

Ninety-three mice survived to the end of the experiment. Fourteen TAC mice died during the six week experiment [total mortality of 13% (14/107)]. There were 2-4 mice that died in each of the TAC groups.

CV8814 and CV8972 Inhibited Cardiac Remodeling in TAC Mice

TAC mice were treated with TMZ, NA, CV-8814, or CV-8972 for 6 weeks. TMZ, CV-8814 and CV-8972 significantly reduced the heart weight to body weight ratio in TAC mice, compared with TAC control (TAC+TMZ/TAC+CV-8814/TAC+CV-8972 vs. TAC=7.6±0.4/7.6±0.4/7.4±0.3 mg/g vs. 9.1±0.5 mg/g; p<0.05 in TAC+TMZ vs. TAC; p<0.05 in TAC+CV-8814 vs. TAC; p<0.05 in TAC+CV-8972 vs. TAC), suggesting that TMZ, CV-8814 and CV-8972 inhibited cardiac remodeling induced by left ventricular overload. Furthermore, TMZ, CV-8814 and CV-8972 prevented an increase in cardiac mass (TAC+TMZ/TAC+CV8814/TAC+CV8972 vs. TAC=224±12/227±12/225±11 mg vs. 270±14 mg; p<0.05 in TAC+TMZ vs. TAC; p<0.05 in TAC+CV8814 vs. TAC; p<0.05 in TAC+CV8972 vs. TAC). These results suggest that TMZ, CV-8814 and CV-8972 were effective in blocking cardiac hypertrophy. In contrast, NA had no significant effect on cardiac remodeling in TAC mice.

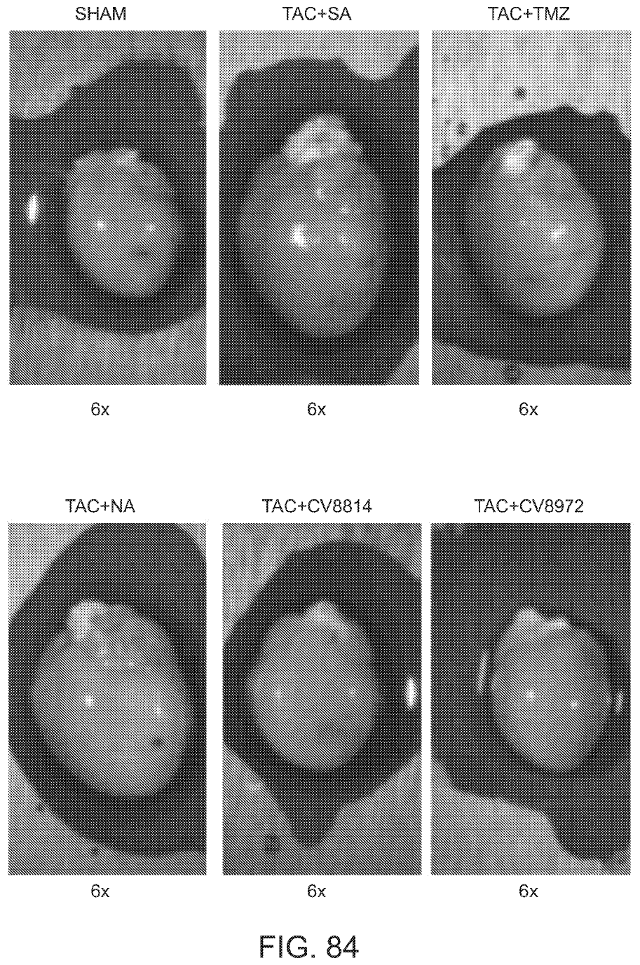

FIG. 84 shows hearts from mice six weeks after a sham procedure (SHAM), TAC followed by saline administration (TAC+SA), TAC followed by trimetazidine administration (TMZ), TAC followed by nicotinic acid administration (TAC+NA), TAC followed by CV-8814 administration (TAC+CV8814), or TAC followed by CV-8972 administration (TAC+CV8972).

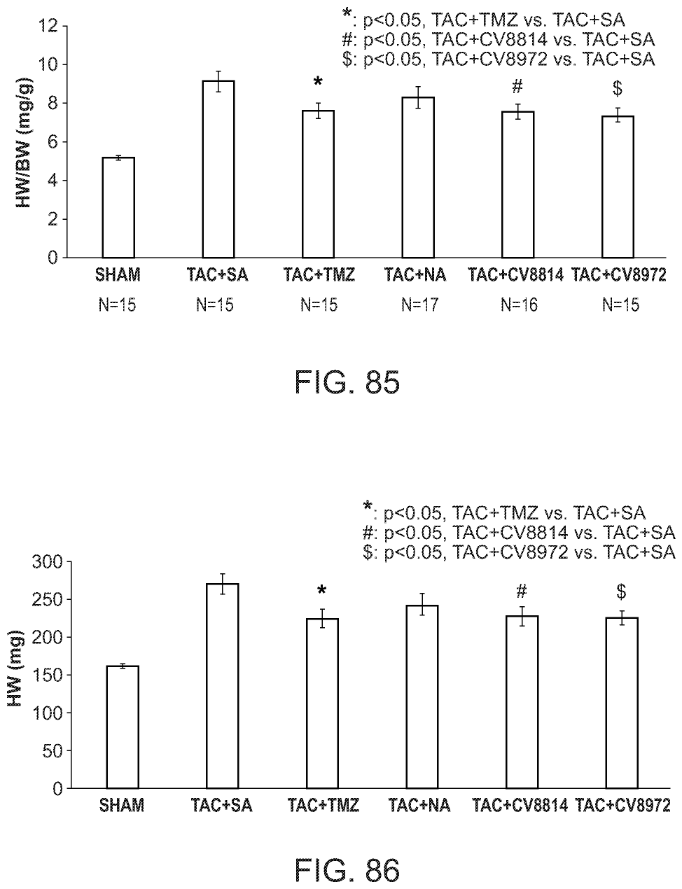

FIG. 85 is of graph of heart weight relative to body weight six weeks after transverse aortic constriction. Treatments are as indicated in relation to FIG. 84.

FIG. 86 is graph of heart weight six weeks after transverse aortic constriction. Treatments are as indicated in relation to FIG. 84.

CV8814 and CV8972 Improved Left Ventricular Contractility in TAC Mice

Left ventricular pressure in TAC mice was directly measured with a Mikro-tip catheter. TMZ, CV-8814, and CV-8972 decreased left ventricular developed pressure (LVDP) at the end of the 6-week treatment period compared with the control TAC group (TAC+TMZ/TAC+CV8814/TAC+CV8972 vs. TAC=137±10/123±8/116±9 mm Hg vs. 173±8 mm Hg; p<0.01 in TAC+TMZ vs. TAC; p<0.01 in TAC+CV-8814 vs. TAC; p<0.01 in TAC+CV-8972 vs. TAC). However, no improvement was observed in the NA group (TAC+NA vs. TAC=141±20 vs. 173±8 mm Hg; p>0.05). Moreover, TMZ, CV-8814, and CV-8972 decreased+dp/dtm (TAC+TMZ/TAC+CV-8814/TAC+CV-8972 vs. TAC=5157±615/4572±268/4541±395 mm Hg/sec vs. 5798±362 mm Hg/sec; p<0.05 in TAC+TMZ vs. TAC; p<0.01 in TAC+CV-8814 vs. TAC; p<0.01 in TAC+CV-8972 vs. TAC). These data suggest that TMZ, CV-8814, and CV-8972 treatment all improved left ventricular function in TAC mice. Furthermore, -dp/dtm was improved by CV-8972 (TAC+CV-8972/TAC=−4126±339 mm Hg/sec vs. −5697±417 mm Hg/sec, p<0.01), suggesting that CV-8972 also improved left ventricular relaxation in TAC mice.

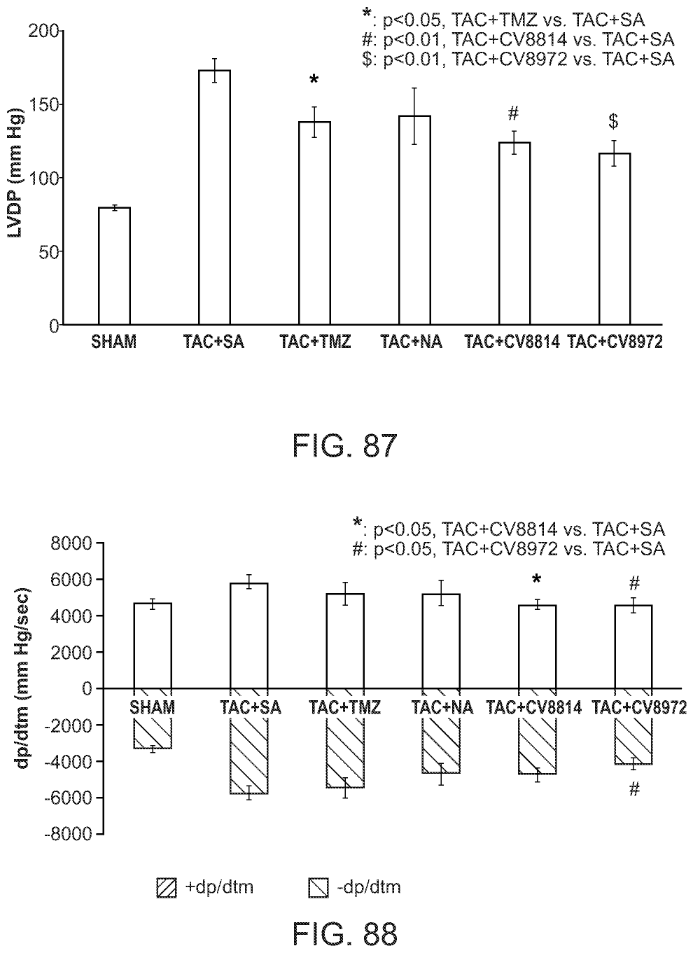

FIG. 87 is a graph of left ventricular developed pressure at six weeks after transverse aortic constriction. Treatments are as indicated in relation to FIG. 84.

FIG. 88 is a graph of the rate of left ventricle pressure rise at six weeks after transverse aortic constriction. Treatments are as indicated in relation to FIG. 84.

CV8814 and CV8972 Improved Cardiac Function in TAC Mice

Echocardiography was performed on all mice at 24-hour, 3-week, and 6-week time-points after TAC. Compared with the control TAC group, CV-8814 and CV-8972 significantly increased left ventricular FS (TAC+CV-8814/TAC+CV-8972 vs. TAC=46%±2%/47%±3% vs. 37%±3%; p<0.05 in TAC+CV-8814 vs. TAC; p<0.05 in TAC+CV-8972 vs. TAC) at 3-weeks after TAC. From 3-weeks to 6-weeks, FS declined further in the TAC group. However, the effect of CV-8814 and CV-8972 was sustained to the end of the experiment (TAC+CV-8814/TAC+CV-8972 vs. TAC=44%±3%/46%±3% vs. 34%±3%; p<0.05 in TAC+CV-8814 vs. TAC; p<0.05 in TAC+CV-8972 vs. TAC). Like FS, EF was significantly increased in CV-8814 and CV-8972-treated groups at 3-weeks after TAC, and the protective effect was sustained to the end of the study in both CV-8814 and CV-8972 treated groups.

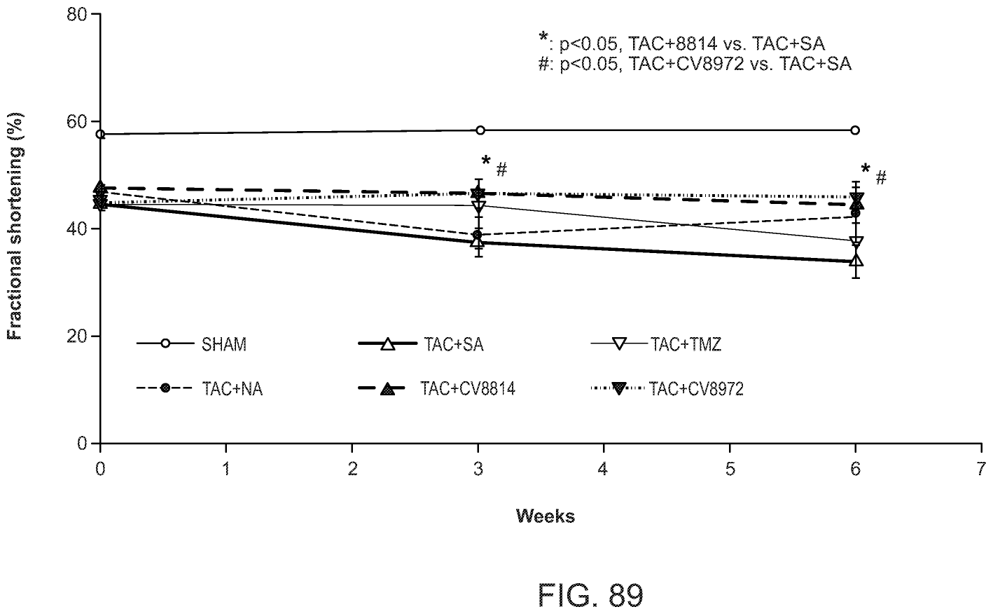

FIG. 89 is a graph of fractional shortening (FS) at indicated time points after transverse aortic constriction. Treatments are as indicated in relation to FIG. 84.

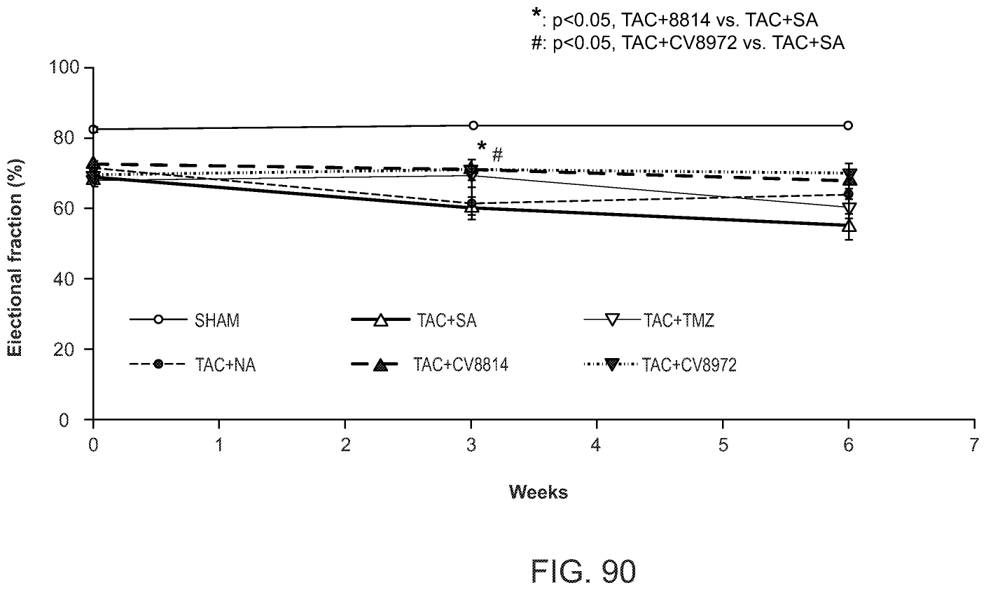

FIG. 90 is a graph of ejection fraction (EF) at indicated time points after transverse aortic constriction. Treatments are as indicated in relation to FIG. 84.

To examine the effect of TMZ, NA, CV-8814, and CV-8972 on left ventricular remodeling, interventricular septal dimension (IVSd) and left ventricular mass (LVM) were measured. CV-8814 and CV-8972 significantly decreased IVSd at 3-weeks (TAC+CV-8814/TAC+CV-8972 vs. TAC=1.27±0.02/1.29±0.04 mm vs. 1.37±0.02 mm; p<0.05 in TAC+CV-8814 vs. TAC; p<0.05 in TAC+CV-8972 vs. TAC) and at 6-weeks (TAC+CV-8814/TAC+CV-8972 vs. TAC=1.26±0.04/1.26±0.04 mm vs. 1.35±0.02 mm; p<0.05 in TAC+CV-8814 vs. TAC; p<0.05 in TAC+CV-8972 vs. TAC). Consistent with IVSd, LV mass in the CV-8814 and CV-8972 groups was also significantly decreased at 3-weeks (TAC+CV-8814/TAC+CV-8972 vs. TAC=152±9/154±8 mg vs. 178±8 mg; p<0.05 in TAC+CV-8814 vs. TAC; p<0.05 in TAC+CV-8972 vs. TAC). CV-8972 also significantly decreased LV mass at 6-weeks (TAC+CV-8972 vs. TAC=156±10 mg vs. 195±12 mg; p<0.05). TMZ decreased LV mass at 3-weeks (TAC+TMZ vs. TAC=153±8 mg vs. 178±8 mg; p<0.05), but its effect was not significant at 6-weeks (TAC+TMZ vs. TAC=176±15 mg vs. 195±12 mg; p>0.05. Although NA decreased IVSd in TAC mice, it had no effect on LV mass. These results suggest that TMZ, CV-8814, and CV-8972 treatment inhibited cardiac remodeling in TAC mice. The effects of CV-8972 were sustained through the 6-week treatment period and its activity appears more robust than CV-8814 or TMZ.

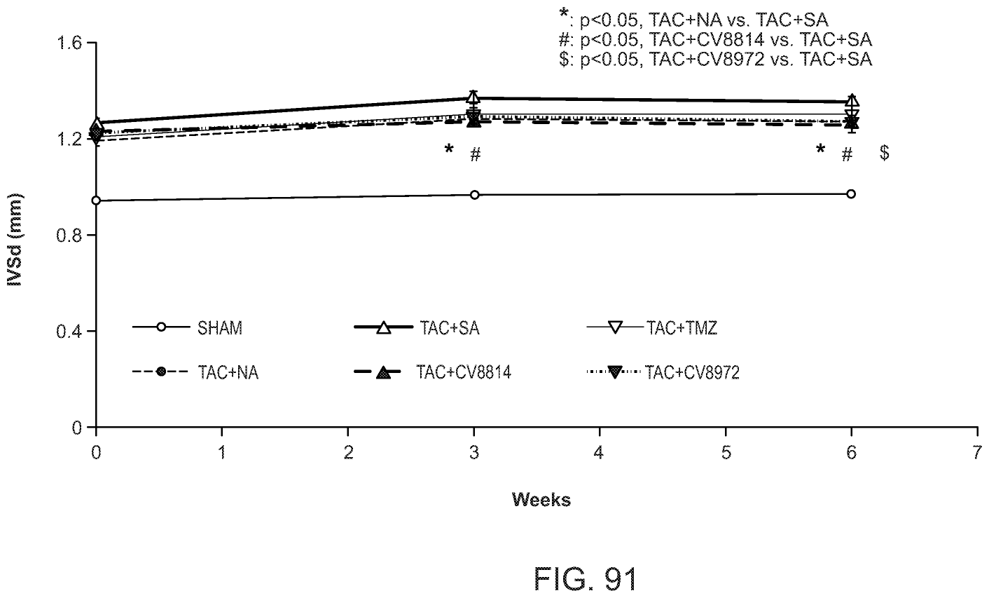

FIG. 91 is a graph of intraventricular septal dimension at indicated time points after transverse aortic constriction. Treatments are as indicated in relation to FIG. 84.

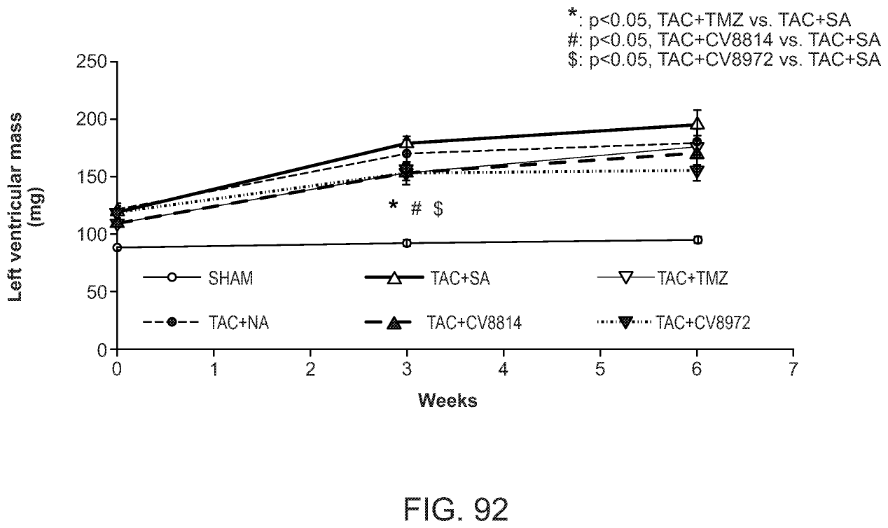

FIG. 92 is a graph of left ventricular mass at indicated time points after transverse aortic constriction. Treatments are as indicated in relation to FIG. 84.

To assess the effect of TMZ, NA, CV-8814, and CV-8972 on diastolic function, IVRT was measured in TAC mice. Consistent with the FS and EF data, CV-8814 and CV-8972 inhibited prolongation of IVRT at 3-weeks (TAC+CV-8814/TAC+CV-8972 vs. TAC=33±1/32±1 ms vs. 36±1 ms; p<0.05 in TAC+CV-8814 vs. TAC; p<0.05 in TAC+CV-8972 vs. TAC). At 6-weeks, the CV-8814 effect was sustained (TAC+CV8814 vs. TAC=28±2 ms vs. 35±1 ms, p<0.01). CV-8972 also decreased IVRT (TAC+CV8972 vs. TAC=31±2 ms vs. 35±1 ms, p=0.06). NA was shown to shorten IVRT (TAC+NA vs. TAC=29±2 ms vs. 35±1 ms, p<0.05). TMZ had no effect on IVRT (TAC+TMZ vs. TAC=36±1 ms vs. 35±1 ms, p>0.05). These results suggest that CV-8814, CV-8972 and NA inhibited diastolic dysfunction in TAC mice.

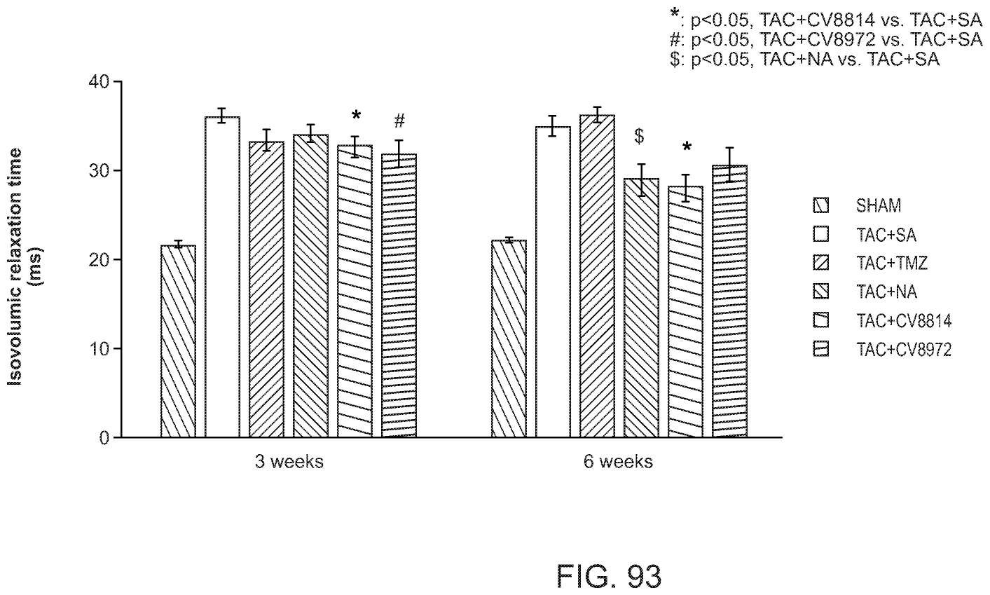

FIG. 93 is a graph of isovolumic relaxation time at indicated time points after transverse aortic constriction. Treatments are as indicated in relation to FIG. 84.

CV8814 and CV8972 Inhibited Cardiac Fibrosis in TAC Mice

Hearts were collected at the end of the experiment and cross-sectioned for Masson's trichrome staining. Both CV-8814 and CV-8972 significantly suppressed cardiac fibrosis in TAC mice (TAC+CV-8814/TAC+CV-8972 vs. TAC=6.6±0.0.6%/6.6±0.6% vs. 10.7±1%; p<0.01). Neither TMZ nor NA had a statistically significant on cardiac fibrosis (TAC+TMZ/TAC+NA vs. TAC=7.6±1%/8.2±1% vs. 10.7±1%; p=0.08 in TAC+TMZ vs. TAC; P>0.05 in TAC+NA vs. TAC). These results provide additional evidence that CV-8814 and CV-8972 inhibited ventricular remodeling.

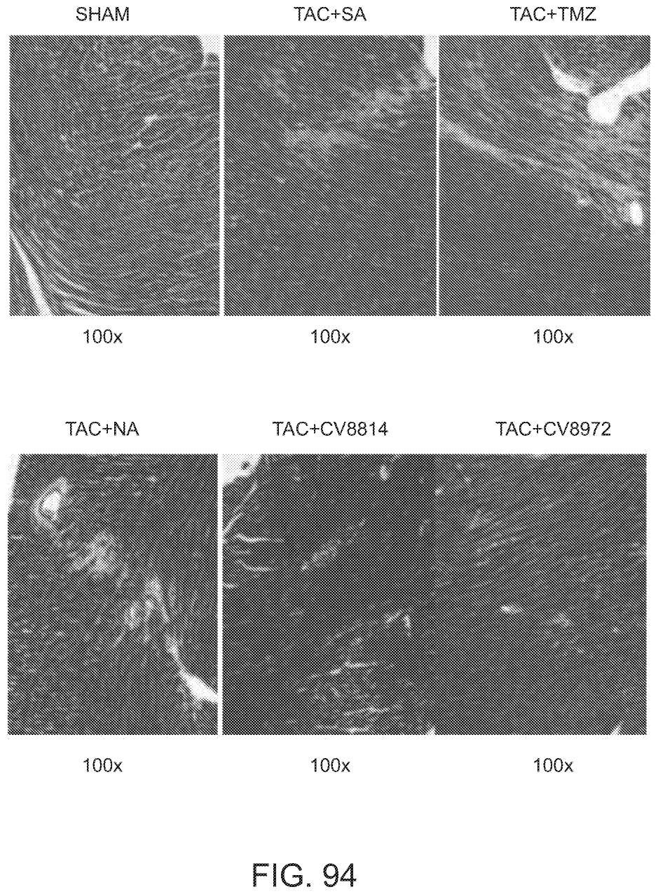

FIG. 94 shows microscopic images of cardiac tissue t six weeks after transverse aortic constriction. Upper left panel, sham TAC procedure; upper middle panel, TAC followed by saline; upper right panel, TAC followed by trimetazidine administration; lower left panel, TAC followed by nicotinic acid administration; lower middle panel, TAC followed by CV-8814 administration; and lower right panel, TAC followed by CV-8972 administration.

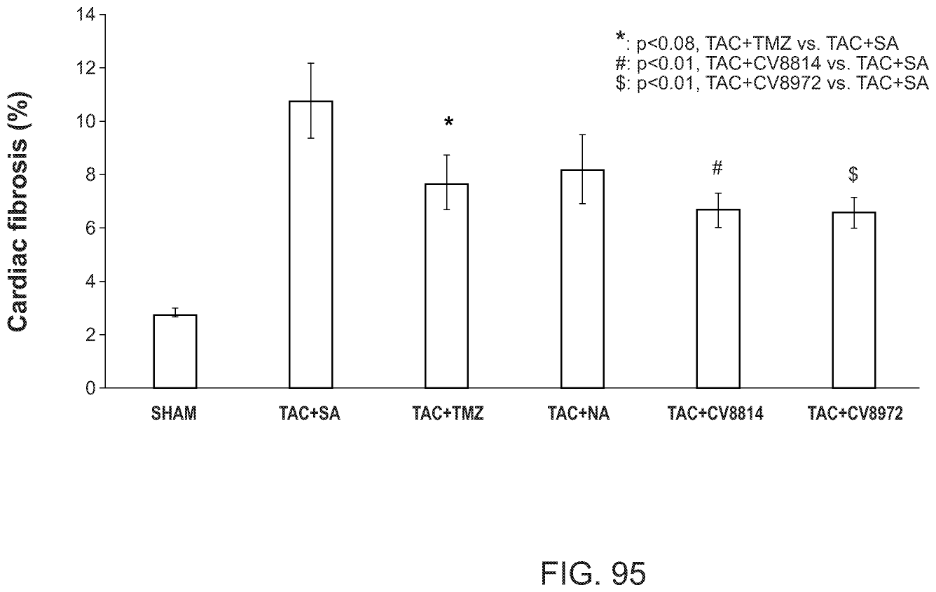

FIG. 95 is a graph showing the level of cardiac fibrosis at six weeks after transverse aortic constriction. Treatments are as indicated in relation to FIG. 84.

Conclusions

Taken together, TMZ, CV-8814 and CV-8972 effectively inhibited cardiac remodeling in TAC mice, and CV-8814 and CV-8972 improved cardiac functions. NA had no effect on cardiac remodeling in TAC mice.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A method of altering cardiac remodeling in a subject, the method comprising providing to a subject that has developed cardiac remodeling or is at risk of developing cardiac remodeling a compound that shifts cellular metabolism from fatty acid oxidation to glucose oxidation.

2. The method of claim 1, wherein the compound that shifts cellular metabolism from fatty acid oxidation is selected from the group consisting of trimetazidine, etomoxir, perhexiline, a PPAR agonist, a malonyl CoA decarboxylase inhibitor, and analogs, derivatives, and prodrugs thereof.

3. The method of claim 2, wherein the compound that shifts cellular metabolism from fatty acid oxidation is trimetazidine or an analog, derivative, or prodrug thereof.

4. The method of claim 1, wherein the compound that shifts cellular metabolism from fatty acid oxidation is represented by formula (IV): 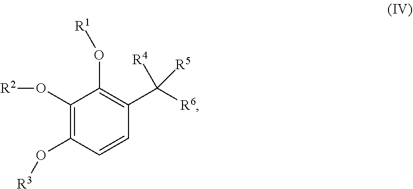
wherein:
R¹, R², and R³ are independently selected from the group consisting of H and a (C₁-C₄)alkyl group;
R⁴ and R⁵ together are ═O, —O(CH₂)ₘO—, or —(CH₂)ₘ—, wherein m=2-4, or R⁴ is H and R⁵ is OR¹⁴, SR¹⁴, or (CH₂CH₂O)ₙH, wherein R¹⁴ is H or a (C₁-C₄)alkyl group and n=1-15; and
R⁶ is a single or multi-ring structure optionally substituted at one or more ring positions by a heteroatom, wherein each ring position optionally comprises one or more substituents.

5. The method of claim 4, wherein:
R⁶ comprises at least one substituent;
the at least one substituent comprises (CH₂CH₂O)ₓ; and
x=1-15.

6. The method of claim 5, wherein the compound that shifts cellular metabolism from fatty acid oxidation is represented by formula (IX):

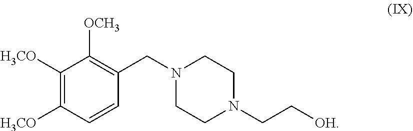 [SMILES: COc1ccc(CN2CCN(CCO)CC2)c(OC)c1OC]

7. The method of claim 4, wherein:
R⁶ comprises at least one substituent; and
the at least one sub stituent comprises a NAD⁺ precursor molecule.

8. The method of claim 7, wherein the NAD⁺ precursor molecule is selected from the group consisting of nicotinic acid, nicotinamide, and nicotinamide riboside.

9. The method of claim 8, wherein the NAD⁺ precursor molecule is nicotinic acid.

10. The method of claim 9, wherein the compound that shifts cellular metabolism from fatty acid oxidation is represented by formula (X):

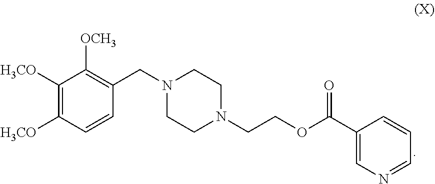 [SMILES: COc1ccc(CN2CCN(CCOC(=O)c3cccnc3)CC2)c(OC)c1OC]

11. The method of claim 1, wherein the compound that shifts cellular metabolism from fatty acid oxidation is represented by formula (VII):
A-C (VII),
wherein:
A comprises a molecule that shifts cellular metabolism from fatty acid oxidation to glucose oxidation; and
C is a NAD⁺ precursor molecule.

12. The method of claim 11, wherein C is covalently linked to A.

13. The method of claim 12, wherein A is PEGylated with an ethylene glycol moiety.

14. The method of claim 13, wherein the ethylene glycol moiety comprises (CH₂CH₂O)ₓ, wherein x=1-15.

15. The method of claim 14, wherein the covalent linkage is via the ethylene glycol moiety.

16. The method of claim 14, wherein the covalent linkage is not via the ethylene glycol moiety.

17. The method of claim 11, wherein A is selected from the group consisting of trimetazidine, etomoxir, perhexiline, a PPAR agonist, a malonyl CoA decarboxylase inhibitor, and dichloroacetate.

18. The method of claim 11, wherein C is selected from the group consisting of nicotinic acid, nicotinamide, and nicotinamide riboside.

19. The method of claim 18, wherein C is nicotinic acid.

20. The method of claim 11, wherein A is a PEGylated form of trimetazidine.

21-36. (canceled)
